SEQUENCE LISTING (13 sequences)

SEQ ID NO: 1 - RNA, 21 nt, synthetic construct
  source 1..21: mol_type = other RNA
gatgatgtttgaaactatttt

SEQ ID NO: 2 - RNA, 21 nt, synthetic construct
  source 1..21: mol_type = other RNA
aatagtttcaaacatcatctt

SEQ ID NO: 3 - RNA, 21 nt, synthetic construct
  source 1..21: mol_type = other RNA
gctactcgttaattatcaatt

SEQ ID NO: 4 - RNA, 21 nt, synthetic construct
  source 1..21: mol_type = other RNA
ttgataattaacgagtagctt

SEQ ID NO: 5 - RNA, 21 nt, synthetic construct
  source 1..21: mol_type = other RNA
ccaaattccattatgacaatt

SEQ ID NO: 6 - RNA, 21 nt, synthetic construct
  source 1..21: mol_type = other RNA
ttgtcataatggaatttggtt

SEQ ID NO: 7 - RNA, 15 nt, synthetic construct
  source 1..15: mol_type = other RNA
cagtaaagagattaa

SEQ ID NO: 8 - RNA, 20 nt, synthetic construct
  source 1..20: mol_type = other RNA
ttaatctctttactgatata

SEQ ID NO: 9 - RNA, 19 nt, synthetic construct
  source 1..19: mol_type = other RNA
acctgatcattatagataa

SEQ ID NO: 10 - RNA, 21 nt, synthetic construct
  source 1..21: mol_type = other RNA
ttatctataatgatcaggttt

SEQ ID NO: 11 - RNA, 22 nt, synthetic construct
  source 1..22: mol_type = other RNA
ggtttgtaggatataggatttt

SEQ ID NO: 12 - RNA, 21 nt, synthetic construct
  source 1..21: mol_type = other RNA
aatcctatatcctacaacctt

SEQ ID NO: 13 - DNA, 20 bp, synthetic construct
  source 1..20: mol_type = other DNA
ctgctagcctctggatttga

(19) United States
(12) Patent Application Publication
(10) Pub. No.: US 2025/0376487 A1
(43) Pub. Date: Dec. 11, 2025

(54) LIPID-MODIFIED NUCLEIC ACID COMPOUNDS AND METHODS

(71) Applicant: Novartis AG, Basel (CH)

(72) Inventors: Arthur Suckow, San Diego, CA (US); Fabio Tucci, San Diego, CA (US)

(21) Appl. No.: 19/220,791

(22) Filed: May 28, 2025

Related U.S. Application Data
(63) Continuation of application No. 17/058,562, filed on Nov. 24, 2020, now abandoned.
(60) Provisional application No. 62/678,013, filed on May 30, 2018.
(60) Provisional application No. 62/793,597, filed on Jan. 17, 2019.

(51) Int. Cl. C07H 21/00; A61K 31/713; A61K 47/54

(52) U.S. Cl. CPC C07H 21/00; A61K 31/713; A61K 47/543

(57) ABSTRACT

Disclosed herein, inter alia, are lipid-modified nucleic acid compounds, their preparation, and their use.

### CROSS-REFERENCES TO RELATED APPLICATIONS

This application is a continuation of prior application Ser. No. 17/058,562, which is a 35 U.S.C. 371 U.S. National Phase Application of International Application Serial No. PCT/US2019/034724 filed May 30, 2019, which claims the benefit of priority to U.S. Provisional Patent Application No. 62/678,013 filed May 30, 2018, and U.S. Provisional Patent Application No. 62/793,597 filed Jan. 17, 2019, the disclosures of which are incorporated herein by reference in there entirety and for all purposes

### SEQUENCE LISTING

The instant application contains Sequence Listings which have been submitted electronically in XML format and are hereby incorporated by reference in their entirety. Said XML copy, created on May 27, 2025, is named PAT059568_US_PCT Sequence listing.xml and is 12000 bytes in size.

### BACKGROUND

### Field

The present disclosure relates to the field of biologically active nucleic acid compounds. More specifically, the present disclosure relates to lipid-modified nucleic acid compounds, their preparation, and their use.

### Background

Delivering therapeutic nucleic acids into cells remains a challenging area of research. Thus, there is a need for improved nucleic acid compounds and strategies of introducing such compounds into cells.

### BRIEF SUMMARY

Provided herein, inter alia, are compounds, or lipid-modified nucleic acid compounds, having the following structure:

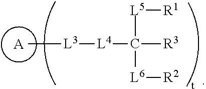

A is an oligonucleotide, a nucleic acid, a polynucleotide, a nucleotide or analog thereof or a nucleoside or analog thereof. In embodiments, A is an oligonucleotide. In embodiments, A is a nucleic acid. In embodiments, A is a polynucleotide. In embodiments, A is a nucleotide or analog thereof. In embodiments, A is a nucleoside or analog thereof.

L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁵ is -L^{5A}-L^{5B}-L^{5C}-L^{5D}-L^{5E}- and L⁶ is -L^{6A}-L^{6B}-L^{6C}-L^{6D}-L^{6E}-. L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl; and R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

t is an integer from 1 to 5.

In embodiments, provided herein is a lipid-conjugated compound having the structure of Formula I:

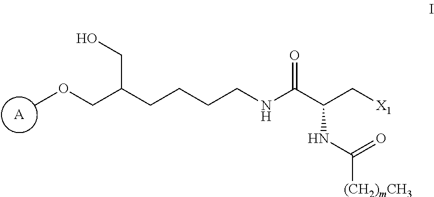

or a pharmaceutically acceptable salt thereof, wherein: A, X₁ and m have any of the values described herein.

In embodiments, provided herein is a lipid-conjugated compound having the structure of Formula II:

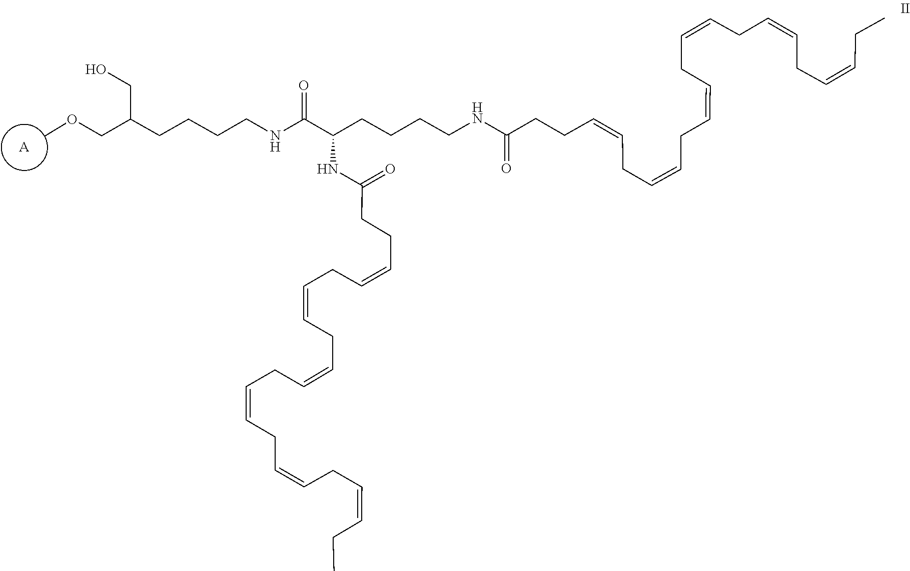

or a pharmaceutically acceptable salt thereof, wherein A has any of the values described herein.

In embodiments, provided herein is a lipid-conjugated compound having the structure of Formula III:

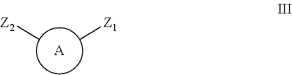

or a pharmaceutically acceptable salt thereof, wherein: A, Z₁ and Z₂ have any of the values described herein.

In embodiments, provided herein is a cell containing a compound as disclosed and described herein.

In embodiments, provided herein is a method of introducing a modified double-stranded oligonucleotide into a cell in vitro, comprising contacting the cell with a compound as disclosed and described herein under free uptake conditions.

In embodiments, provided herein is a method of introducing a modified double-stranded oligonucleotide ex vivo, comprising contacting the cells with a compound as disclosed and described herein under free uptake conditions.

### BRIEF DESCRIPTION OF THE DRAWINGS

FIG. 1 illustrates the structures of DHA-conjugated siRNAs synthesized.

FIG. 2 illustrates the structures of DTx-01-08-conjugated siRNAs synthesized.

FIG. 3 illustrates the structures of PTEN siRNA synthesized with C10 to C22 saturated fatty acids attached.

FIG. 4 illustrates the structures of C16 LCFA-conjugated siRNAs synthesized.

FIG. 5 illustrates the structures of PTEN siRNA synthesized with LCFA conjugation at both the 3′ and 5′ positions.

FIG. 6 illustrates the structures of synthesized PTEN siRNAs with conjugated C16 LCFAs containing terminal COOH groups.

FIG. 7 illustrates the structures of DTx-01-08-conjugated DTxO-0038, DTxO-0033, and DTXO-0034 siRNAs synthesized.

FIG. 8 illustrates the structures of DTxO-0003 siRNA conjugated to a motif having one or more unsaturated LCFAs.

FIG. 9 illustrates the structures of DTxO-0003 siRNA conjugated to a motif having a rigid linker.

FIG. 10 illustrates the structures of DTxO-0003 siRNA conjugated to a motif having three LCFAs.

FIG. 11 illustrates the structures of DTxO-0003 siRNA or DTxO-0038 siRNA conjugated to the DTx-01-08 motif, at the 5′ end of the passenger strand or 3′ end of the guide strand.

FIG. 12A illustrates the structures of the DTxO-0003 siRNA conjugated to the DTx-01-50, DTx-01-51, DTx-01-52, DTx-01-53, DTx-01-54, or DTx-01-55 motif.

FIG. 12B illustrates the structures of the DTxO-0003 siRNA conjugated to the DTx-03-50, DTx-03-51, DTx-03-52, DTx-03-53, DTx-03-54, or DTx-03-55 motif.

FIG. 12C illustrates the structures of the DTxO-0003 siRNA conjugated to the DTx-06-50, DTx-06-51, DTx-06-52, DTx-06-53, DTx-06-54, or DTx-06-55 motif.

FIG. 13 illustrates the percent of PTEN mRNA expression relative to a PBS control in HEK293 cells after transfection at various concentrations of Compounds 2, 7, 8, 26, and 1 for 48 hours.

FIG. 14 illustrates the percent of PTEN mRNA expression relative to a PBS control in HEK293 cells after the cells were exposed to various concentrations of Compounds 2, 7, 8, 26, and 1 under free uptake conditions for 48 hours.

FIG. 15 illustrates the percent of PTEN mRNA expression relative to a PBS control in HUVEC cells after the cells were exposed to various concentrations of Compounds 2, 7, 8, 26, and 1 under free uptake conditions for 48 hours.

FIG. 16 show a comparison of the effects of a conjugate comprising a rigid linker structure or a conjugate comprising three LCFAs on PTEN mRNA expression following transfection of compounds into HEK293 cells for 48 hours.

FIG. 17 show a comparison of the effects of a conjugate comprising a rigid linker structure or a conjugate comprising three LCFAs on PTEN mRNA expression following free uptake of compounds in HUVEC cells for 48 hours.

FIG. 18 illustrates the percent of PTEN mRNA expression relative to a PBS control in HEK293 cells after transfection at various concentrations of Compounds 2, 9, and 1 for 48 hours.

FIG. 19 illustrates the percent of PTEN mRNA expression relative to a PBS control in HUVEC cells after the cells were exposed to various concentrations of Compounds 2, 9, and 1 under free uptake conditions for 48 hours.

FIG. 20 shows the effects of compounds with a conjugate moiety attached to the 5′ terminus or the 3′ terminus of the passenger strand of two different siRNAs following transfection into HEK293 cells for 48 hours.

FIG. 21 shows the effects of compounds with a conjugate moiety attached to the 5′ terminus or the 3′ terminus of the passenger strand of two different siRNAs, following free uptake into HUVEC cells for 48 hours.

FIG. 22 illustrates the percent of PTEN mRNA expression relative to a PBS control in HEK293 cells after transfection at various concentrations of Compounds 2, 25, 24, and 1 for 48 hours.

FIG. 23 illustrates the percent of PTEN mRNA expression relative to a PBS control in NIH3T3 cells after transfection at various concentrations of Compounds 2, 25, 24, and 1 for 48 hours.

FIG. 24 illustrates the percent of PTEN mRNA expression relative to a PBS control in HUVEC cells after the cells were exposed to various concentrations of Compounds 2, 25, 24, and 1 under free uptake conditions for 48 hours.

FIG. 25 illustrates the percent of PTEN mRNA expression relative to a PBS control in HUVEC cells after the cells were exposed to various concentrations of Compounds 2, 25, 24, and 1 under free uptake conditions for 96 hours.

FIG. 26 illustrates the percent of PTEN mRNA expression relative to a PBS control in HEK293 cells after the cells were exposed to various concentrations of Compounds 2, 25, 24, and 1 under free uptake conditions for 48 hours.

FIG. 27 illustrates the percent of PTEN mRNA expression relative to a PBS control in HEK293 cells after the cells were exposed to various concentrations of Compounds 2, 25, 24, and 1 under free uptake conditions for 96 hours.

FIG. 28 illustrates the percent of PTEN mRNA expression relative to a PBS control in NIH3T3 cells after the cells were exposed to various concentrations of Compounds 2, 25, 24, and 1 under free uptake conditions for 48 hours.

FIG. 29 illustrates the percent of PTEN mRNA expression relative to a PBS control in NIH3T3 cells after the cells were exposed to various concentrations of Compounds 2, 25, 24, and 1 under free uptake conditions for 96 hours.

FIG. 30 illustrates the percent of PTEN mRNA expression relative to a PBS control in HEK293 cells after transfection at various concentrations of Compounds 2, 20, 21, and 23 for 48 hours.

FIG. 31 illustrates the percent of PTEN mRNA expression relative to a PBS control in HUVEC cells after the cells were exposed to various concentrations of Compounds 1, 2, 20, 21, and 23 under free uptake conditions for 48 hours.

FIG. 32 shows a comparison of the effects of conjugates containing saturated or unsaturated fatty acids on PTEN mRNA expression following transfection into HEK293 cells.

FIG. 33 shows a comparison of the effects of conjugates containing saturated or unsaturated fatty acids on PTEN mRNA expression following free uptake into HUVEC cells.

FIG. 34 illustrates the percent of PTEN mRNA expression relative to a PBS control in HEK293 cells after transfection at various concentrations of Compounds 2, 10, 11, 12, and 1 for 48 hours.

FIG. 35 illustrates the percent of PTEN mRNA expression relative to a PBS control in HEK293 cells after transfection at various concentrations of Compounds 2, 13, 14, 15, and 1 for 48 hours.

FIG. 36 illustrates the percent of PTEN mRNA expression relative to a PBS control in HUVEC cells after the cells were exposed to various concentrations of Compounds 2, 10, 11, 12, and 1 under free uptake conditions for 48 hours.

FIG. 37 illustrates the percent of PTEN mRNA expression relative to a PBS control in HUVEC cells after the cells were exposed to various concentrations of Compounds 2, 13, 14, 15, and 1 under free uptake conditions for 48 hours.

FIG. 38 illustrates the percent of PTEN mRNA expression relative to a PBS control in HEK293 cells after transfection at various concentrations of Compounds 2, 16, 17, 18, and 1 for 48 hours.

FIG. 39 illustrates the percent of PTEN mRNA expression relative to a PBS control in HEK293 cells after the cells were exposed to various concentrations of Compounds 2, 16, 17, 18, and 1 under free uptake conditions for 48 hours.

FIG. 40 illustrates the percent of PTEN mRNA expression relative to a PBS control in differentiated SH-SY5Y cells after the cells were exposed to various concentrations of Compounds 2, 16, 17, 18, and 1 under free uptake conditions for 48 hours.

FIG. 41 illustrates the percent of PTEN mRNA expression relative to a PBS control in HUVEC cells after the cells were exposed to various concentrations of Compounds 2, 16, 17, 18, and 1 under free uptake conditions for 48 hours.

FIG. 42 illustrates the percent of PTEN mRNA expression relative to a PBS control in HUVEC cells after the cells were exposed to various concentrations of Compounds 2, 16, 17, 18, and 1 under free uptake conditions for 96 hours.

FIG. 43 illustrates the percent of PTEN mRNA expression relative to a PBS control in primary rat neurons after the cells were exposed to various concentrations of Compounds 2, 16, 17, 18, and 1 under free uptake conditions for 96 hours.

FIG. 44 illustrates the percent of PTEN mRNA expression relative to a PBS control in primary rat neurons after the cells were exposed to various concentrations of Compounds 2, 16, 17, 18, and 1 under free uptake conditions for 7 days.

FIG. 45A illustrates the percent of VEGFR1 expression relative to a PBS control in HUVEC cells after transfection at various concentrations of Compounds 3 and 1 for 48 hours.

FIG. 45B illustrates the percent of PTEN mRNA expression relative to a PBS control in HUVEC cells after transfection at various concentrations of Compounds 3 and 1 for 48 hours.

FIG. 46A illustrates the percent of VEGFR2 relative to a PBS control in HUVEC cells after transfection at various concentrations of Compounds 5 and 1 for 48 hours.

FIG. 46B illustrates the percent of PTEN relative to a PBS control in HUVEC cells after transfection at various concentrations of Compounds 5 and 1 for 48 hours.

FIG. 47 illustrates the percent of VEGFR1 mRNA expression relative to a PBS control in HUVEC cells after the cells were exposed to various concentrations of Compounds 4 and 3 under free uptake conditions for 48 hours.

FIG. 48 illustrates the percent of VEGFR2 mRNA expression relative to a PBS control in HUVEC cells after the cells were exposed to various concentrations of Compounds 6 and 5 under free uptake conditions for 48 hours.

FIG. 49 illustrates the percent of HTT mRNA expression relative to a PBS control in undifferentiated SH-SY5Y cells after transfection at various concentrations of Compounds 29, 28, 27, 2, and 1 for 48 hours.

FIG. 50 illustrates the percent of HTT mRNA expression relative to a PBS control in undifferentiated SH-SY5Y cells after the cells were exposed to various concentrations of Compounds 29, 28, 27, 2, and 1 under free uptake conditions for 48 hours.

FIG. 51 illustrates the percent of HTT mRNA expression relative to a PBS control in differentiated SH-SY5Y cells after the cells were exposed to various concentrations of Compounds 29, 28, 27, 2, and 1 under free uptake conditions for 48 hours.

FIG. 52 illustrates the percent of PTEN mRNA expression relative to a PBS control in differentiated 3T3L1 adipocytes after the cells were exposed to various concentrations of Compounds 2 and 1 under free uptake conditions for 48 hours

FIG. 53 illustrates the percent of PTEN mRNA expression relative to a PBS control in trabecular meshwork after the cells were exposed to various concentrations of Compounds 2 and 1 under free uptake conditions for 48 hours.

FIG. 54 illustrates the percent of PTEN mRNA expression relative to a PBS control in differentiated primary human skeletal muscle cells after the cells were exposed to various concentrations of Compounds 2 and 1 under free uptake conditions for 96 hours.

FIG. 55 illustrates the percent of PTEN mRNA expression relative to a PBS control in primary human hepatocytes after the cells were exposed to various concentrations of Compounds 1, 2, 7, 8, and 9 under free uptake conditions for 48 hours.

FIG. 56 shows the percent of PTEN mRNA expression of Compounds 1, 2, 7, 8, and 9 relative to a PBS control in primary human adipocytes 7 days after incubation.

FIG. 57 illustrates the percent of PTEN mRNA expression relative to a PBS control in differentiated primary human skeletal muscle cells after the cells were exposed to various concentrations of Compounds 1, 2, 7, 8, and 9 under free uptake conditions for 96 hours.

FIG. 58 illustrates the percent of PTEN mRNA expression relative to a PBS control in primary human stellate cells after the cells were exposed to various concentrations of Compounds 1, 2, 7, 8, and 9 under free uptake conditions for 48 hours.

FIG. 59 illustrates the percent of PTEN mRNA expression relative to a PBS control in human T cells after the cells were exposed to various concentrations of Compounds 2 and 9 under free uptake conditions for 96 hours.

FIG. 60 shows the percent of PTEN mRNA expression seven days following intravitreal injection of Compound 2 and Compound 37, at varying doses, into mice.

FIG. 61 shows quantitative in situ hybridization (RNAscope) seven days following intravitreal injection of Compound 2 in rats. (ONL, Outer nuclear layer; INL, Inner Nuclear Layer; GCL, Ganglion Cell Layer; 10×, 10× magnification; 40×, 40× magnification).

FIG. 62 shows the percent of PTEN mRNA expression seven days following intravitreal injection of Compound 2 into rats.

FIG. 63 shows the percent of PTEN mRNA expression following transfection of conjugated (Compound 2) and unconjugated (Compound 30) PTEN siRNA into HEK293 cells at varying doses for 48 hours.

FIG. 64 shows the percent mRNA expression seven days following intravitreal injection of Compound 2 and 33 into mice. (1 Way ANOVA, Tukey Post-hoc; ***p<0.001, ****p<0.0001, N.S., not significant).

FIG. 65 shows the percent HTT mRNA expression seven days following intravitreal injection of Compounds 2 and 29 into mice. (1 Way ANOVA, Tukey Post-hoc; *p<0.05, ****p<0.0001, N.S., not significant).

FIG. 66 shows the percent VEGFR2 mRNA expression following transfection of unconjugated VEGFR2 siRNAs, Compounds 31 and 32, into BEND cells at varying doses for 48 hours.

FIG. 67 shows the percent VEGFR2 mRNA expression seven days following intravitreal injection of Compounds 2, 34 and 35 into mice. (1 Way ANOVA, Tukey Post-hoc; ***p<0.001, ****p<0.0001, N.S., not significant).

FIG. 68 shows the percent VEGFR2 mRNA expression seven days following intravitreal injection of Compounds 2, and 34 into rats. (1 Way ANOVA, Tukey Post-hoc; ****p<0.0001, N.S., not significant).

FIG. 69 shows the percent PTEN mRNA expression seven days following intravitreal injection of Compounds 2, 20, 21 and 1 into mice. (1 Way ANOVA, Tukey Post-hoc; ***p<0.001, ****p<0.0001, N.S., not significant).

FIG. 70 shows the percent PTEN mRNA expression seven days following intravitreal injection of Compounds 11, 12, 2, 13 and 1 into mice. (1 Way ANOVA, Tukey Post-hoc; **p<0.01, ****p<0.0001, N.S., not significant).

FIG. 71 shows the percent PTEN mRNA expression seven days following intravitreal injection of Compounds 1 and 2 into mice.

FIG. 72 shows PTEN mRNA expression in the liver seven days following either subcutaneous (SQ) or intravenous (IV) administration of Compound 33 to C57Bl/6 mice.

FIG. 73 shows PTEN mRNA expression in muscle, heart, fat, lung, liver, kidney and spleen tissues seven days following intravenous administration of Compound 33 to C57Bl/6 mice.

FIG. 74 illustrates the percent of PTEN mRNA expression relative to a PBS control in HEK293 cells after transfection at various concentrations of Compounds 2, 12, 54, 55, and 1 for 48 hours.

FIG. 75 illustrates the percent of PTEN mRNA expression relative to a PBS control in HEK293 cells after transfection at various concentrations of Compounds 2, 13, 56, 57, and 1 for 48 hours.

FIG. 76 illustrates the percent of PTEN mRNA expression relative to a PBS control in HEK293 cells after transfection at various concentrations of Compounds 12, 13, 58, 59, and 1 for 48 hours.

FIG. 77 illustrates the percent of PTEN mRNA expression relative to a PBS control in HUVEC cells after the cells were exposed to various concentrations of Compounds 2, 12, 54, 55, and 1 under free uptake conditions for 48 hours.

FIG. 78 illustrates the percent of PTEN mRNA expression relative to a PBS control in HUVEC cells after the cells were exposed to various concentrations of Compounds 2, 13, 56, 57, and 1 under free uptake conditions for 48 hours.

FIG. 79 illustrates the percent of PTEN mRNA expression relative to a PBS control in HUVEC cells after the cells were exposed to various concentrations of Compounds 12, 13, 58, 59, and 1 under free uptake conditions for 48 hours.

FIG. 80 illustrates the structures of Compounds 72 to 83 having various combinations of saturated and unsaturated long chain fatty acid motifs conjugated to the 3′ end of the passenger strand of an siRNA.

FIG. 81 illustrates the structures of Compounds 84 to 95 having various combinations of saturated and unsaturated long chain fatty acid motifs conjugated to the 3′ end of the passenger strand of an siRNA.

FIG. 82 illustrates the structures of Compounds 96 to 107 having various combinations of saturated and unsaturated long chain fatty acid motifs conjugated to the 3′ end of the passenger strand of an siRNA.

FIG. 83 illustrates the structures of Compounds 108 through 113 having various combinations of saturated and unsaturated long chain fatty acid motifs conjugated to the 3′ end of an siRNA.

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3 dihydrobenzofuran 2 yl, 2,3 dihydrobenzofuran 3 yl, indolin 1 yl, indolin 2 yl, indolin 3 yl, 2,3 dihydrobenzothien 2 yl, decahydroquinolinyl, decahydroisoquinolinyl, octahydro 1H indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

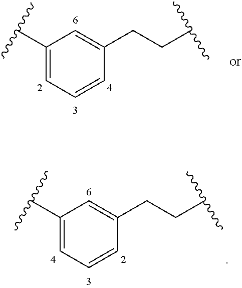 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR″, ═O, ═NR″, ═N—OR″, —NR″R″, —SR″, -halogen, —SiR″R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR″SO₂R″, —NR″C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds provided herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds provided herein may exist in tautomeric forms, all such tautomeric forms of the compounds being within the scope of the present disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of the present disclosure.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds provided herein, whether radioactive or not, are included within the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently. The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Description of compounds of provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Contacting” is used in accordance with its plain ordinary meaning and refers to the process of allowing at least two distinct species (e.g. chemical compounds, biomolecules or cells) to become sufficiently proximal to react, interact or physically touch. For example, contacting includes the process of allowing a compound to become sufficiently proximal to a cell to bind to a cell-surface receptor.

As used herein, “contacting a cell” refers to a condition in which a compound or other composition of matter is in direct contact with a cell, or is close enough to induce a desired biological effect in a cell.

The term “free uptake conditions” as used herein refer to conditions in which unmodified oligonucleotides do not substantially enter a cell. For example, such free uptake conditions can be conditions in which there are little or no transfection reagents, electroporation techniques or other conditions used to promote compound entry into cells. Free uptake conditions can be conditions in which siRNA lacking lipid conjugation substantially does not enter cells, such as incubation in standard media under standard conditions for the particular type of cell. An example of standard media conditions for free uptake can be fetal bovine serum (FBS) in a range from 0.5% to 10%, for example 1% to 5%. In other examples, the standard media is serum free.

The term “activator,” refers to a compound, composition, or substance capable of detectably increasing the expression or activity of a given gene or protein. For example, an activator may increase expression or activity 10%, 20%, 30%, 40%, 50%, 60%, 70%, 80%, 90% or more in comparison to a control in the absence of the activator.

As defined herein, the term “inhibition”, “inhibit”, “inhibiting” and the like mean negatively affecting (e.g. decreasing) activity or function relative to the activity or function in the absence of the inhibitor. In embodiments inhibition means negatively affecting (e.g. decreasing) the concentration or levels of a biomolecule, such as a protein or mRNA, relative to the concentration or level of the biomolecule in the absence of the inhibitor. For example, inhibition includes decreasing the level of mRNA expression in a cell. In embodiments, inhibition refers to a reduction in the activity of a particular biomolecule target, such as a protein target or an mRNA target. Thus, inhibition includes, at least in part, partially or totally blocking stimulation, decreasing, preventing, or delaying activation, or inactivating, desensitizing, or down-regulating signal transduction or enzymatic activity or the amount of a biomolecule. In embodiments, inhibition refers to a reduction of activity of a target biomolecule resulting from a direct interaction (e.g. an inhibitor binds to a target protein). In embodiments, inhibition refers to a reduction of activity of a target biomolecule from an indirect interaction (e.g. an inhibitor binds to a protein that activates a target protein, thereby preventing target protein activation).

The term “inhibitor” also refers to a compound, composition, or substance capable of detectably decreasing the expression or activity of a given gene or protein. For example, an inhibitor may decrease expression or activity 10%, 20%, 30%, 40%, 50%, 60%, 70%, 80%, 90% or more in comparison to a control in the absence of the inhibitor. Inhibitors include, for example, synthetic or biological molecules, such as oligonucleotides.

The terms “expression” and “gene expression” as used herein refer to the steps involved in the translation of a nucleic acid into a protein, including mRNA expression and protein expression. Expression can be detected using conventional techniques for detecting nucleic acids or proteins (e.g., PCR, ELISA, Southern blotting, Western blotting, flow cytometry, FISH, immunofluorescence, immunohistochemistry).

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist.

The term “cell” is used herein in its ordinary sense as understood by a person of ordinary skill in the art. A cell may be prokaryotic or eukaryotic. Prokaryotic cells include but are not limited to bacteria. Eukaryotic cells include but are not limited to yeast cells, plant cells, and animal cells, including human cells. A cell can be identified by well-known methods in the art including, for example, presence of an intact membrane, staining by a particular dye, ability to produce progeny or, in the case of a gamete, ability to combine with a second gamete to produce a viable offspring. In embodiments, the cell may be from an immortalized cell line. In embodiments, the cell may be a primary cell. In embodiments, a cell is in vitro. In embodiments, a cell is in vivo. In embodiments, a cell is ex vivo.

The term “in vivo” used herein means a process that takes place within a subject's body.

The term “subject” used herein means a human or non-human animal selected for treatment or therapy. In embodiments, a subject is a human.

The term “ex vivo” used herein means a process that takes place in vitro in isolated tissue or cells where the treated tissue or cells comprise primary cells. As is known in the art, any medium used in this process can be aqueous and non-toxic so as not to render the tissue or cells non-viable. In embodiments, the ex vivo process takes place in vitro using primary cells.

The term “administration” means providing a pharmaceutical agent or composition to a subject, and includes administration performed by a medical professional and self-administration.

The term “therapy” means the application of one or more specific procedures used for the amelioration of at least one indicator or a disease or condition. In embodiments, the specific procedure is the administration of one or more pharmaceutical agents.

The term “modulate” is used herein in its ordinary sense as understood by a person of ordinary skill in the art, and thus refers to the act of changing or varying one or more properties. For example, in the context of a modulator's effects on a target molecule, to modulate means to change by increasing or decreasing a property or function of the target molecule or the amount of the target molecule. A modulator of a disease decreases a symptom, cause, or characteristic of the targeted disease.

The terms “nucleic acid,” “oligonucleotide,” and “polynucleotide” refer to compounds containing at least two nucleotide monomers covalently linked together. The terms include single-stranded and double-stranded nucleic acids, nucleic acids, oligonucleotides, and polynucleotides, including single-stranded DNA, double-stranded DNA, single-stranded RNA, double-stranded RNA, single-stranded and double-stranded molecules containing both DNA and RNA nucleotides, and modified versions thereof. Oligonucleotides refer to shorter length polymers, and are typically from about 5, 6, 7, 8, 9, 10, 12, 15, 25, 30, 40, 50 or more nucleotides in length, up to about 100 nucleotides in length. Nucleic acids and polynucleotides are typically nucleotide polymers of longer lengths, e.g., 200, 300, 500, 1000, 2000, 3000, 5000, 7000, 10,000. A “residue” of a nucleic acid, oligonucleotide, or polynucleotide refers to a nucleotide monomer of that compound. “Residue” and “monomer” are used interchangeably herein. In embodiments, the oligonucleotide may be used in RNA silencing. In embodiments, the oligonucleotide may comprise DNA, locked nucleic acids (LNA), bicyclic nucleic acids (BNA), or phosphorodiamidate morpholino oligomer (PMO), or modification thereof and the like. In embodiments, the oligonucleotide comprises one or more 2′-O-methoxy ethyl residues, 2′-O-methyl residues, and/or 2′-fluoro residues. In embodiments, the oligonucleotide comprises phosphorothioate linkages.

Non-limiting examples of oligonucleotides include double-stranded oligonucleotides, modified double-stranded oligonucleotides, single-stranded oligonucleotides, modified single-stranded oligonucleotides, antisense oligonucleotides, siRNAs, microRNA mimics, stem-loop structures, single-strand siRNAs, RNaseH oligonucleotides, anti-microRNA oligonucleotides, steric blocking oligonucleotides, CRISPR guide RNAs, and aptamers.

Non-limiting examples of polynucleotides include a gene, a gene fragment, an exon, an intron, intergenic DNA (including, without limitation, heterochromatic DNA), messenger RNA (mRNA), a long non-coding RNA, transfer RNA, ribosomal RNA, a ribozyme, cDNA, a recombinant polynucleotide, a branched polynucleotide, a plasmid, a vector, isolated DNA of a sequence, and an isolated RNA of a sequence. Polynucleotides useful in the methods of the disclosure may include natural nucleic acid sequences and variants thereof, artificial nucleic acid sequences, or a combination of such sequences.

“Nucleoside,” as used herein, refers to a glycosyl compound consisting of a nucleobase and a 5-membered ring sugar (e.g., either ribose or deoxyribose). Nucleosides may comprise bases such as A, C, G, T, U, or analogues thereof. Nucleosides may be modified at the base and/or and the sugar. In an embodiment, the nucleoside is a deoxyribonucleoside. In another embodiment, the nucleoside is a ribonucleoside.

“Nucleotide,” as used herein, refers to a nucleoside-5′-polyphosphate compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may comprise bases such as A, C, G, T, U, or analogues thereof, and may comprise 2, 3, 4, 5, 6, 7, 8, or more phosphates in the phosphate group. Nucleotides may be modified at one or more of the base, sugar, or phosphate group. A nucleotide may have a ligand attached, either directly or through a linker. In an embodiment, the nucleotide is a deoxyribonucleotide. In another embodiment, the nucleotide is a ribonucleotide.

As used herein, “nucleotide analogue” shall mean an analogue of A, G, C, T or U (that is, an analogue of a nucleotide comprising the base A, G, C, T or U), comprising a phosphate group, which may be recognized by DNA or RNA polymerase (whichever is applicable) and incorporated into a strand of DNA or RNA (whichever is appropriate). Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

The terms “base” in the context of oligonucleotides, nucleic acids or polynucleotides, and “nucleobase” as used herein refers to a purine or pyrimidine compound or a derivative thereof, that may be a constituent of nucleic acid (i.e. DNA or RNA, or a derivative thereof). In embodiments, the nucleobase is a derivative of a naturally occurring DNA or RNA base (e.g., a base analogue). In embodiments, the nucleobase is a derivative of a naturally occurring DNA or RNA base (e.g., a base analogue), which may be optionally subsituted. In embodiments, the nucleobase is a hybridizing base. In embodiments, the nucleobase is a hybridizing base, which may be optionally substituted. In embodiments, the nucleobase hybridizes to a complementary base. In embodiments, the nucleobase is capable of forming at least one hydrogen bond with a complementary nucleobase (e.g., adenine hydrogen bonds with thymine, adenine hydrogen bonds with uracil, or guanine pairs with cytosine). Non-limiting examples of the nucleobase includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the nucleobase is adenine, guanine, hypoxanthine, xanthine, theobromine, caffeine, uric acid, or isoguanine, which may be optionally substituted or modified. In embodiments, the nucleobase is

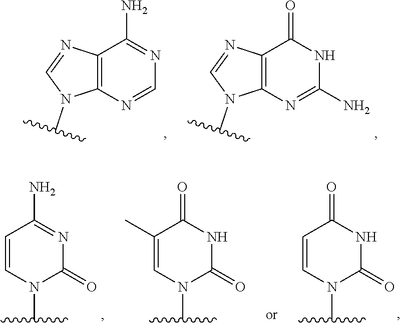

which may be optionally substituted or modified.

Oligonucleotides, nucleic acids and polynucleotides can include nonspecific sequences. As used herein, the term “nonspecific sequence” refers to a sequence that contains a series of residues that are not designed to be complementary to or are only partially complementary to any other sequence. By way of example, two strands of a double-stranded oligonucleotide may hybridize in a way that results in one or more short (e.g. two) nucleotide overhangs at one or both termini of the duplex. As another example, a nonspecific nucleic acid sequence is a sequence of nucleic acid residues that does not function as an inhibitory nucleic acid when contacted with a cell or organism.

The term “double-stranded oligonucleotide” as used herein refers to an oligonucleotide with nucleobase sequence that is sufficiently complementary to form a duplex structure. Double-stranded oligonucleotides may comprise structures formed from annealing a first oligonucleotide to a second, complementary oligonucleotide. Double-stranded oligonucleotides may be fully complementary over the length of both oligonucleotides. Alternatively, double-stranded oligonucleotide may have a short nucleotide overhang at one or both ends of the duplex structure. Such double-stranded oligonucleotides include siRNAs and microRNA mimics. Double-stranded oligonucleotides may also include a single oligonucleotide with sufficient length and self-complementarity to form a duplex structure. Such double-stranded oligonucleotides include stem-loop structures. A double-stranded oligonucleotide may include one or more modifications relative to a naturally occurring terminus, sugar, nucleobase, and/or inteRucleoside linkage.

The term “modified double-stranded oligonucleotide” as used herein refers to a double-stranded oligonucleotide comprising one or more modifications relative to a naturally occurring terminus, sugar, nucleobase, and/or inteRucleoside linkage. In the case of a double-stranded oligonucleotide comprising two separate, complementary oligonucleotides, one or both strands may comprise one or more modifications relative to a naturally occurring terminus, sugar, nucleobase, and/or inteRucleoside linkage.

The terms “small interfering RNA,” “short interfering RNA,” “silencing RNA,” and “siRNA” are used interchangeably herein to refer to a class of double-stranded oligonucleotide which interferes with the expression of specific genes by facilitating mRNA degradation before translation, i.e. through the RNA interference pathway. siRNAs comprise a guide strand, which is complementary to the target mRNA and is incorporated into the RNA-induced silencing complex (RISC) and a passenger strand, which is complementary to the guide strand and is typically degraded. Typically, siRNA molecules are about 15-50 nucleotides in length, and more typically 20-30 base nucleotides in length, 20-25 nucleotides in length or 24-29 nucleotides in length. In embodiments, siRNAs are about 18-25 nucleotides in length. An siRNA may include one or more modifications relative to a naturally occurring terminus, sugar, nucleobase, and/or inteRucleoside linkage.

The term “microRNA mimic” as used herein refers to a synthetic version of a naturally occurring microRNA. A microRNA mimic comprises a guide strand, which is complementary to one or more target mRNAs, and a passenger strand which is complementary to the guide strand. In naturally occurring microRNAs, the guide strand is typically only partially complementary to its target mRNA(s), and the passenger strand is only partially complementary to the guide strand. A microRNA mimic may comprise nucleobase sequences having 100% identity to the naturally occurring microRNA or may comprise a nucleobase sequences less than 100% identical to the naturally occurring microRNA. For example, a microRNA mimic may comprise a passenger strand that is 100% complementary to the guide strand. A microRNA mimic may include one or more modifications relative to a naturally occurring terminus, sugar, nucleobase, and/or inteRucleoside linkage.

The term “single-stranded oligonucleotide” as used herein refers to an oligonucleotide that is not hybridized to a complementary strand. A single-stranded oligonucleotide may include one or more modifications relative to a naturally occurring terminus, sugar, nucleobase, and/or internucleoside linkage. Single-stranded oligonucleotides include antisense oligonucleotides. Single-stranded oligonucleotides also include aptapmers which are single-stranded oligonucleotides that fold into a well-defined secondary structure.

The term “modified single-stranded oligonucleotide” as used herein refers to a single-stranded oligonucleotide that is not hybridized to a complementary strand and comprises one or more modifications relative to a naturally occurring terminus, sugar, nucleobase, and/or internucleoside linkage. Modified single-stranded oligonucleotides include modified antisense oligonucleotides and aptamers.

An “antisense oligonucleotide” as referred to herein is a single-stranded oligonucleotide that is complementary to, and thus capable of selectively hybridizing to, at least a portion of a specific target nucleic acid and is further capable of reducing transcription of the target nucleic acid (e.g. mRNA from DNA), reducing the translation of the target nucleic acid (e.g. mRNA), altering transcript splicing, or otherwise interfering with the endogenous activity of the target nucleic acid. Typically, antisense oligonucleotides are between 15 and 25 bases in length. An antisense oligonucleotide may comprise one or more modifications to a naturally occurring terminus, sugar, nucleobase, and/or internucleoside linkage Antisense oligonucleotides include, without limitation, anti-microRNA oligonucleotides (oligonucleotides complementary to microRNAs), steric blocking oligonucleotides (oligonucleotides that interfere with target RNA activity without degrading the target RNA), and RNaseH oligonucleotides (oligonucleotides chemically modified to elicit RNaseH-mediated degradation of a target RNA).

A nucleic acid, oligonucleotide, or polynucleotide is “modified” if one or more of the termini, phosphodiester linkages, sugars, or bases is altered from its natural form (e.g., altered from the common form in DNA or RNA, altered to form a nucleotide analogue). For example, a nucleic acid is modified if one or more of its phosphodiester linkages is replaced by a phosphoramidate, phosphorothioate, phosphorodithioate, boranophosphonate, or O-methylphosphoroamidite linkage (see, e.g., Eckstein, Oligonucleotides and Analogues: A Practical Approach, Oxford University Press). Modified nucleic acids, oligonucleotides, and polynucleotides include those with positive backbones; non-ionic backbones, and non-ribose backbones, such as those described in U.S. Pat. Nos. 5,235,033 and 5,034,506, and Chapters 6 and 7, ASC Symposium Series 580, Carbohydrate Modifications in Antisense Research, Sanghui & Cook, eds. Modified nucleic acids, oligonucleotides, and polynucleotides also include nucleic acids, oligonucleotides, and polynucleotides where one or more of the residues contain a chemically altered ribose sugar, such as 2′-O-methyl-ribose, 2′-deoxy-2′-fluoro-ribose, and ribose “locked” by a covalent linkage between the 2′ and 4′ carbons. “Bicyclic nucleic acid” or “BNA” residues comprise a covalent linkage between the 2′ hydroxyl group of the sugar ring is connected to the 4′ carbon of the sugar ring which essentially “locks” the structure into a rigid conformation. A bicyclic nucleic acid residue comprising a methyleneoxy (4′-CH₂—O-2′) bridge between the 2′ hydroxyl group and 4′ carbon of the ribose is a “locked nucleic acid” or “LNA”. A bicyclic nucleic acid residue comprising a 4′-CH(CH₃)—O-2′ bridge is a “constrained ethyl” or “cEt” residue. An “unlocked nucleic acid” or “UNA” residue is an acyclic nucleoside derivative lacking the bond between the 2′ carbon and 3′ carbon of the sugar ring. Further, modified nucleic acids, oligonucleotides, and polynucleotides may be modified at one or both of the 5′ terminus and 3′ terminus. For example, an oligonucleotide may comprise a 5′-(E)-vinylphosphonate group at a terminus. Nucleic acid modifications may be done for a variety of reasons, e.g., to increase the stability and half-life of such molecules in physiological environments, or to prevent immune stimulation.

In embodiments, an oligonucleotide may consist of, consist essentially of, or comprise a single strand of locked nucleic acids (LNA), or modification thereof. In embodiments, the oligonucleotide may consist of, consist essentially of, or comprise a single strand of phosphorodiamidate morpholino oligomer (PMO), or modification thereof. In embodiments, the oligonucleotide may comprise at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, of DNA, siRNA, mRNA, locked nucleic acids (LNA), bicyclic nucleic acids (BNA), or phosphorodiamidate morpholino oligomer (PMO), or modification thereof and the like, or the oligonucleotide may comprise an amount of DNA, siRNA, mRNA, locked nucleic acids (LNA), bicyclic nucleic acids (BNA), or phosphorodiamidate morpholino oligomer (PMO), or modification thereof and the like within a range defined by any of two of the preceding values. In embodiments, the oligonucleotide may comprise at least 1% and less than 20%, 19%, 18%, 17%, 16%, 15%, 14%, 13%, 12%, 11%, 10%, 9%, 8%, 7%, 6%, 5%, or 4% of 2′-O-methoxy ethyl/phosphorothioate (MOE).

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that participate in nucleobase-pairing (i.e., about 60% complementarity, preferably 65%, 70%, 75%, 80%, 85%, 90%, 91%, 92%, 93%, 94%, 95%, 96%, 97%, 98%, 99%, or higher complementarity over a specified region).

“Hybridize” shall mean the annealing of one single-stranded nucleic acid (such as a primer) to another nucleic acid based on the well-understood principle of sequence complementarity. In an embodiment the other nucleic acid is a single-stranded nucleic acid. The propensity for hybridization between nucleic acids depends on the temperature and ionic strength of their miliu, the length of the nucleic acids and the degree of complementarity. The effect of these parameters on hybridization is described in, for example, Sambrook J, Fritsch E F, Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith.

A particular nucleic acid sequence also encompasses “splice variants.” Similarly, a particular protein encoded by a nucleic acid encompasses any protein encoded by a splice variant of that nucleic acid. “Splice variants,” are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see. e.g., NCBI web site or the like). Ibis definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see. e.g., Current Protocols in Molecular Biology (Ausubel et al., eds. 1995 supplement)).

### Compounds and Methods

In an aspect, inter alia, are compounds, or lipid-modified oligonucleotide compounds, having the following structure:

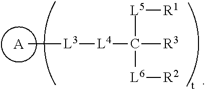

A is an oligonucleotide, a nucleic acid, a polynucleotide, a nucleotide or analog thereof or a nucleoside or analog thereof. In embodiments, A is an oligonucleotide. In embodiments, A is a nucleic acid. In embodiments, A is a polynucleotide. In embodiments, A is a nucleotide or analog thereof. In embodiments, A is a nucleoside or analog thereof.

L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

L⁵ is -L^{5A}-L^{5B}-L^{5C}-L^{5D}-L^{5E}- and L⁶ is -L^{6A}-L^{6B}-L^{6C}-L^{6D}-L^{6E}-. L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. In embodiments, R¹ and R² are independently unsubstituted C₁-C₂₀ alkyl, wherein at least one of R¹ and R² is unsubstituted C₉-C₁₉ alkyl.

R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

t is an integer from 1 to 5.

In embodiments, t is 1. In embodiments, t is 2. In embodiment, t is 3. In embodiments, t is 4. In embodiment t is 5.

In embodiments, A is a double-stranded oligonucleotide, or single-stranded oligonucleotide. In embodiments, A is a double-stranded oligonucleotide. In embodiments, A is a single-stranded oligonucleotide. In embodiments, A is a modified oligonucleotide. In embodiments, A is a modified double-stranded oligonucleotide, modified single-stranded oligonucleotide. In embodiments, A is a modified double-stranded oligonucleotide. In embodiments, A is a modified single-stranded oligonucleotide.

In embodiments, A is an siRNA, a microRNA mimic, a stem-loop structure, a single-stranded siRNA, an RNaseH oligonucleotide, an anti-microRNA oligonucleotide, a steric blocking oligonucleotide, a CRISPR guide RNA, or an aptamer.

In embodiments, one L³ is attached to a 3′ carbon of the double-stranded oligonucleotide or single-stranded oligonucleotide. In embodiments, one L³ is attached to a 3′ carbon of double-stranded oligonucleotide. In embodiments, one L³ is attached to a 3′ carbon of single-stranded oligonucleotide. In embodiments, one L³ is attached to the 3′ carbon of a 3′ terminal nucleotide of the double-stranded oligonucleotide or single-stranded oligonucleotide. In embodiments, one L³ is attached to the 3′ carbon of a 3′ terminal nucleotide of the double-stranded oligonucleotide. In embodiments, one L³ is attached to the 3′ carbon of the 3′ terminal nucleotide of the single-stranded oligonucleotide.

In embodiments, one L³ is attached to a 5′ carbon of the double-stranded oligonucleotide or single-stranded oligonucleotide. In embodiments, one L³ is attached to a 5′ carbon of the double-stranded oligonucleotide. In embodiments, one L³ is attached to a 5′ carbon of the single-stranded oligonucleotide. In embodiments, one L³ is attached to the 5′ carbon of a 5′ terminal nucleotide of a double-stranded oligonucleotide or single-stranded oligonucleotide. In embodiments, one L³ is attached to the 5′ carbon of a 5′ terminal nucleotide of the double-stranded oligonucleotide. In embodiments, one L³ is attached to the 5′ carbon of the 5′ terminal nucleotide of the single-stranded oligonucleotide.

In embodiments, one L³ is attached to a 2′ carbon of a nucleotide of the double-stranded oligonucleotide. In embodiments, one L³ is attached to a 2′ carbon of a nucleotide of the single-stranded oligonucleotide. In embodiments, the 2′ carbon is the 2′ carbon of an internal nucleotide.

In embodiments, one L³ is attached to a nucleobase of the double-stranded oligonucleotide or single-stranded oligonucleotide. In embodiments, one L³ is attached to a nucleobase of the double-stranded oligonucleotide. In embodiments, one L³ is attached to a nucleobase of the single-stranded oligonucleotide.

In embodiments, L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene or substituted or unsubstituted heteroalkylene. In embodiments, L³ is independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene or substituted or unsubstituted heteroalkylene. In embodiments, L⁴ is independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene or substituted or unsubstituted heteroalkylene.

In embodiments, L³ is independently a bond. In embodiments, L³ is independently —NH—. In embodiments, L³ is independently —O—. In embodiments, L³ is independently —S—. In embodiments, L³ is independently —C(O)—. In embodiments, L³ is independently —NHC(O)—. In embodiments, L³ is independently —NHC(O)NH—. In embodiments, L³ is independently —C(O)O—. In embodiments, L³ is independently —OC(O)—. In embodiments, L³ is independently —C(O)NH—. In embodiments, L³ is independently —OPO₂—O—. In embodiments, L³ is independently substituted or unsubstituted alkylene. In embodiments, L³ is independently substituted or unsubstituted heteroalkylene.

In embodiments, L³ is independently substituted or unsubstituted alkylene (e.g., C₁-C₂₀, C₁-C₁₂, C₁-C₈, C₁-C₆, C₁-C₄, or C₁-C₂). In embodiments, L³ is independently substituted alkylene (e.g., C₁-C₂₀, C₁-C₁₂, C₁-C₈, C₁-C₆, C₁-C₄, or C₁-C₂). In embodiments, L³ is independently unsubstituted alkylene (e.g., C₁-C₂₀, C₁-C₁₂, C₁-C₈, C₁-C₆, C₁-C₄, or C₁-C₂). In embodiments, L³ is independently substituted or unsubstituted C₁-C₂₀ alkylene. In embodiments, L³ is independently substituted C₁-C₂₀ alkylene. In embodiments, L³ is independently unsubstituted C₁-C₂₀ alkylene. In embodiments, L³ is independently substituted or unsubstituted C₁-C₁₂ alkylene. In embodiments, L³ is independently substituted C₁-C₁₂ alkylene. In embodiments, L³ is independently unsubstituted C₁-C₁₂alkylene. In embodiments, L³ is independently substituted or unsubstituted C₁-C₆ alkylene. In embodiments, L³ is independently substituted C₁-C₈ alkylene. In embodiments, L³ is independently unsubstituted C₁-C₈ alkylene. In embodiments, L³ is independently substituted or unsubstituted C₁-C₆ alkylene. In embodiments, L³ is independently substituted C₁-C₆alkylene. In embodiments, L³ is independently unsubstituted C₁-C₆ alkylene. In embodiments, L³ is independently substituted or unsubstituted C₁-C₄ alkylene. In embodiments, L³ is independently substituted C₁-C₄alkylene. In embodiments, L³ is independently unsubstituted C₁-C₄ alkylene. In embodiments, L³ is independently substituted or unsubstituted ethylene. In embodiments, L³ is independently substituted ethylene. In embodiments, L³ is independently unsubstituted ethylene. In embodiments, L³ is independently substituted or unsubstituted methylene. In embodiments, L³ is independently substituted methylene. In embodiments, L³ is independently unsubstituted methylene.

In embodiments, L³ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L³ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L³ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L³ is independently substituted or unsubstituted 2 to 20 membered heteroalkylene. In embodiments, L³ is independently substituted 2 to 20 membered heteroalkylene. In embodiments, L³ is independently unsubstituted 2 to 20 membered heteroalkylene. In embodiments, L³ is independently substituted or unsubstituted 2 to 8 membered heteroalkylene. In embodiments, L³ is independently substituted 2 to 8 membered heteroalkylene. In embodiments, L³ is independently unsubstituted 2 to 8 membered heteroalkylene. In embodiments, L³ is independently substituted or unsubstituted 2 to 6 membered heteroalkylene. In embodiments, L³ is independently substituted 2 to 6 membered heteroalkylene. In embodiments, L³ is independently unsubstituted 2 to 6 membered heteroalkylene. In embodiments, L³ is independently substituted or unsubstituted 4 to 6 membered heteroalkylene. In embodiments, L³ is independently substituted 4 to 6 membered heteroalkylene. In embodiments, L³ is independently unsubstituted 4 to 6 membered heteroalkylene. In embodiments, L³ is independently substituted or unsubstituted 2 to 3 membered heteroalkylene. In embodiments, L³ is independently substituted 2 to 3 membered heteroalkylene. In embodiments, L³ is independently unsubstituted 2 to 3 membered heteroalkylene. In embodiments, L³ is independently substituted or unsubstituted 4 to 5 membered heteroalkylene. In embodiments, L³ is independently substituted 4 to 5 membered heteroalkylene. In embodiments, L³ is independently unsubstituted 4 to 5 membered heteroalkylene.

In embodiments, L⁴ is independently a bond. In embodiments, L⁴ is independently —NH—. In embodiments, L⁴ is independently —O—. In embodiments, L⁴ is independently —S—. In embodiments, L⁴ is independently —C(O)—. In embodiments, L⁴ is independently —NHC(O)—. In embodiments, L⁴ is independently —NHC(O)NH—. In embodiments, L⁴ is independently —C(O)O—. In embodiments, L⁴ is independently —OC(O)—. In embodiments, L⁴ is independently —C(O)NH—. In embodiments, L⁴ is independently —OPO₂—O—. In embodiments, L⁴ is independently substituted or unsubstituted alkylene. In embodiments, L⁴ is independently substituted or unsubstituted heteroalkylene.

In embodiments, L⁴ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently substituted alkylene (e.g., C₁-C₂₀, C₁-C₁₂, C₁-C₈, C₁-C₆, C₁-C₄, or C₁-C₂). In embodiments, L⁴ is independently unsubstituted alkylene (e.g., C₁-C₂₀, C₁-C₁₂, C₁-C₈, C₁-C₆, C₁-C₄, or C₁-C₂). In embodiments, L⁴ is independently substituted or unsubstituted C₁-C₂₀ alkylene. In embodiments, L⁴ is independently substituted C₁-C₂₀ alkylene. In embodiments, L⁴ is independently unsubstituted C₁-C₂₀ alkylene. In embodiments, L⁴ is independently substituted or unsubstituted C₁-C₁₂ alkylene. In embodiments, L⁴ is independently substituted C₁-C₁₂ alkylene. In embodiments, L⁴ is independently unsubstituted C₁-C₁₂alkylene. In embodiments, L⁴ is independently substituted or unsubstituted C₁-C₈ alkylene. In embodiments, L⁴ is independently substituted C₁-C₈ alkylene. In embodiments, L⁴ is independently unsubstituted C₁-C₈ alkylene. In embodiments, L⁴ is independently substituted or unsubstituted C₁-C₆ alkylene. In embodiments, L⁴ is independently substituted C₁-C₆ alkylene. In embodiments, L⁴ is independently unsubstituted C₁-C₆ alkylene. In embodiments, L⁴ is independently substituted or unsubstituted C₁-C₄ alkylene. In embodiments, L⁴ is independently substituted C₁-C₄alkylene. In embodiments, L⁴ is independently unsubstituted C₁-C₄ alkylene. In embodiments, L⁴ is independently substituted or unsubstituted ethylene. In embodiments, L⁴ is independently substituted ethylene. In embodiments, L⁴ is independently unsubstituted ethylene. In embodiments, L⁴ is independently substituted or unsubstituted methylene. In embodiments, L⁴ is independently substituted methylene. In embodiments, L⁴ is independently unsubstituted methylene.

In embodiments, L⁴ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁴ is independently substituted or unsubstituted 2 to 20 membered heteroalkylene. In embodiments, L⁴ is independently substituted 2 to 20 membered heteroalkylene. In embodiments, L⁴ is independently unsubstituted 2 to 20 membered heteroalkylene. In embodiments, L⁴ is independently substituted or unsubstituted 2 to 8 membered heteroalkylene. In embodiments, L⁴ is independently substituted 2 to 8 membered heteroalkylene. In embodiments, L⁴ is independently unsubstituted 2 to 8 membered heteroalkylene. In embodiments, L⁴ is independently substituted or unsubstituted 2 to 6 membered heteroalkylene. In embodiments, L⁴ is independently substituted 2 to 6 membered heteroalkylene. In embodiments, L⁴ is independently unsubstituted 2 to 6 membered heteroalkylene. In embodiments, L⁴ is independently substituted or unsubstituted 4 to 6 membered heteroalkylene. In embodiments, L⁴ is independently substituted 4 to 6 membered heteroalkylene. In embodiments, L⁴ is independently unsubstituted 4 to 6 membered heteroalkylene. In embodiments, L⁴ is independently substituted or unsubstituted 2 to 3 membered heteroalkylene. In embodiments, L⁴ is independently substituted 2 to 3 membered heteroalkylene. In embodiments, L⁴ is independently unsubstituted 2 to 3 membered heteroalkylene. In embodiments, L⁴ is independently substituted or unsubstituted 4 to 5 membered heteroalkylene. In embodiments, L⁴ is independently substituted 4 to 5 membered heteroalkylene. In embodiments, L⁴ is independently unsubstituted 4 to 5 membered heteroalkylene.

In embodiments, L³ is independently

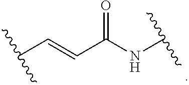 [SMILES: */C=C/C(=O)N*]

In embodiments, L³ is independently —OPO₂—O—. In embodiments, L³ is independently —O—.

In embodiments, L⁴ is independently substituted or unsubstituted alkylene or substituted or unsubstituted heteroalkylene. In embodiments, L⁴ is independently -L⁷-NH—C(O)— or -L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁴ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁴ is independently substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently unsubstituted heteroalkylene (e.g., 2 to 20 membered, 2 to 12 membered, 2 to 10 membered, 2 to 8 membered, 2 to 6 membered, or 2 to 4 membered).

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂). In embodiments, L⁷ is independently substituted or unsubstituted C₁-C₂₀ alkylene. In embodiments, L⁷ is independently substituted C₁-C₂₀ alkylene. In embodiments, L⁷ is independently hydroxy(OH)-substituted C₁-C₂₀ alkylene. In embodiments, L⁷ is independently hydroxymethyl-substituted C₁-C₂₀ alkylene. In embodiments, L⁷ is independently unsubstituted C₁-C₂₀ alkylene. In embodiments, L⁷ is independently substituted or unsubstituted C₁-C₁₂ alkylene. In embodiments, L⁷ is independently substituted C₁-C₁₂ alkylene. In embodiments, L⁷ is independently hydroxy(OH)-substituted C₁-C₁₂ alkylene. In embodiments, L⁷ is independently hydroxymethyl-substituted C₁-C₁₂ alkylene. In embodiments, L⁷ is independently unsubstituted C₁-C₁₂ alkylene. In embodiments, L⁷ is independently substituted or unsubstituted C₁-C₆ alkylene. In embodiments, L⁷ is independently substituted C₁-C₆ alkylene. In embodiments, L⁷ is independently hydroxy(OH)-substituted C₁-C₆ alkylene. In embodiments, L⁷ is independently hydroxymethyl-substituted C₁-C₆ alkylene. In embodiments, L⁷ is independently unsubstituted C₁-C₆ alkylene. In embodiments, L⁷ is independently substituted or unsubstituted C₁-C₆ alkylene. In embodiments, L⁷ is independently substituted C₁-C₆ alkylene. In embodiments, L⁷ is independently hydroxy(OH)-substituted C₁-C₆ alkylene. In embodiments, L⁷ is independently hydroxymethyl-substituted C₁-C₆ alkylene. In embodiments, L⁷ is independently unsubstituted C₁-C₆ alkylene. In embodiments, L⁷ is independently substituted or unsubstituted C₁-C₄ alkylene. In embodiments, L⁷ is independently substituted C₁-C₄ alkylene. In embodiments, L⁷ is independently hydroxy(OH)-substituted C₁-C₄alkylene. In embodiments, L⁷ is independently hydroxymethyl-substituted C₁-C₄ alkylene. In embodiments, L⁷ is independently unsubstituted C₁-C₄ alkylene. In embodiments, L⁷ is independently substituted or unsubstituted C₁-C₂ alkylene. In embodiments, L⁷ is independently substituted C₁-C₂ alkylene. In embodiments, L⁷ is independently hydroxy(OH)-substituted C₁-C₂ alkylene. In embodiments, L⁷ is independently hydroxymethyl-substituted C₁-C₂ alkylene. In embodiments, L⁷ is independently unsubstituted C₁-C₂alkylene.

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)— or -L⁷-C(O)—NH—; and L⁷ is independently substituted or unsubstituted C₁-C₈ alkylene. In embodiments, L⁴ is independently -L⁷-NH—C(O)— or -L⁷-C(O)—NH—; and L⁷ is independently substituted C₁-C₈ alkylene. In embodiments, L⁴ is independently -L⁷-NH—C(O)— or -L⁷-C(O)—NH—; and L⁷ is independently hydroxy(OH)-substituted C₁-C₈ alkylene. In embodiments, L⁴ is independently -L⁷-NH—C(O)— or -L⁷-C(O)—NH—; and L⁷ is independently hydroxymethyl-substituted C₁-C₈ alkylene. In embodiments, L⁴ is independently -L⁷-NH—C(O)— or -L⁷-C(O)—NH—; and L⁷ is independently unsubstituted C₁-C₈ alkylene.

In embodiments, L⁴ is independently -L⁷-NH—C(O)— or -L⁷-C(O)—NH—; and L⁷ is independently substituted or unsubstituted C₃-C₈ alkylene. In embodiments, L⁴ is independently -L⁷-NH—C(O)— or -L⁷-C(O)—NH—; and L⁷ is independently substituted C₃-C₈ alkylene. In embodiments, L⁴ is independently -L⁷-NH—C(O)— or -L⁷-C(O)—NH—; and L⁷ is independently hydroxy(OH)-substituted C₃-C₈ alkylene. In embodiments, L⁴ is independently -L⁷-NH—C(O)— or -L⁷-C(O)—NH—; and L⁷ is independently hydroxymethyl-substituted C₃-C₈ alkylene. In embodiments, L⁴ is independently -L⁷-NH—C(O)— or -L⁷-C(O)—NH—; and L⁷ is independently unsubstituted C₃-C₈ alkylene.

In embodiments, L⁴ is independently -L⁷-NH—C(O)— or -L⁷-C(O)—NH—; and L⁷ is independently substituted or unsubstituted C₅-C₈alkylene. In embodiments, L⁴ is independently -L⁷-NH—C(O)— or -L⁷-C(O)—NH—; and L⁷ is independently substituted C₅-C₈alkylene. In embodiments, L⁴ is independently -L⁷-NH—C(O)— or -L⁷-C(O)—NH—; and L⁷ is independently hydroxy(OH)-substituted C₅-C₈alkylene. In embodiments, L⁴ is independently -L⁷-NH—C(O)— or -L⁷-C(O)—NH—; and L⁷ is independently hydroxymethyl-substituted C₅-C₈alkylene. In embodiments, L⁴ is independently -L⁷-NH—C(O)— or -L⁷-C(O)—NH—; and L⁷ is independently unsubstituted C₅-C₈alkylene.

In embodiments, L⁴ is independently -L⁷-NH—C(O)— or -L⁷-C(O)—NH—; and L⁷ is independently substituted or unsubstituted octylene. In embodiments, L⁴ is independently -L⁷-NH—C(O)— or -L⁷-C(O)—NH—; and L⁷ is independently substituted octylene. In embodiments, L⁴ is independently -L⁷-NH—C(O)— or -L⁷-C(O)—NH—; and L⁷ is independently hydroxy(OH)-substituted octylene. In embodiments, L⁴ is independently -L⁷-NH—C(O)— or -L⁷-C(O)—NH—; and L⁷ is independently unsubstituted octylene. In embodiments, L⁴ is independently -L⁷-NH—C(O)— and L⁷ is independently hydroxy(OH)-substituted octylene. In embodiments, L⁴ is independently -L⁷-NH—C(O)— and L⁷ is independently hydroxymethyl-substituted octylene. In embodiments, L⁴ is independently -L⁷-NH—C(O)— and L⁷ is independently unsubstituted octylene.

In embodiments, L⁴ is independently -L⁷-NH—C(O)— or -L⁷-C(O)—NH—; and L⁷ is independently substituted or unsubstituted heptylene. In embodiments, L⁴ is independently -L⁷-NH—C(O)— or -L⁷-C(O)—NH—; and L⁷ is independently substituted heptylene. In embodiments, L⁴ is independently -L⁷-NH—C(O)— or -L⁷-C(O)—NH—; and L⁷ is independently hydroxy(OH)-substituted heptylene. In embodiments, L⁴ is independently -L⁷-NH—C(O)— or -L⁷-C(O)—NH—; and L⁷ is independently unsubstituted heptylene. In embodiments, L⁴ is independently -L⁷-NH—C(O)— and L⁷ is independently hydroxy(OH)-substituted heptylene. In embodiments, L⁴ is independently -L⁷-NH—C(O)— and L⁷ is independently hydroxymethyl-substituted heptylene. In embodiments, L⁴ is independently -L⁷-NH—C(O)— and L⁷ is independently unsubstituted heptylene.

In embodiments, L⁴ is independently -L⁷-NH—C(O)— or -L⁷-C(O)—NH—; and L⁷ is independently substituted or unsubstituted hexylene. In embodiments, L⁴ is independently -L⁷-NH—C(O)— or -L⁷-C(O)—NH—; and L⁷ is independently substituted hexylene. In embodiments, L⁴ is independently -L⁷-NH—C(O)— or -L⁷-C(O)—NH—; and L⁷ is independently hydroxy(OH)-substituted hexylene. In embodiments, L⁴ is independently -L⁷-NH—C(O)— or -L⁷-C(O)—NH—; and L⁷ is independently unsubstituted hexylene. In embodiments, L⁴ is independently -L⁷-NH—C(O)— and L⁷ is independently hydroxy(OH)-substituted hexylene. In embodiments, L⁴ is independently -L⁷-NH—C(O)— and L⁷ is independently hydroxymethyl-substituted hexylene. In embodiments, L⁴ is independently -L⁷-NH—C(O)— and L⁷ is independently unsubstituted hexylene.

In embodiments, L⁴ is independently -L⁷-NH—C(O)— or -L⁷-C(O)—NH—; and L⁷ is independently substituted or unsubstituted pentylene. In embodiments, L⁴ is independently -L⁷-NH—C(O)— or -L⁷-C(O)—NH—; and L⁷ is independently substituted pentylene. In embodiments, L⁴ is independently -L⁷-NH—C(O)— or -L⁷-C(O)—NH—; and L⁷ is independently hydroxy(OH)-substituted pentylene. In embodiments, L⁴ is independently -L⁷-NH—C(O)— or -L⁷-C(O)—NH—; and L⁷ is independently unsubstituted pentylene. In embodiments, L⁴ is independently -L⁷-NH—C(O)— and L⁷ is independently hydroxy(OH)-substituted pentylene. In embodiments, L⁴ is independently -L⁷-NH—C(O)— and L⁷ is independently hydroxymethyl-substituted pentylene. In embodiments, L⁴ is independently -L⁷-NH—C(O)— and L⁷ is independently unsubstituted pentylene.

In embodiments, L⁴ is independently

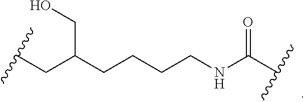 [SMILES: *CC(CO)CCCCNC(*)=O]

In embodiments, L⁴ is independently

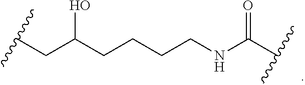 [SMILES: *CC(O)CCCCNC(*)=O]

In embodiments, L⁴ is independently

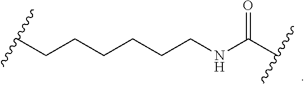 [SMILES: *CCCCCCNC(*)=O]

In embodiments, L⁴ is independently

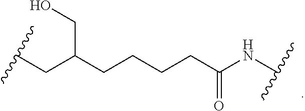 [SMILES: *CC(CO)CCCCC(=O)N*]

In embodiments, L⁴ is independently

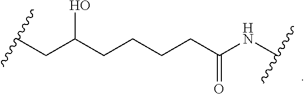 [SMILES: *CC(O)CCCCC(=O)N*]

In embodiments, L⁴ is independently

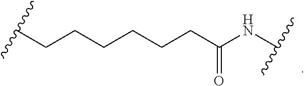 [SMILES: *CCCCCCC(=O)N*]

In embodiments, L⁴ is independently

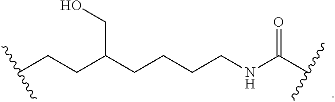 [SMILES: *CCC(CO)CCCCNC(*)=O]

In embodiments, L⁴ is independently

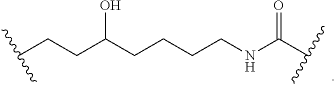 [SMILES: *CCC(O)CCCCNC(*)=O]

In embodiments, L⁴ is independently

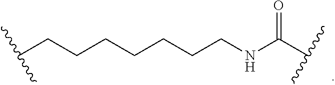 [SMILES: *CCCCCCCNC(*)=O]

In embodiments, L⁴ is independently

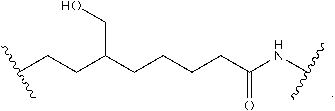 [SMILES: *CCC(CO)CCCCC(=O)N*]

In embodiments, L⁴ is independently

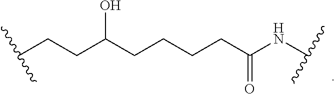 [SMILES: *CCC(O)CCCCC(=O)N*]

In embodiments, L⁴ is independently

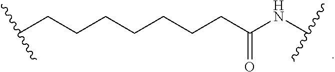 [SMILES: *CCCCCCCC(=O)N*]

In embodiments, -L³-L⁴- is independently -L⁷-NH—C(O)— or -L⁷-C(O)—NH—. In embodiments, L⁷ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered).

In embodiments, L⁷ is independently substituted or unsubstituted 2 to 20 membered heteroalkylene. In embodiments, L⁷ is independently substituted 2 to 20 membered heteroalkylene. In embodiments, L⁷ is independently oxo-substituted 2 to 20 membered heteroalkylene. In embodiments, L⁷ is independently unsubstituted 2 to 20 membered heteroalkylene. In embodiments, L⁷ is independently substituted or unsubstituted 2 to 12 membered heteroalkylene. In embodiments, L⁷ is independently substituted 2 to 12 membered heteroalkylene. In embodiments, L⁷ is independently oxo-substituted 2 to 12 membered heteroalkylene. In embodiments, L⁷ is independently unsubstituted 2 to 12 membered heteroalkylene. In embodiments, L⁷ is independently substituted or unsubstituted 2 to 10 membered heteroalkylene. In embodiments, L⁷ is independently substituted 2 to 10 membered heteroalkylene. In embodiments, L⁷ is independently oxo-substituted 2 to 10 membered heteroalkylene. In embodiments, L⁷ is independently unsubstituted 2 to 10 membered heteroalkylene. In embodiments, L⁷ is independently substituted or unsubstituted 2 to 8 membered heteroalkylene. In embodiments, L⁷ is independently substituted 2 to 8 membered heteroalkylene. In embodiments, L⁷ is independently oxo-substituted 2 to 8 membered heteroalkylene. In embodiments, L⁷ is independently unsubstituted 2 to 8 membered heteroalkylene. In embodiments, L⁷ is independently substituted or unsubstituted 2 to 6 membered heteroalkylene. In embodiments, L⁷ is independently substituted 2 to 6 membered heteroalkylene. In embodiments, L⁷ is independently oxo-substituted 2 to 6 membered heteroalkylene. In embodiments, L⁷ is independently unsubstituted 2 to 6 membered heteroalkylene. In embodiments, L⁷ is independently substituted or unsubstituted 2 to 4 membered heteroalkylene. In embodiments, L⁷ is independently substituted 2 to 4 membered heteroalkylene. In embodiments, L⁷ is independently oxo-substituted 2 to 4 membered heteroalkylene. In embodiments, L⁷ is independently unsubstituted 2 to 4 membered heteroalkylene.

In embodiments, L⁷ is independently substituted or unsubstituted 2 to 20 membered heteroalkenylene. In embodiments, L⁷ is independently substituted 2 to 20 membered heteroalkenylene. In embodiments, L⁷ is independently oxo-substituted 2 to 20 membered heteroalkenylene. In embodiments, L⁷ is independently unsubstituted 2 to 20 membered heteroalkenylene. In embodiments, L⁷ is independently substituted or unsubstituted 2 to 12 membered heteroalkenylene. In embodiments, L⁷ is independently substituted 2 to 12 membered heteroalkenylene. In embodiments, L⁷ is independently oxo-substituted 2 to 12 membered heteroalkenylene. In embodiments, L⁷ is independently unsubstituted 2 to 12 membered heteroalkenylene. In embodiments, L⁷ is independently substituted or unsubstituted 2 to 10 membered heteroalkenylene. In embodiments, L⁷ is independently substituted 2 to 10 membered heteroalkenylene. In embodiments, L⁷ is independently oxo-substituted 2 to 10 membered heteroalkenylene. In embodiments, L⁷ is independently unsubstituted 2 to 10 membered heteroalkenylene. In embodiments, L⁷ is independently substituted or unsubstituted 2 to 8 membered heteroalkenylene. In embodiments, L⁷ is independently substituted 2 to 8 membered heteroalkenylene. In embodiments, L⁷ is independently oxo-substituted 2 to 8 membered heteroalkenylene. In embodiments, L⁷ is independently unsubstituted 2 to 8 membered heteroalkenylene. In embodiments, L⁷ is independently substituted or unsubstituted 2 to 6 membered heteroalkenylene. In embodiments, L⁷ is independently substituted 2 to 6 membered heteroalkenylene. In embodiments, L⁷ is independently oxo-substituted 2 to 6 membered heteroalkenylene. In embodiments, L⁷ is independently unsubstituted 2 to 6 membered heteroalkenylene. In embodiments, L⁷ is independently substituted or unsubstituted 2 to 4 membered heteroalkenylene. In embodiments, L⁷ is independently substituted 2 to 4 membered heteroalkenylene. In embodiments, L⁷ is independently oxo-substituted 2 to 4 membered heteroalkenylene. In embodiments, L⁷ is independently unsubstituted 2 to 4 membered heteroalkenylene.

In embodiments, -L³-L⁴- is independently —O-L⁷-NH—C(O)— or —O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)— or —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently-O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently-O-L⁷-C(O)—NH—; and L⁷ is independently substituted or unsubstituted C₁-C₈ alkylene. In embodiments, -L³-L⁴- is independently-O-L⁷-C(O)—NH—; and L⁷ is independently substituted C₁-C₈ alkylene. In embodiments, -L³-L⁴- is independently-O-L⁷-C(O)—NH—; and L⁷ is independently hydroxy(OH)-substituted C₁-C₈ alkylene. In embodiments, -L³-L⁴- is independently-O-L⁷-C(O)—NH— and L⁷ is independently hydroxymethyl-substituted C₁-C₈ alkylene. In embodiments, -L³-L⁴- is independently-O-L⁷-C(O)—NH—; and L⁷ is independently unsubstituted C₁-C₈ alkylene.

In embodiments, -L³-L⁴- is independently-O-L⁷-C(O)—NH—; and L⁷ is independently substituted or unsubstituted C₃-C₈ alkylene. In embodiments, -L³-L⁴- is independently-O-L⁷-C(O)—NH—; and L⁷ is independently substituted C₃-C₈ alkylene. In embodiments, -L³-L⁴- is independently-O-L⁷-C(O)—NH—; and L⁷ is independently hydroxy(OH)-substituted C₃-C₈ alkylene. In embodiments, -L³-L⁴- is independently-O-L⁷-C(O)—NH— and L⁷ is independently hydroxymethyl-substituted C₃-C₈ alkylene. In embodiments, -L³-L⁴- is independently-O-L⁷-C(O)—NH—; and L⁷ is independently unsubstituted C₃-C₈ alkylene.

In embodiments, -L³-L⁴- is independently-O-L⁷-C(O)—NH—; and L⁷ is independently substituted or unsubstituted C₅-C₈ alkylene. In embodiments, -L³-L⁴- is independently-O-L⁷-C(O)—NH—; and L⁷ is independently substituted C₅-C₈ alkylene. In embodiments, -L³-L⁴- is independently-O-L⁷-C(O)—NH—; and L⁷ is independently hydroxy(OH)-substituted C₅-C₈ alkylene. In embodiments, -L³-L⁴- is independently-O-L⁷-C(O)—NH— and L⁷ is independently hydroxymethyl-substituted C₅-C₈ alkylene. In embodiments, -L³-L⁴- is independently-O-L⁷-C(O)—NH—; and L⁷ is independently unsubstituted C₅-C₈ alkylene.

In embodiments, -L³-L⁴- is independently —O-L⁷-NH—C(O)—; and L⁷ is independently substituted or unsubstituted C₁-C₈ alkylene. In embodiments, -L³-L⁴- is independently —O-L⁷-NH—C(O)—; and L⁷ is independently substituted C₁-C₈ alkylene. In embodiments, -L³-L⁴- is independently —O-L⁷-NH—C(O)—; and L⁷ is independently hydroxy(OH)-substituted C₁-C₈ alkylene. In embodiments, -L³-L⁴- is independently —O-L⁷-NH—C(O)—; and L⁷ is independently hydroxymethyl-substituted C₁-C₈ alkylene. In embodiments, -L³-L⁴- is independently —O-L⁷-NH—C(O)—; and L⁷ is independently unsubstituted C₁-C₈ alkylene.

In embodiments, -L³-L⁴- is independently —O-L⁷-NH—C(O)—; and L⁷ is independently substituted or unsubstituted C₃-C₈ alkylene. In embodiments, -L³-L⁴- is independently —O-L⁷-NH—C(O)—; and L⁷ is independently substituted C₃-C₈ alkylene. In embodiments, -L³-L⁴- is independently —O-L⁷-NH—C(O)—; and L⁷ is independently hydroxy(OH)-substituted C₃-C₈ alkylene. In embodiments, -L³-L⁴- is independently —O-L⁷-NH—C(O)—; and L⁷ is independently hydroxymethyl-substituted C₃-C₈ alkylene. In embodiments, -L³-L⁴- is independently —O-L⁷-NH—C(O)—; and L⁷ is independently unsubstituted C₃-C₈ alkylene.

In embodiments, -L³-L⁴- is independently —O-L⁷-NH—C(O)—; and L⁷ is independently substituted or unsubstituted C₅-C₈ alkylene. In embodiments, -L³-L⁴- is independently —O-L⁷-NH—C(O)—; and L⁷ is independently substituted C₅-C₈ alkylene. In embodiments, -L³-L⁴- is independently —O-L⁷-NH—C(O)—; and L⁷ is independently hydroxy(OH)-substituted C₅-C₈ alkylene. In embodiments, -L³-L⁴- is independently —O-L⁷-NH—C(O)—; and L⁷ is independently hydroxymethyl-substituted C₅-C₈ alkylene. In embodiments, -L³-L⁴- is independently —O-L⁷-NH—C(O)—; and L⁷ is independently unsubstituted C₅-C₈ alkylene.

In embodiments, -L³-L⁴- is independently

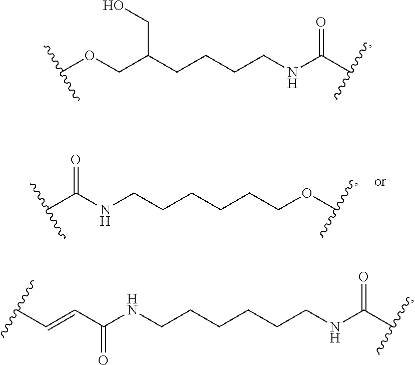 [SMILES: *OCC(CO)CCCCNC(*)=O ; *OCCCCCCNC(*)=O ; */C=C/C(=O)NCCCCCCNC(*)=O]

In embodiments, -L³-L⁴- is independently

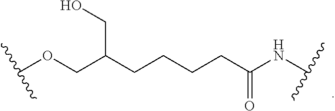 [SMILES: *NC(=O)CCCCC(CO)CO*]

In embodiments, -L³-L⁴- is independently

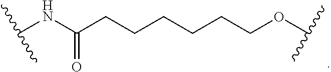 [SMILES: *NC(=O)CCCCCCO*]

In embodiments, -L³-L⁴- is independently

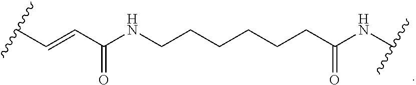 [SMILES: */C=C/C(=O)NCCCCCCC(=O)N*]

In embodiments, -L³-L⁴- is independently —OPO₂—O-L⁷-NH—C(O)— or —OPO₂—O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)— or —OPO₂—O-L⁷-C(O)—NH—; and L⁷ is independently substituted or unsubstituted alkylene. In embodiments, -L³-L⁴- is independently —OPO₂—O-L⁷-NH—C(O)—; and L⁷ is independently substituted or unsubstituted alkylene. In embodiments, -L³-L⁴- is independently —OPO₂—O-L⁷-C(O)—NH—; and L⁷ is independently substituted or unsubstituted alkylene. In embodiments, -L³-L⁴- is independently —OPO₂—O-L⁷-NH—C(O)— or —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OPO₂—O-L⁷-C(O)—NH—; and L⁷ is independently substituted or unsubstituted C₁-C₈ alkylene. In embodiments, -L³-L⁴- is independently —OPO₂—O-L⁷-C(O)—NH—; and L⁷ is independently substituted C₁-C₈ alkylene. In embodiments, -L³-L⁴- is independently —OPO₂—O-L⁷-C(O)—NH—; and L⁷ is independently hydroxy(OH)-substituted C₁-C₈ alkylene. In embodiments, -L³-L⁴- is independently —OPO₂—O-L⁷-C(O)—NH—; and L⁷ is independently hydroxymethyl-substituted C₁-C₈ alkylene. In embodiments, -L³-L⁴- is independently —OPO₂—O-L⁷-C(O)—NH—; and L⁷ is independently unsubstituted C₁-C₈ alkylene.

In embodiments, -L³-L⁴- is independently —OPO₂—O-L⁷-C(O)—NH—; and L⁷ is independently substituted or unsubstituted C₃-C₈ alkylene. In embodiments, -L³-L⁴- is independently —OPO₂—O-L⁷-C(O)—NH—; and L⁷ is independently substituted C₃-C₈ alkylene. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is independently —OPO₂—O-L⁷-C(O)—NH—; and L⁷ is independently hydroxymethyl-substituted C₃-C₈ alkylene. In embodiments, -L³-L⁴- is independently —OPO₂—O-L⁷-C(O)—NH—; and L⁷ is independently unsubstituted C₃-C₈ alkylene.

In embodiments, -L³-L⁴- is independently —OPO₂—O-L⁷-C(O)—NH—; and L⁷ is independently substituted or unsubstituted C₅-C₈ alkylene. In embodiments, -L³-L⁴- is independently —OPO₂—O-L⁷-C(O)—NH—; and L⁷ is independently substituted C₃-C₈ alkylene. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is independently —OPO₂—O-L⁷-C(O)—NH—; and L⁷ is independently hydroxymethyl-substituted C₅-C₈ alkylene. In embodiments, -L³-L⁴- is independently —OPO₂—O-L⁷-C(O)—NH—; and L⁷ is independently unsubstituted C₁-C₈ alkylene.

In embodiments, -L³-L⁴- is independently —OPO₂—O-L⁷-NH—C(O)—; and L⁷ is independently substituted or unsubstituted C₁-C₈ alkylene. In embodiments, -L³-L⁴- is independently —OPO₂—O-L⁷-NH—C(O)—; and L⁷ is independently substituted C₃-C₈ alkylene. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is independently —OPO₂—O-L⁷-NH—C(O)—; and L⁷ is independently hydroxymethyl-substituted C₃-C₈ alkylene. In embodiments, -L³-L⁴- is independently —OPO₂—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₃-C₈ alkylene. In embodiments, -L³-L⁴- is independently —OPO₂—O-L⁷-NH—C(O)—; and L⁷ is independently substituted C₃-C₈ alkylene. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is independently —OPO₂—O-L⁷-NH—C(O)—; and L⁷ is independently hydroxymethyl-substituted C₃—C₈ alkylene. In embodiments, -L³-L⁴- is independently —OPO₂—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₅-C₈ alkylene. In embodiments, -L³-L⁴- is independently —OPO₂—O-L⁷-NH—C(O)—; and L⁷ is independently substituted C₅-C₈ alkylene. In embodiments, -L³-L⁴- is independently —OPO₂—O-L⁷-NH—C(O)—; and L⁷ is independently hydroxy(OH)-substituted C₅-C₈ alkylene. In embodiments, -L³-L⁴- is independently —OPO₂—O-L⁷-NH—C(O)—; and L⁷ is independently hydroxymethyl-substituted C₅-C₈ alkylene. In embodiments, -L³-L⁴- is independently —OPO₂—O-L⁷-NH—C(O)—; and L⁷ is independently unsubstituted C₅-C₈ alkylene.

In embodiments, -L³-L⁴- is independently

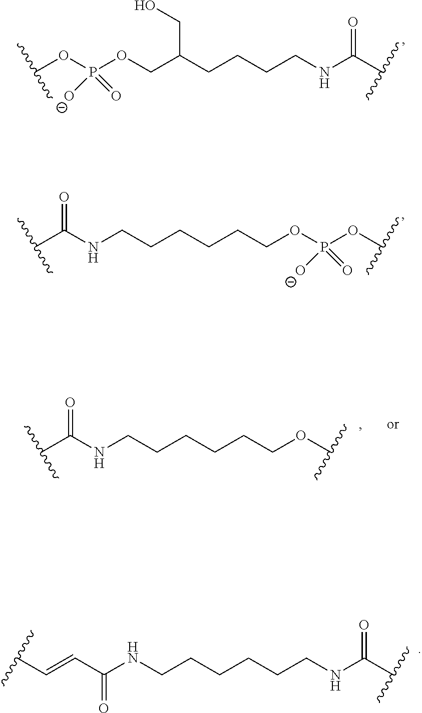 [SMILES: *OP(=O)([O-])OCC(CO)CCCCNC(*)=O ; *OP(=O)([O-])OCCCCCCNC(*)=O ; *OCCCCCCNC(*)=O ; */C=C/C(=O)NCCCCCCNC(*)=O]

In embodiments, -L³-L⁴- is independently

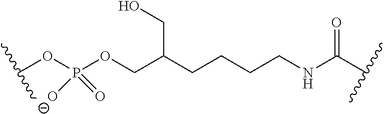 [SMILES: *OP(=O)([O-])OCC(CO)CCCCNC(*)=O]

and is attached to a 3′ carbon of the double-stranded oligonucleotide or single-stranded oligonucleotide.
In embodiments, -L³-L⁴- is independently

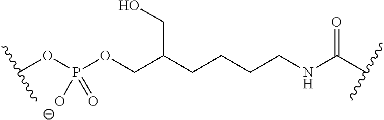 [SMILES: *OP(=O)([O-])OCC(CO)CCCCNC(*)=O]

and is attached to a 5′ carbon of the double-stranded oligonucleotide or single-stranded oligonucleotide. In embodiments, -L³-L⁴- is independently

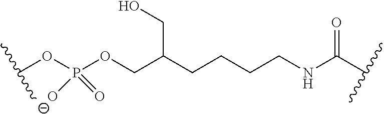 [SMILES: *OP(=O)([O-])OCC(CO)CCCCNC(*)=O]

and is attached to a 2′ carbon of the double-stranded oligonucleotide or single-stranded oligonucleotide. In embodiments, -L³-L⁴- is independently

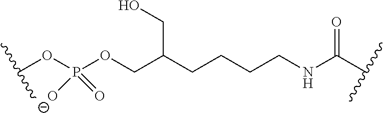 [SMILES: *OP(=O)([O-])OCC(CO)CCCCNC(*)=O]

and is attached to a nucleobase of the double-stranded oligonucleotide or single-stranded oligonucleotide. In embodiments, -L³-L⁴- is independently

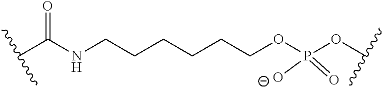 [SMILES: *OP(=O)([O-])OCCCCCCNC(*)=O]

and is attached to a 3′ carbon of the double-stranded oligonucleotide or single-stranded oligonucleotide. In embodiments, -L³-L⁴- is independently

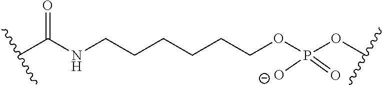 [SMILES: *OP(=O)([O-])OCCCCCCNC(*)=O]

and is attached to a 5′ carbon of the double-stranded oligonucleotide or single-stranded oligonucleotide. In embodiments, -L³-L⁴- is independently

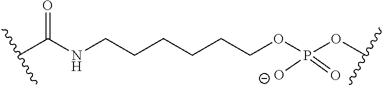 [SMILES: *OP(=O)([O-])OCCCCCCNC(*)=O]

and is attached to a 2′ carbon of the double-stranded oligonucleotide or single-stranded oligonucleotide. In embodiments, -L³-L⁴- is independently

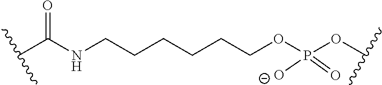 [SMILES: *OP(=O)([O-])OCCCCCCNC(*)=O]

and is attached to a nucleobase of the double-stranded oligonucleotide or single-stranded oligonucleotide. In embodiments, -L³-L⁴- is independently

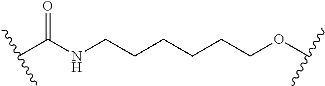 [SMILES: *OCCCCCCNC(*)=O]

and is attached to a 3′ carbon of the double-stranded oligonucleotide or single-stranded oligonucleotide. In embodiments, -L³-L⁴- is independently

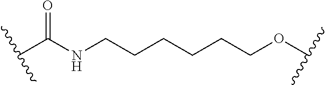 [SMILES: *OCCCCCCNC(*)=O]

and is attached to a 5′ carbon of the double-stranded oligonucleotide or single-stranded oligonucleotide. In embodiments, -L³-L⁴- is independently

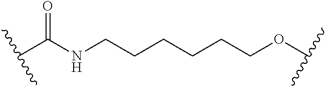 [SMILES: *OCCCCCCNC(*)=O]

and is attached to a 2′ carbon of the double-stranded oligonucleotide or single-stranded oligonucleotide. In embodiments, -L³-L⁴- is independently

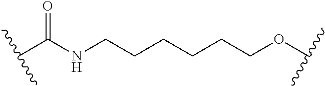 [SMILES: *OCCCCCCNC(*)=O]

and is attached to a nucleobase of the double-stranded oligonucleotide or single-stranded oligonucleotide. In embodiments, -L³-L⁴- is independently

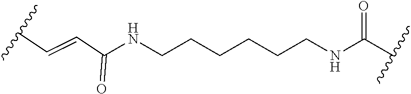 [SMILES: */C=C/C(=O)NCCCCCCNC(*)=O]

and is attached to a 3′ carbon of the double-stranded oligonucleotide or single-stranded oligonucleotide. In embodiments, -L³-L⁴- is independently

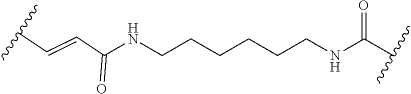 [SMILES: */C=C/C(=O)NCCCCCCNC(*)=O]

and is attached to a 5′ carbon of the double-stranded oligonucleotide or single-stranded oligonucleotide. In embodiments, -L³-L⁴- is independently

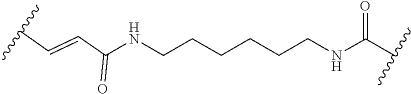 [SMILES: */C=C/C(=O)NCCCCCCNC(*)=O]

and is attached to a 2′ carbon of the double-stranded oligonucleotide or single-stranded oligonucleotide. In embodiments, -L³-L⁴- is independently

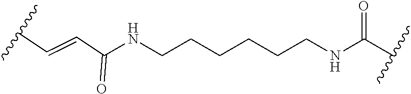 [SMILES: */C=C/C(=O)NCCCCCCNC(*)=O]

and is attached to a nucleobase of the double-stranded oligonucleotide or single-stranded oligonucleotide.

In embodiments, R³ is independently hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen. In embodiments, R³ is independently —NH₂. In embodiments, R³ is independently —OH. In embodiments, R³ is independently —SH. In embodiments, R³ is independently —C(O)H. In embodiments, R³ is independently —C(O)NH₂. In embodiments, R³ is independently —NHC(O)H. In embodiments, R³ is independently —NHC(O)OH. In embodiments, R³ is independently —NHC(O)NH₂. In embodiments, R³ is independently —C(O)OH. In embodiments, R³ is independently —OC(O)H. In embodiments, R³ is independently —N₃.

In embodiments, R³ is independently substituted or unsubstituted alkyl (e.g., C₁-C₂₀, C₁-C₁₂, C₁-C₈, C₁-C₆, C₁-C₄, or C₁-C₂). In embodiments, R³ is independently substituted or unsubstituted C₁-C₂₀ alkyl. In embodiments, R³ is independently substituted C₁-C₂₀ alkyl. In embodiments, R³ is independently unsubstituted C₁-C₂₀ alkyl. In embodiments, R³ is independently substituted or unsubstituted C₁-C₁₂ alkyl. In embodiments, R³ is independently substituted C₁-C₁₂ alkyl. In embodiments, R³ is independently unsubstituted C₁-C₁₂ alkyl. In embodiments, R³ is independently substituted or unsubstituted C₁-C₈ alkyl. In embodiments, R³ is independently substituted C₁-C₈ alkyl. In embodiments, R³ is independently unsubstituted C₁—C₈ alkyl. In embodiments, R³ is independently substituted or unsubstituted C₁-C₆ alkyl. In embodiments, R³ is independently substituted C₁-C₆ alkyl. In embodiments, R³ is independently unsubstituted C₁-C₆ alkyl. In embodiments, R³ is independently substituted or unsubstituted C₁-C₄ alkyl. In embodiments, R³ is independently substituted C₁-C₄ alkyl. In embodiments, R³ is independently unsubstituted C₁-C₄ alkyl. In embodiments, R³ is independently substituted or unsubstituted ethyl. In embodiments, R³ is independently substituted ethyl. In embodiments, R³ is independently unsubstituted ethyl. In embodiments, R³ is independently substituted or unsubstituted methyl. In embodiments, R³ is independently substituted methyl. In embodiments, R³ is independently unsubstituted methyl.

In embodiments, L⁶ is independently —NHC(O)—. In embodiments, L⁶ is independently —C(O)NH—. In embodiments, L⁶ is independently substituted or unsubstituted alkylene. In embodiments, L⁶ is independently substituted or unsubstituted heteroalkylene.

In embodiments, L⁶ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁶ is independently unsubstituted alkylene (e.g., C₁-C₂₀, C₁-C₁₂, C₁-C₈, C₁-C₆, C₁-C₄, or C₁-C₂). In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, L⁶ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L^{6A} is independently a bond or unsubstituted alkylene; L^{6B} is independently a bond, —NHC(O)—, or unsubstituted arylene; L^{6C} is independently a bond, unsubstituted alkylene, or unsubstituted arylene; L^{6D} is independently a bond or unsubstituted alkylene; and L^{6E} is independently a bond or —NHC(O)—. In embodiments, L^{6A} is independently a bond or unsubstituted alkylene. In embodiments, L^{6B} is independently a bond, —NHC(O)—, or unsubstituted arylene. In embodiments, L^{6C} is independently a bond, unsubstituted alkylene, or unsubstituted arylene. In embodiments, L^{6D} is independently a bond or unsubstituted alkylene. In embodiments, L^{6E} is independently a bond or —NHC(O)—.

In embodiments, L^{6A} is independently a bond or unsubstituted alkylene (e.g., C₁-C₂₀, C₁-C₁₂, C₁-C₈, C₁-C₆, C₁-C₄, or C₁-C₂). In embodiments, L^{6A} is independently unsubstituted C₁-C₂₀ alkylene. In embodiments, L^{6A} is independently unsubstituted C₁-C₁₂ alkylene. In embodiments, L^{6A} is independently unsubstituted C₁-C₈ alkylene. In embodiments, L^{6A} is independently unsubstituted C₁-C₆ alkylene. In embodiments, L^{6A} is independently unsubstituted C₁-C₄ alkylene. In embodiments, L^{6A} is independently unsubstituted ethylene. In embodiments, L^{6A} is independently unsubstituted methylene. In embodiments, L^{6A} is independently a bond.

In embodiments, L^{6B} is independently a bond. In embodiments, L^{6B} is independently —NHC(O)—. In embodiments, L^{6B} is independently unsubstituted arylene (e.g., C₆-C₁₂, C₆-C₁₀, or phenyl). In embodiments, L^{6B} is independently unsubstituted C₆-C₁₂ arylene. In embodiments, L^{6B} is independently unsubstituted C₆-C₁₀ arylene. In embodiments, L^{6B} is independently unsubstituted phenylene. In embodiments, L^{6B} is independently unsubstituted naphthylene.

In embodiments, L^{6C} is independently a bond or unsubstituted alkylene (e.g., C₁-C₂₀, C₁-C₁₂, C₁-C₈, C₁-C₆, C₁-C₄, or C₁-C₂). In embodiments, L^{6C} is independently unsubstituted C₁-C₂₀ alkylene. In embodiments, L^{6C} is independently unsubstituted C₁-C₁₂ alkylene. In embodiments, L^{6C} is independently unsubstituted C₁-C₈ alkylene. L^{6C} is independently unsubstituted C₂-C₈ alkynylene. In embodiments, L^{6C} is independently unsubstituted C₁-C₆ alkylene. In embodiments, L^{6C} is independently unsubstituted C₁-C₄ alkylene. In embodiments, L^{6C} is independently unsubstituted ethylene. In embodiments, L^{6C} is independently unsubstituted methylene. In embodiments, L^{6C} is independently a bond or unsubstituted alkynylene (e.g., C₂-C₂₀, C₂-C₁₂, C₂-C₈, C₂-C₆, C₂-C₄, or C₂-C₂). In embodiments, L^{6C} is independently unsubstituted C₂-C₂₀ alkynylene. In embodiments, L^{6C} is independently unsubstituted C₂-C₁₂ alkynylene. In embodiments, L^{6C} is independently unsubstituted C₂-C₈ alkynylene. In embodiments, L^{6C} is independently unsubstituted C₂-C₆ alkynylene. In embodiments, L^{6C} is independently unsubstituted C₂-C₄ alkynylene. In embodiments, L^{6C} is independently unsubstituted ethynylene. In embodiments, L^{6C} is independently unsubstituted arylene (e.g., C₆-C₁₂, C₆-C₁₀, or phenyl). In embodiments, L^{6C} is independently unsubstituted C₆-C₁₂ arylene. In embodiments, L^{6C} is independently unsubstituted C₆-C₁₀ arylene. In embodiments, L^{6C} is independently unsubstituted phenylene. In embodiments, L^{6C} is independently unsubstituted naphthylene. In embodiments, L^{6C} is independently a bond.

In embodiments, L^{6D} is independently a bond or unsubstituted alkylene (e.g., C₁-C₂₀, C₁-C₁₂, C₁-C₈, C₁-C₆, C₁-C₄, or C₁-C₂). In embodiments, L^{6D} is independently unsubstituted C₁—C₂₀ alkylene. In embodiments, L^{6D} is independently unsubstituted C₁-C₁₂ alkylene. In embodiments, L^{6A} is independently unsubstituted C₁-C₈ alkylene. In embodiments, L^{6D} is independently unsubstituted C₁-C₆ alkylene. In embodiments, L^{6D} is independently unsubstituted C₁-C₄ alkylene. In embodiments, L^{6D} is independently unsubstituted ethylene. In embodiments, L^{6D} is independently unsubstituted methylene. In embodiments, L^{6D} is independently a bond.

In embodiments, L^{6E} is independently a bond. In embodiments, L^{6E} is independently —NHC(O)—.

In embodiments, L^{6A} is independently a bond or unsubstituted C₁-C₈ alkylene. In embodiments, L^{6B} is independently a bond, —NHC(O)—, or unsubstituted phenylene. In embodiments, L^{6C} is independently a bond, unsubstituted C₂-C₈ alkynylene, or unsubstituted phenylene. In embodiments, L^{6D} is independently a bond or unsubstituted C₁-C₈ alkylene. In embodiments, L^{6E} is independently a bond or —NHC(O)—.

In embodiments, L⁶ is independently a bond,

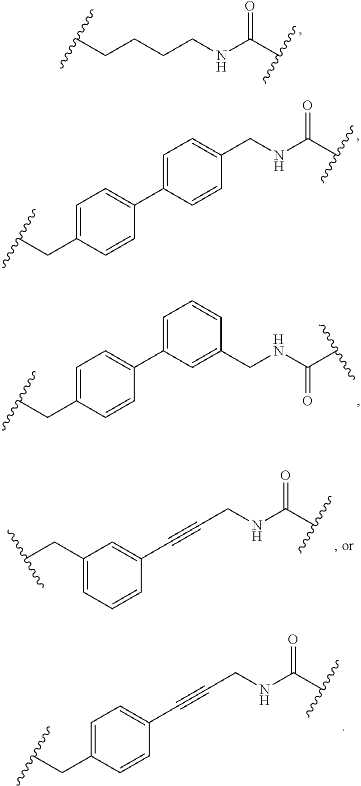 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁶ is independently a bond. In embodiments, L⁶ is independently

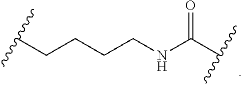 [SMILES: *CCCCNC(*)=O]

In embodiments, L⁶ is independently

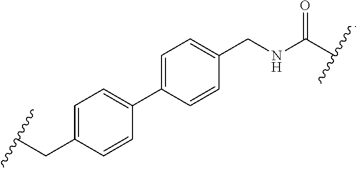 [SMILES: *Cc1ccc(-c2ccc(CNC(*)=O)cc2)cc1]

In embodiments, L⁶ is independently

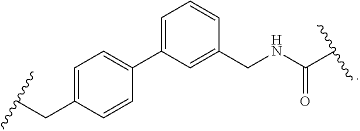 [SMILES: *Cc1ccc(-c2cccc(CNC(*)=O)c2)cc1]

In embodiments, L⁶ is independently

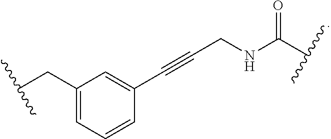 [SMILES: *Cc1cccc(C#CCNC(*)=O)c1]

In embodiments, L⁶ is independently

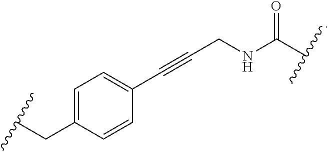 [SMILES: *Cc1ccc(C#CCNC(*)=O)cc1]

In embodiments, L⁵ is independently —NHC(O)—. In embodiments, L⁵ is independently —C(O)NH—. In embodiments, L⁵ is independently substituted or unsubstituted alkylene. In embodiments, L⁵ is independently substituted or unsubstituted heteroalkylene.

In embodiments, L⁵ is independently substituted or unsubstituted alkylene (e.g., C₁-C₂₀, C₁-C₁₂, C₁-C₈, C₁-C₆, C₁-C₄, or C₁-C₂). In embodiments, L⁵ is independently substituted alkylene (e.g., C₁-C₂₀, C₁-C₁₂, C₁-C₈, C₁-C₆, C₁-C₄, or C₁-C₂). In embodiments, L⁵ is independently unsubstituted alkylene (e.g., C₁-C₂₀, C₁-C₁₂, C₁-C₈, C₁-C₆, C₁-C₄, or C₁-C₂). In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene.

In embodiments, L⁵ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L^{5A} is independently a bond or unsubstituted alkylene; L^{5B} is independently a bond, —NHC(O)—, or unsubstituted arylene; L^{5C} is independently a bond, unsubstituted alkylene, or unsubstituted arylene; L^{5D} is independently a bond or unsubstituted alkylene; and L^{5E} is independently a bond or —NHC(O)—. In embodiments, L^{5A} is independently a bond or unsubstituted alkylene. In embodiments, L^{5B} is independently a bond, —NHC(O)—, or unsubstituted arylene. In embodiments, L^{5C} is independently a bond, unsubstituted alkylene, or unsubstituted arylene. In embodiments, L^{5D} is independently a bond or unsubstituted alkylene. In embodiments, L^{5E} is independently a bond or —NHC(O)—.

In embodiments, L^{5A} is independently a bond or unsubstituted alkylene (e.g., C₁-C₂₀, C₁-C₁₂, C₁-C₈, C₁-C₆, C₁-C₄, or C₁-C₂). In embodiments, L^{5A} is independently unsubstituted C₁-C₂₀ alkylene. In embodiments, L^{5A} is independently unsubstituted C₁-C₁₂ alkylene. In embodiments, L^{5A} is independently unsubstituted C₁-C₈ alkylene. In embodiments, L^{5A} is independently unsubstituted C₁-C₆ alkylene. In embodiments, L^{5A} is independently unsubstituted C₁-C₄ alkylene. In embodiments, L^{5A} is independently unsubstituted ethylene. In embodiments, L^{5A} is independently unsubstituted methylene. In embodiments, L^{5A} is independently a bond.

In embodiments, L^{5B} is independently a bond. In embodiments, L^{5B} is independently —NHC(O)—. In embodiments, L^{5B} is independently unsubstituted arylene (e.g., C₆-C₁₂, C₆-C₁₀, or phenyl). In embodiments, L^{5B} is independently unsubstituted C₆-C₁₂ arylene. In embodiments, L^{5B} is independently unsubstituted C₆-C₁₀ arylene. In embodiments, L^{5B} is independently unsubstituted phenylene. In embodiments, L^{5B} is independently unsubstituted naphthylene.

In embodiments, L^{5C} is independently a bond or unsubstituted alkylene (e.g., C₁-C₂₀, C₁-C₁₂, C₁-C₈, C₁-C₆, C₁-C₄, or C₁-C₂). In embodiments, L^{5C} is independently unsubstituted C₁-C₂₀ alkylene. In embodiments, L^{5C} is independently unsubstituted C₁-C₁₂ alkylene. In embodiments, L^{5C} is independently unsubstituted C₁-C₈ alkylene. L^{5C} is independently unsubstituted C₂-C₈ alkynylene. In embodiments, L^{5C} is independently unsubstituted C₁-C₆ alkylene. In embodiments, L^{5C} is independently unsubstituted C₁-C₄ alkylene. In embodiments, L^{5C} is independently unsubstituted ethylene. In embodiments, L^{5C} is independently unsubstituted methylene. In embodiments, L^{5C} is independently a bond or unsubstituted alkynylene (e.g., C₂-C₂₀, C₂-C₁₂, C₂-C₈, C₂-C₆, C₂-C₄, or C₂-C₂). In embodiments, L^{5C} is independently unsubstituted C₂-C₂₀ alkynylene. In embodiments, L^{5C} is independently unsubstituted C₂-C₁₂ alkynylene. In embodiments, L^{5C} is independently unsubstituted C₂-C₈ alkynylene. In embodiments, L^{5C} is independently unsubstituted C₂-C₆ alkynylene. In embodiments, L^{5C} is independently unsubstituted C₂-C₄ alkynylene. In embodiments, L^{5C} is independently unsubstituted ethynylene. In embodiments, L^{5C} is independently unsubstituted arylene (e.g., C₆-C₁₂, C₆-C₁₀, or phenyl). In embodiments, L^{5C} is independently unsubstituted C₆-C₁₂ arylene. In embodiments, L^{5C} is independently unsubstituted C₆-C₁₀ arylene. In embodiments, L^{5C} is independently unsubstituted phenylene. In embodiments, L^{5C} is independently unsubstituted naphthylene. In embodiments, L^{5C} is independently a bond.

In embodiments, L^{5D} is independently a bond or unsubstituted alkylene (e.g., C₁-C₂₀, C₁-C₁₂, C₁-C₈, C₁-C₆, C₁-C₄, or C₁-C₂). In embodiments, L^{5D} is independently unsubstituted C₁-C₂₀ alkylene. In embodiments, L^{5D} is independently unsubstituted C₁-C₁₂ alkylene. In embodiments, L^{5A} is independently unsubstituted C₁-C₈ alkylene. In embodiments, L^{5D} is independently unsubstituted C₁-C₆ alkylene. In embodiments, L^{5D} is independently unsubstituted C₁-C₄ alkylene. In embodiments, L^{5D} is independently unsubstituted ethylene. In embodiments, L^{5D} is independently unsubstituted methylene. In embodiments, L^{5D} is independently a bond.

In embodiments, L^{5E} is independently a bond. In embodiments, L^{5E} is independently —NHC(O)—.

In embodiments, L^{5A} is independently a bond or unsubstituted C₁-C₈ alkylene. In embodiments, L^{5B} is independently a bond, —NHC(O)—, or unsubstituted phenylene. In embodiments, L^{5C} is independently a bond, unsubstituted C₂-C₈ alkynylene, or unsubstituted phenylene. In embodiments, L^{5D} is independently a bond or unsubstituted C₁-C₈ alkylene. In embodiments, L^{5E} is independently a bond or —NHC(O)—.

In embodiments, L⁵ is independently a bond,

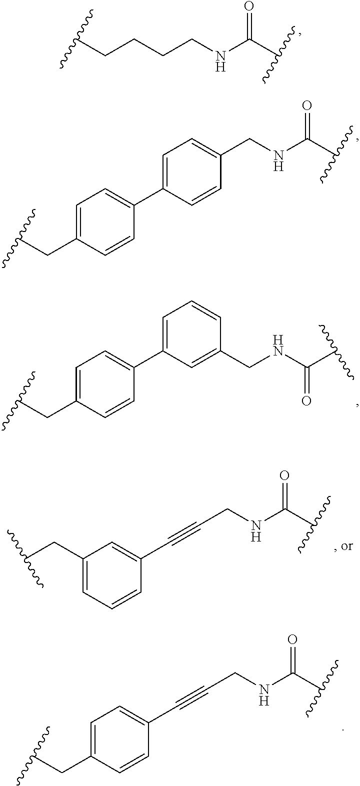 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁵ is independently a bond. In embodiments, L⁵ is independently

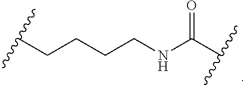 [SMILES: *CCCCNC(*)=O]

In embodiments, L⁵ is independently

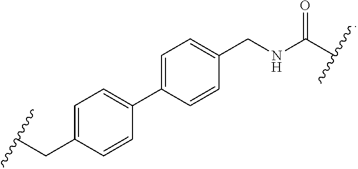 [SMILES: *Cc1ccc(-c2ccc(CNC(*)=O)cc2)cc1]

In embodiments, L⁵ is independently

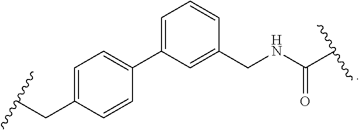 [SMILES: *Cc1ccc(-c2cccc(CNC(*)=O)c2)cc1]

In embodiments, L⁵ is independently

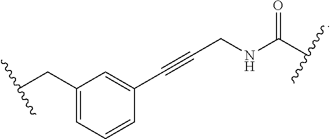 [SMILES: *Cc1cccc(C#CCNC(*)=O)c1]

In embodiments, L⁵ is independently

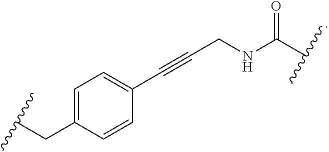 [SMILES: *Cc1ccc(C#CCNC(*)=O)cc1]

In embodiments, R¹ is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R¹ is unsubstituted C₁-C₁₇ alkyl. In embodiments, R¹ is unsubstituted C₁₁-C₁₇ alkyl. In embodiments, R¹ is unsubstituted C₁₃-C₁₇ alkyl. In embodiments, R¹ is unsubstituted C₁₅ alkyl. In embodiments, R¹ is unsubstituted unbranched C₁-C₁₇ alkyl. In embodiments, R¹ is unsubstituted unbranched C₁₁-C₁₇ alkyl. In embodiments, R¹ is unsubstituted unbranched C₁₃-C₁₇ alkyl. In embodiments, R¹ is unsubstituted unbranched C₁₅ alkyl. In embodiments, R¹ is unsubstituted unbranched saturated C₁-C₁₇ alkyl. In embodiments, R¹ is unsubstituted unbranched saturated C₁₁-C₁₇ alkyl. In embodiments, R¹ is unsubstituted unbranched saturated C₁₃-C₁₇ alkyl. In embodiments, R¹ is unsubstituted unbranched saturated C₁₅ alkyl. In embodiments, R¹ is unsubstituted unbranched unsaturated C₁-C₁₇ alkyl. In embodiments, R¹ is unsubstituted unbranched unsaturated C₁₁-C₁₇ alkyl. In embodiments, R¹ is unsubstituted unbranched unsaturated C₁₃-C₁₇ alkyl. In embodiments, R¹ is unsubstituted unbranched unsaturated C₁₅ alkyl.

In embodiments, R² is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R² is unsubstituted C₁-C₁₇ alkyl. In embodiments, R² is unsubstituted C₁₁-C₁₇ alkyl. In embodiments, R² is unsubstituted C₁₃-C₁₇ alkyl. In embodiments, R² is unsubstituted C₁₅ alkyl. In embodiments, R² is unsubstituted unbranched C₁-C₁₇ alkyl. In embodiments, R² is unsubstituted unbranched C₁₁-C₁₇ alkyl. In embodiments, R² is unsubstituted unbranched C₁₃-C₁₇ alkyl. In embodiments, R² is unsubstituted unbranched C₁₅ alkyl. In embodiments, R² is unsubstituted unbranched saturated C₁-C₁₇ alkyl. In embodiments, R³ is unsubstituted unbranched saturated C₁₁-C₁₇ alkyl. In embodiments, R² is unsubstituted unbranched saturated C₁₃-C₁₇ alkyl. In embodiments, R² is unsubstituted unbranched saturated C₁₅ alkyl. In embodiments, R² is unsubstituted unbranched unsaturated C₁-C₁₇ alkyl. In embodiments, R² is unsubstituted unbranched unsaturated C₁₁-C₁₇ alkyl. In embodiments, R² is unsubstituted unbranched unsaturated C₁₃-C₁₇ alkyl. In embodiments, R² is unsubstituted unbranched unsaturated C₁₅ alkyl.

In embodiments, at least one of R¹ and R² is unsubstituted C₁-C₁₉ alkyl. In embodiments, at least one of R¹ and R² is unsubstituted C₉-C₁₉ alkyl. In embodiments, at least one of R¹ and R² is unsubstituted C₁₁-C₁₉ alkyl. In embodiments, at least one of R¹ and R² is unsubstituted C₁₃-C₁₉ alkyl.

In embodiments, R¹ is unsubstituted C₁-C₁₉ alkyl. In embodiments, R¹ is unsubstituted C₉-C₁₉ alkyl. In embodiments, R¹ is unsubstituted C₁₁-C₁₉ alkyl. In embodiments, R¹ is unsubstituted C₁₃-C₁₉ alkyl. In embodiments, R¹ is unsubstituted unbranched C₁-C₁₉ alkyl. In embodiments, R¹ is unsubstituted unbranched C₉-C₁₉ alkyl. In embodiments, R¹ is unsubstituted unbranched C₁₁-C₁₉ alkyl. In embodiments, R¹ is unsubstituted unbranched C₁₃-C₁₉ alkyl. In embodiments, R¹ is unsubstituted unbranched saturated C₁-C₁₉ alkyl. In embodiments, R¹ is unsubstituted unbranched saturated C₉-C₁₉ alkyl. In embodiments, R¹ is unsubstituted unbranched saturated C₁₁-C₁₉ alkyl. In embodiments, R¹ is unsubstituted unbranched saturated C₁₃-C₁₉ alkyl. In embodiments, R¹ is unsubstituted unbranched unsaturated C₁-C₁₉ alkyl. In embodiments, R¹ is unsubstituted unbranched unsaturated C₉-C₁₉ alkyl. In embodiments, R¹ is unsubstituted unbranched unsaturated C₁₁-C₁₉ alkyl. In embodiments, R¹ is unsubstituted unbranched unsaturated C₁₃-C₁₉ alkyl.

In embodiments, R² is unsubstituted C₁-C₁₉ alkyl. In embodiments, R² is unsubstituted C₉-C₁₉ alkyl. In embodiments, R² is unsubstituted C₁₁-C₁₉ alkyl. In embodiments, R² is unsubstituted C₁₃-C₁₉ alkyl. In embodiments, R² is unsubstituted unbranched C₁-C₁₉ alkyl. In embodiments, R² is unsubstituted unbranched C₉-C₁₉ alkyl. In embodiments, R² is unsubstituted unbranched C₁₁-C₁₉ alkyl. In embodiments, R² is unsubstituted unbranched C₁₃-C₁₉ alkyl. In embodiments, R² is unsubstituted unbranched saturated C₁-C₁₉ alkyl. In embodiments, R² is unsubstituted unbranched saturated C₉-C₁₉ alkyl. In embodiments, R² is unsubstituted unbranched saturated C₁₁-C₁₉ alkyl. In embodiments, R² is unsubstituted unbranched saturated C₁₃-C₁₉ alkyl. In embodiments, R² is unsubstituted unbranched unsaturated C₁-C₁₉ alkyl. In embodiments, R² is unsubstituted unbranched unsaturated C₉-C₁₉ alkyl. In embodiments, R² is unsubstituted unbranched unsaturated C₁₁-C₁₉ alkyl. In embodiments, R² is unsubstituted unbranched unsaturated C₁₃-C₁₉ alkyl.

In embodiments, the oligonucleotide is an antisense oligonucleotide. In embodiments, the oligonucleotide is an siRNA. In embodiments, the oligonucleotide is a microRNA mimic. In embodiments, the oligonucleotide is a stem-loop structure. In embodiments, the oligonucleotide is a single-stranded siRNA. In embodiments, the oligonucleotide is an RNaseH oligonucleotide. In embodiments, the oligonucleotide is an anti-microRNA oligonucleotide. In embodiments, the oligonucleotide is a steric blocking oligonucleotide. In embodiments, the oligonucleotide is an aptamer. In embodiments, the oligonucleotide is a CRISPR guide RNA.

In embodiments, the oligonucleotide is a modified oligonucleotide.

In embodiments, the oligonucleotide includes a nucleotide analog.

In embodiments, the oligonucleotide includes a locked nucleic acid (LNA) residue, constrained ethyl (cEt) residue, bicyclic nucleic acid (BNA) residue, unlocked nucleic acid (UNA) residue, phosphorodiamidate morpholino oligomer (PMO) monomer, peptide nucleic acid (PNA) monomer, 2′-O-methyl (2′-OMe) residue, 2′-O-methyoxyethyl residue, 2′-deoxy-2′-fluoro residue, 2′-O-methoxy ethyl/phosphorothioate residue, phosphoramidate, phosphorodiamidate, phosphorothioate, phosphorodithioate, phosphonocarboxylic acid, phosphonocarboxylate, phosphonoacetic acid, phosphonoformic acid, methyl phosphonate, boron phosphonate, or O-methylphosphoroamidite. In embodiments, the oligonucleotide includes a bicyclic nucleic acid (BNA) residue. In embodiments, the bicyclic nucleic acid residue is a locked nucleic acid (LNA). In embodiments, the bicyclic nucleic acid (BNA) residue is a constrained ethyl (cEt) residue. In embodiments, the oligonucleotide includes an unlocked nucleic acid (UNA) residue. In embodiments, the oligonucleotide includes a phosphorodiamidate morpholino oligomer (PMO) monomer. In embodiments, the oligonucleotide includes a peptide nucleic acid (PNA) monomer. In embodiments, the oligonucleotide includes a 2′-O-methyl (2′-OMe) residue. In embodiments, the oligonucleotide includes a 2′-O-methyoxyethyl residue. In embodiments, the oligonucleotide includes a 2′-deoxy-2′-fluoro residue. In embodiments, the oligonucleotide includes a 2′-O-methoxy ethyl/phosphorothioate residue. In embodiments, the oligonucleotide includes a phosphoramidate. In embodiments, the oligonucleotide includes a phosphorodiamidate. In embodiments, the oligonucleotide includes a phosphorothioate. In embodiments, the oligonucleotide includes a phosphorodithioate. In embodiments, the oligonucleotide includes a phosphonocarboxylic acid. In embodiments, the oligonucleotide includes a phosphonocarboxylate. In embodiments, the oligonucleotide includes a phosphonoacetic acid. In embodiments, the oligonucleotide includes a phosphonoformic acid. In embodiments, the oligonucleotide includes a methyl phosphonate. In embodiments, the oligonucleotide includes a boron phosphonate. In embodiments, the oligonucleotide includes an O-methylphosphoroamidite.

In embodiments, provided herein are compounds having the structure of Formula I:

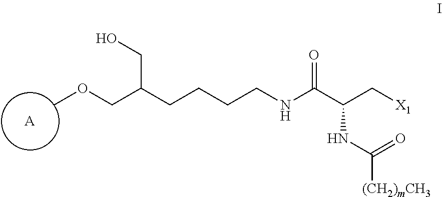

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to the lipid-containing moiety at the 3′end of one strand of the modified double-stranded oligonucleotide or the 3′ end of the modified single-stranded oligonucleotide,

X₁ is

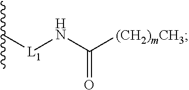

L₁ is (CH₂)ₙ—, —(CH₂)ₙL₂(CH₂)ₙ— or a bond; L₂ is —C(═O)NH—, and wherein each m is independently an integer from 10 to 18 and wherein each n is independently an integer from 1 to 6. In embodiments, X₁ is:

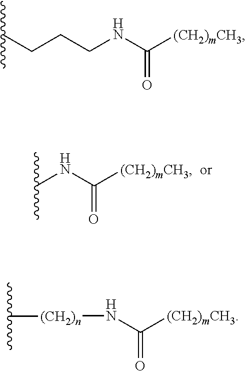

In embodiments, X₁ is

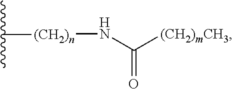

each m is 10, and n is 3. In embodiments, X₁ is

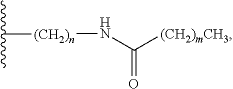

each m is 11, and n is 3. In embodiments, X₁ is

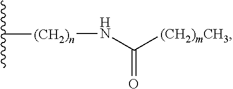

each m is 12, and n is 3. In embodiments, X₁ is

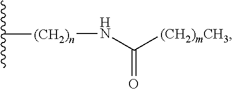

each m is 13, and n is 3. In embodiments, X₁ is

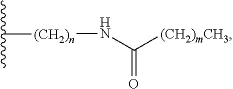

each m is 14, and n is 3. In embodiments, X₁ is

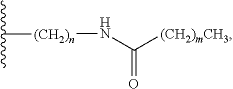

each m is 15, and n is 3. In embodiments, X₁ is

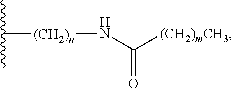

each m is 16, and n is 3. In embodiments, X₁ is

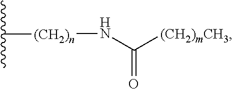

each m is 17, and n is 3. In embodiments, X₁ is

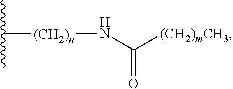

each m is 18, and n is 3. In embodiments, X₁ is

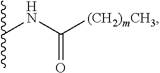

each m is 10. In embodiments, X₁ is

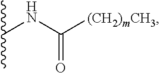

and each m is 11. In embodiments, X₁ is

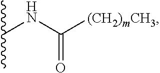

and each m is 12. In embodiments, X₁ is

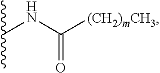

and each m is 13. In embodiments, X₁ is

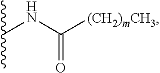

and each m is 14. In embodiments, X₁ is

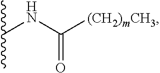

and each m is 15. In embodiments, X₁ is

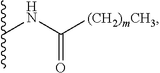

and each m is 16. In embodiments, X₁ is

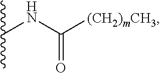

and each m is 17. In embodiments, X₁ is

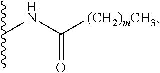

and each m is 18.

In embodiments, X₁ is

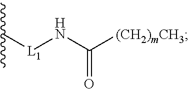

L₁ is —(CH₂)₃C(═O)NH(CH₂)₅—; and each m is 10. In embodiments, X₁ is

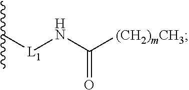

L₁ is —(CH₂)₃C(═O)NH(CH₂)₅—; and each m is 11. In embodiments, X₁ is

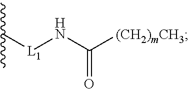

L₁ is —(CH₂)₃C(═O)NH(CH₂)₅—; and each m is 12. In embodiments, X₁ is

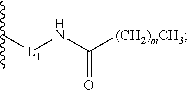

L₁ is —(CH₂)₃C(═O)NH(CH₂)₅—; and each m is 13. In embodiments, X₁ is

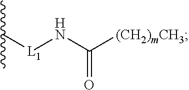

L₁ is —(CH₂)₃C(═O)NH(CH₂)₅—; and each m is 14. In embodiments, X₁ is

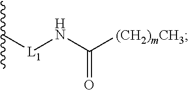

L₁ is —(CH₂)₃C(═O)NH(CH₂)₅—; and each m is 15. In embodiments, X₁ is

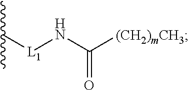

L₁ is —(CH₂)₃C(═O)NH(CH₂)₅—; and each m is 16. In embodiments, X₁ is

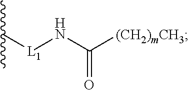

L₁ is —(CH₂)₃C(═O)NH(CH₂)₅—; and each m is 17. In embodiments, X₁ is

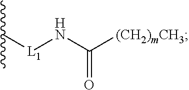

L₁ is —(CH₂)₃C(═O)NH(CH₂)₅—; and each m is 18.

In embodiments, L₁ is a bond; and each m is independently an integer from 10 to 16. In embodiments, L₁ is a bond; and each m is independently an integer from 12 to 16. In embodiments, L₁ is a bond; and each m is independently an integer from 12 to 14. In embodiments, L₁ is a bond; and each m is 14. In embodiments, L₁ is —(CH₂)ₙL₂(CH₂)ₙ—; L₂ is —C(═O)NH—; each m is independently an integer from 10 to 16; and each n is independently an integer from 1 to 6. In embodiments, L₁ is —(CH₂)ₙL₂(CH₂)ₙ—; L₂ is —C(═O)NH—; each m is independently an integer from 12 to 16; and each n is independently an integer from 1 to 6. In embodiments, L₁ is —(CH₂)ₙL₂(CH₂)ₙ—; L₂ is —C(═O)NH—; each m is independently an integer from 12 to 14; and each n is independently an integer from 1 to 6. In embodiments, L₁ is —(CH₂)ₙL₂(CH₂)ₙ—; L₂ is —C(═O)NH—; each m is independently 14; and each n is independently an integer from 1 to 6. In embodiments, L₁ is —(CH₂)₃C(═O)NH(CH₂)₅—; and each m is independently an integer from 10 to 16. In embodiments, L₁ is —(CH₂)₃C(═O)NH(CH₂)₅—; and each m is independently an integer from 12 to 16. In embodiments, L₁ is —(CH₂)₃C(═O)NH(CH₂)₅—; and each m is independently an integer from 12 to 14. In embodiments, L₁ is —(CH₂)₃C(═O)NH(CH₂)₅—; and each m is 14. In embodiments, each m is 14.

In embodiments, provided herein are compounds having the structure of Formula Ia:

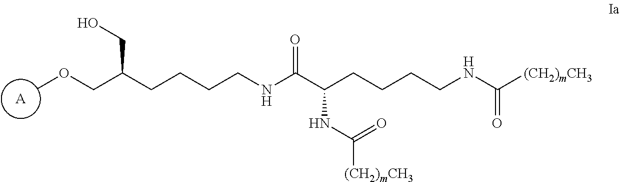

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to the lipid-containing moiety at the 3′end of one strand of the modified double-stranded oligonucleotide or the 3′ end of the modified single-stranded oligonucleotide, and wherein m is an integer from 10 to 18. The portion of above Formula Ia represented by:

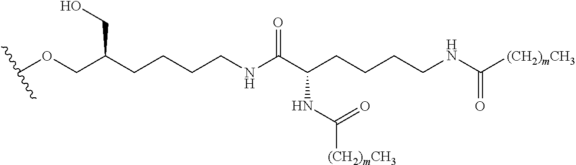

is the lipid-containing moiety portion of Formula Ia.

In embodiments, provided herein are compounds having the structure of Formula Ib:

cet

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to the lipid-containing moiety at the 3′end of one strand of the modified double-stranded oligonucleotide or the 3′ end of the modified single-stranded oligonucleotide, and wherein m is an integer from 10 to 18. The portion of above Formula Ib represented by:

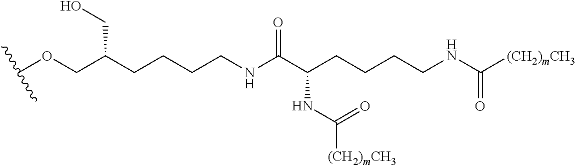

is the lipid-containing moiety portion of Formula Ib.

In embodiments of the compounds having the structure of Formulae I, Ia, or Ib, each m is an integer from 12 to 16. In embodiments, each m is an integer from 12 to 14. In embodiments, each m is 10, L₁ is —(CH₂)ₙ—, and n is 3. In embodiments, each m is 11, L₁ is —(CH₂)ₙ—, and n is 3. In embodiments, each m is 12, L₁ is —(CH₂)ₙ—, and n is 3. In embodiments, each m is 13, L₁ is —(CH₂)ₙ—, and n is 3. In embodiments, each m is 14, L₁ is —(CH₂)ₙ—, and n is 3. In embodiments, each m is 15, L₁ is —(CH₂)ₙ—, and n is 3. In embodiments, each m is 16, L₁ is —(CH₂)ₙ—, and n is 3. In embodiments, each m is 17, L₁ is —(CH₂)ₙ—, and n is 3. In embodiments, each m is 18, L₁ is —(CH₂)ₙ—, and n is 3.

In embodiments, provided herein is a lipid-conjugated compound having the structure of Formula II:

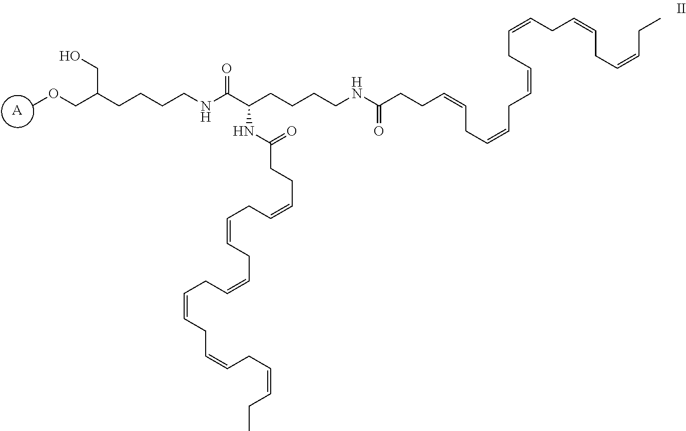

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded oligonucleotide. The portion of above Formula II represented by:

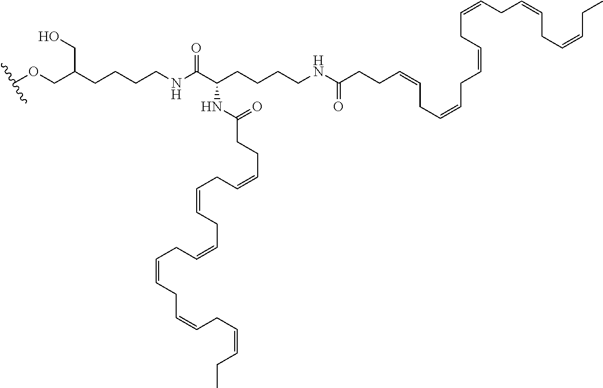 [SMILES: *OCC(CO)CCCCNC(=O)[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC]

is the lipid-containing moiety portion of Formula II.

In embodiments, provided herein is to a lipid-conjugated compound having the structure of Formula IIa:

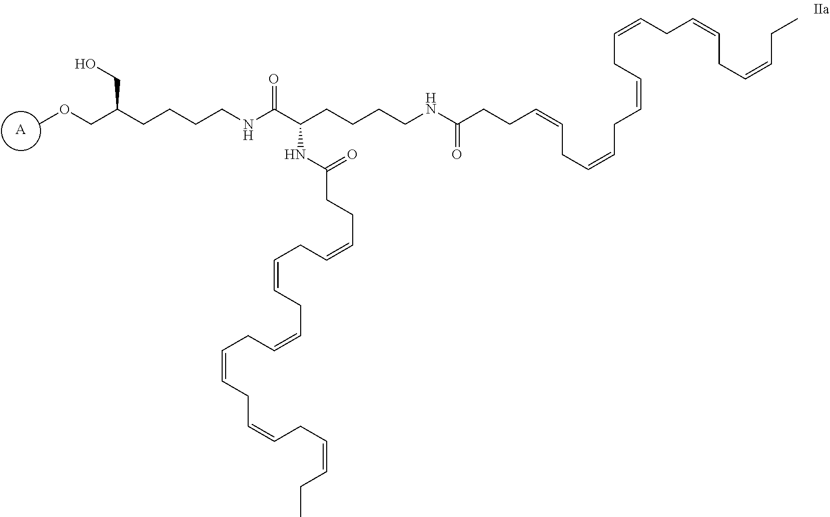

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded oligonucleotide. The portion of above Formula IIa represented by:

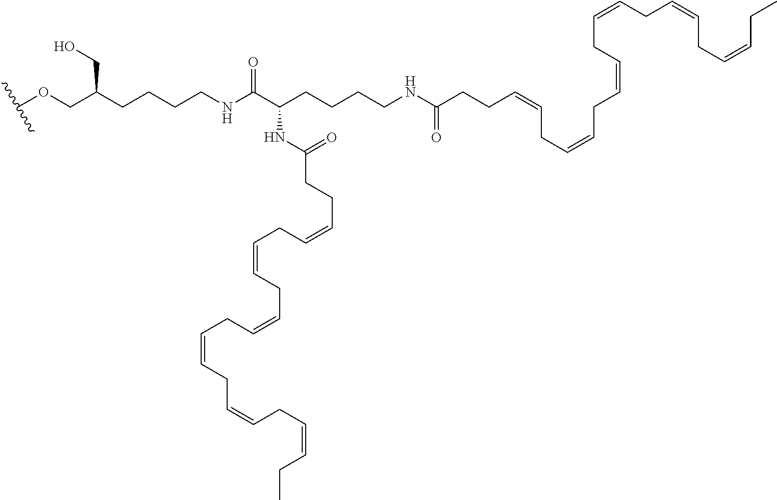 [SMILES: *OC[C@@H](CO)CCCCNC(=O)[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC]

is the lipid-containing moiety portion of Formula IIa.

In embodiments, provided herein is a lipid-conjugated compound having the structure of Formula IIb:

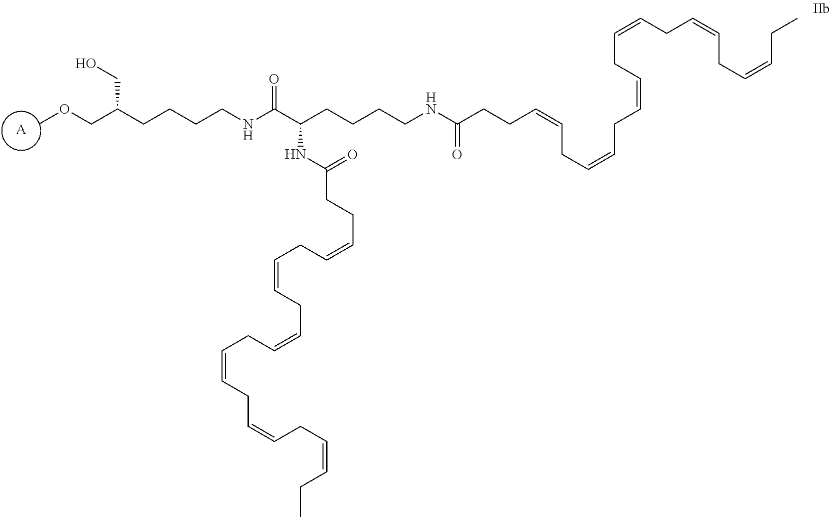

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded oligonucleotide. The portion of above Formula IIb represented by:

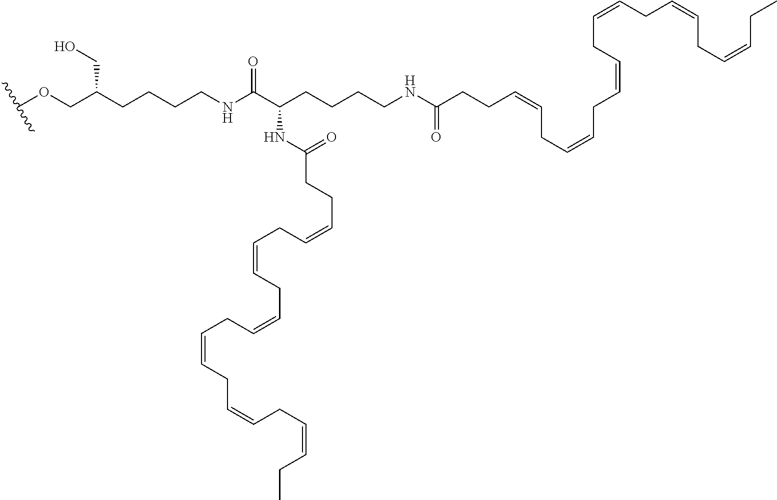 [SMILES: *OC[C@H](CO)CCCCNC(=O)[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC]

is the lipid-containing moiety portion of Formula IIb.

In embodiments, provided herein is a lipid-conjugated compound having the structure of Formula III:

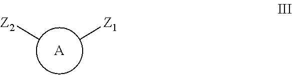

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to Z₁ at the 3′ end of one strand of the modified double-stranded oligonucleotide or the 3′ end of the modified single-stranded oligonucleotide, where Z₁ is

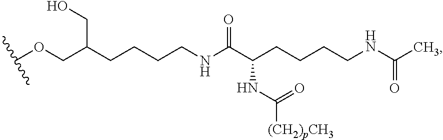

wherein p is an integer from 10 to 18, and
wherein the modified double-stranded oligonucleotide is conjugated to Z₂ at the 5′ end of one strand of the modified double-stranded oligonucleotide or the 5′ end of the modified single-stranded oligonucleotide, where Z₂ is

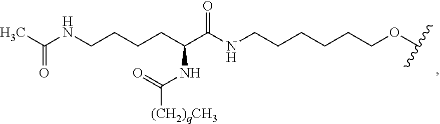

wherein q is an integer from 10 to 18. In embodiments p is 14; and q is 14.

In embodiments, provided herein is a lipid-conjugated compound having the structure of Formula IIIa:

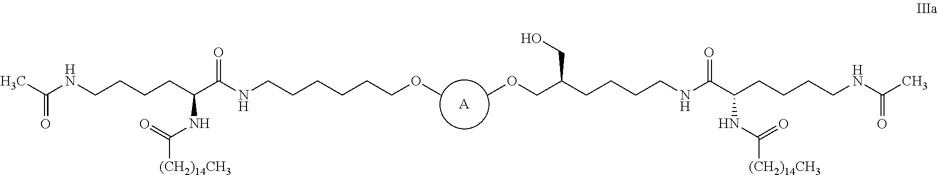

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

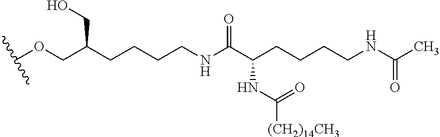

at the 3′ end of one strand of the modified double-stranded oligonucleotide or the 3′ end of the modified single-stranded oligonucleotide, and wherein the modified double-stranded oligonucleotide or modified single-stranded oligonucleotide is conjugated to a lipid-containing moiety

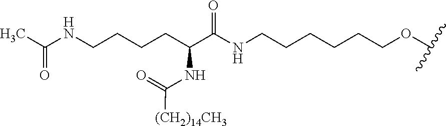

at the 5′ end of one strand of the modified double-stranded oligonucleotide or the 5′ end of the modified single-stranded oligonucleotide.

In embodiments, provided herein is a lipid-conjugated compound having the structure of Formula IIIb:

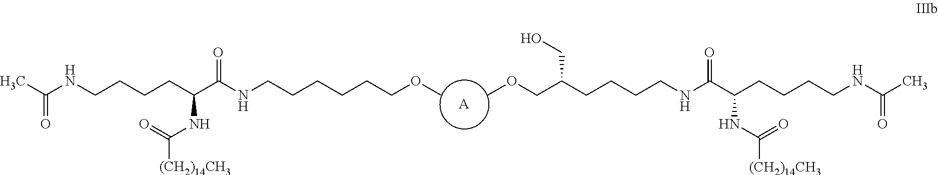

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

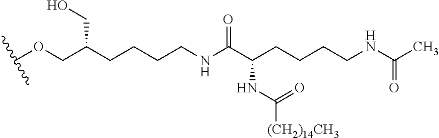

at the 3′ end of one strand of the modified double-stranded oligonucleotide or the 3′ end of the modified single-stranded oligonucleotide, and wherein the modified double-stranded oligonucleotide or single-stranded oligonucleotide is conjugated to a lipid-containing moiety

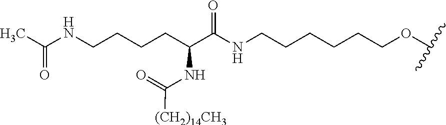

at the 5′ end of one strand of the modified double-stranded oligonucleotide or the 5′ end of the modified single-stranded oligonucleotide.

In embodiments, L₁ is a bond, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L₁ is substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L₁ is substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), or substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L₁ is unsubstituted alkylene (e.g., C₁-C₂₀, C₁-C₁₂, C₁-C₈, C₁-C₆, C₁-C₄, or C₁-C₂), or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, when L₁ is substituted, L₁ is substituted with a substituent group. In embodiments, when L₁ is substituted, L₁ is substituted with a size-limited substituent group. In embodiments, when L₁ is substituted, L₁ is substituted with a lower substituent group.

In embodiments, L₁ is a bond. In embodiments, L₁ is —(CH₂)ₙ—, or —(CH₂)ₙL₂(CH₂)ₙ—. In embodiments, L₁ is —(CH₂)ₙ—. In embodiments, L₁ is —(CH₂)ₙL₂(CH₂)ₙ—. In embodiments, n is 1 to 6. In embodiments, n is 1 to 5. In embodiments, n is 1 to 4. In embodiments, n is 1 to 3. In embodiments, n is 1 to 2. In embodiments, n is 1. In embodiments, n is 2. In embodiments, n is 3. In embodiments, n is 4. In embodiments, n is 5. In embodiments, n is 6.

In embodiments, each occurrence of n (i.e. n′ and n″) may be the same or different. In embodiments, each occurrence of (i.e. n′ and n″) may be the same. In embodiments, each occurrence of n (i.e. n′ and n″) may be different. In embodiments, n is 1 to 6. In embodiments, n′ is 1 to 5. In embodiments, n′ is 1 to 4. In embodiments, n′ is 1 to 3. In embodiments, n′ is 1 to 2. In embodiments, n′ is 1. In embodiments, n′ is 2. In embodiments, n′ is 3. In embodiments, n′ is 4. In embodiments, n′ is 5. In embodiments, n′ is 6. In embodiments, n″ is 1 to 6. In embodiments, n″ is 1 to 5. In embodiments, n″ is 1 to 4. In embodiments, n″ is 1 to 3. In embodiments, n″ is 1 to 2. In embodiments, n″ is 1. In embodiments, n″ is 2. In embodiments, n″ is 3. In embodiments, n″ is 4. In embodiments, n″ is 5. In embodiments, n″ is 6.

In embodiments, m is 10 to 18. In embodiments, m is 10 to 17. In embodiments, m is 10 to 16. In embodiments, m is 10 to 15. In embodiments, m is 10 to 14. In embodiments, m is 10 to 13. In embodiments, m is 10 to 12. In embodiments, m is 10 to 11. In embodiments, m is 10. In embodiments, m is 11. In embodiments, m is 12. In embodiments, m is 13. In embodiments, m is 14. In embodiments, m is 15. In embodiments, m is 16. In embodiments, m is 17. In embodiments, m is 18.

In embodiments, L₂ is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur). In embodiments, L₂ is —C(═O)NH—. In embodiments, L₂ is —C(═O)O—. In embodiments, L₂ is —OC(═O)O—. In embodiments, L₂ is —NHC(═O)O—. In embodiments, L₂ is —NHC(═O)NH—. In embodiments, L₂ is —C(═S)NH—. In embodiments, L₂ is —C(═O)S—. In embodiments, L₂ is —NH—. In embodiments, L₂ is O (oxygen). In embodiments, L₂ is S (sulfur).

L³ is independently a bond, —NH—, —O—, —S—, —C(O)—, —NHC(O)—, —NHC(O)NH—, —C(O)O—, —OC(O)—, —C(O)NH—, —OPO₂—O—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L³ is independently a bond, —NH—, —O—, —S—, —C(O)—, —NHC(O)—, —NHC(O)NH—, —C(O)O—, —OC(O)—, —C(O)NH—OPO₂—O—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L³ is independently a bond, —NH—, —O—, —S—, —C(O)—, —NHC(O)—, —NHC(O)NH—, —C(O)O—, —OC(O)—, —C(O)NH—, —OPO₂—O—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L³ is substituted, L³ is substituted with a substituent group. In embodiments, when L³ is substituted, L³ is substituted with a size-limited substituent group. In embodiments, when L³ is substituted, L³ is substituted with a lower substituent group.

L⁴ is independently a bond, —NH—, —O—, —S—, —C(O)—, —NHC(O)—, —NHC(O)NH—, —C(O)O—, —OC(O)—, —C(O)NH—, —OPO₂—O—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁴ is a bond, —NH—, —O—, —S—, —C(O)—, —NHC(O)—, —NHC(O)NH—, —C(O)O—, —OC(O)—, —C(O)NH—, —OPO₂—O—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁴ is a bond, —NH—, —O—, —S—, —C(O)—, —NHC(O)—, —NHC(O)NH—, —C(O)O—, —OC(O)—, —C(O)NH—, —OPO₂—O—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁴ is substituted, L⁴ is substituted with a substituent group. In embodiments, when L⁴ is substituted, L⁴ is substituted with a size-limited substituent group. In embodiments, when L⁴ is substituted, L⁴ is substituted with a lower substituent group.

L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁵ is substituted, L⁵ is substituted with a substituent group. In embodiments, when L⁵ is substituted, L⁵ is substituted with a size-limited substituent group. In embodiments, when L⁵ is substituted, L⁵ is substituted with a lower substituent group.

L^{5A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5A} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5A} is substituted, L^{5A} is substituted with a substituent group. In embodiments, when L^{5A} is substituted, L^{5A} is substituted with a size-limited substituent group. In embodiments, when L^{5A} is substituted, L^{5A} is substituted with a lower substituent group.

L^{5B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5B} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5B} is substituted, L^{5B} is substituted with a substituent group. In embodiments, when L^{5B} is substituted, L^{5B} is substituted with a size-limited substituent group. In embodiments, when L^{5B} is substituted, L^{5B} is substituted with a lower substituent group.

L^{5C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5C} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5C} is substituted, L^{5C} is substituted with a substituent group. In embodiments, when L^{5C} is substituted, L^{5C} is substituted with a size-limited substituent group. In embodiments, when L^{5C} is substituted, L^{5C} is substituted with a lower substituent group.

L^{5D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5D} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5D} is substituted, L^{5D} is substituted with a substituent group. In embodiments, when L^{5D} is substituted, L^{5D} is substituted with a size-limited substituent group. In embodiments, when L^{5D} is substituted, L^{5D} is substituted with a lower substituent group.

L^{5E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5E} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5E} is substituted, L^{5E} is substituted with a substituent group. In embodiments, when L^{5E}is substituted, L^{5E} is substituted with a size-limited substituent group. In embodiments, when L^{5E} is substituted, L^{5E} is substituted with a lower substituent group.

L⁶ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁶ is substituted, L⁶ is substituted with a substituent group. In embodiments, when L⁶ is substituted, L⁶ is substituted with a size-limited substituent group. In embodiments, when L⁶ is substituted, L⁶ is substituted with a lower substituent group.

L^{6A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6A} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6A} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6A} is substituted, L^{6A} is substituted with a substituent group. In embodiments, when L^{6A} is substituted, L^{6A} is substituted with a size-limited substituent group. In embodiments, when L^{6A} is substituted, L^{6A} is substituted with a lower substituent group.

L^{6B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6B} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6B} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6B} is substituted, L^{6B} is substituted with a substituent group. In embodiments, when L^{6B} is substituted, L^{6B} is substituted with a size-limited substituent group. In embodiments, when L^{6B} is substituted, L^{6B} is substituted with a lower substituent group.

L^{6C} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶c is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶c is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6C} is substituted, L^{6C} is substituted with a substituent group. In embodiments, when L^{6C} is substituted, L^{6C} is substituted with a size-limited substituent group. In embodiments, when L^{6C} is substituted, L^{6C} is substituted with a lower substituent group.

L^{6D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄—C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6D} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6D} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6D} is substituted, L^{6D} is substituted with a substituent group. In embodiments, when L^{6D} is substituted, L^{6D} is substituted with a size-limited substituent group. In embodiments, when L^{6D} is substituted, L^{6D} is substituted with a lower substituent group.

L^{6E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6E} is a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6E} is a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6E} is substituted, L^{6E} is substituted with a substituent group. In embodiments, when L^{6E} is substituted, L^{6E} is substituted with a size-limited substituent group. In embodiments, when L^{6E} is substituted, L^{6E} is substituted with a lower substituent group.

In embodiments, L⁷ is independently substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂). In embodiments, L⁷ is independently substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁷ is independently substituted (e.g., substituted with a substituent group, a size-limited substituent group, or lower substituent group) or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered). In embodiments, when L⁷ is substituted, L⁷ is substituted with a substituent group. In embodiments, when L⁷ is substituted, L⁷ is substituted with a size-limited substituent group. In embodiments, when L⁷ is substituted, L⁷ is substituted with a lower substituent group.

In embodiments, R¹ is unsubstituted alkyl (e.g., C₁-C₂₅, C₁-C₂₀, C₁-C₁₂, C₁-C₈, C₁-C₆, C₁-C₄, or C₁-C₂). In embodiments, R¹ is unsubstituted C₁-C₂₅ alkyl. In embodiments, R¹ is unsubstituted C₁-C₂₀ alkyl. In embodiments, R¹ is unsubstituted C₁-C₁₂ alkyl. In embodiments, R¹ is unsubstituted C₁-C₈ alkyl. In embodiments, R¹ is unsubstituted C₁-C₆ alkyl. In embodiments, R¹ is unsubstituted C₁-C₄ alkyl. In embodiments, R¹ is unsubstituted C₁-C₂ alkyl.

In embodiments, R¹ is unsubstituted branched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched C₁-C₂₅ alkyl. In embodiments, R¹ is unsubstituted branched C₁-C₂₀ alkyl. In embodiments, R¹ is unsubstituted branched C₁-C₁₂ alkyl. In embodiments, R¹ is unsubstituted branched C₁-C₈ alkyl. In embodiments, R¹ is unsubstituted branched C₁-C₆ alkyl. In embodiments, R¹ is unsubstituted branched C₁-C₄ alkyl. In embodiments, R¹ is unsubstituted branched C₁-C₂ alkyl.

In embodiments, R¹ is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched C₁-C₂₅ alkyl. In embodiments, R¹ is unsubstituted unbranched C₁-C₂₀ alkyl. In embodiments, R¹ is unsubstituted unbranched C₁-C₁₂ alkyl. In embodiments, R¹ is unsubstituted unbranched C₁-C₈ alkyl. In embodiments, R¹ is unsubstituted unbranched C₁-C₆ alkyl. In embodiments, R¹ is unsubstituted unbranched C₁-C₄ alkyl. In embodiments, R¹ is unsubstituted unbranched C₁-C₂ alkyl.

In embodiments, R¹ is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched saturated C₁-C₂₅ alkyl. In embodiments, R¹ is unsubstituted branched saturated C₁-C₂₀ alkyl. In embodiments, R¹ is unsubstituted branched saturated C₁-C₁₂ alkyl. In embodiments, R¹ is unsubstituted branched saturated C₁-C₈ alkyl. In embodiments, R¹ is unsubstituted branched saturated C₁-C₆ alkyl. In embodiments, R¹ is unsubstituted branched saturated C₁-C₄ alkyl. In embodiments, R¹ is unsubstituted branched saturated C₁-C₂ alkyl.

In embodiments, R¹ is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched unsaturated C₁-C₂₅ alkyl. In embodiments, R¹ is unsubstituted branched unsaturated C₁-C₂₀ alkyl. In embodiments, R¹ is unsubstituted branched unsaturated C₁-C₁₂ alkyl. In embodiments, R¹ is unsubstituted branched unsaturated C₁-C₈ alkyl. In embodiments, R¹ is unsubstituted branched unsaturated C₁-C₆ alkyl. In embodiments, R¹ is unsubstituted branched unsaturated C₁-C₄ alkyl. In embodiments, R¹ is unsubstituted branched saturated C₁-C₂ alkyl.

In embodiments, R¹ is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched saturated C₁-C₂₅ alkyl. In embodiments, R¹ is unsubstituted unbranched saturated C₁-C₂₀ alkyl. In embodiments, R¹ is unsubstituted unbranched saturated C₁-C₁₂ alkyl. In embodiments, R¹ is unsubstituted unbranched saturated C₁-C₈ alkyl. In embodiments, R¹ is unsubstituted unbranched saturated C₁-C₆ alkyl. In embodiments, R¹ is unsubstituted unbranched saturated C₁-C₄ alkyl. In embodiments, R¹ is unsubstituted unbranched saturated C₁-C₂ alkyl.

In embodiments, R¹ is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched unsaturated C₁-C₂₅ alkyl. In embodiments, R¹ is unsubstituted unbranched unsaturated C₁-C₂₀ alkyl. In embodiments, R¹ is unsubstituted unbranched unsaturated C₁-C₁₂ alkyl. In embodiments, R¹ is unsubstituted unbranched unsaturated C₁-C₈ alkyl. In embodiments, R¹ is unsubstituted unbranched unsaturated C₁-C₆ alkyl. In embodiments, R¹ is unsubstituted unbranched unsaturated C₁-C₄ alkyl. In embodiments, R¹ is unsubstituted unbranched unsaturated C₁-C₂ alkyl.

In embodiments, R¹ is unsubstituted C₉-C₁₉ alkyl. In embodiments, R¹ is unsubstituted branched C₉-C₁₉ alkyl. In embodiments, R¹ is unsubstituted unbranched C₉-C₁₉ alkyl. In embodiments, R¹ is unsubstituted branched saturated C₉-C₁₉ alkyl. In embodiments, R¹ is unsubstituted branched unsaturated C₉-C₁₉ alkyl. In embodiments, R¹ is unsubstituted unbranched saturated C₉-C₁₉ alkyl. In embodiments, R¹ is unsubstituted unbranched unsaturated C₉-C₁₉ alkyl.

In embodiments, R² is unsubstituted alkyl (e.g., C₁-C₂₅, C₁-C₂₀, C₁-C₁₂, C₁-C₈, C₁-C₆, C₁-C₄, or C₁-C₂). In embodiments, R² is unsubstituted C₁-C₂₅ alkyl. In embodiments, R² is unsubstituted C₁-C₂₀ alkyl. In embodiments, R² is unsubstituted C₁-C₁₂ alkyl. In embodiments, R² is unsubstituted C₁-C₈ alkyl. In embodiments, R² is unsubstituted C₁-C₆ alkyl. In embodiments, R² is unsubstituted C₁-C₄ alkyl. In embodiments, R² is unsubstituted C₁-C₂ alkyl.

In embodiments, R² is unsubstituted branched alkyl (e.g., C₁-C₂₅, C₁-C₂₀, C₁-C₁₂, C₁-C₈, C₁-C₆, C₁-C₄, or C₁-C₂). In embodiments, R² is unsubstituted branched C₁-C₂₅ alkyl. In embodiments, R² is unsubstituted branched C₁-C₂₀ alkyl. In embodiments, R² is unsubstituted branched C₁-C₁₂ alkyl. In embodiments, R² is unsubstituted branched C₁-C₈ alkyl. In embodiments, R² is unsubstituted branched C₁-C₆ alkyl. In embodiments, R² is unsubstituted branched C₁-C₄ alkyl. In embodiments, R² is unsubstituted branched C₁-C₂ alkyl.

In embodiments, R² is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched C₁-C₂₅ alkyl. In embodiments, R² is unsubstituted unbranched C₁-C₂₀ alkyl. In embodiments, R² is unsubstituted unbranched C₁-C₁₂ alkyl. In embodiments, R² is unsubstituted unbranched C₁-C₈ alkyl. In embodiments, R² is unsubstituted unbranched C₁-C₆ alkyl. In embodiments, R² is unsubstituted unbranched C₁-C₄ alkyl. In embodiments, R² is unsubstituted unbranched C₁-C₂ alkyl.

In embodiments, R² is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched saturated C₁-C₂₅ alkyl. In embodiments, R² is unsubstituted branched saturated C₁-C₂₀ alkyl. In embodiments, R² is unsubstituted branched saturated C₁-C₁₂ alkyl. In embodiments, R² is unsubstituted branched saturated C₁-C₈ alkyl. In embodiments, R² is unsubstituted branched saturated C₁-C₆ alkyl. In embodiments, R² is unsubstituted branched saturated C₁-C₄ alkyl. In embodiments, R² is unsubstituted branched saturated C₁-C₂ alkyl.

In embodiments, R² is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched unsaturated C₁-C₂₅ alkyl. In embodiments, R² is unsubstituted branched unsaturated C₁-C₂₀ alkyl. In embodiments, R² is unsubstituted branched unsaturated C₁-C₁₂ alkyl. In embodiments, R² is unsubstituted branched unsaturated C₁-C₈ alkyl. In embodiments, R² is unsubstituted branched unsaturated C₁-C₆ alkyl. In embodiments, R² is unsubstituted branched unsaturated C₁-C₄ alkyl. In embodiments, R² is unsubstituted branched saturated C₁-C₂ alkyl.

In embodiments, R² is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched saturated C₁-C₂₅ alkyl. In embodiments, R² is unsubstituted unbranched saturated C₁-C₂₀ alkyl. In embodiments, R² is unsubstituted unbranched saturated C₁-C₁₂ alkyl. In embodiments, R² is unsubstituted unbranched saturated C₁-C₈ alkyl. In embodiments, R² is unsubstituted unbranched saturated C₁-C₆ alkyl. In embodiments, R² is unsubstituted unbranched saturated C₁-C₄ alkyl. In embodiments, R² is unsubstituted unbranched saturated C₁-C₂ alkyl.

In embodiments, R² is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched unsaturated C₁-C₂₅ alkyl. In embodiments, R² is unsubstituted unbranched unsaturated C₁-C₂₀ alkyl. In embodiments, R² is unsubstituted unbranched unsaturated C₁-C₁₂ alkyl. In embodiments, R² is unsubstituted unbranched unsaturated C₁-C₈ alkyl. In embodiments, R² is unsubstituted unbranched unsaturated C₁-C₆ alkyl. In embodiments, R² is unsubstituted unbranched unsaturated C₁-C₄ alkyl. In embodiments, R² is unsubstituted unbranched unsaturated C₁-C₂ alkyl.

In embodiments, R² is unsubstituted C₉-C₁₉ alkyl. In embodiments, R² is unsubstituted branched C₉-C₁₉ alkyl. In embodiments, R² is unsubstituted unbranched C₉-C₁₉ alkyl. In embodiments, R² is unsubstituted branched saturated C₉-C₁₉ alkyl. In embodiments, R² is unsubstituted branched unsaturated C₉-C₁₉ alkyl. In embodiments, R² is unsubstituted unbranched saturated C₉-C₁₉ alkyl. In embodiments, R² is unsubstituted unbranched unsaturated C₉-C₁₉ alkyl.

In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R³ is substituted, R³ is substituted with a substituent group. In embodiments, when R³ is substituted, R³ is substituted with a size-limited substituent group. In embodiments, when R³ is substituted, R³ is substituted with a lower substituent group.

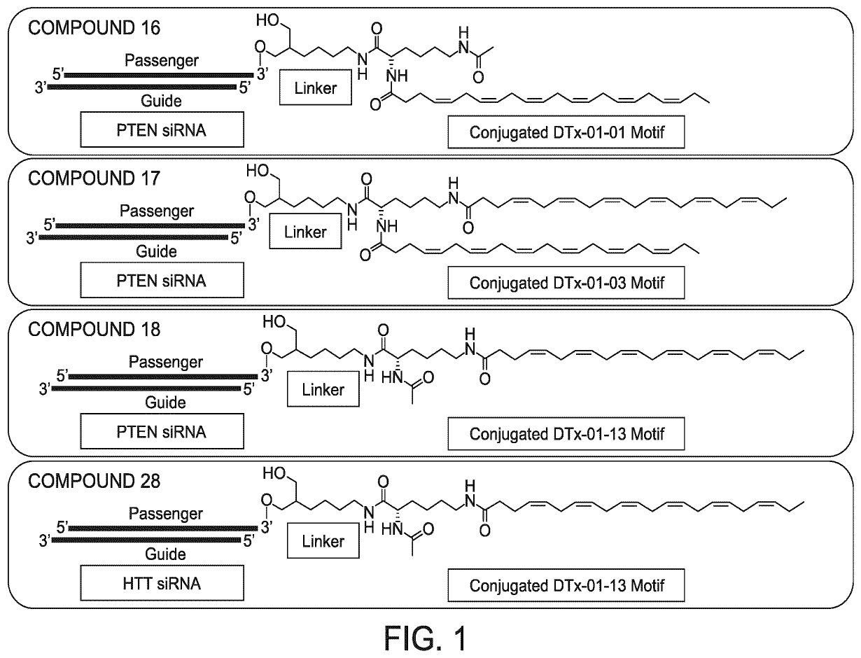

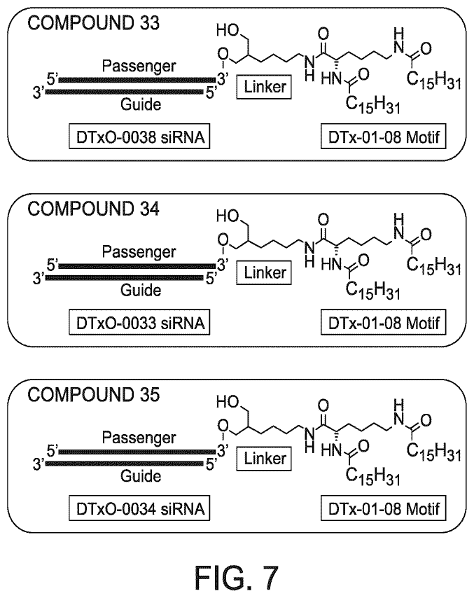

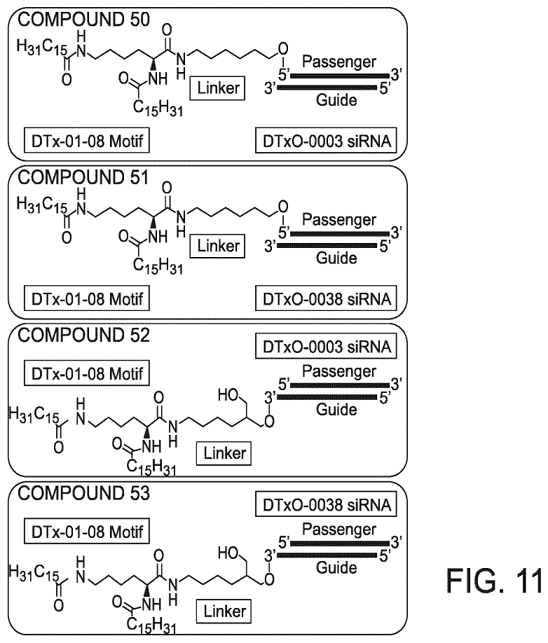

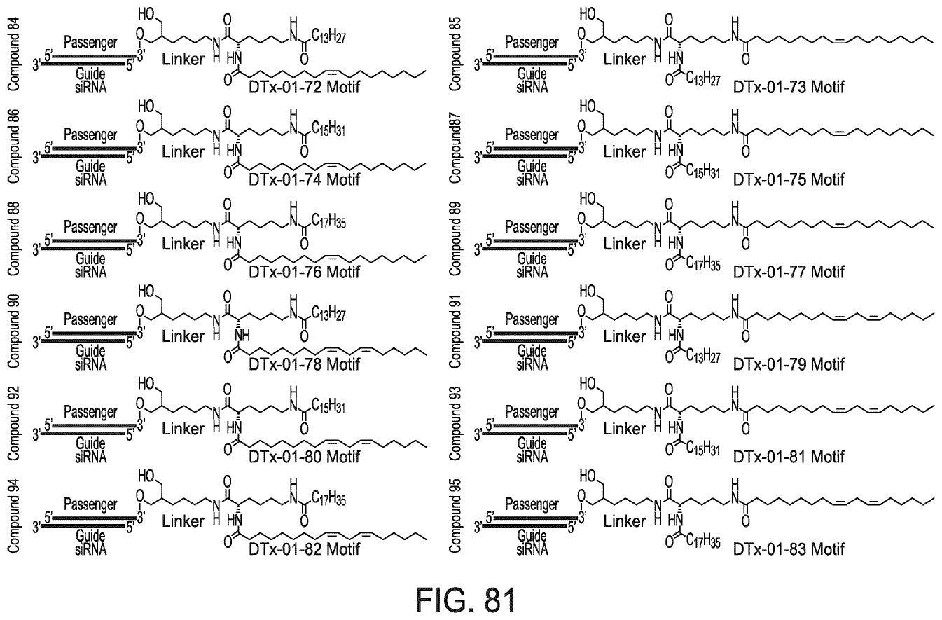

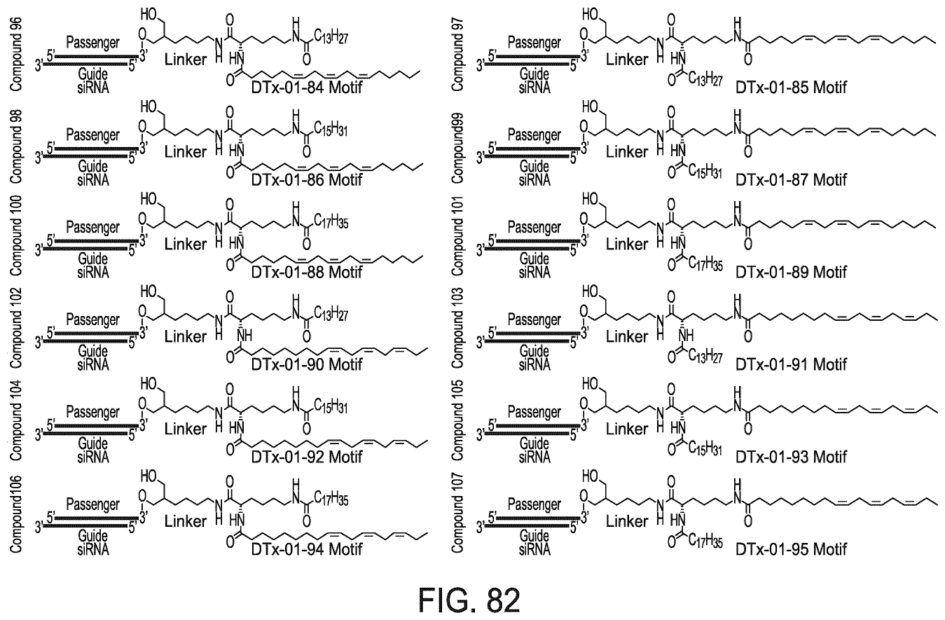

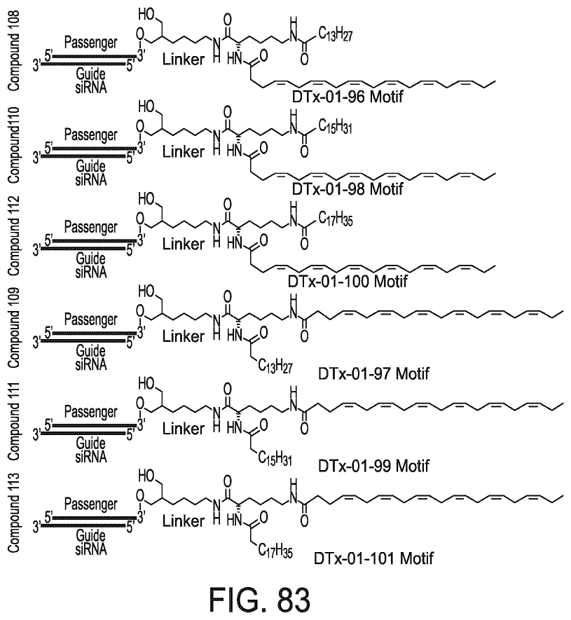

In embodiments, the lipid-modified nucleic acid compound includes a motif described herein, including in any aspects, embodiments, claims, figures (e.g., FIGS. 1-83, particularly FIGS. 1-12, and FIGS. 80-83), tables (e.g., Table 1), examples, or schemes (e.g., Schemes I, II, and III). In embodiments, the lipid-modified nucleic acid compound includes a motif selected from any one of the motifs in Table 1 below. In embodiments, the lipid-modified nucleic acid compound includes a DTx-01-01 motif in Table 1. In embodiments, the lipid-modified nucleic acid compound includes a DTx-01-03 motif 1 of Table 1. In embodiments, the lipid-modified nucleic acid compound includes a DTx-01-06 motif in Table 1. In embodiments, the lipid-modified nucleic acid compound includes a DTx-01-07 motif in Table 1. In embodiments, the lipid-modified nucleic acid compound includes a DTx-01-08 motif in Table 1. In embodiments, the lipid-modified nucleic acid compound includes a DTx-01-09 motif in Table 1. In embodiments, the lipid-modified nucleic acid compound includes a DTx-01-11 motif in Table 1. In embodiments, the lipid-modified nucleic acid compound includes a DTx-01-12 motif in Table 1. In embodiments, the lipid-modified nucleic acid compound includes a DTx-01-13 motif in Table 1. In embodiments, the lipid-modified nucleic acid compound includes a DTx-01-30 motif in Table 1. In embodiments, the lipid-modified nucleic acid compound includes a DTx-01-31 motif in Table 1. In embodiments, the lipid-modified nucleic acid compound includes a DTx-01-32 motif in Table 1. In embodiments, the lipid-modified nucleic acid compound includes a DTx-01-33 motif in Table 1. In embodiments, the lipid-modified nucleic acid compound includes a DTx-01-34 motif in Table 1. In embodiments, the lipid-modified nucleic acid compound includes a DTx-01-35 motif in Table 1. In embodiments, the lipid-modified nucleic acid compound includes a DTx-01-36 motif in Table 1. In embodiments, the lipid-modified nucleic acid compound includes a DTx-01-39 motif in Table 1. In embodiments, the lipid-modified nucleic acid compound includes a DTx-01-43 motif in Table 1. In embodiments, the lipid-modified nucleic acid compound includes a DTx-01-44 motif in Table 1. In embodiments, the lipid-modified nucleic acid compound includes a DTx-01-45 motif in Table 1. In embodiments, the lipid-modified nucleic acid compound includes a DTx-01-46 motif in Table 1. In embodiments, the lipid-modified nucleic acid compound includes a DTx-01-50 motif in Table 1. In embodiments, the lipid-modified nucleic acid compound includes a DTx-01-51 motif in Table 1. In embodiments, the lipid-modified nucleic acid compound includes a DTx-01-52 motif in Table 1. In embodiments, the lipid-modified nucleic acid compound includes a DTx-01-53 motif in Table 1. In embodiments, the lipid-modified nucleic acid compound includes a DTx-01-54 motif in Table 1. In embodiments, the lipid-modified nucleic acid compound includes a DTx-01-55 motif in Table 1. In embodiments, the lipid-modified nucleic acid compound includes a DTx-03-06 motif in Table 1. In embodiments, the lipid-modified nucleic acid compound includes a DTx-03-50 motif in Table 1. In embodiments, the lipid-modified nucleic acid compound includes a DTx-03-51 motif in Table 1. In embodiments, the lipid-modified nucleic acid compound includes a DTx-03-52 motif in Table 1. In embodiments, the lipid-modified nucleic acid compound includes a DTx-03-53 motif in Table 1. In embodiments, the lipid-modified nucleic acid compound includes a DTx-03-54 motif in Table 1. In embodiments, the lipid-modified nucleic acid compound includes a DTx-03-55 motif in Table 1. In embodiments, the lipid-modified nucleic acid compound includes a DTx-04-01 motif in Table 1. In embodiments, the lipid-modified nucleic acid compound includes a DTx-05-01 motif in Table 1. In embodiments, the lipid-modified nucleic acid compound includes a DTx-06-06 motif in Table 1. In embodiments, the lipid-modified nucleic acid compound includes a DTx-06-50 motif in Table 1. In embodiments, the lipid-modified nucleic acid compound includes a DTx-06-51 motif in Table 1. In embodiments, the lipid-modified nucleic acid compound includes a DTx-06-52 motif in Table 1. In embodiments, the lipid-modified nucleic acid compound includes a DTx-06-53 motif in Table 1. In embodiments, the lipid-modified nucleic acid compound includes a DTx-06-54 motif in Table 1. In embodiments, the lipid-modified nucleic acid compound includes a DTx-06-55 motif in Table 1. In embodiments, the lipid-modified nucleic acid compound includes a DTx-08-01 motif in Table 1. In embodiments, the lipid-modified nucleic acid compound includes a DTx-09-01 motif in Table 1. In embodiments, the lipid-modified nucleic acid compound includes a DTx-10-01 motif in Table 1. In embodiments, the lipid-modified nucleic acid compound includes a DTx-11-01 motif in Table 1. In embodiments, the lipid-modified nucleic acid compound includes a DTx-01-60 motif in Table 1. In embodiments, the lipid-modified nucleic acid compound includes a DTx-01-61 motif in Table 1. In embodiments, the lipid-modified nucleic acid compound includes a DTx-01-62 motif in Table 1. In embodiments, the lipid-modified nucleic acid compound includes a DTx-01-63 motif in Table 1. In embodiments, the lipid-modified nucleic acid compound includes a DTx-01-64 motif in Table 1. In embodiments, the lipid-modified nucleic acid compound includes a DTx-01-65 motif in Table 1. In embodiments, the lipid-modified nucleic acid compound includes a DTx-01-66 motif in Table 1. In embodiments, the lipid-modified nucleic acid compound includes a DTx-01-67 motif in Table 1. In embodiments, the lipid-modified nucleic acid compound includes a DTx-01-68 motif in Table 1. In embodiments, the lipid-modified nucleic acid compound includes a DTx-01-69 motif in Table 1. In embodiments, the lipid-modified nucleic acid compound includes a DTx-01-70 motif in Table 1. In embodiments, the lipid-modified nucleic acid compound includes a DTx-01-71 motif in Table 1. In embodiments, the lipid-modified nucleic acid compound includes a DTx-01-72 motif in Table 1. In embodiments, the lipid-modified nucleic acid compound includes a DTx-01-73 motif in Table 1. In embodiments, the lipid-modified nucleic acid compound includes a DTx-01-74 motif in Table 1. In embodiments, the lipid-modified nucleic acid compound includes a DTx-01-75 motif in Table 1. In embodiments, the lipid-modified nucleic acid compound includes a DTx-01-76 motif in Table 1. In embodiments, the lipid-modified nucleic acid compound includes a DTx-01-77 motif in Table 1. In embodiments, the lipid-modified nucleic acid compound includes a DTx-01-78 motif in Table 1. In embodiments, the lipid-modified nucleic acid compound includes a DTx-01-79 motif in Table 1. In embodiments, the lipid-modified nucleic acid compound includes a DTx-01-80 motif in Table 1. In embodiments, the lipid-modified nucleic acid compound includes a DTx-01-81 motif in Table 1. In embodiments, the lipid-modified nucleic acid compound includes a DTx-01-82 motif in Table 1. In embodiments, the lipid-modified nucleic acid compound includes a DTx-01-83 motif in Table 1. In embodiments, the lipid-modified nucleic acid compound includes a DTx-01-84 motif in Table 1. In embodiments, the lipid-modified nucleic acid compound includes a DTx-01-85 motif in Table 1. In embodiments, the lipid-modified nucleic acid compound includes a DTx-01-86 motif in Table 1. In embodiments, the lipid-modified nucleic acid compound includes a DTx-01-87 motif in Table 1. In embodiments, the lipid-modified nucleic acid compound includes a DTx-01-88 motif in Table 1. In embodiments, the lipid-modified nucleic acid compound includes a DTx-01-89 motif in Table 1. In embodiments, the lipid-modified nucleic acid compound includes a DTx-01-90 motif in Table 1. In embodiments, the lipid-modified nucleic acid compound includes a DTx-01-91 motif in Table 1. In embodiments, the lipid-modified nucleic acid compound includes a DTx-01-92 motif in Table 1. In embodiments, the lipid-modified nucleic acid compound includes a DTx-01-93 motif in Table 1. In embodiments, the lipid-modified nucleic acid compound includes a DTx-01-94 motif in Table 1. In embodiments, the lipid-modified nucleic acid compound includes a DTx-01-95 motif in Table 1. In embodiments, the lipid-modified nucleic acid compound includes a DTx-01-96 motif in Table 1. In embodiments, the lipid-modified nucleic acid compound includes a DTx-01-97 motif in Table 1. In embodiments, the lipid-modified nucleic acid compound includes a DTx-01-98 motif in Table 1. In embodiments, the lipid-modified nucleic acid compound includes a DTx-01-99 motif in Table 1. In embodiments, the lipid-modified nucleic acid compound includes a DTx-01-100 motif in Table 1. In embodiments, the lipid-modified nucleic acid compound includes a DTx-01-101 motif in Table 1.

In embodiments of the compounds having the structure of Formulae I, Ia, Ib, II, IIa, IIb, III, IIIa, or IIIb, the modified double-stranded oligonucleotide is conjugated at either of its 3′ ends to the lipid-containing moiety portion of the compound. In embodiments, the modified double-stranded oligonucleotide is conjugated at the 3′end of its guide strand to the lipid-containing moiety portion. In embodiments, the modified double-stranded oligonucleotide is conjugated at the 3′end of its passenger strand to the lipid-containing moiety portion.

In embodiments of the compounds having the structure of Formulae I, Ia, Ib, II, IIa, IIb, III, IIIa, or IIIb, the modified double-stranded oligonucleotide is conjugated at either of its 5′ ends to the lipid-containing moiety portion of the compound. In embodiments, the modified double-stranded oligonucleotide is conjugated at the 5′end of its guide strand to the lipid-containing moiety portion. In embodiments, the modified double-stranded oligonucleotide is conjugated at the 5′end of its passenger strand to the lipid-containing moiety portion.

In embodiments of having the structure of Formulae I, Ia, Ib, II, IIa, or IIb, the conjugation to the 3′-end occurs through a phosphodiester bond. In embodiments of having the structure of Formulae I, Ia, Ib, II, IIa, or IIb, the conjugation to the 5′-end occurs through a phosphodiester bond.

In embodiments of Formulae III, IIIa, or IIIb, A is a modified double-stranded oligonucleotide, Z₁ is conjugated to the 3′ end of the passenger strand of the modified double-stranded oligonucleotide, and Z₂ is conjugated to the 5′ end of the passenger strand of the modified double-stranded oligonucleotide.

In embodiments of Formulae III, IIIa, or IIIb, A is a modified double-stranded oligonucleotide, Z₁ is conjugated to the 3′ end of the guide strand of the modified double-stranded oligonucleotide, and Z₂ is conjugated to the 5′ end of the passenger strand of the modified double-stranded oligonucleotide.

In embodiments, provided herein are methods of introducing the modified double-stranded oligonucleotide into a cell in vitro by contacting the cell under free uptake conditions with the lipid-conjugated compound of Formulae I, Ia, Ib, II, IIa, IIb, III, IIIa, or IIIb, or a corresponding pharmaceutically acceptable salt thereof. In embodiments, the compound is in direct contact with a cell. In embodiments, the cell is a mammalian cell. In embodiments, the cell is a human cell. In embodiments, the cell is a mouse cell. In embodiments, the cell is a fibroblast cell. In embodiments, the cell is a NIH3T3 cell. In embodiments, the cell is a kidney cell. In embodiments, the cell is a HEK293 cell. In embodiments, the cell is an endothelial cell. In embodiments, the cell is a HUVEC cell. In embodiments, the cell is an adipose cell. In embodiments, the cell is a differentiated 3T3L1 cell. In embodiments, the cell is a macrophage cell. In embodiments, the cell is a RAW264.7 cell. In embodiments, the cell is a neuronal cell. In embodiments, the cell is a primary rat neuron. In embodiments, the cell is a SH-SY5Y cell. In embodiments, the cell is a muscle cell. In embodiments, the cell is a differentiated primary human skeletal muscle cell. In embodiments, the cell is a cell of the trabecular meshwork. In embodiments, the cell may be from an immortalized cell line. In embodiments, the cell may be from primary cells. In embodiments, the cell is an adipocyte cell. In embodiments, the cell is a human adipocyte cell. In embodiments, the cell is a hepatocyte cell. In embodiments, the cell is a human hepatocyte cell. In embodiments, the cell is a T cell.

In embodiments, provided herein are methods of introducing the modified double-stranded oligonucleotide into a cell in vivo by intravitreal injection of the lipid-conjugated compound of Formulae I, Ia, Ib, II, IIa, IIb, III, IIIa, or IIIb or a corresponding pharmaceutically acceptable salt thereof. In embodiments, the cell is an eye cell. In embodiments, the eye cell is a photoreceptor, a bipolar cell, a ganglion cell, a horizontal cell, an amacrine cell, a corneal epithelial cell, a corneal endothelium cell, a corneal stromal cell. In embodiments, the corneal epithelium cell is a basal cell, a wing cell, or a squamous cell.

In embodiments, provided herein are methods of introducing the modified double-stranded oligonucleotide into a cell in vivo by intrathecal administration. In embodiments, provided herein are methods of introducing the modified double-stranded oligonucleotide into a cell by intraventricular administration.

In embodiments, provided herein are methods of introducing the modified double-stranded oligonucleotide into a cell in vivo by contacting systemic administration of the lipid-conjugated compound of Formulae I, Ia, Ib, II, IIa, IIb, III, IIIa, or IIIb, or a corresponding pharmaceutically acceptable salt thereof.

In embodiments, provided herein are methods of introducing any of the lipid-conjugated compounds Formulae I, Ia, Ib, II, IIa, IIb, III, IIIa, or IIIb, or a pharmaceutically acceptable salt thereof, into a cell. In embodiments, the cell is in vitro. In embodiments, the cell is ex vivo. In embodiments, the cell is in vivo.

In embodiments, provided herein are methods of administering any of the lipid-conjugated compounds of Formulae I, Ia, Ib, II, IIa, IIb, III, IIIa, or IIIb, or a corresponding pharmaceutically acceptable salt thereof, to a subject. The subject may have a disease or disorder of the eye, brain, liver, kidney, heart, adipose tissue, lung, muscle or spleen.

In embodiments, the disease or disorder of the eye is blepharitis, cataracts, chalazion, conjunctivitis, diabetic retinopathy, dry eye, glaucoma, keratitis, keratoconus, macular degeneration, ocular allergies, ocular hypertension, pinguecula, presbyopia, pterygium, retinoblastoma, subconjunctival hemorrhage, or Uveitis.

In embodiments, the disease or disorder is a neurological disease or disorder, a metabolic disease or disorder, an inflammatory disease or disorder. In embodiments, the subject has cancer.

In any of the embodiments related to administration in vivo or to a subject, the administration is systemic administration, which may include, without limitation, subcutaneous administration, intravenous administration, intramuscular administration, and oral administration. In any of the embodiments related to administration in vivo or to a subject, the administration is local administration, which may include, without limitation, intravitreal administration, intrathecal administration, and intraventricular administration.

In embodiments, provided herein is a method of introducing a modified double-stranded oligonucleotide ex vivo, comprising contacting the cells with a compound of Formulae I, Ia, Ib, II, IIa, IIb, III, IIIa, or IIIb or a corresponding pharmaceutically acceptable salt thereof under free uptake conditions. In embodiments, the cells are neurons, TBM cells, skeletal muscle cells, adipocyte cells or hepatocyte cells.

In embodiments, provided herein is a cell containing a compound having the structure of Formulae I, Ia, Ib, II, IIa, IIb, III, IIIa, or IIIb or a corresponding pharmaceutically acceptable salt thereof. In embodiments, the cell is a mammalian cell. In embodiments, the cell is a human cell. In embodiments, the cell is a mouse cell. In embodiments, the cell is a fibroblast cell. In embodiments, the cell is a NIH3T3 cell. In embodiments, the cell is a kidney cell. In embodiments, the cell is a HEK293 cell. In embodiments, the cell is an endothelial cell. In embodiments, the cell is a HUVEC cell. In embodiments, the cell is an adipose cell. In embodiments, the cell is a differentiated 3T3L1 cell. In embodiments, the cell is a macrophage cell. In embodiments, the cell is a RAW264.7 cell. In embodiments, the cell is a neuronal cell. In embodiments, the cell is a primary rat neuron. In embodiments, the cell is a SH-SY5Y cell. In embodiments, the cell is a muscle cell. In embodiments, the cell is a differentiated primary human skeletal muscle cell. In embodiments, the cell is a cell of the trabecular meshwork. In embodiments, the cell may be from an immortalized cell line. In embodiments, the cell may be from primary cells. In embodiments, the cell is an adipocyte cell. In embodiments, the cell is a human adipocyte cell. In embodiments, the cell is a hepatocyte cell. In embodiments, the cell is a human hepatocyte cell. In embodiments, the cell is a primary human adipocyte cell. In embodiments, the cell is a primary HUVEC cell. In embodiments, the cell is a primary human hepatocyte cell.

In embodiments the cell contains a compound having the structure of Formula III:

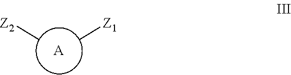

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to Z₁ at the 3′ end of one strand of the modified double-stranded oligonucleotide or the 3′ end of the modified single-stranded oligonucleotide, where Z₁ is

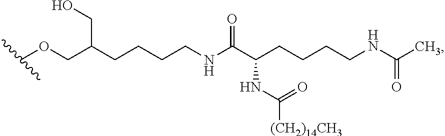

and
wherein the modified double-stranded oligonucleotide is conjugated to Z₂ at the 5′ end of one strand of the modified double-stranded oligonucleotide or the 5′ end of the modified single-stranded oligonucleotide, where Z₂ is

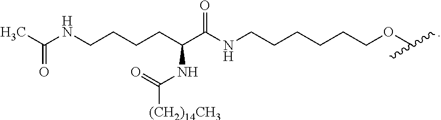

In embodiments the cell contains a compound having the structure of Formula IIIa:

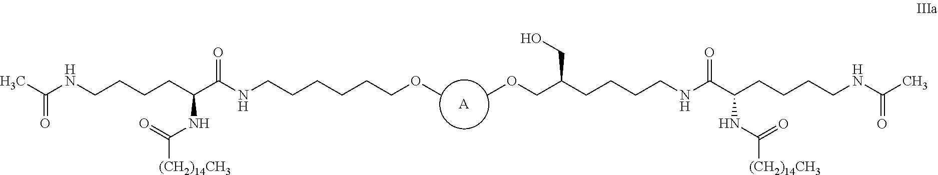

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

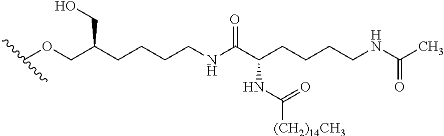

at the 3′ end of one strand of the modified double-stranded oligonucleotide or the 3′ end of the modified single-stranded oligonucleotide, and wherein the modified double-stranded oligonucleotide or modified single-stranded oligonucleotide is conjugated to a lipid-containing moiety

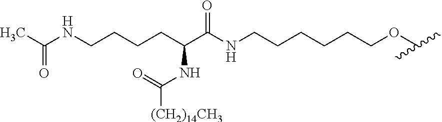

at the 5′ end of one strand of the modified double-stranded oligonucleotide or the 5′ end of the modified single-stranded oligonucleotide.

In embodiments the cell contains a compound having the structure of Formula IIIb:

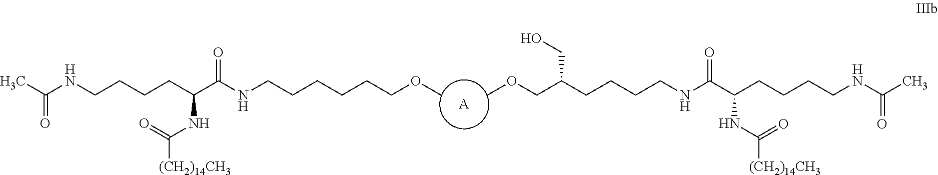

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

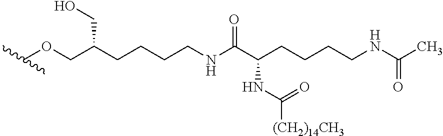

at the 3′ end of one strand of the modified double-stranded oligonucleotide or the 3′ end of the modified single-stranded oligonucleotide, and wherein the modified double-stranded oligonucleotide or single-stranded oligonucleotide is conjugated to a lipid-containing moiety

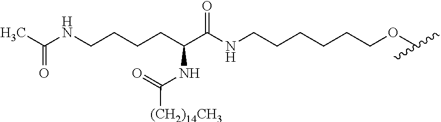

at the 5′ end of one strand of the modified double-stranded oligonucleotide or the 5′ end of the modified single-stranded oligonucleotide.

In embodiments of the cell containing a compound having the structure of Formulae I, Ia, Ib, II, IIa, IIb, III, IIIa, or IIIb, the cell is a mammalian cell. In embodiments, the cell is a human cell. In embodiments, the cell is an endothelial cell. In embodiments, the cell is a HUVEC cell.

In embodiments of a cell containing a compound having the structure of Formulae I, Ia, Ib, II, IIa, IIb, III, IIIa, or IIIb, the modified double-stranded oligonucleotide is conjugated at either of its 3′ ends to the lipid-containing moiety portion of the compound. In embodiments, the modified double-stranded oligonucleotide is conjugated at the 3′end of its guide strand to the lipid-containing moiety portion. In embodiments, the modified double-stranded oligonucleotide is conjugated at the 3′end of its passenger strand to the lipid-containing moiety portion.

In embodiments of a cell containing a compound having the structure of Formulae I, Ia, Ib, II, IIa, IIb, III, IIIa, or IIIb, the conjugation occurs through a phosphodiester bond.

In embodiments of a cell containing a compound having the structure of Formulae I, Ia, Ib, II, IIa, IIb, III, IIIa, or IIIb, the modified double-stranded oligonucleotide is conjugated at either of its 5′ ends to the lipid-containing moiety portion of the compound. In embodiments, the modified double-stranded oligonucleotide is conjugated at the 5′end of its guide strand to the lipid-containing moiety portion. In embodiments, the modified double-stranded oligonucleotide is conjugated at the 5′end of its passenger strand to the lipid-containing moiety portion.

In embodiments of a cell containing a compound having the structure of Formulae I, Ia, Ib, II, IIa, IIb, III, IIIa, or IIIb, the conjugation occurs through a phosphodiester bond.

In embodiments, provided herein are methods of introducing a modified double-stranded oligonucleotide into a human umbilical vein endothelial cell, NIH3T3 cell, RAW264.7 cell, a HEK293 cell or SH-SY5Y cell in vitro, comprising contacting the cell under free uptake conditions with a compound having the structure of Formula I, Ia, Ib, II, IIa, IIb, III, IIIa, or IIIb or a corresponding pharmaceutically acceptable salt thereof. In embodiments of the method, the compound may be:

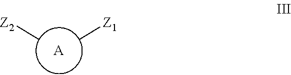

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to Z₁ at the 3′ end of one strand of the modified double-stranded oligonucleotide or the 3′ end of the modified single-stranded oligonucleotide, where Z₁ is

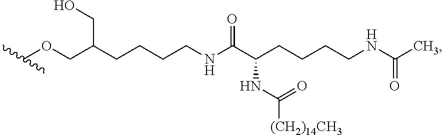

and wherein the modified double-stranded oligonucleotide or modified single-stranded oligonucleotide is conjugated to Z₂ at the 5′ end of one strand of the modified double-stranded oligonucleotide or the 5′ end of the modified single-stranded oligonucleotide, where Z₂ is

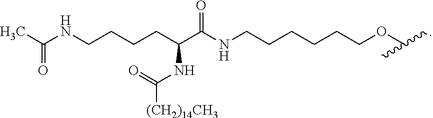

In embodiments of the method, the compound may be:

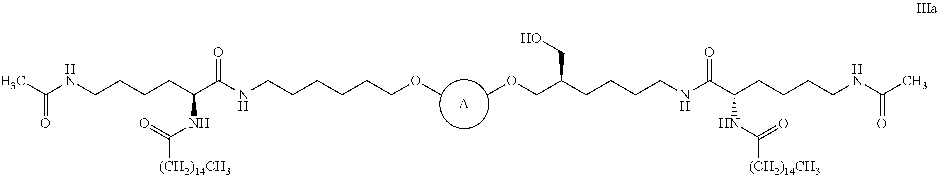

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

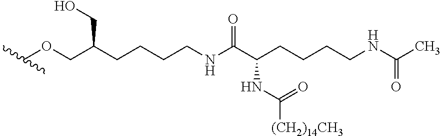

at the 3′ end of one strand of the modified double-stranded oligonucleotide or the 3′ end of the modified single-stranded oligonucleotide, and wherein the modified double-stranded oligonucleotide or single-stranded oligonucleotide is conjugated to a lipid-containing moiety

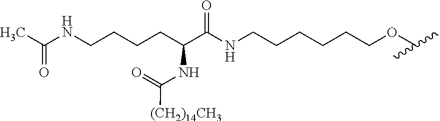

at the 5′ end of one strand of the modified double-stranded oligonucleotide or the 5′ end of the modified single-stranded oligonucleotide.

In embodiments of the method, the compound may be:

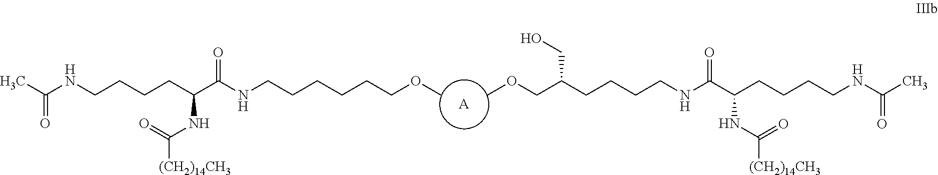

or a pharmaceutically acceptable salt thereof, wherein A is a modified double-stranded oligonucleotide or modified single-stranded oligonucleotide, wherein the modified double-stranded oligonucleotide or modified single-stranded oligonucleotide is conjugated to a lipid-containing moiety

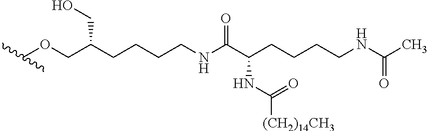

at the 3′ end of one strand of the modified double-stranded oligonucleotide or the 3′ end of the modified single-stranded oligonucleotide, and wherein the modified double-stranded oligonucleotide is conjugated to a lipid-containing moiety

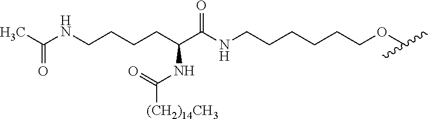

at the 5′ end of one strand of the modified double-stranded oligonucleotide or the 5′ end of the modified single-stranded oligonucleotide.

In embodiments of methods of introducing a modified double-stranded oligonucleotide into a human umbilical vein endothelial cell, NIH3T3 cell, RAW264.7 cell, a HEK293 cell or SH-SY5Y cell in vitro, comprising contacting the cell under free uptake conditions with a compound having the structure of Formula III, IIIa, or IIIb, the modified double-stranded oligonucleotide is conjugated at either of its 3′ ends to the lipid-containing moiety portion of the compound. In embodiments, the modified double-stranded oligonucleotide is conjugated at the 3′end of its guide strand to the lipid-containing moiety portion. In embodiments, the modified double-stranded oligonucleotide is conjugated at the 3′end of its passenger strand to the lipid-containing moiety portion.

In embodiments of methods of introducing a modified double-stranded oligonucleotide into a human umbilical vein endothelial cell, NIH3T3 cell, RAW264.7 cell, a HEK293 cell or SH-SY5Y cell in vitro, comprising contacting the cell under free uptake conditions with a compound having the structure of Formula III, IIIa, or IIIb, the conjugation occurs through a phosphodiester bond.

In embodiments of methods of introducing a modified double-stranded oligonucleotide into a human umbilical vein endothelial cell, NIH3T3 cell, RAW264.7 cell, a HEK293 cell or SH-SY5Y cell in vitro, comprising contacting the cell under free uptake conditions with a compound having the structure of Formula III, IIIa, or IIIb, the modified double-stranded oligonucleotide is conjugated at either of its 5′ ends to the lipid-containing moiety portion of the compound. In embodiments, the modified double-stranded oligonucleotide is conjugated at the 5′end of its guide strand to the lipid-containing moiety portion. In embodiments, the modified double-stranded oligonucleotide is conjugated at the 5′end of its passenger strand to the lipid-containing moiety portion.

In embodiments of methods of introducing a modified double-stranded oligonucleotide into a human umbilical vein endothelial cell, NIH3T3 cell, RAW264.7 cell, a HEK293 cell or SH-SY5Y cell in vitro, comprising contacting the cell under free uptake conditions with a compound having the structure of Formula III, IIIa, or IIIb, the conjugation occurs through a phosphodiester bond.

In embodiments, the modified double-stranded oligonucleotide is a small interfering RNA (siRNA). In embodiments, the modified double-stranded oligonucleotide is a microRNA mimic.

In embodiments, the modified single-stranded oligonucleotide is targeted to a messenger RNA. In embodiments, the modified single-stranded oligonucleotide is an RNaseH oligonucleotide, which is dependent on RNaseH for cleavage of the mRNA to which it is complementary. In embodiments, the modified single-stranded oligonucleotide is a single-stranded siRNA. In embodiments, the modified single-stranded oligonucleotide is targeted to a microRNA. In embodiments, the modified single-stranded oligonucleotide is targeted to a long non-coding RNA.

In embodiments, the modified double-stranded oligonucleotide contains at least one phosphorothioate linkage. In some such embodiments, the modified double-stranded oligonucleotide contains two to thirteen phosphorothioate linkages. In some particular embodiments, the modified double-stranded oligonucleotide contains four phosphorothioate linkages. In some particular embodiments, the modified double-stranded oligonucleotide contains two phosphorothioate linkages at the 3′ end of the guide strand and two phosphorothioate linkages at the 3′end of the passenger strand. In some particular embodiments, the modified double-stranded oligonucleotide contains two phosphorothioate linkages at the 5′ end of the guide strand and two phosphorothioate linkages at the 3′end of the passenger strand. In some particular embodiments, the modified double-stranded oligonucleotide contains five phosphorothioate linkages. In some particular embodiments, the modified double-stranded oligonucleotide contains six phosphorothioate linkages. In some particular embodiments, the modified double-stranded oligonucleotide contains seven phosphorothioate linkages. In some particular embodiments, the modified double-stranded oligonucleotide contains eight phosphorothioate linkages. In some particular embodiments, the modified double-stranded oligonucleotide contains nine phosphorothioate linkages. In some particular embodiments, the modified double-stranded oligonucleotide contains ten phosphorothioate linkages. In some particular embodiments, the modified double-stranded oligonucleotide contains eleven phosphorothioate linkages. In some particular embodiments, the modified double-stranded oligonucleotide contains twelve phosphorothioate linkages. In some particular embodiments, the modified double-stranded oligonucleotide contains thirteen phosphorothioate linkages. In some particular embodiments, the modified double-stranded oligonucleotide contains two phosphorothioate linkages at the 3′ end of the guide strand, seven phosphorothioate linkages at the 5′ end of the guide strand, two phosphorothioate linkages at the 3′end of the passenger strand, and two phosphorothioate linkages at the 5′end of the passenger strand.

In embodiments, the modified double-stranded oligonucleotide contains at least one phosphoroamidate linkage. In embodiments, the modified double-stranded oligonucleotide contains at least one phosphorodithioate linkage. In embodiments, the modified double-stranded oligonucleotide contains at least one boranophosphonate linkage. In embodiments, the modified double-stranded oligonucleotide contains at least one O-methylphosphoroamidite linkage. In embodiments, the modified double-stranded oligonucleotide contains a positive backbone. In embodiments, the modified double-stranded oligonucleotide contains a non-ionic backbone.

In embodiments, the modified double-stranded oligonucleotide contains at least one 2′-O-methyl residue. In embodiments, the at least one 2′-O-methyl residue is present on the guide strand, the passenger strand, or both the guide strand and the passenger strand. In embodiments, the modified double-stranded oligonucleotide contains at least one 2′-deoxy-2′-fluoro residue. In embodiments, the at least one 2′-deoxy-2′-fluoro residue is present on the guide strand, the passenger strand, or both the guide strand and the passenger strand. In embodiments, the modified double-stranded oligonucleotide contains 2′-O-methyl residues alternating with 2′-deoxy-2′-fluoro residues. In embodiments, such alternating residues are present on the guide strand, the passenger strand, or both the guide strand and the passenger strand. In embodiments, the modified double-stranded oligonucleotide contains three 2′-O-methyl residues on the passenger strand and three 2′-deoxy-2′-fluoro residues on the guide strand. In embodiments, every residue in the modified double-stranded oligonucleotide is either a 2′-O-methyl residue or a 2′-deoxy-2′-fluoro residue. In embodiments, the modified double-stranded oligonucleotide contains at least one residue wherein the ribose is locked by a covalent linkage between the 2′ and 4′ carbons, i.e. the residue is a bicyclic nucleic acid (BNA) residue. In embodiments, the bicyclic nucleic acid is a locked nucleic acid (LNA) residue. In embodiments, the bicyclic nucleic acid residue is a constrained ethyl (cEt) residue, also known as cEt residue. In embodiments, the modified double-stranded oligonucleotide includes an unlocked nucleic acid (UNA) residue. In embodiments, the modified double-stranded oligonucleotide contains a non-ribose backbone. In embodiments, the modified double-stranded oligonucleotide contains a single strand of locked nucleic acids (LNA), bicyclic nucleic acids (BNA), e.g. cEt, UNA, or a phosphorodiamidate morpholino oligomer (PMO), or modification thereof. In embodiments, the modified double-stranded oligonucleotide contains a single strand comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, of DNA, siRNA, mRNA, locked nucleic acids (LNA), bicyclic nucleic acids (BNA), e.g. cEt, UNA, or phosphorodiamidate morpholino oligomer (PMO), or modification thereof and the like, or the oligonucleotide may comprise an amount of DNA, siRNA, mRNA, locked nucleic acids (LNA), bicyclic nucleic acids (BNA), e.g. cEt, UNA, or phosphorodiamidate morpholino oligomer (PMO), or modification thereof and the like within a range defined by any of two of the preceding values. In embodiments, the modified double-stranded oligonucleotide contains a single strand comprising at least 1% and less than 20%, 19%, 18%, 17%, 16%, 15%, 14%, 13%, 12%, 11%, 10%, 9%, 8%, 7%, 6%, 5%, or 4% of 2′-O-methoxy ethyl/phosphorothioate (MOE).

In embodiments, the modified double-stranded oligonucleotide comprises a 5′-(E)-vinylphosphonate group at the 5′ end of the guide strand. In embodiments, the modified double-stranded oligonucleotide is an siRNA comprising a 5′-(E)-vinylphosphonate group at the 5′ end of the guide strand. In embodiments, the modified double-stranded oligonucleotide is an microRNA mimic comprising a 5′-(E)-vinylphosphonate group at the 5′ end of the guide strand. In embodiments, the modified single-stranded oligonucleotide comprises a 5′-(E)-vinylphosphonate group at the 5′ end of the oligonucleotide. In embodiments, the modified single-stranded oligonucleotide is a single-stranded siRNA comprising a 5′-(E)-vinylphosphonate group at the 5′ end.

Any of the modified single-stranded oligonucleotides disclosed herein may comprise one or more nucleoside sugar modifications selected from a 2′-O-methoxy ethyl residue, a bicyclic nucleic acid residue, a 2′-O-methyl residue, and a 2′-fluoro residue. In embodiments, the bicyclic nucleic acid residue is a locked nucleic acid residue. In embodiments, the bicyclic nucleic acid residue is a cEt residue. Any of the modified single-stranded nucleic acids (e.g., oligonucleotides) disclosed herein may comprise one or more phosphorothioate linkages. In embodiments, each linkage of a modified single-stranded oligonucleotide is a phosphorothioate linkage.

In embodiments, the double-stranded oligonucleotide is a small interfering RNA (siRNA). In embodiments, the double-stranded oligonucleotide is a microRNA mimic.

In embodiments, the single-stranded oligonucleotide is targeted to a messenger RNA. In embodiments, the single-stranded oligonucleotide is an RNaseH oligonucleotide, which is dependent on RNaseH for cleavage of the mRNA to which it is complementary. In embodiments, the single-stranded oligonucleotide is a single-stranded siRNA. In embodiments, the single-stranded oligonucleotide is targeted to a microRNA. In embodiments, the single-stranded oligonucleotide is targeted to a long non-coding RNA.

In embodiments, the double-stranded oligonucleotide contains at least one phosphorothioate linkage. In some such embodiments, the double-stranded oligonucleotide contains two to thirteen phosphorothioate linkages. In some particular embodiments, the double-stranded oligonucleotide contains four phosphorothioate linkages. In some particular embodiments, the double-stranded oligonucleotide contains two phosphorothioate linkages at the 3′ end of the guide strand and two phosphorothioate linkages at the 3′end of the passenger strand. In some particular embodiments, the double-stranded oligonucleotide contains two phosphorothioate linkages at the 5′ end of the guide strand and two phosphorothioate linkages at the 3′end of the passenger strand. In some particular embodiments, the double-stranded oligonucleotide contains five phosphorothioate linkages. In some particular embodiments, the double-stranded oligonucleotide contains six phosphorothioate linkages. In some particular embodiments, the double-stranded oligonucleotide contains seven phosphorothioate linkages. In some particular embodiments, the double-stranded oligonucleotide contains eight phosphorothioate linkages. In some particular embodiments, the double-stranded oligonucleotide contains nine phosphorothioate linkages. In some particular embodiments, the double-stranded oligonucleotide contains ten phosphorothioate linkages. In some particular embodiments, the double-stranded oligonucleotide contains eleven phosphorothioate linkages. In some particular embodiments, the double-stranded oligonucleotide contains twelve phosphorothioate linkages. In some particular embodiments, the double-stranded oligonucleotide contains thirteen phosphorothioate linkages. In some particular embodiments, the double-stranded oligonucleotide contains two phosphorothioate linkages at the 3′ end of the guide strand, seven phosphorothioate linkages at the 5′ end of the guide strand, two phosphorothioate linkages at the 3′end of the passenger strand, and two phosphorothioate linkages at the 5′end of the passenger strand.

In embodiments, the double-stranded oligonucleotide contains at least one phosphoroamidate linkage. In embodiments, the double-stranded oligonucleotide contains at least one phosphorodithioate linkage. In embodiments, the double-stranded oligonucleotide contains at least one boranophosphonate linkage. In embodiments, the double-stranded oligonucleotide contains at least one O-methylphosphoroamidite linkage. In embodiments, the double-stranded oligonucleotide contains a positive backbone. In embodiments, the double-stranded oligonucleotide contains a non-ionic backbone.

In embodiments, the double-stranded oligonucleotide contains at least one 2′-O-methyl residue. In embodiments, the at least one 2′-O-methyl residue is present on the guide strand, the passenger strand, or both the guide strand and the passenger strand. In embodiments, the double-stranded oligonucleotide contains at least one 2′-deoxy-2′-fluoro residue. In embodiments, the at least one 2′-deoxy-2′-fluoro residue is present on the guide strand, the passenger strand, or both the guide strand and the passenger strand. In embodiments, the double-stranded oligonucleotide contains 2′-O-methyl residues alternating with 2′-deoxy-2′-fluoro residues. In embodiments, such alternating residues are present on the guide strand, the passenger strand, or both the guide strand and the passenger strand. In embodiments, the double-stranded oligonucleotide contains three 2′-O-methyl residues on the passenger strand and three 2′-deoxy-2′-fluoro residues on the guide strand. In embodiments, every residue in the double-stranded oligonucleotide is either a 2′-O-methyl residue or a 2′-deoxy-2′-fluoro residue. In embodiments, the double-stranded oligonucleotide contains at least one residue wherein the ribose is locked by a covalent linkage between the 2′ and 4′ carbons, i.e. the residue is a bicyclic nucleic acid (BNA) residue. In embodiments, the bicyclic nucleic acid is a locked nucleic acid (LNA) residue. In embodiments, the bicyclic nucleic acid residue is a constrained ethyl (cEt) residue, also known as cEt residue. In embodiments, the double-stranded oligonucleotide includes an unlocked nucleic acid (UNA) residue. In embodiments, the double-stranded oligonucleotide contains a non-ribose backbone. In embodiments, the double-stranded oligonucleotide contains a single strand of locked nucleic acids (LNA), bicyclic nucleic acids (BNA), e.g. cEt, UNA, or a phosphorodiamidate morpholino oligomer (PMO), or modification thereof. In embodiments, the double-stranded oligonucleotide contains a single strand comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, of DNA, siRNA, mRNA, locked nucleic acids (LNA), bicyclic nucleic acids (BNA), e.g. cEt, UNA, or phosphorodiamidate morpholino oligomer (PMO), or modification thereof and the like, or the oligonucleotide may comprise an amount of DNA, siRNA, mRNA, locked nucleic acids (LNA), bicyclic nucleic acids (BNA), e.g. cEt, UNA, or phosphorodiamidate morpholino oligomer (PMO), or modification thereof and the like within a range defined by any of two of the preceding values. In embodiments, the double-stranded oligonucleotide contains a single strand comprising at least 1% and less than 20%, 19%, 18%, 17%, 16%, 15%, 14%, 13%, 12%, 11%, 10%, 9%, 8%, 7%, 6%, 5%, or 4% of 2′-O-methoxy ethyl/phosphorothioate (MOE).

In embodiments, the double-stranded oligonucleotide comprises a 5′-(E)-vinylphosphonate group at the 5′ end of the guide strand. In embodiments, the double-stranded oligonucleotide is an siRNA comprising a 5′-(E)-vinylphosphonate group at the 5′ end of the guide strand. In embodiments, the double-stranded oligonucleotide is an microRNA mimic comprising a 5′-(E)-vinylphosphonate group at the 5′ end of the guide strand. In embodiments, the single-stranded oligonucleotide comprises a 5′-(E)-vinylphosphonate group at the 5′ end of the oligonucleotide. In embodiments, the single-stranded oligonucleotide is a single-stranded siRNA comprising a 5′-(E)-vinylphosphonate group at the 5′ end.

Any of the single-stranded oligonucleotides disclosed herein may comprise one or more nucleoside sugar modifications selected from a 2′-O-methoxy ethyl residue, a bicyclic nucleic acid residue, a 2′-O-methyl residue, and a 2′-fluoro residue. In embodiments, the bicyclic nucleic acid residue is a locked nucleic acid residue. In embodiments, the bicyclic nucleic acid residue is a cEt residue. Any of the single-stranded nucleic acids (e.g., oligonucleotides) disclosed herein may comprise one or more phosphorothioate linkages. In embodiments, each linkage of a single-stranded oligonucleotide is a phosphorothioate linkage.

In embodiments, a compound as disclosed and described herein may act as an inhibitor. In embodiments, a compound as disclosed and described herein may act as an inhibitor of gene expression. In embodiments, a compound as disclosed and described herein may act as an inhibitor of protein expression. In embodiments, a compound or composition comprising a compound as disclosed and described herein may act as an inhibitor of gene expression in the presence of an activator of gene expression. In embodiments, a compound as disclosed and described herein may act as an inhibitor of protein expression in the presence of an activator of gene expression. In embodiments, a compound or composition comprising a compound as disclosed and described herein may act as an inhibitor of protein expression in the presence of an activator of protein expression. In embodiments, a compound as disclosed and described herein may act as an inhibitor in vitro or ex vivo. In embodiments, a compound may act as an inhibitor in vitro using a primary cell. In embodiments, a compound may act as an inhibitor in vitro using an immortalized cell. In embodiments, the compound may decrease expression or activity 10%, 20%, 30%, 40%, 50%, 60%, 70%, 80%, 90% or more, or within a range defined by any of two of the preceding values, in comparison to a control in the absence of the inhibitor. In embodiments, the compound may decrease expression or activity 10%, 20%, 30%, 40%, 50%, 60%, 70%, 80%, 90% or more, or within a range defined by any of two of the preceding values, in comparison to a control in the presence of an activator of gene expression. In embodiments, the compound may decrease expression or activity 10%, 20%, 30%, 40%, 50%, 60%, 70%, 80%, 90% or more, or within a range defined by any of two of the preceding values, in comparison to a control in the presence of an activator of protein expression.

### EMBODIMENTS

### Embodiments P

Embodiment P1. A lipid-conjugated compound having the structure of Formula I:

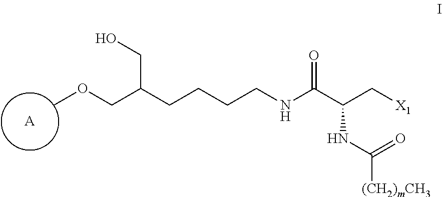

or a pharmaceutically acceptable salt thereof, wherein:
A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded oligonucleotide;
X¹ is

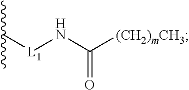

L₁ is 13 (CH₂)ₙ—, —(CH₂)ₙL₂(CH₂)ₙ—, or a bond;
L₂ is —C(═O)NH—, —C(═O)O—, —OC(═O)O—, —NHC(═O)O—, —NHC(═O)NH—, —C(═S)NH—, —C(═O)S—, —NH—, O (oxygen), S (sulfur), and wherein each m is independently an integer from 10 to 18 and wherein each n is independently an integer from 1 to 6.

Embodiment P2. The compound of Embodiment P1, wherein each m is 10, L₁ is —(CH₂)ₙ—, and n is 3.

Embodiment P3. The compound of Embodiment P1, wherein each m is 11, L₁ is —(CH₂)ₙ—, and n is 3.

Embodiment P4. The compound of Embodiment P1, wherein each m is 12, L₁ is —(CH₂)ₙ—, and n is 3.

Embodiment P5. The compound of Embodiment P1, wherein each m is 13, L₁ is —(CH₂)ₙ—, and n is 3.

Embodiment P6. The compound of Embodiment P1, wherein each m is 14, L₁ is —(CH₂)ₙ—, and n is 3.

Embodiment P7. The compound of Embodiment P1, wherein each m is 15, L₁ is —(CH₂)ₙ—, and n is 3.

Embodiment P8. The compound of Embodiment P1, wherein each m is 16, L₁ is —(CH₂)ₙ—, and n is 3.

Embodiment P9. The compound of Embodiment P1, wherein each m is 17, L₁ is —(CH₂)ₙ—, and n is 3.

Embodiment P10. The compound of Embodiment P1, wherein each m is 18, L₁ is —(CH₂)ₙ—, and n is 3.

Embodiment P11. The compound of Embodiment P1, wherein each m is independently an integer from 12 to 16; and wherein each n is independently an integer from 1 to 6.

Embodiment P12. The compound of Embodiment P1, wherein each m is independently an integer from 12 to 14; and wherein each n is independently an integer from 1 to 6.

Embodiment P13. The compound of Embodiment P1, wherein L₁ is a bond; and each m is independently an integer from 12 to 16.

Embodiment P14. The compound of Embodiment P1, wherein L₁ is —(CH₂)₃C(═O)NH(CH₂)₅—; and each m is independently an integer from 12 to 16.

Embodiment P15. The compound of Embodiment P13 or P14, wherein each m is 14.

Embodiment P16. A lipid-conjugated compound having the structure of Formula II:

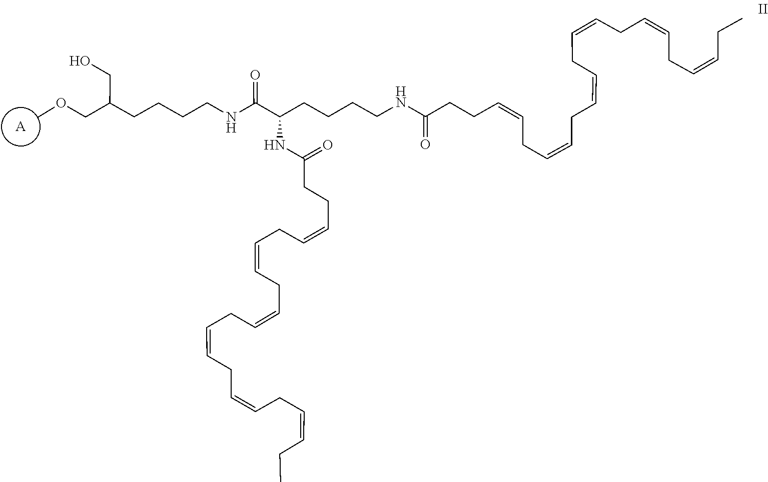

or a pharmaceutically acceptable salt thereof, wherein:
A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded oligonucleotide.

Embodiment P17. A lipid-conjugated compound having the structure of Formula III

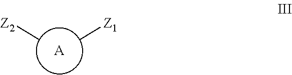

or a pharmaceutically acceptable salt thereof, wherein:
A is a modified double-stranded oligonucleotide or modified single-stranded oligonucleotide, wherein the modified double-stranded oligonucleotide or modified single-stranded oligonucleotide is conjugated to Z₁ at the 3′ end of one strand of the modified double-stranded oligonucleotide or the 3′ end of the modified single-stranded oligonucleotide, where Z₁ is

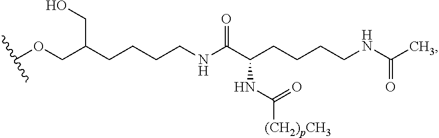

wherein p is an integer from 10 to 18, and
wherein the modified double-stranded oligonucleotide or modified single-stranded oligonucleotide is conjugated to Z₂ at the 5′ end of one strand of the modified double-stranded oligonucleotide or the 5′ end of the modified single-stranded oligonucleotide, where Z₂ is

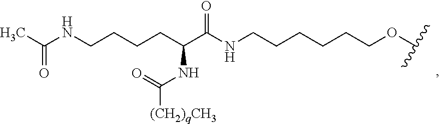

wherein q is an integer from 10 to 18.

Embodiment P18. The compound of Embodiment P17, wherein p is 14; and q is 14.

Embodiment P19. The compound of any one of Embodiments P1 to P18, wherein the modified double-stranded oligonucleotide contains at least one phosphorothioate linkage.

Embodiment P20. The compound of any one of Embodiments P1 to P19, wherein the modified double-stranded oligonucleotide contains at least one 2′-O-methyl residue.

Embodiment P21. The compound of any one of Embodiments P1 to P20, wherein the modified double-stranded oligonucleotide contains at least one 2′-deoxy-2′-fluoro residue.

Embodiment P22. The compound of any one of Embodiments P1 to P21, wherein the modified double-stranded oligonucleotide comprises a single strand of a DNA, siRNA, mRNA, locked nucleic acids (LNA), bridged nucleic acids (BNA), or phosphorodiamidate morpholino oligomer (PMO), or modification thereof.

Embodiment P23. The compound of Embodiment P22, wherein the modified double-stranded oligonucleotide comprises a single strand of locked nucleic acids (LNA), or modification thereof.

Embodiment P24. The compound of Embodiment P22, wherein the modified double-stranded oligonucleotide comprises a single strand of phosphorodiamidate morpholino oligomer (PMO), or modification thereof.

Embodiment P25. The compound of any one of Embodiments P1 to P24, wherein the lipid moiety is attached to the 3′ end of the passenger strand.

Embodiment P26. The compound of any one of Embodiments P1 to P25, wherein the oligonucleotide comprises at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, of DNA, siRNA, mRNA, locked nucleic acids (LNA), bridged nucleic acids (BNA), or phosphorodiamidate morpholino oligomer (PMO), or modification thereof, or the oligonucleotide may comprise an amount of DNA, siRNA, mRNA, locked nucleic acids (LNA), bridged nucleic acids (BNA), or phosphorodiamidate morpholino oligomer (PMO), or modification thereof within a range defined by any of two of the preceding values.

Embodiment P27. The compound of any one of Embodiments P1 to P25, wherein the oligonucleotide comprises at least 1% and less than 20%, 19%, 18%, 17%, 16%, 15%, 14%, 13%, 12%, 11%, 10%, 9%, 8%, 7%, 6%, 5%, or 4% of 2′-O-methoxy ethyl/phosphorothioate (MOE).

Embodiment P28. A cell containing the compound of any one of Embodiments P1 to P27.

Embodiment P29. The cell of Embodiment P28, wherein the cell is a primary cell.

Embodiment P30. The cell of Embodiment P29, wherein the cell is an adipocyte cell, a hepatocyte cell, a fibroblast cell, an endothelial cell, a kidney cell, a human umbilical vein endothelial cell (HUVEC), an adipose cell, a macrophage cell, a neuronal cell, a muscle cell, or a differentiated primary human skeletal muscle cell.

Embodiment P31. The cell of Embodiment P30, wherein the cell is a human umbilical vein endothelial cell.

Embodiment P32. The cell of Embodiment P28, wherein the cell is an immortalized cell.

Embodiment P33. The cell of Embodiment P32, wherein the cell is a NIH3T3 cell, a differentiated 3T3L1 cell, a RAW264.7 cell, or a SH-SY5Y cell.

Embodiment P34. The cell of Embodiment P28 or P30, wherein the cell is an adipocyte cell or a hepatocyte cell.

Embodiment P35. A method of introducing a modified double-stranded oligonucleotide into a cell in vitro, comprising contacting the cell with the compound of any one of Embodiments P1 to P27 under free uptake conditions.

Embodiment P36. The method of Embodiment P35, wherein the method is ex vivo and the cell is a primary cell.

Embodiment P37. The method of Embodiment P36, wherein the cell is an adipocyte cell, a hepatocyte cell, a fibroblast cell, an endothelial cell, a kidney cell, a human umbilical vein endothelial cell (HUVEC), an adipose cell, a macrophage cell, a neuronal cell, a rat neuron, a muscle cell, or a differentiated primary human skeletal muscle cell.

Embodiment P38. The method of Embodiment P36, wherein the cell is a human umbilical vein endothelial cell.

Embodiment P39. The method of Embodiment P35, wherein the cell is an immortalized cell.

Embodiment P40. The method of Embodiment P39, wherein the cell is a NIH3T3 cell, a differentiated 3T3L1 cell, a RAW264.7 cell, or a SH-SY5Y cell.

Embodiment P41. The method of Embodiment P35 or P37, wherein the cell is an adipocyte cell or a hepatocyte cell.

Embodiment P42. A method of introducing a modified double-stranded oligonucleotide ex vivo, comprising: obtaining cells; and contacting the cells with the compound of any one of Embodiments P1 to P27 under free uptake conditions.

Embodiment P43. The method of Embodiment P42, wherein the cells are neurons, TBM cells, skeletal muscle cells, adipocyte cells or a hepatocyte cells.

Embodiment P44. The method of Embodiment P42, wherein the cells are human umbilical vein endothelial cells.

### Embodiments Q

Embodiment Q1. A compound having the structure:

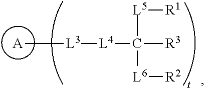

wherein
A is an oligonucleotide;
L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-.
L⁶ is -L^{6A}-L^{6B}-L^{6C}-L^{6D}-L^{6E}-.

L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
t is an integer from 1 to 5.

Embodiment Q2. The compound of Embodiment Q1, wherein t is 1.

Embodiment Q3. The compound of Embodiment Q1, wherein t is 2.

Embodiment Q4. The compound of Embodiment Q1, wherein t is 3.

Embodiment Q5. The compound of one of Embodiments Q1 to Q4, wherein A is a double-stranded oligonucleotide, or a single-stranded oligonucleotide.

Embodiment Q6. The compound of one of Embodiments Q1 to Q5, wherein the oligonucleotide of A is modified.

Embodiment Q7. The compound of one of Embodiments Q5 to Q6, wherein one L³ is attached to a 3′ carbon of the double-stranded oligonucleotide or single-stranded oligonucleotide.

Embodiment Q8. The compound of one of Embodiments Q5 to Q7, wherein one L³ is attached to a 5′ carbon of the double-stranded oligonucleotide or single-stranded oligonucleotide.

Embodiment Q9. The compound of one of Embodiments Q5 to Q8, wherein one L³ is attached to a nucleobase of the double-stranded oligonucleotide or single-stranded oligonucleotide.

Embodiment Q10. The compound of one of Embodiments Q1 to Q9, wherein L³ and L₄ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene or substituted or unsubstituted heteroalkylene.

Embodiment Q11. The compound of one of Embodiments Q1 to Q10, wherein L³ is independently

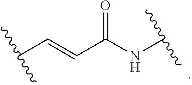 [SMILES: */C=C/C(=O)N*]

Embodiment Q12. The compound of one of Embodiments Q1 to Q10, wherein L³ is independently —OPO₂—O—.

Embodiment Q13. The compound of one of Embodiments Q1 to Q10, wherein L³ is independently —O—.

Embodiment Q14. The compound of one of Embodiments Q1 to Q13, wherein L⁴ is independently substituted or unsubstituted alkylene or substituted or unsubstituted hetoeroalkylene.

Embodiment Q15. The compound of one of Embodiments Q1 to Q13, wherein L⁴ is independently -L⁷-NH—C(O)— or -L⁷-C(O)—NH—, wherein L⁷ is substituted or unsubstituted alkylene.

Embodiment Q16. The compound of one of Embodiments Q1 to Q13, wherein L⁴ is independently

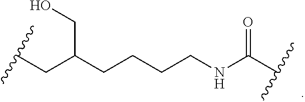 [SMILES: *CC(CO)CCCCNC(*)=O]

Embodiment Q17. The compound of one of Embodiments Q1 to Q13, wherein L⁴ is independently

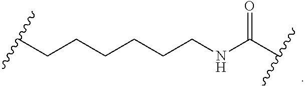 [SMILES: *CCCCCCNC(*)=O]

Embodiment Q18. The compound of one of Embodiments Q1 to Q17, wherein -L³-L⁴- is independently —O-L⁷-NH—C(O)— or —O-L⁷-C(O)—NH—, wherein L⁷ is independently substituted or unsubstituted alkylene, substituted or unsubstituted heteroalkylene, or substituted or unsubstituted heteroalkenylene.

Embodiment Q19. The compound of one of Embodiments Q1 to Q17, wherein -L³-L⁴- is independently —O-L⁷-NH—C(O)—, wherein L⁷ is independently substituted or unsubstituted C₅-C₈ alkylene.

Embodiment Q20. The compound of one of Embodiments Q1 to Q17, wherein -L³-L⁴- is independently

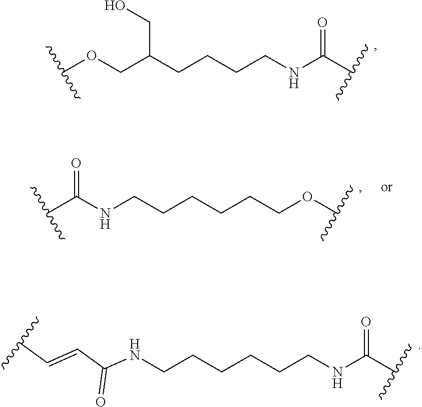 [SMILES: *OCC(CO)CCCCNC(*)=O ; *OCCCCCCNC(*)=O ; */C=C/C(=O)NCCCCCCNC(*)=O]

Embodiment Q21. The compound of one of Embodiments Q1 to Q17, wherein -L³-L⁴- is independently —OPO₂—O-L⁷-NH—C(O)— or —OPO₂—O-L⁷-C(O)—NH—, wherein L⁷ is independently substituted or unsubstituted alkylene.

Embodiment Q22. The compound of one of Embodiments Q1 to Q17, wherein -L³-L⁴- is independently —OPO₂—O-L⁷-NH—C(O)—, wherein L⁷ is independently substituted or unsubstituted C₅-C₈ alkylene.

Embodiment Q23. The compound of one of Embodiments Q1 to Q17, wherein -L³-L⁴- is independently

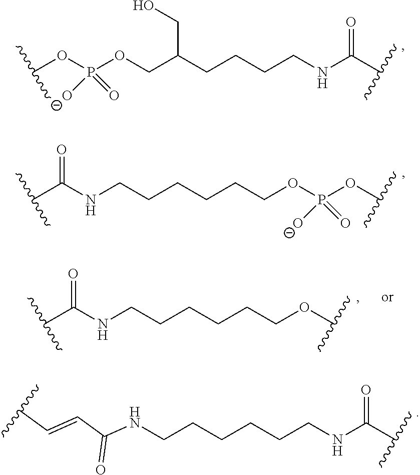 [SMILES: *OP(=O)([O-])OCC(CO)CCCCNC(*)=O ; *OP(=O)([O-])OCCCCCCNC(*)=O ; *OCCCCCCNC(*)=O ; */C=C/C(=O)NCCCCCCNC(*)=O]

Embodiment Q24. The compound of one of Embodiments Q1 to Q17, wherein an -L³-L⁴- is independently

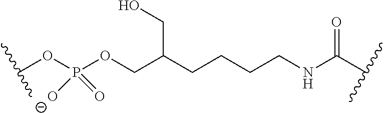 [SMILES: *OP(=O)([O-])OCC(CO)CCCCNC(*)=O]

and is attached to a 3′ carbon of the double-stranded oligonucleotide or single-stranded oligonucleotide.

Embodiment Q25. The compound of one of Embodiments Q1 to Q24, wherein an -L₃-L₄- is independently

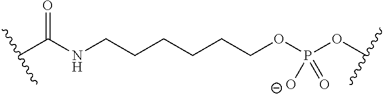 [SMILES: *OP(=O)([O-])OCCCCCCNC(*)=O]

and is attached to a 5′ carbon of the double-stranded oligonucleotide or single-stranded oligonucleotide.

Embodiment Q26. The compound of one of Embodiments Q1 to Q25, wherein an -L³-L⁴- is independently

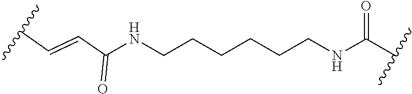 [SMILES: */C=C/C(=O)NCCCCCCNC(*)=O]

and is attached to a nucleotide base of the double-stranded nucleic acid or single-stranded nucleic acid.

Embodiment Q27. The compound of one of Embodiments Q1 to Q26, wherein R³ is independently hydrogen.

Embodiment Q28. The compound of one of Embodiments Q1 to Q27, wherein L⁶ is independently —NHC(O)—, —C(O)NH—,substituted or unsubstituted alkylene, or substituted or unsubstituted heteroalkylene.

Embodiment Q29. The compound of one of Embodiments Q1 to Q27, wherein L⁶ is independently —NHC(O)—.

Embodiment Q30. The compound of one of Embodiments Q1 to Q27, wherein
L^{6A} is independently a bond or unsubstituted alkylene;
L^{6B} is independently a bond, —NHC(O)—, or unsubstituted arylene;
L^{6C} is independently a bond, unsubstituted alkylene, or unsubstituted arylene;
L^{6D} is independently a bond or unsubstituted alkylene; and
L^{6E} is independently a bond or —NHC(O)—.

Embodiment Q31. The compound of one of Embodiments Q1 to Q27, wherein
L^{6A} is independently a bond or unsubstituted C₁-C₈ alkylene;
L^{6B} is independently a bond, —NHC(O)—, or unsubstituted phenylene;
L^{6C} is independently a bond, unsubstituted C₂-C₈ alkynylene, or unsubstituted phenylene;
L^{6D} is independently a bond or unsubstituted C₁-C₈ alkylene; and
L^{6E} is independently a bond or —NHC(O)—.

Embodiment Q32. The compound of one of Embodiments Q1 to Q27, wherein L⁶ is independently a bond,

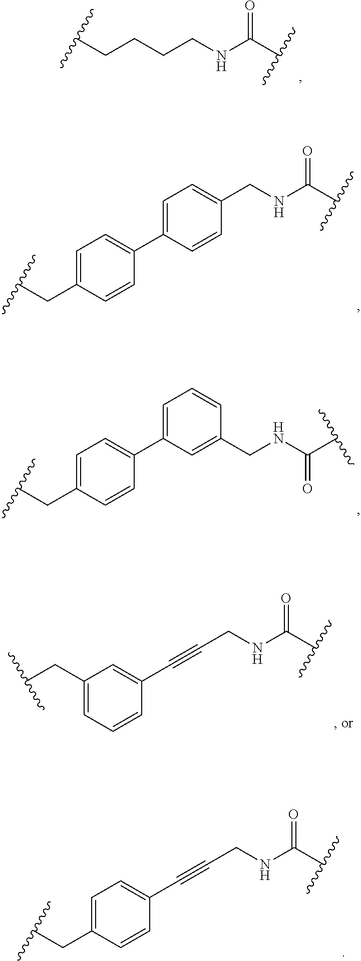 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment Q33. The compound of one of Embodiments Q1 to Q32, wherein L⁵ is independently —NHC(O)—, —C(O)NH—,substituted or unsubstituted alkylene, or substituted or unsubstituted heteroalkylene.

Embodiment Q34. The compound of one of Embodiments Q1 to Q32, wherein L⁵ is independently —NHC(O)—.

Embodiment Q35. The compound of one of Embodiments Q1 to Q32, wherein
L^{5A} is independently a bond or unsubstituted alkylene;
L^{5B} is independently a bond, —NHC(O)—, or unsubstituted arylene;
L^{5C} is independently a bond, unsubstituted alkylene, or unsubstituted arylene;
L^{5D} is independently a bond or unsubstituted alkylene; and
L^{5E} is independently a bond or —NHC(O)—.

Embodiment Q36. The compound of one of Embodiments Q1 to Q32, wherein
L^{5A} is independently a bond or unsubstituted C₁-C₈ alkylene;
L^{5B} is independently a bond, —NHC(O)—, or unsubstituted phenylene;
L^{5C} is independently a bond, unsubstituted C₂-C₈ alkynylene, or unsubstituted phenylene;
L^{5D} is independently a bond or unsubstituted C₁-C₈ alkylene; and
L^{5E} is independently a bond or —NHC(O)—.

Embodiment Q37. The compound of one of Embodiments Q1 to Q32, wherein L⁵ is independently a bond,

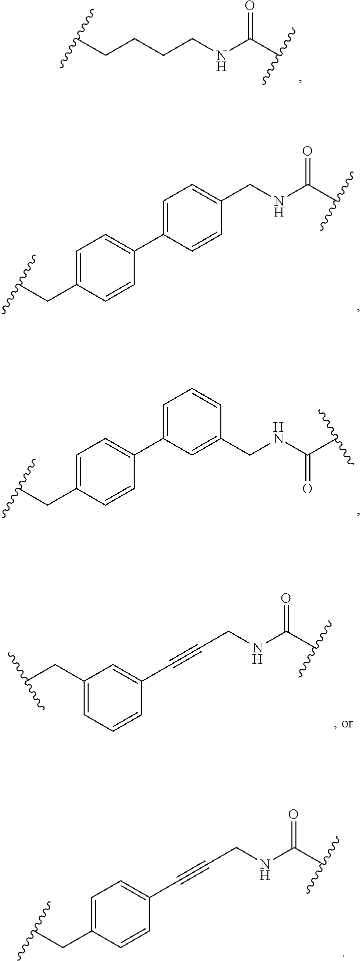 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment Q38. The compound of one of Embodiments Q1 to Q37, wherein R¹ is unsubstituted C₁-C₁₇ alkyl.

Embodiment Q39. The compound of one of Embodiments Q1 to Q37, wherein R¹ is unsubstituted C₁₁-C₁₇ alkyl.

Embodiment Q40. The compound of one of Embodiments Q1 to Q37, wherein R¹ is unsubstituted C₁₃-C₁₇ alkyl.

Embodiment Q41. The compound of one of Embodiments Q1 to Q37, wherein R¹ is unsubstituted C₁₅ alkyl.

Embodiment Q42. The compound of one of Embodiments Q1 to Q37, wherein R¹ is unsubstituted unbranched C₁-C₁₇ alkyl.

Embodiment Q43. The compound of one of Embodiments Q1 to Q37, wherein R¹ is unsubstituted unbranched C₁₁-C₁₇ alkyl.

Embodiment Q44. The compound of one of Embodiments Q1 to Q37, wherein R¹ is unsubstituted unbranched C₁₃-C₁₇ alkyl.

Embodiment Q45. The compound of one of Embodiments Q1 to Q37, wherein R¹ is unsubstituted unbranched C₁₅ alkyl.

Embodiment Q46. The compound of one of Embodiments Q1 to Q37, wherein R¹ is unsubstituted unbranched saturated C₁-C₁₇ alkyl.

Embodiment Q47. The compound of one of Embodiments Q1 to Q37, wherein R¹ is unsubstituted unbranched saturated C₁₁-C₁₇ alkyl.

Embodiment Q48. The compound of one of Embodiments Q1 to Q37, wherein R¹ is unsubstituted unbranched saturated C₁₃-C₁₇ alkyl.

Embodiment Q49. The compound of one of Embodiments Q1 to Q37, wherein R¹ is unsubstituted unbranched saturated C₁₅ alkyl.

Embodiment Q50. The compound of one of Embodiments Q1 to Q49, wherein R² is unsubstituted C₁-C₁₇ alkyl.

Embodiment Q51. The compound of one of Embodiments Q1 to Q49, wherein R² is unsubstituted C₁₁-C₁₇ alkyl.

Embodiment Q52. The compound of one of Embodiments Q1 to Q49, wherein R² is unsubstituted C₁₃-C₁₇ alkyl.

Embodiment Q53. The compound of one of Embodiments Q1 to Q49, wherein R² is unsubstituted C₁₅ alkyl.

Embodiment Q54. The compound of one of Embodiments Q1 to Q49, wherein R² is unsubstituted unbranched C₁-C₁₇ alkyl.

Embodiment Q55. The compound of one of Embodiments Q1 to Q49, wherein R² is unsubstituted unbranched C₁₁-C₁₇ alkyl.

Embodiment Q56. The compound of one of Embodiments Q1 to Q49, wherein R² is unsubstituted unbranched C₁₃-C₁₇ alkyl.

Embodiment Q57. The compound of one of Embodiments Q1 to Q49, wherein R² is unsubstituted unbranched C₁₅ alkyl.

Embodiment Q58. The compound of one of Embodiments Q1 to Q49, wherein R² is unsubstituted unbranched saturated C₁-C₁₇ alkyl.

Embodiment Q59 The compound of one of Embodiments Q1 to Q49, wherein R¹ is unsubstituted unbranched saturated C₁₁-C₁₇ alkyl.

Embodiment Q60. The compound of one of Embodiments Q1 to Q49, wherein R² is unsubstituted unbranched saturated C₁₃-C₁₇ alkyl.

Embodiment Q61. The compound of one of Embodiments Q1 to Q49, wherein R² is unsubstituted unbranched saturated C₁₅ alkyl.

Embodiment Q62. The compound of one of Embodiments Q1 to Q61, wherein the oligonucleotide is an siRNA, a microRNA mimic, a stem-loop structure, a single-stranded siRNA, an RNaseH oligonucleotide, an anti-microRNA oligonucleotide, a steric blocking oligonucleotide, a CRISPR guide RNA, or an aptamer.

Embodiment Q63. The compound of one of Embodiments Q1 to Q62, wherein the oligonucleotide is modified.

Embodiment Q64. The compound of one of Embodiments Q1 to Q62, wherein the oligonucleotide comprises a nucleotide analog.

Embodiment Q65. The compound of one of Embodiments Q1 to Q63, wherein the oligonucleotide comprises a locked nucleic acid (LNA) residue, bicyclic nucleic acid (BNA) residue, constrained ethyl (cEt) residue, unlocked nucleic acid (UNA) residue, phosphorodiamidate morpholino oligomer (PMO) monomer, peptide nucleic acid (PNA) monomer, 2′-O-methyl (2′-OMe) residue, 2′-O-methyoxyethyl residue, 2′-deoxy-2′-fluoro residue, 2′-O-methoxy ethyl/phosphorothioate residue, phosphoramidate, phosphorodiamidate, phosphorothioate, phosphorodithioate, phosphonocarboxylic acid, phosphonocarboxylate, phosphonoacetic acid, phosphonoformic acid, methyl phosphonate, boron phosphonate, or O-methylphosphoroamidite.

Embodiment Q66. The compound of Embodiment Q1, wherein the compound is a lipid-conjugated compound having the structure of Formula I:

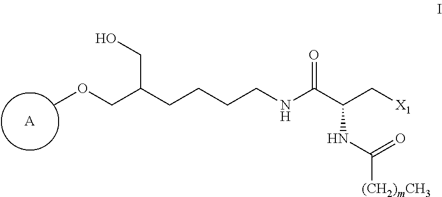

or a pharmaceutically acceptable salt thereof, wherein:
A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded nucleic acid;
X₁ is

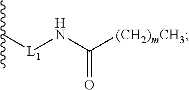

L₁ is —(CH₂)ₙ—, —(CH₂)ₙL₂(CH₂)ₙ—, or a bond;
L₂ is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6.

Embodiment Q67. The compound of Embodiment Q66, wherein each m is 10, L₁ is —(CH₂)ₙ—, and n is 3.

Embodiment Q68. The compound of Embodiment Q66, wherein each m is 11, L₁ is —(CH₂)ₙ—, and n is 3.

Embodiment Q69. The compound of Embodiment Q66, wherein each m is 12, L₁ is —(CH₂)ₙ—, and n is 3.

Embodiment Q70. The compound of Embodiment Q66, wherein each m is 13, L₁ is —(CH₂)ₙ—, and n is 3.

Embodiment Q71. The compound of Embodiment Q66, wherein each m is 14, L₁ is —(CH₂)ₙ—, and n is 3.

Embodiment Q72. The compound of Embodiment Q66, wherein each m is 15, L₁ is —(CH₂)ₙ—, and n is 3.

Embodiment Q73. The compound of Embodiment Q66, wherein each m is 16, L₁ is —(CH₂)ₙ—, and n is 3.

Embodiment Q74. The compound of Embodiment Q66, wherein each m is 17, L₁ is —(CH₂)ₙ—, and n is 3.

Embodiment Q75. The compound of Embodiment Q66, wherein each m is 18, L₁ is —(CH₂)ₙ—, and n is 3.

Embodiment Q76. The compound of Embodiment Q66, wherein each m is independently an integer from 12 to 16; and wherein each n is independently an integer from 1 to 6.

Embodiment Q77. The compound of Embodiment Q66, wherein each m is independently an integer from 12 to 14; and wherein each n is independently an integer from 1 to 6.

Embodiment Q78. The compound of Embodiment Q66, wherein L₁ is a bond; and each m is independently an integer from 12 to 16.

Embodiment Q79. The compound of Embodiment Q66, wherein L₁ is —(CH₂)₃C(═O)NH(CH₂)₅—; and each m is independently an integer from 12 to 16.

Embodiment Q80. The compound of one of Embodiments Q78 to Q79, wherein each m is 14.

Embodiment Q81. The compound of one of Embodiments Q66 to 80, wherein the modified double-stranded oligonucleotide or modified single-stranded oligonucleotide contains at least one phosphorothioate linkage.

Embodiment Q82. The compound of one of Embodiments Q66 to Q81, wherein the modified double-stranded oligonucleotide or modified single-stranded oligonucleotide contains at least one 2′-O-methyl residue.

Embodiment Q83. The compound of one of Embodiments Q66 to Q82, wherein the modified double-stranded oligonucleotide or modified single-stranded oligonucleotide contains at least one 2′-deoxy-2′-fluoro residue.

Embodiment Q84. The compound of one of Embodiments Q66 to Q83, wherein the modified double-stranded oligonucleotide or modified single-stranded oligonucleotide comprises a bicyclic nucleic acids (BNA) residue.

Embodiment Q85. The compound of Embodiment Q84, wherein oligonucleotide bicyclic nucleic acid residue is a locked nucleic acid (LNA) residue or constrained ethyl (cEt) residue.

Embodiment Q86. The compound of Embodiment Q66 to Q84, wherein the modified double-stranded oligonucleotide or modified single-stranded oligonucleotide comprises a phosphorodiamidate morpholino oligomer (PMO) monomer.

Embodiment Q87. The compound of one of Embodiments Q66 to Q86, wherein the modified double-stranded oligonucleotide is an siRNA or microRNA mimic.

Embodiment Q88. The compound of Embodiment Q87, wherein the lipid moiety is attached to the 3′ end of the passenger strand of the siRNA or microRNA mimic.

Embodiment Q89. The compound of one of Embodiments Q66 to Q86, wherein A is an antisense oligonucleotide.

Embodiment Q90. A cell containing the compound of any one of Embodiments Q1 to Q89.

Embodiment Q91. The cell of Embodiment Q90, wherein the cell is a primary cell.

Embodiment Q92. The cell of Embodiment Q91, wherein the cell is an adipocyte cell, a hepatocyte cell, a fibroblast cell, an endothelial cell, a kidney cell, a human umbilical vein endothelial cell (HUVEC), an adipose cell, a macrophage cell, a neuronal cell, a muscle cell, or a differentiated primary human skeletal muscle cell.

Embodiment Q93. The cell of Embodiment Q92, wherein the cell is a human umbilical vein endothelial cell.

Embodiment Q94. The cell of Embodiment Q90, wherein the cell is an immortalized cell.

Embodiment Q95. The cell of Embodiment Q94, wherein the cell is a NIH3T3 cell, a differentiated 3T3L1 cell, a RAW264.7 cell, or a SH-SY5Y cell.

Embodiment Q96. The cell of one of Embodiments Q90 to Q92, wherein the cell is an adipocyte cell or a hepatocyte cell.

Embodiment Q97. A method of introducing an oligonucleotide into a cell, the method comprising contacting said cell with the compound of any one of Embodiments Q1 to Q89.

Embodiment Q98. A method of introducing an oligonucleotide into a cell in vitro, comprising contacting the cell with the compound of any one of Embodiments Q1 to Q89 under free uptake conditions.

Embodiment Q99. The method of Embodiment Q98, wherein the method is ex vivo and the cell is a primary cell.

Embodiment Q100. The method of Embodiment Q99, wherein the cell is an adipocyte cell, a hepatocyte cell, a fibroblast cell, an endothelial cell, a kidney cell, a human umbilical vein endothelial cell (HUVEC), an adipose cell, a macrophage cell, a neuronal cell, a rat neuron, a muscle cell, or a differentiated primary human skeletal muscle cell.

Embodiment Q101. The method of Embodiment Q99, wherein the cell is a human umbilical vein endothelial cell.

Embodiment Q102. The method of Embodiment Q98, wherein the cell is an immortalized cell.

Embodiment Q103. The method of Embodiment Q102, wherein the cell is a NIH3T3 cell, a differentiated 3T3L1 cell, a RAW264.7 cell, or a SH-SY5Y cell.

Embodiment Q104. The method of Embodiment Q98 or Q100, wherein the cell is an adipocyte cell or a hepatocyte cell.

Embodiment Q105. A method of introducing an oligonucleotide into a cell ex vivo, comprising: obtaining cells; and contacting the cells with the compound of any one of Embodiments Q1 to Q89 under free uptake conditions.

Embodiment Q106. The method of Embodiment Q105, wherein the cells are neurons, TBM cells, skeletal muscle cells, adipocyte cells or hepatocyte cells.

Embodiment Q107. The method of Embodiment Q105, wherein the cells are human umbilical vein endothelial cells.

Embodiment Q108. A method of introducing an oligonucleotide into a cell in vivo, comprising contacting the cell with the compound of any one of Embodiments Q1 to Q89.

Embodiment Q109. The method of Embodiment Q108, wherein the cell is an adipocyte cell, a hepatocyte cell, a fibroblast cell, an endothelial cell, a kidney cell, an adipose cell, a macrophage cell, a neuronal cell, a muscle cell, or a skeletal muscle cell.

Embodiment Q110. A method comprising contacting a cell with a compound of any one of Embodiments Q1 to Q89.

Embodiment Q111. The method of Embodiment Q110, wherein contacting occurs in vitro.

Embodiment Q112. The method of Embodiment Q110, wherein the contacting occurs ex vivo.

Embodiment Q113. The method of Embodiment Q110, wherein the contacting occurs in vivo.

Embodiment Q114. A method comprising administering to a subject a compound of any one of compounds Q1 to Q89.

Embodiment Q115. The method of Embodiment Q114, wherein the subject has a disease or disorder of the eye, liver, kidney, heart, adipose tissue, lung, muscle or spleen.

Embodiment Q116. A compound of any one of Embodiments Q1 to Q89, for use in therapy.

Embodiment Q117. A compound of any one of Embodiments Q1 to Q89, for use in the preparation of a medicament.

Embodiment Q118. A method of introducing an oligonucleotide into a cell within a subject, the method comprising administering to said subject the compound of any one of Embodiments Q1 to Q89.

Embodiment Q119. A cell comprising the compound of any one of Embodiments Q1 to Q89.

Embodiment Q120. A pharmaceutical composition comprising a pharmaceutically acceptable excipient and the compound of any one of Embodiments Q1 to Q89.

### EXAMPLES

The following examples will further describe the present disclosure, and are used for the purposes of illustration only, and should not be considered as limiting.

The compounds disclosed herein may be synthesized by methods described below, or by modification of these methods. Ways of modifying the methodology include, among others, temperature, solvent, reagents, etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include, e.g., those described in R. Larock, Comprehensive Organic Transformations, VCH Publishers, 1989, or L. Paquette, ed., Encyclopedia of Reagentsfor Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Syntheses of Lipid Motifs

### Synthesis of DTx-01-01

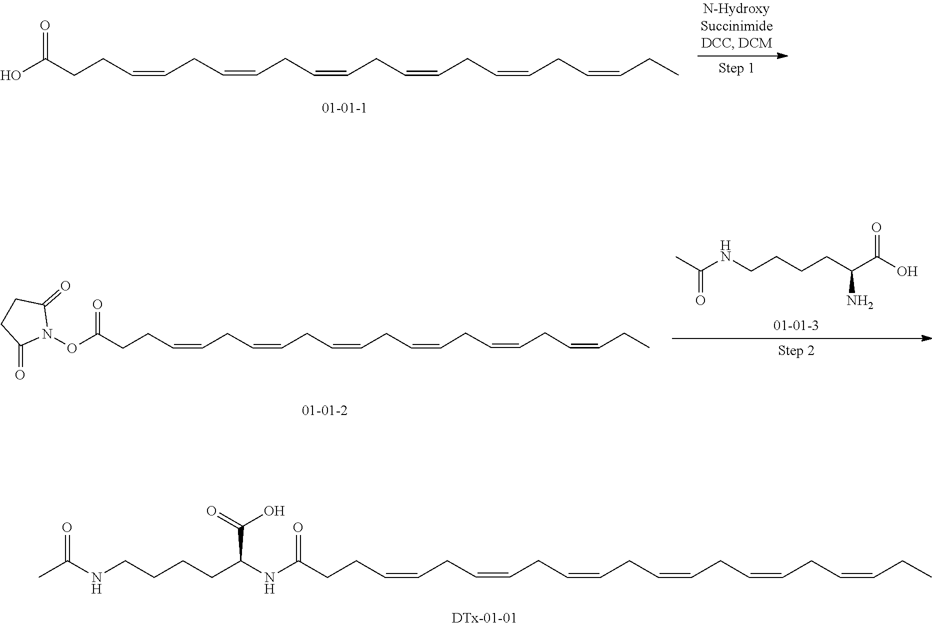

### Step 1: Synthesis of Intermediate 01-01-2

To a stirred solution of 01-01-1 (5.0 g, 0.015 mol) in DCM (500 mL) at RT was added DMAP (0.17 g, 0.0015 mol), DCC (4.86 g, 0.016 mol), followed by N-hydroxysuccinimide (1.92 g, 0.016 mol). The resulting mixture was stirred at RT. After 16 h, the reaction mixture was filtered through a sintered funnel. The filtrate was evaporated to yield crude 01-01-2 as a pale-yellow liquid (6.0 g, 92.5%), which was used in the next step without further purification.

### Step 2: Synthesis of Lipid Motif DTx-01-01

To a stirred solution of 01-01-3 (1.3 g, 0.006 mol) in DMF (20 mL) at RT was added slowly Et₃N (3 mL, 0.020 mol) and then 01-01-2 (2.93 g, 0.007 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-01, which was purified by column chromatography (3% MeOH in DCM) to afford lipid motif DTx-01-01 as a viscous, brown liquid (1.3 g, 51%). LCMS m/z (M+H)⁺: 499.4; ¹H-NMR (400 MHz, DMSO-d6): δ 0.92 (t, J=7.6 Hz, 3H), 1.24-1.66 (m, 10H), 1.82 (s, 3H), 2.02-2.33 (m, 7H), 2.73-2.98 (m, 9H), 3.94 (br s, 1H), 5.27-5.34 (m, 10H), 7.70 (br s, 1H), 7.78 (br s, 1H).

### Synthesis of DTx-01-03

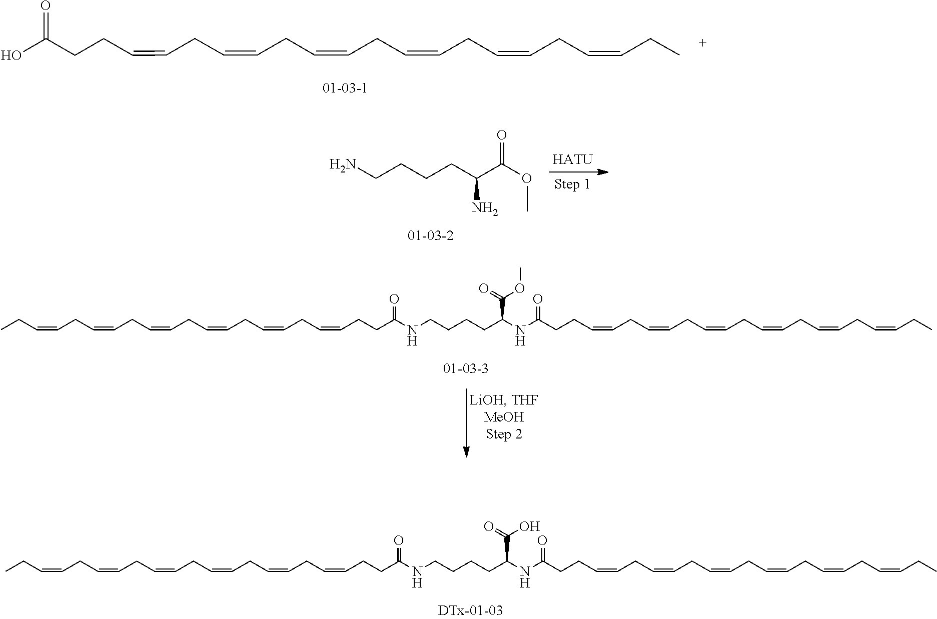

### Step 1: Synthesis of Intermediate 01-03-3

To a stirred solution of 01-03-1 (15 g, 0.045 mol) in DMF (300 mL) at RT was added slowly DIPEA (39.86 mL, 0.11 mol), HATU (17.1 g, 0.045 mol), and 01-03-2 (3.6 g, 0.022 mol). The resulting mixture was stirred at RT. After 16 h, the reaction mixture was quenched with ice water dropwise and extracted with DCM. The combined organic extract was washed with ice water, brine, dried over Na₂SO₄, and then evaporated to yield crude 01-03-3, which was purified by column chromatography (20% EtOAc in petroleum ether) to afford 01-03-3 as a viscous, pale brown liquid (11.2 g, 63.7%).

### Step 2: Synthesis of Lipid Motif DTx-01-03

To a stirred solution of 01-03-3 (10 g, 0.012 mol) in MeOH (100 mL) at 0° C. was added slowly LiOH (1.07 g, 0.025 mol) in water (50 mL). The resulting mixture was stirred at RT. After 4h, ice water was added dropwise to the reaction mixture. The mixture was acidified with 1.5 M HCl and then extracted with DCM. The combined organic extract was washed with ice water, brine, dried over Na₂SO₄, and then evaporated to yield crude DTx-01-03, which was purified by column chromatography (3% MeOH in DCM) to afford lipid motif DTx-01-03 as a viscous, pale brown liquid (7.5 g, 77%). LCMS m/z (M+H)⁺: 767.5; ¹H-NMR (400 MHz, DMSO-d6): δ 0.954 (t, J=3.6 Hz, 6H), 1.23-1.66 (m, 8H), 1.99-2.33 (m, 12H), 2.69-2.82 (m, 22H), 4.13 (t, J=3.6 Hz, 1H), 5.25-5.36 (m, 22H), 7.76 (t, J=5.2 Hz, 1H), 8.03 (d, J=7.6 Hz, 1H), 12.5 (br s, 1H).

### Synthesis of Lipid Motif DTx-01-06

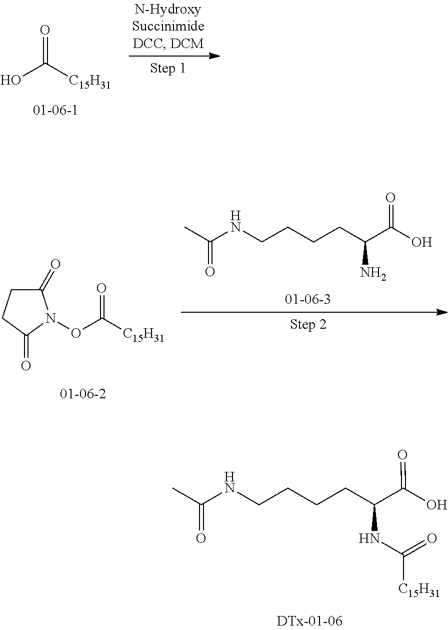

### Step 1: Synthesis of Intermediate 01-06-2

To a stirred solution of linear fatty acid 01-06-1 (5.0 g, 0.018 mol) in DCM (100 mL) at RT was added DMAP (0.208 g, 0.0018 mol), DCC (5.22 g, 0.018 mol), and then N-hydroxysuccinimide (2.07 g, 0.018 mol). The resulting mixture was stirred at RT. After 16 h, the reaction mixture was filtered through a sintered funnel. The filtrate was evaporated to yield crude 01-06-2 as an off-white solid (6.0 g, 88%), which was used in the next step without further purification.

### Step 2: Synthesis of Lipid Motif DTx-01-06

To a stirred solution of 01-06-3 (1.02 g, 0.054 mol) in DMF (40 mL) at RT was added slowly Et₃N (2.3 mL, 0.016 mol) and 01-06-2 (2 g, 0.047 mol). The resulting mixture was stirred at RT. After 16 h, the reaction mixture was quenched with ice water dropwise and then extracted with EtOAc. The combined organic extract was washed with chilled water, brine, dried over Na₂SO₄, and then evaporated to yield crude DTx-01-06, which was purified by column chromatography (3% MeOH in DCM) to afford lipid motif DTx-01-06 as an off-white solid (2.0 g, 88%). MS (ESI) m/z (M+H)⁺: 427.4; ¹H-NMR (400 MHz, DMSO-d6): δ 0.97 (t, J=7.2 Hz, 3H), 1.36-1.77 (m, 31H), 1.83 (s, 3H), 2.09 (t, J=6.4 Hz, 2H), 2.98 (d, J=6.0 Hz, 2H), 5.57 (d, J=8.0 Hz, 2H), 7.79 (br s, 1H), 7.97 (d, J=7.6 Hz, 1H).

### Synthesis of the Methyl Ester of Lipid Motif DTx-01-07 (DTx-01-07-OMe)

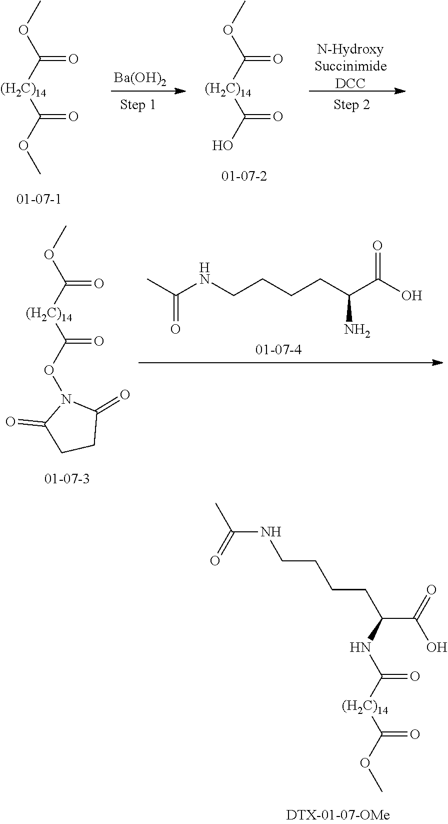

### Step 1: Synthesis of Intermediate 01-07-2

To a stirred solution of 01-07-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water. The quenched reaction was acidified with 1.5 M HCl and then extracted with EtOAc. The combined organic extract was washed with water, brine, dried over Na₂SO₄, and then evaporated to yield crude 01-07-2. Purification by column chromatography (15% EtOAc in petroleum ether) afforded 01-07-2 as an off-white solid (15.2 g, 79.5%).

### Step 2: Synthesis of Intermediate 01-07-3

To a stirred solution of 01-07-2 (5.0 g, 0.016 mol) in DCM (500 mL) at RT was added DMAP (0.182 g, 0.0016 mol) and DCC (4.98 g, 0.016 mol), followed by N-hydroxy succinimide (2.1 g, 0.016 mol). The resulting mixture was stirred at RT. After 16 h, the reaction mixture was filtered through sintered funnel. The filtrate was evaporated to yield crude 01-07-3 as a pale-yellow liquid (5.0 g, 75%), which was used in the next step without further purification.

### Step 3: Synthesis of Lipid Motif DTx-01-07

To a stirred solution of 01-07-4 (0.94 g, 0.005 mol) in DMF (40 mL) at RT was added slowly Et₃N (2.12 mL, 0.015 mol) and then 01-07-3 (2.0 g, 0.005 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-07-OMe, which was purified by column chromatography (3% MeOH in DCM) to afford the methyl ester of lipid motif DTx-01-07 (i.e., DTx-01-07-OMe) as an off-white solid (2.0 g, 84%). LCMS m/z (M+H)⁺: 471.4; ¹H-NMR (400 MHz, DMSO-d6): δ 1.47-1.67 (m, 30H), 1.77 (s, 3H), 2.09 (t, J=7.2 Hz, 2H), 2.28 (d, J=7.2 Hz, 2H), 2.99 (q, J=6.4 Hz, 2H), 3.57 (s, 3H), 4.11 (t, J=4.8 Hz, 1H), 7.79 (br s, 1H), 7.97 (d, J=7.6 Hz, 1H).

### Synthesis of Lipid Motif DTx-01-08

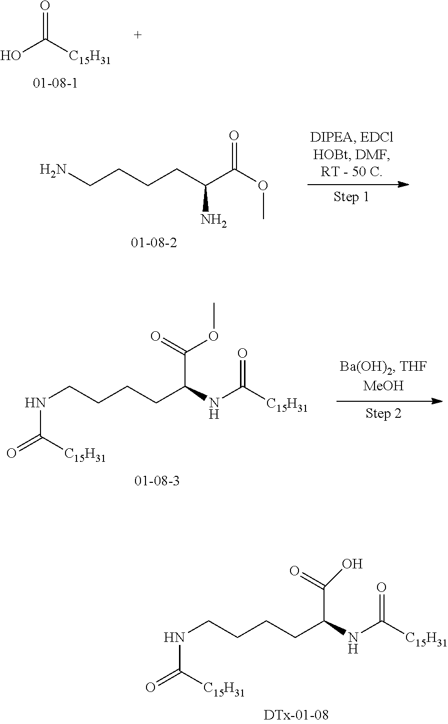

### Step 1: Synthesis of Compound 01-08-3

To a stirred solution of linear fatty acid 01-08-1 (25.58 g, 0.099 mol) in DMF (500 mL) at RT was added DIPEA (42.66 mL, 0.245 mol) and compound 01-08-2 (8.0 g, 0.049 mol), followed by EDCl (18.97 g, 0.099 mol) and HOBt (13.37 g, 0.099 mol). The resulting mixture was stirred at 50° C. After 16 h, the reaction mixture was quenched with ice water and extracted with DCM. The combined organic extract was washed with water, brine, dried over Na₂SO₄, and then evaporated to give crude 01-08-3, which was recrystallized (20% MTBE in petroleum ether) to afford 01-08-3 as an off-white solid (18 g, 56%).

### Step 2: Synthesis of Lipid Motif DTx-01-08

To a stirred solution of 01-08-3 (10 g, 0.0156 mol) in MeOH and THF (1:1; 200 mL) at RT was added slowly Ba(OH)₂ (9.92 g, 0.031 mol, dissolved in MeOH). The resulting mixture was stirred at RT. After 6 h, the reaction mixture was quenched with ice water dropwise, and then acidified with 1.5 M HCl. The mixture was filtered, and the precipitate was recrystallized (MTBE in petroleum ether) to afford lipid motif DTx-01-08 as an off-white solid (7.2 g, 74.2%). MS (ESI) m/z (M+H)⁺: 623.6; ¹H-NMR (400 MHz, CDCl₃): δ 0.868 (m, 6H), 1.25-1.69 (m, 58H), 2.03 (t, J=7.2 Hz, 2H), 2.11 (t, J=7.6 Hz, 2H), 2.99 (q, J=8.4 Hz, 2H), 4.15-4.20 (m, 1H), 7.42 (br s, 1H), 7.65 (d, J=7.6 Hz, 1H), 12.09 (br s, 1H).

### Synthesis of the Methyl Ester of Lipid Motif DTx-01-09 (DTx-01-09-OMe)

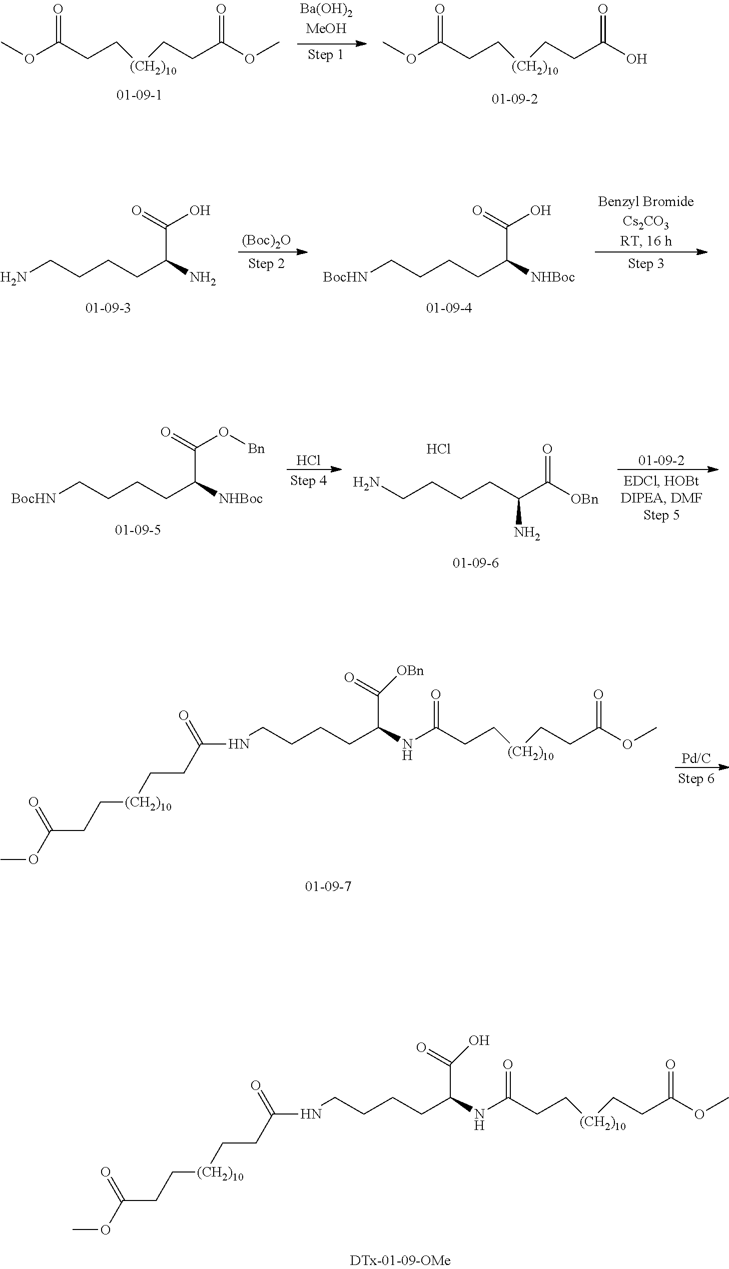

### Step 1: Synthesis of Intermediate 01-09-2

To a stirred solution of 01-09-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water, acidified with 1.5 M HCl, and extracted with EtOAc. The combined organic extract was washed with water, brine, dried over Na₂SO₄, and then evaporated to yield crude 01-09-2, which was purified by column chromatography (15% EtOAc in petroleum ether) to afford product 01-09-2 as an off-white solid (15.2 g, 79.5%).

### Step 2: Synthesis of Intermediate 01-09-4

To a stirred solution of 01-09-3 (15 g, 0.102 mol) in 1,4-dioxane (100 mL) and water (50 mL) at RT was added slowly NaHCO₃ (18.98 g, 0.226 mol) and BOC anhydride (49.2 mL, 0.226 mol). The resulting mixture was stirred at RT. After 16 h, the reaction mixture was quenched with ice water dropwise and extracted with DCM. The combined organic extract was washed with ice water, brine, dried over Na₂SO₄, and then evaporated to yield crude 01-09-4, which was purified by column chromatography (30% EtOAc in petroleum ether) to afford 01-09-4 as viscous, pale yellow liquid (20 g, 56%).

### Step 3: Synthesis of Intermediate 01-09-5

To a stirred solution of 01-09-4 (15 g, 0.043 mol) in DMF (150 mL) at RT was added slowly Cs₂CO₃ (14 g, 0.043 mol) and benzyl bromide (5.6 mL, 0.047 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude 01-09-5, which was purified by column chromatography (18% EtOAc in petroleum ether) to afford the 01-09-5 as a viscous, colorless liquid (15.2 g, 77%).

### Step 4: Synthesis of Intermediate 01-09-6

To a stirred solution of 01-09-5 (10 g, 0.022 mol) in 1,4-dioxane (50 mL) at RT was added slowly 4 M HCl in 1,4-dioxane (23 mL, 0.091 mol). The resulting mixture was stirred at RT. After 16 h, the reaction mixture was concentrated under reduced pressure. The residue was purified by trituration in diethyl ether, affording 01-09-6 as an off-white solid (15.2 g, 79.5%).

### Step 5: Synthesis of Intermediate 01-09-7

To a stirred solution of 01-09-6 (7.0 g, 0.025 mol) in DMF (100 mL) at RT was added slowly DIPEA (22.4 mL, 0.128 mol), 01-09-2 (15.05 g, 0.05 mol), EDCl (9.5 g, 0.05 mol), and HOBt (6.75 g, 0.05 mol). The resulting mixture was stirred at 50° C. After 16 h, the reaction mixture was quenched with ice water dropwise and extracted with DCM. The combined organic extract was washed with ice water, brine, dried over Na₂SO₄, and then evaporated to give crude 01-09-7. Recrystallization (MTBE in petroleum ether) yielded 01-09-7 as an off-white solid (10 g, 49.7%)

### Step 6: Synthesis of Lipid Motif DTx-01-09

To a stirred solution of 01-09-7 (10 g, 0.099 mol) in THF (100 mL) and EtOAc (100 mL) at RT was added 10% Pd/C (1.0 g). The resulting mixture was stirred at RT under 3 kg/Cm² hydrogen pressure. After 16 h, the mixture was filtered through celite, and the filtrate was evaporated to yield crude DTx-01-09-OMe. Recrystallization (20% MTBE in petroleum ether) afforded the methyl ester of lipid motif DTx-01-09 (i.e., DTx-01-09-OMe) as a pale yellow solid (5.3 g, 60%). LCMS m/z (M+H)⁺: 711.5; ¹H-NMR (400 MHz, CDCl₃): δ 1.23-1.52 (m, 55H), 2.01 (t, J=9.6 Hz, 2H), 2.08-2.11 (m, 2H), 2.28 (t, J=9.6 Hz, 4H), 2.99 (q, J=8.4 Hz, 2H), 3.57 (s, 6H), 4.11-4.12 (m, 1H), 7.72 (t, J=5.2 Hz, 1H), 7.96 (d, J=7.6 Hz, 1H).

### Synthesis of Lipid Motif DTx-01-11

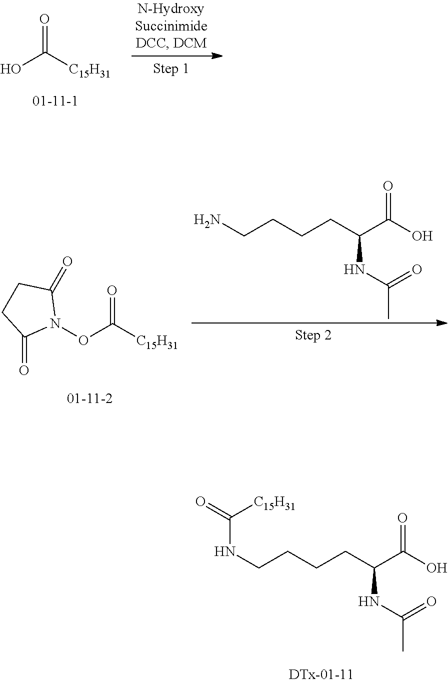

### Step 1: Synthesis of Intermediate 01-11-2

To a stirred solution of linear fatty acid 01-11-1 (5.0 g, 0.018 mol) in DCM (100 mL) at RT was added DMAP (0.208 g, 0.0018 mol) and DCC (5.22 g, 0.018 mol), followed by N-hydroxysuccinimide (2.07 g, 0.018 mol). The resulting mixture was stirred at RT. After 16 h, the reaction mixture was filtered through a sintered funnel. Evaporation of the filtrate yielded crude 01-11-2 as an off-white solid (6.0 g, 88%), which was used directly in the next step without further purification.

### Step 2: Synthesis of Lipid Motif DTx-01-11

To a stirred solution of 01-11-3 (2.05 g, 0.01 mol) in DMF (80 mL) at RT was added slowly Et₃N (4.6 mL, 0.032 mol) and 01-11-2 (4.0 g, 0.01 mol). The resulting mixture was stirred at RT. After 16 h, the reaction mixture was quenched with ice water dropwise and then extracted with EtOAc. The combined organic extract was washed with ice water, brine, dried over Na₂SO₄, and then evaporated to yield crude DTx-01-11, which was purified by column chromatography (3% MeOH in DCM) to afford lipid motif DTx-01-11 as an off-white solid (3.1 g, 66.5%). MS (ESI) m/z (M+H)⁺: 427.4; ¹H-NMR (400 MHz, DMSO-d6): δ 0.85 (t, J=6.8 Hz, 3H), 1.23-1.73 (m, 31H), 1.83 (s, 3H), 2.02 (t, J=7.2 Hz, 2H), 3.00 (q, J=6.0 Hz, 2H), 4.10 (dd, J=8.4, 4.4 Hz, 2H), 7.74 (d, J=5.2 Hz, 1H), 8.07 (br s, 1H), 12.45 (br s, 1H).

### Synthesis of the Methyl Ester of Lipid Motif DTx-01-12 (DTx-01-12-OMe)

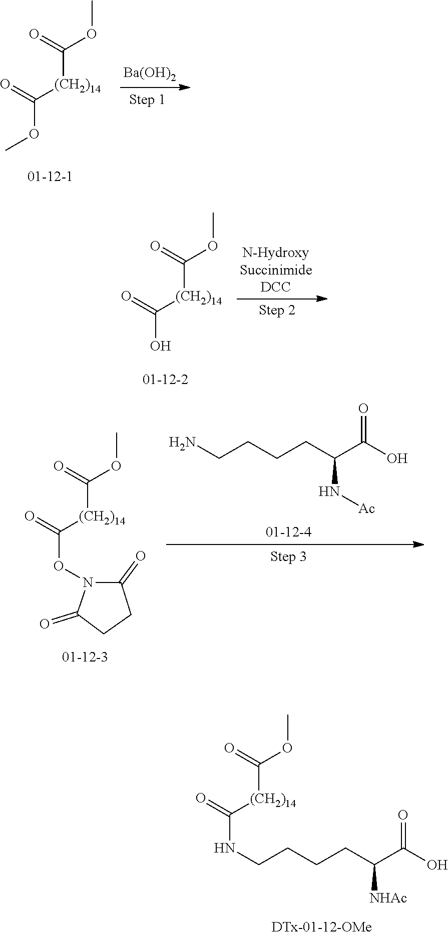

### Step 1: Synthesis of Intermediate 01-12-2

To a stirred solution of 01-12-1 (15 g, 0.063 mol) in MeOH (100 mL) at RT was added slowly Ba(OH)₂ (20 g, 0.063 mol). The resulting mixture was stirred at RT. After 16 h, the reaction mixture was quenched with ice water, acidified with 1.5 M HCl, and extracted with EtOAc. The combined organic extract was washed with water, brine, dried over Na₂SO₄, and then evaporated to yield crude 01-12-2. Purification by column chromatography (15% EtOAc in petroleum ether) afforded 01-12-2 as an off-white solid (15.2 g, 79.5%).

### Step 2: Synthesis of Intermediate 01-12-3

To a stirred solution of 01-12-2 (5.0 g, 0.016 mol) in DCM (500 mL) at RT was added DMAP (0.182 g, 0.0016 mol) and DCC (4.98 g, 0.016 mol), followed by N-hydroxysuccinimide (2.1 g, 0.016 mol). The resulting mixture was stirred at RT. After 16 h, the reaction mixture was filtered through a sintered funnel. The filtrate was evaporated to yield crude 01-12-3 as a pale yellow liquid (5.0 g, 75%), which was used directly in the next step without further purification.

### Step 3: Synthesis of Lipid Motif DTx-01-12

To a stirred solution of 01-12-4 (0.94 g, 0.005 mol) in DMF (40 mL) at RT was added slowly Et₃N (2.12 mL, 0.015 mol), 01-12-3 (2.0 g, 0.05 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-12-OMe. Purification by column chromatography (3% MeOH in DCM) afforded the methyl ester of lipid motif DTx-01-12 (i.e., DTx-01-12-OMe) as an off-white solid (1.5 g, 63.2%). LCMS m/z (M+H)⁺: 471.4; ¹H-NMR (400 MHz, DMSO-d6): δ 1.22-1.66 (m, 30H), 1.83 (s, 3H), 2.01 (t, J=7.6 Hz, 2H), 2.27 (d, J=7.2 Hz, 2H), 2.99 (q, J=6.4 Hz, 2H), 3.57 (s, 3H), 4.10 (t, J=4.8 Hz, 1H), 7.72 (t, J=5.2 Hz, 1H), 8.06 (d, J=8.0 Hz, 1H), 12.47 (br s, 1H).

### Synthesis of Lipid Motif DTx-01-13

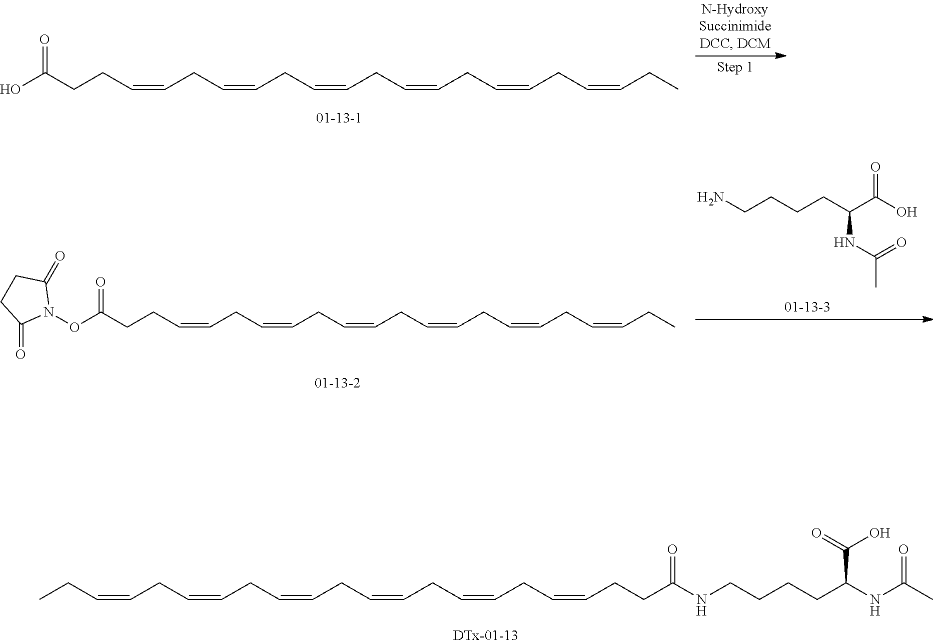

### Step 1: Synthesis of Intermediate 01-13-2

To a stirred solution of 01-13-1 (5.0 g, 0.015 mol) in DCM (500 mL) at RT was added DMAP (0.17 g, 0.0015 mol) and DCC (4.86 g, 0.016 mol), followed by N-hydroxysuccinimide (1.92 g, 0.016 mol). The resulting mixture was stirred at RT. After 16 h, the reaction mixture was filtered through a sintered funnel, and the filtrate was evaporated to yield crude 01-13-2 as a pale yellow liquid (6.0 g, 92.5%). The crude intermediate was used directly in the next step without further purification.

### Step 2: Synthesis of Lipid Motif DTx-01-13

To a stirred solution of 01-13-3 (1.3 g, 0.006 mol) in DMF (20 mL) at RT was added slowly Et₃N (3 mL, 0.020 mol) and 01-13-2 (2.93 g, 0.007 mol). The resulting mixture was stirred at RT. After 16 h, the reaction mixture was quenched with ice water dropwise and extracted with EtOAc. The combined organic extract was washed with ice water, brine, dried over Na₂SO₄, and then evaporated to yield crude DTx-01-13, which was purified by column chromatography (3% MeOH in DCM) to afford lipid motif DTx-01-13 as a viscous, brown liquid (2.1 g, 61%). LCMS m/z (M+H)⁺: 499.4; ¹H-NMR (400 MHz, DMSO-d6): δ 0.90 (t, J=7.2 Hz, 3H), 1.22-1.67 (m, 7H), 1.75 (s, 3H), 1.98-2.27 (m, 7H), 2.73-2.95 (m, 9H), 2.96 (dd, J=12.4, 6.4 Hz, 2H), 4.06-4.09 (m, 1H), 5.23-5.37 (m, 10H), 7.79 (br s, 1H), 7.91 (t, J=7.6 Hz, 1H).

### Synthesis of Lipid Motif DTx-01-30

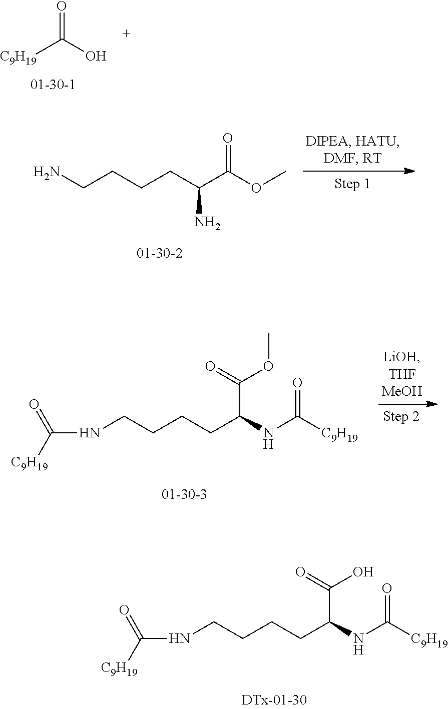

### Step 1: Synthesis of Intermediate 01-30-3

To a stirred solution of 01-30-2 (3 g, 0.01 mol) in DMF (50 mL) at RT was added slowly DIPEA (13.8 mL, 0.077 mol), linear fatty acid 01-30-1 (4.4 g, 0.0154 mol), and HATU (5.87 g, 0.0154 mol). The resulting mixture was stirred at RT. After 16 h, the reaction mixture was quenched with ice water. The precipitate was isolated by filtration, and then dried in vacuo to afford 01-30-3 as an off-white solid (3.2 g, 53.15%).

### Step 2: Synthesis of Lipid Motif DTx-01-30

To a stirred solution of 01-30-3 (3.2 g, 0.0068 mol) in MeOH (30 mL), THF (30 mL), and water (3 mL), was added LiOH H₂O (0.86 g, 0.0251 mol). The resulting reaction mixture was stirred 16 h. Subsequently, the reaction mixture was concentrated under vacuum and then neutralized with 1.5 N HCl. The precipitate was isolated via filtration, washed with water, and dried under vacuum to yield crude DTx-01-30. Recrystallization (80% DCM in hexane) afforded lipid motif DTx-01-30 as an off-white solid (2.2 g, 73.3%). LCMS m/z (M+H)⁺: 455.5; ¹H-NMR (400 MHz, DMSO-d6): δ 0.88-0.92 (t, J=7.2 Hz, 6H), 1.17-1.55 (m, 33H), 1.64 (t, J=7.0 Hz, 1H), 2.00 (t, J=7.2 Hz, 2H), 2.06-2.10 (m, 2H), 2.97-2.99 (m, 2H), 4.11 (t, J=8.4 Hz, 1H), 7.71 (s, 1H), 7.96 (d, J=7.6 Hz, 1H), 12.47 (br s, 1H).

### Synthesis of Lipid Motif DTx-01-31

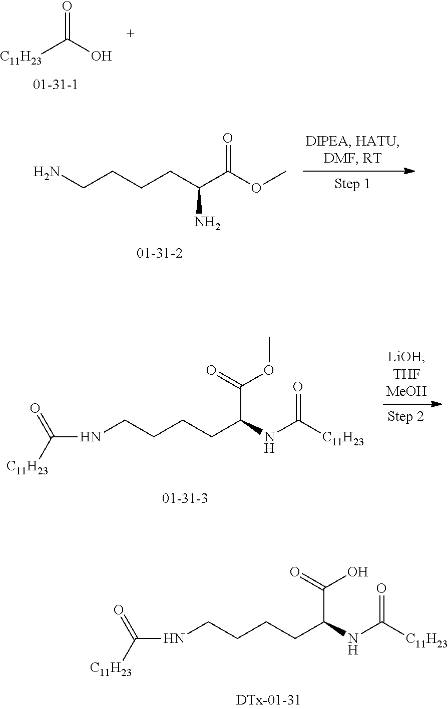

### Step 1: Synthesis of Intermediate 01-31-3

To a stirred solution of 01-31-2 (3 g, 0.0128 mol) in DMF (50 mL) at RT was added slowly DIPEA (13.8 mL, 0.077 mol), linear fatty acid 01-31-1 (3.1 g, 0.0154 mol), and HATU (5.87 g, 0.0154 mol). The resulting mixture was stirred at RT. After 16 h, the reaction mixture was quenched with ice water. Solids were isolated by filtration and dried in vacuo to afford 01-01-3 as an off-white solid (3.4 g 50.7%).

### Step 2: Synthesis of Lipid Motif DTx-01-31

To a stirred solution of 01-01-3 (3 g, 0.0057 mol) in MeOH (10 mL), THF (10 mL), and water (3 mL), was added LiOH H₂O (0.8 g, 0.0019 mol). The reaction mixture was stirred 16 h. Subsequently, the reaction mixture was concentrated under vacuum and then neutralized with 1.5 N HCl. The precipitate was solid was isolated via filtration, washed with water, and dried under vacuum, yielding crude DTx-01-31. Recrystallization (80% DCM in hexane) afforded lipid motif DTx-01-31 as an off-white solid (2.3 g, 79.3%). LCMS m/z (M+H)⁺: 511.5; ¹H-NMR (400 MHz, DMSO-d6): δ 0.86-0.90 (t, J=7.2 Hz, 6H), 1.33-1.54 (m, 42H), 1.64 (t, J=7.9 Hz, 1H), 1.98-2.08 (m, 4H), 2.96 (t, J=6.3 Hz, 2H), 4.02-4.18 (m, 1H), 7.71-7.79 (m, 2H).

### Synthesis of Lipid Motif DTx-01-32

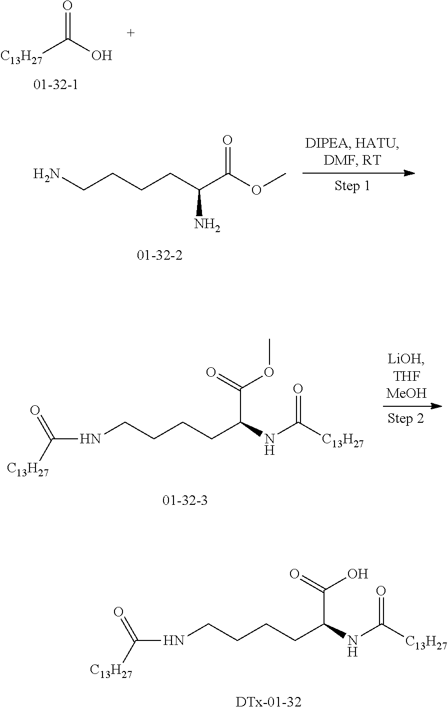

### Step 1: Synthesis of Intermediate 01-32-3

To a stirred solution of 01-32-2 (3 g, 0.01 mol) in DMF (50 mL) at RT was added slowly DIPEA (13.8 mL, 0.077 mol), linear fatty acid 01-32-1 (4.4 g, 0.0154 mol), and HATU (5.87 g, 0.0154 mol). The resulting mixture was stirred at 60° C. After 16 h, the reaction mixture was quenched with ice water, the solids isolated by filtration, and the solids dried under vacuum to afford 01-32-3 as an off-white solid (3.5 g, 53.2%).

### Step 2: Synthesis of Lipid Motif DTx-01-32

To a stirred solution of 01-32-3 (3.5 g, 0.0051 mol) in MeOH (10 mL), THF (10 mL), and water (3 mL), was added LiOH H₂O (0.8 g, 0.0154). The reaction mixture was stirred 16 h. Subsequently, the reaction mixture was concentrated under vacuum and neutralized with 1.5 N HCl. The solids were isolated by filtration, washed with water, and dried under vacuum, affording crude DTx-01-32. Recrystallization (80% DCM in hexane) yielded lipid motif DTx-01-32 as an off-white solid (2.3 g, 79.3%). LCMS m/z (M+H)⁺: 567.2; ¹H-NMR (400 MHz, TFA-d): δ 0.87-0.98 (m, 6H), 1.20-1.58 (m, 41H), 1.74-1.92 (m, 8H), 2.18-2.21 (m, 2H), 2.73 (t, J=7.6 Hz, 2H), 3.05 (t, J=7.6 Hz, 2H), 3.60 (t, J=7.8 Hz, 2H).

### Synthesis of Lipid Motif DTx-01-33

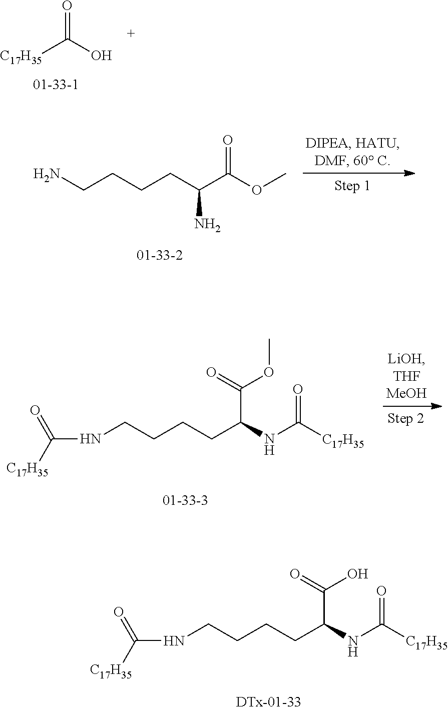

### Step 1: Synthesis of Intermediate 01-33-3

To a stirred solution of 01-33-2 (5 g, 0.0312 mol) in DMF (100 mL) at RT was added slowly DIPEA (32 mL, 0.1872 mol), linear fatty acid 01-33-1 (26.6 g, 0.0936 mol), and HATU (41.5 g, 0.1092 mol) slowly at RT. After 16 h, the reaction mixture was quenched with ice water. Crude 01-33-3 was isolated by filtration from the reaction mixture and dried in vacuo. Purification by trituration with THF afforded 01-33-3 as an off-white solid (8.5 g, 39.5%).

### Step 2: Synthesis of Lipid Motif DTx-01-33

To a stirred solution of 01-33-3 (5 g, 0.0072 mol) in MeOH (75 mL), THF (75 mL), and water (3 mL), was added LiOH H₂O (0.60 g, 0.0144 mol). The reaction mixture was stirred 16 h. Subsequently, the reaction mixture was concentrated under vacuum and neutralized with 1.5 N HCl. The solids were filtered, washed with water, and dried under vacuum, affording crude DTx-01-33. Recrystallization (IPA) yielded lipid motif DTx-01-33 as an off-white solid (2.3 g, 47%). LCMS m/z (M+H)⁺: 680; ¹H-NMR (400 MHz, TFA-d): δ 1.10-1.18 (m, 6H), 1.62-1.80 (m, 57H), 2.06-2.20 (m, 8H), 2.49-2.50 (m, 2H), 2.96-3.01 (m, 2H), 3.32-3.35 (m, 2H), 3.87-3.98 (m, 2H).

### Synthesis of Lipid Motif DTx-01-34

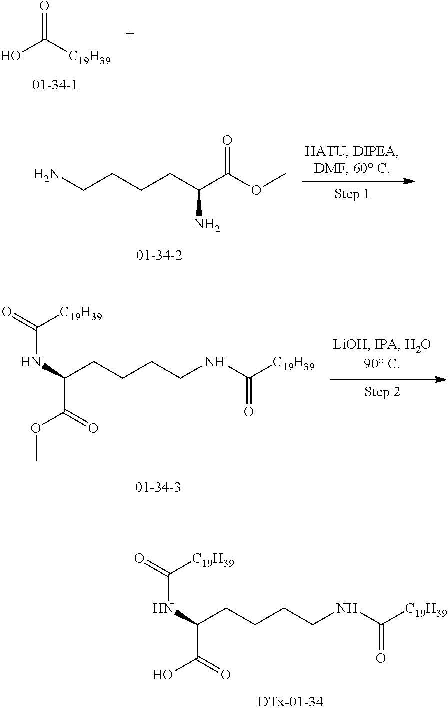

### Step 1: Synthesis of Intermediate 01-34-3

To a stirred solution of 01-34-2 (5 g, 0.0312 mol) in DMF (100 mL) at RT was added slowly DIPEA (32 mL, 0.1872 mol), linear fatty acid 01-34-1 (29.2 g, 0.0936 mol), and HATU (41.5 g, 0.1092 mol). The resulting mixture was stirred at 50° C. After 16 h, the reaction mixture was quenched with ice water, the solids isolated by filtration, and then the solids dried under vacuum. Purification of the solids by trituration with THF afforded 01-34-3 as an off-white solid (10 g, 43%).

### Step 2: Synthesis of Lipid Motif DTx-01-34

To a stirred solution of 01-34-3 (5 g, 0.0066 mol) in 9:1 IPA:water (150 mL) was added LiOH H₂O (0.56 g, 0.0133 mol). The reaction mixture was stirred at 90° C. After 1 h, the reaction mixture was concentrated under vacuum and then neutralized with 1.5 N HCl. The precipitate was isolated via filtration, washed with water, and dried under vacuum. Recrystallization (IPA) of the precipitate afforded lipid motif DTx-01-34 as an off-white solid (3.2 g, 65%). LCMS m/z (M+H)⁺: 736.2; ¹H-NMR (400 MHz, TFA-d): δ 1.13-1.17 (m, 6H), 1.48-1.79 (m, 65H), 2.05-2.19 (m, 8H), 2.48-2.49 (m, 2H), 2.95-2.96 (m, 2H), 3.28-3.34 (m, 2H), 3.85-3.96 (m, 2H).

### Synthesis of Lipid Motif DTx-01-35

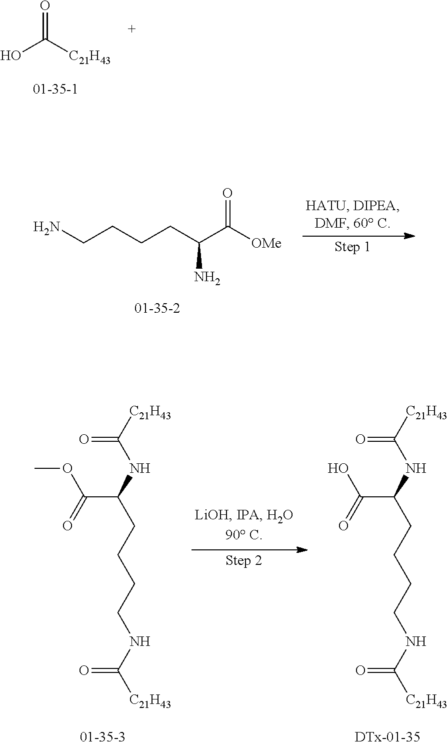

### Step 1: Synthesis of Intermediate 01-35-3

To a stirred solution of 01-35-2 (5 g, 0.0312 mol) in DMF (100 mL) at RT was added slowly DIPEA (32 mL, 0.1872 mol), linear fatty acid 01-35-1 (31.8 g, 0.0936 mol), and HATU (41.5 g, 0.1092 mol). The resulting mixture was stirred at 60° C. After 16 h, the reaction mixture was quenched with ice water, the solids isolated by filtration, and then the solids dried under vacuum. Purification of the solids by trituration with THF yielded 01-35-3 as an off-white solid (7 g, 28%).

### Step 2: Synthesis of Lipid Motif DTx-01-35

To a stirred solution of 01-35-3 (5 g, 0.0062 mol) in 9:1 IPA:water (150 mL) was added LiOH H₂O (0.52 g, 0.0124 mol). The reaction mixture was stirred at 90° C. After 1 h, the reaction mixture was concentrated under vacuum and then neutralized with 1.5 N HCl. The solids were isolated by filtration, washed with water, and dried under vacuum, yielding crude DTx-01-35. Recrystallization in IPA afforded lipid motif DTx-01-35 as an off-white solid (3.1 g, 63%). LCMS m/z (M+H)⁺: 792.2; ¹H-NMR (400 MHz, TFA-d): δ 1.06-1.22 (m, 6H), 1.49-1.88 (m, 73H), 1.99-2.29 (m, 8H), 2.49-2.51 (m, 2H), 2.95-3.10 (m, 2H), 3.32-3.34 (m, 2H), 3.86-3.90 (m, 2H).

### Synthesis of Lipid Motif DTx-03-06

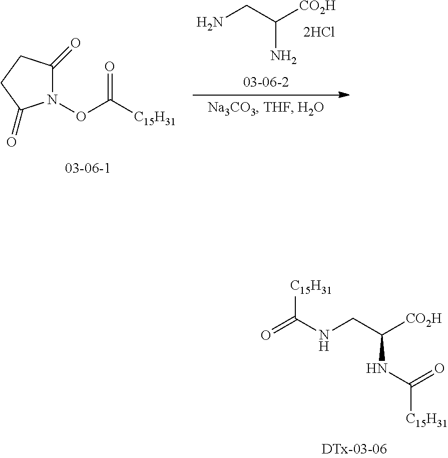

To a stirred solution of 03-06-2 (1.2 g, 0.0068 mol) in 65% aq. EtOH (40 mL) at RT was added slowly Et₃N (4.75 mL, 0.034 mol) and NHS-linear fatty acid 03-06-1 (6.0 g, 0.170 mol). The resulting mixture was stirred at 75° C. After 16 h, the reaction mixture was neutralized with 1.5 N HCl. The precipitate was isolated by filtration, washed with water, and dried. Purification of the precipitate by trituration with DCM afforded lipid motif DTx-03-06 as an off-white solid (2.3 g, 57%). LCMS m/z (M+H)⁺: 581.5; ¹H-NMR (400 MHz, TFA-d): δ 0.78-0.82 (m, 6H), 1.21-1.40 (m, 49H), 1.62-1.79 (m, 4H), 2.35-2.46 (m, 2H), 2.96-2.30 (m, 2H), 3.89-4.03 (m, 2H).

### Synthesis of Lipid Motif DTx-06-06

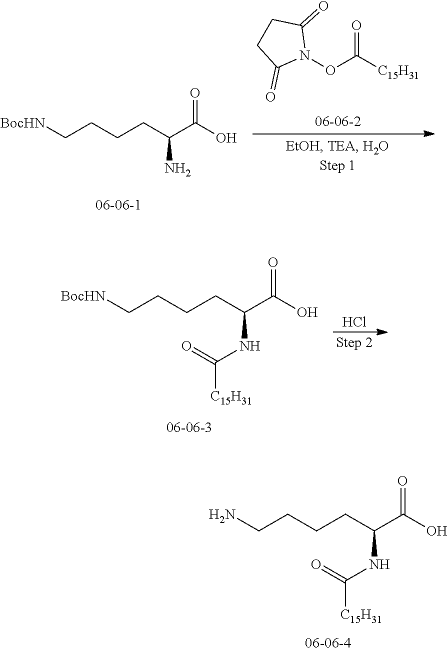

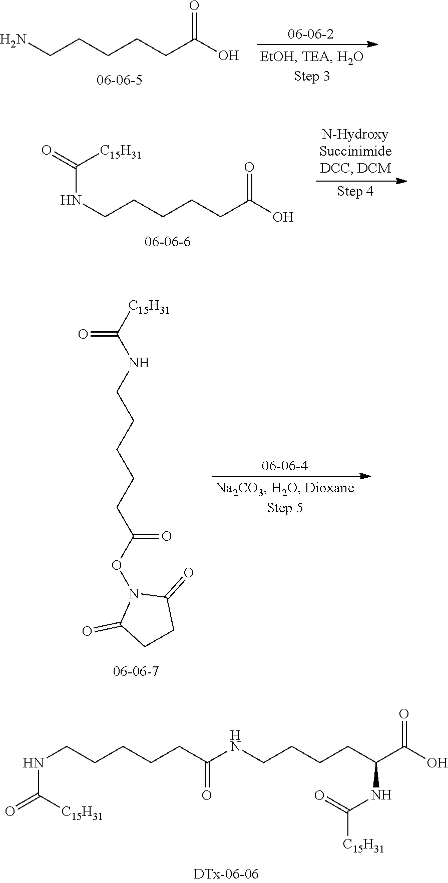

### Step 1: Synthesis of Intermediate 06-06-3

To a stirred solution of 06-06-1 (4.6 g, 0.0169 mol) in 65% aq. EtOH (60 mL) at RT was added slowly Et₃N (5.9 mL, 0.042 mol) and NHS-linear fatty acid 06-06-2 (6 g, 0.00186 mol). The resulting mixture was stirred at 75° C. After 16 h, the reaction mixture was neutralized with 1.5 N HCl. The precipitate was isolated by filtration, washed with water, and dried. Purification of the precipitate by column chromatography (3% MeOH in DCM) afforded 06-06-3 as an off-white solid (5.0 g, 62%).

### Step 2: Synthesis of Intermediate 06-06-4

To a stirred solution of 06-06-3 (7 g, 0.014 mol) in 1,4-dioxane (50 mL) at RT was added slowly 4 M HCl in 1,4-dioxane (50 mL). The resulting mixture was stirred at RT. After 16 h, the reaction mixture was concentrated under reduced pressure to yield crude 06-06-4, which was triturated with diethyl ether to afford 06-06-4 as an off-white solid (4.5 g, 81%).

### Step 3: Synthesis of Intermediate 06-06-6

To a stirred solution of 06-06-5 (5 g, 0.038 mol) in 65% aq. EtOH (40 mL) at RT was added slowly Et₃N (13.3 mL, 0.095 mol) and NHS-linear fatty acid 06-06-2 (13 g, 0.038 mol). The resulting mixture was stirred at 75° C. After 16 h, the reaction mixture was neutralized with 1.5 N HCl. The precipitate was isolated via filtration, washed with water, and dried, affording 06-06-6 as an off-white solid (4.2 g, 30%).

### Step 4: Synthesis of Intermediate 06-06-7

To a stirred solution of 06-06-6 (3.8 g, 0.010 mol) in DCM (80 mL) at RT was added DMAP (0.12 g, 0.001 mol) and DCC (2.1 g, 0.010 mol), followed by N-hydroxysuccinimide (1.17 g, 0.010 mol). The resulting mixture was stirred at RT 16 h. Subsequently, the reaction mixture was filtered through a sintered funnel, and then the filtrate evaporated, yielding crude 06-06-7 as an off-white solid (4.7 g, 100%), which was used in the next step without further purification.

### Step 5: Synthesis of Lipid Motif DTx-06-06

To a stirred solution of 06-06-4 (4 g, 0.009 mol) in 1 M Na₂CO₃ (50 mL) and 1,4-dioxane (100 mL) at RT was added slowly 06-06-7 (4.5 g, 0.096 mol). The resulting mixture was stirred at RT. After 16 h, the reaction mixture was neutralized with 1.5 N HCl. The precipitate was isolated by filtration, washed with water, and dried. Purification of the precipitate by trituration with MeOH afforded lipid motif DTx-06-06 as an off-white solid (2.3 g, 32%). LCMS m/z (M+H)⁺: 737.6; ¹H-NMR (400 MHz, TFA-d): δ 0.77-0.79 (m, 6H), 1.22-1.52 (m, 51H), 1.68-1.81 (m, 11H), 2.10-2.18 (m, 2H), 2.50-2.67 (m, 5H), 2.94-2.98 (m, 2H), 3.49-3.60 (m, 4H).

### Synthesis of Lipid Motif DTx-01-36

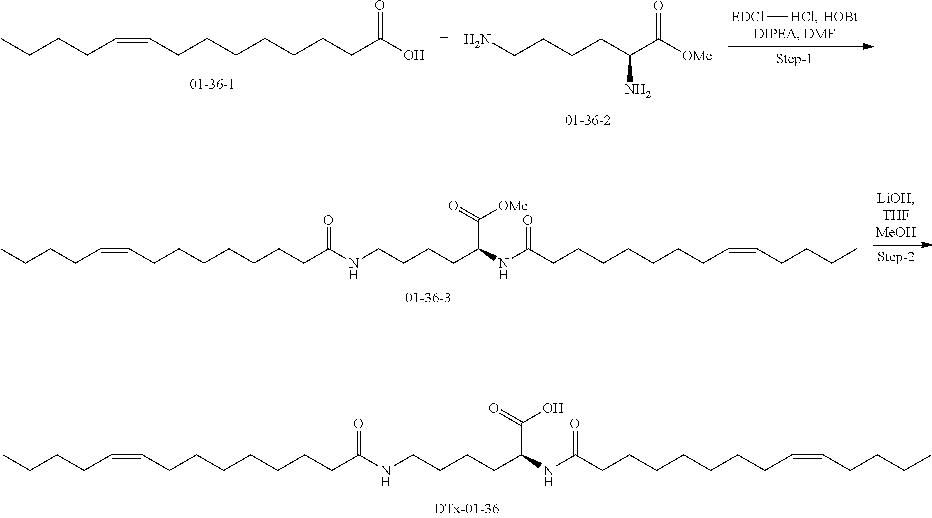

Step 1: To a stirred solution of 01-36-1 (0.73 g, 0.0032 mol) in DMF (6 mL) was added DIPEA (1.16 mL, 0.0064 mol), 01-36-2 (0.3 g, 0.0013 mol) followed by EDCl (0.543 g, 0.0028 mol), HOBt (0.382 g, 0.0028 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-36-3 as an off white solid. (0.54 g, 61%)

Step 2: To a stirred solution of compound 01-36-3 (0.5 g, 0.0009 mol) in MeOH, THF (10 mL; 1:1) and H₂O (0.25 mL) was added LiOH·H₂O (0.071 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-36 as an off white solid. (0.35 g, 73%)

### Analytics of DTx-01-36

¹H-NMR- (400 MHz, DMSO-d6): δ 0.84 (t, J=6.8 Hz, 6H), 1.27-1.66 (m, 35H), 1.98-2.10 (m, 12H), 2.93-2.99 (m, 2H), 4.08-4.14 (m, 1H), 5.27-5.35 (m, 4H), 7.71 (t, J=5.2 Hz, 1H), 7.96 (d, J=7.6 Hz, 1H), 12.49 (bs, 1H). LCMS: 563.5 (M+1).

### Synthesis of Lipid Motif DTx-01-39

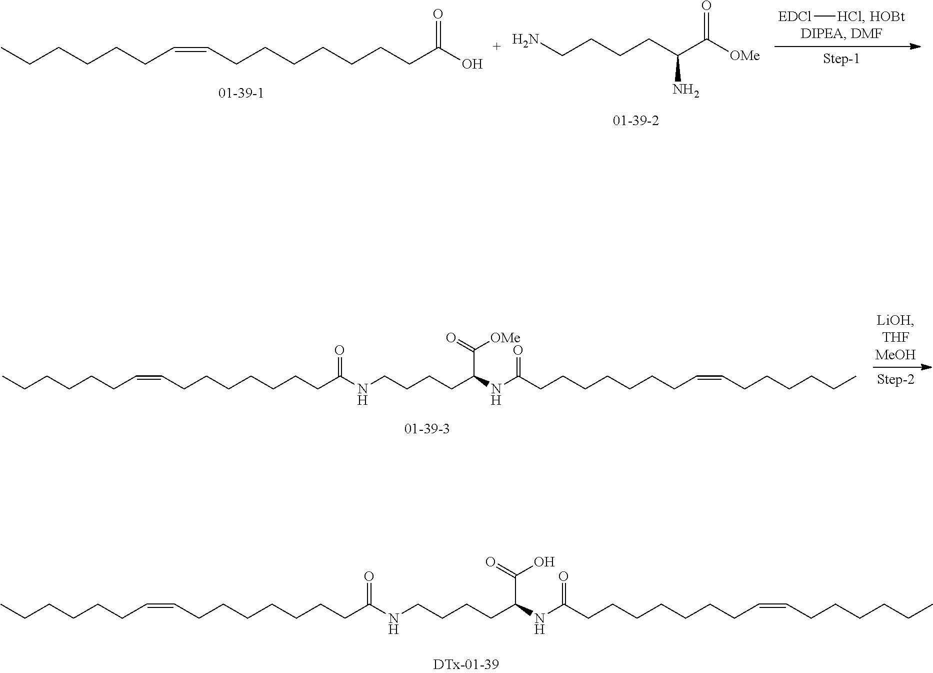

Step 1: To a stirred solution of compound 01-39-1 (2.04 g, 0.0080 mol) in DMF (20 mL) was added DIPEA (2.96 mL, 0.016 mol), compound 01-39-2 (0.75 g, 0.0032) followed by EDCl (1.35 g, 0.0070 mol), HOBt (0.95 g, 0.0070 mol) at RT. The resulting mixture was stirred at 50° C. for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-39-3 as an off white solid. (1.9 g, 79%)

Step 2: To a stirred solution of compound 01-39-3 (1.5 g, 0.0023 mol) in MeOH, THF (30 mL; 1:1) and H₂O (3 mL) was added LiOH·H₂O (0.194 g, 0.0046 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-39 as yellow solid. (1.2 g, 82%)

### Analytics of DTx-01-39

¹H-NMR- (400 MHz, DMSO-d6): δ 0.83 (t, J=6.8 Hz, 6H), 1.23-1.78 (m, 42H), 1.96-2.08 (m, 12H), 2.98 (d, J=5.6 Hz, 2H), 4.08-4.10 (m, 1H), 5.28-5.31 (m, 4H), 7.71 (t, J=5.2 Hz, 1H), 7.95 (d, J=8.4 Hz, 1H), 12.43 (bs, 1H). LCMS: 619.5 (M+1).

### Synthesis of Lipid Motif DTx-01-43

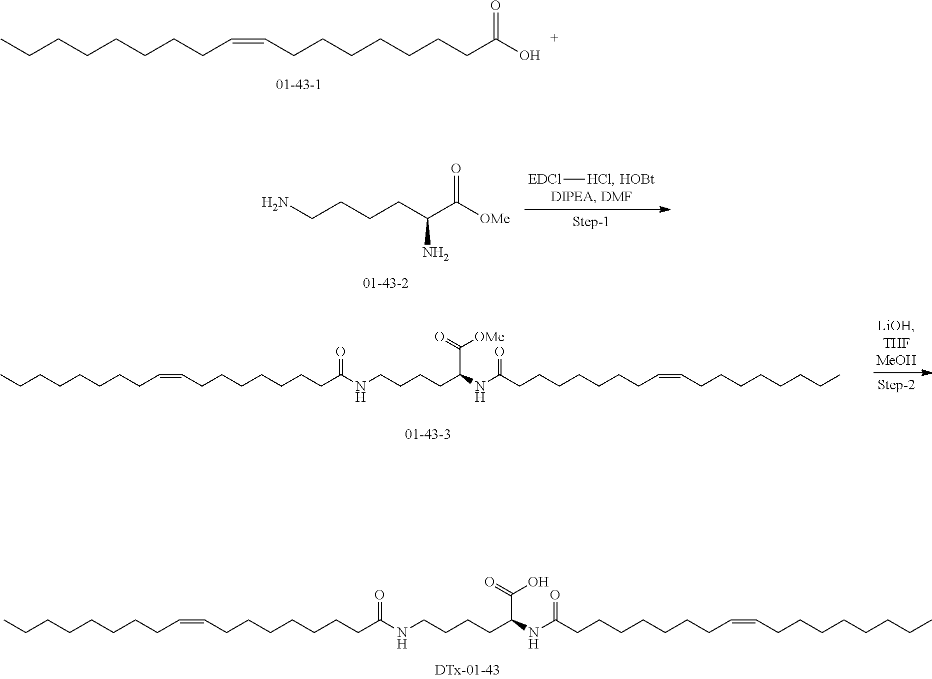

Step 1: To a stirred solution of compound 01-43-1 (3.5 g, 0.0107 mol) in DMF (50 mL) was added DIPEA (3.9 mL, 0.021 mol), compound 01-43-2 dihydrochloride (1 g, 0.0043 mol) followed by EDCl (1.8 g, 0.0094 mol), HOBt (1.2 g, 0.0094 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-43-3 as an off white solid. (2.6 g, 88.7%)

Step 2: To a stirred solution of compound 01-43-3 (2.5 g, 0.0036 mol) in MeOH, THF (40 mL; 1:1) and H₂O (2 mL) was added LiOH·H₂O (0.297 g, 0.0072 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-43 as an off white solid. (2.1 g, 90.6%)

### Analytics of DTx-01-43

¹H-NMR- (400 MHz, DMSO-d6): δ 0.83 (t, J=6.8 Hz, 6H), 1.05-1.65 (m, 48H), 1.96-2.16 (m, 14H), 2.98-2.99 (m, 2H), 4.11-4.16 (m, 1H), 5.29-5.37 (m, 4H), 7.71 (bs, 1H), 7.92 (d, J=6.4 Hz, 1H). LCMS: 676.5 (M+1).

### Synthesis of Lipid Motif DTx-01-44

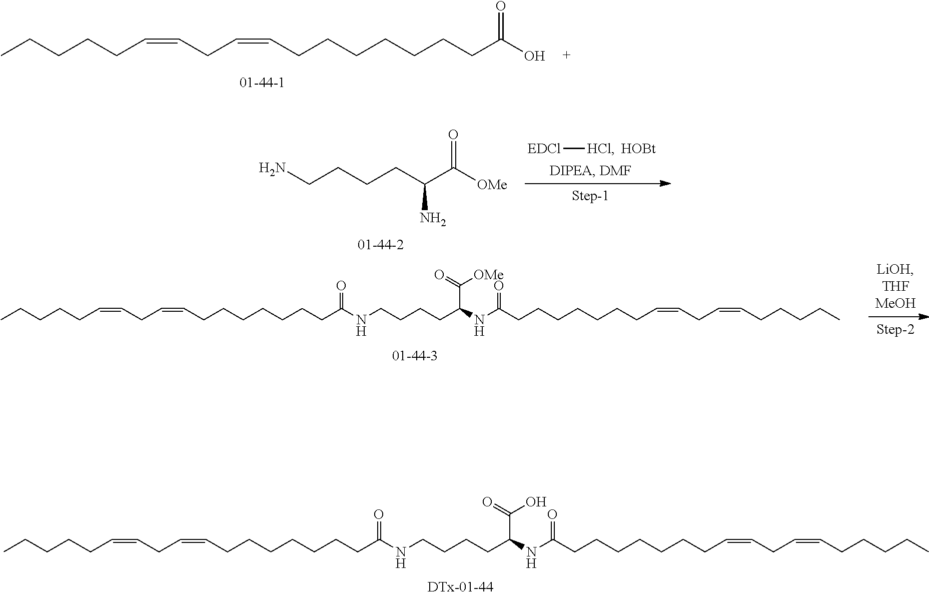

Step 1: To a stirred solution of compound 01-44-1 (5.1 g, 0.0018 mol) in DMF (50 mL) was added DIPEA (6.7 mL, 0.036 mol), compound 01-44-2 (1.7 g, 0.0072 mol) followed by EDCl (3.06 g, 0.016 mol), HOBt (2.16 g, 0.016 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-44-3 as an off white solid. (5 g, 85%)

Step 2: To a stirred solution of compound 01-44-3 (5 g, 0.0072 mol) in MeOH, THF (150 mL; 1:1) and H₂O (3 mL) was added LiOH·H₂O (0.60 g, 0.0144 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-44 as pale yellow viscous liquid. (2.2 g, 45%)

### Analytics of DTx-01-44

¹H-NMR- (400 MHz, DMSO-d6): δ 0.86 (t, J=5.2 Hz, 6H), 1.25-1.70 (m, 38H), 2.01-2.18 (m, 12H), 2.73 (t, J=6.4 Hz, 4H), 2.98-3.00 (m, 2H), 4.12-4.24 (m, 1H), 5.29-5.36 (m, 8H), 7.72 (t, J=5.2 Hz, 1H), 7.95 (d, J=8.0 Hz, 1H), 12.45 (bs, 1H). LCMS: 672.6 (M+1).

### Synthesis of Lipid Motif DTx-01-45

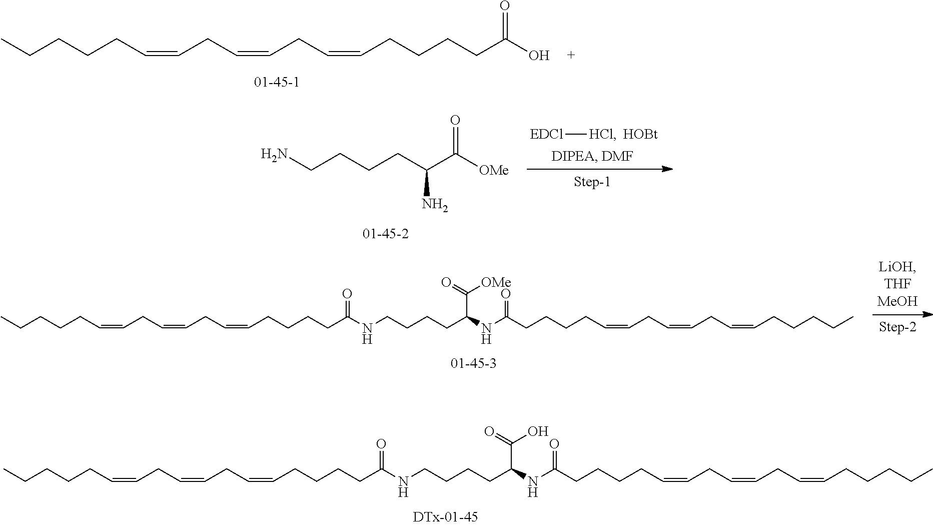

Step 1: To a stirred solution of compound 01-45-1 (0.656 g, 0.0023 mol) in DMF (5 mL) was added DIPEA (1.00 mL, 0.0053 mol), compound 04-45-2 dihydrochloride (0.25 g, 0.0011 mol) followed by EDCl (0.45 g, 0.0023 mol), HOBt (0.318 g, 0.0023 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-45-3 as an off white solid. (0.61 g, 83.56%)

Step 2: To a stirred solution of compound 04-45-3 (0.6 g, 0.0008 mol) in MeOH, THF (12 mL; 1:1) and H₂O (0.6 mL) was added LiOH·H₂O (0.074 g, 0.0018 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-45 as an off white solid. (0.55 g, 94.8%)

### Analytics of DTx-01-45

¹H-NMR- (400 MHz, DMSO-d6): δ 0.86 (t, J=6.0 Hz, 6H), 1.27-1.50 (m, 26H), 2.01-2.10 (m, 12H), 2.77-2.80 (m, 8H), 2.96-2.98 (m, 2H), 3.98-4.01 (m, 1H), 5.32-5.37 (m, 12H), 7.61 (bs, 1H), 7.75 (bs, 1H). LCMS: 668.4 (M+1).

### Synthesis of DTx-01-46

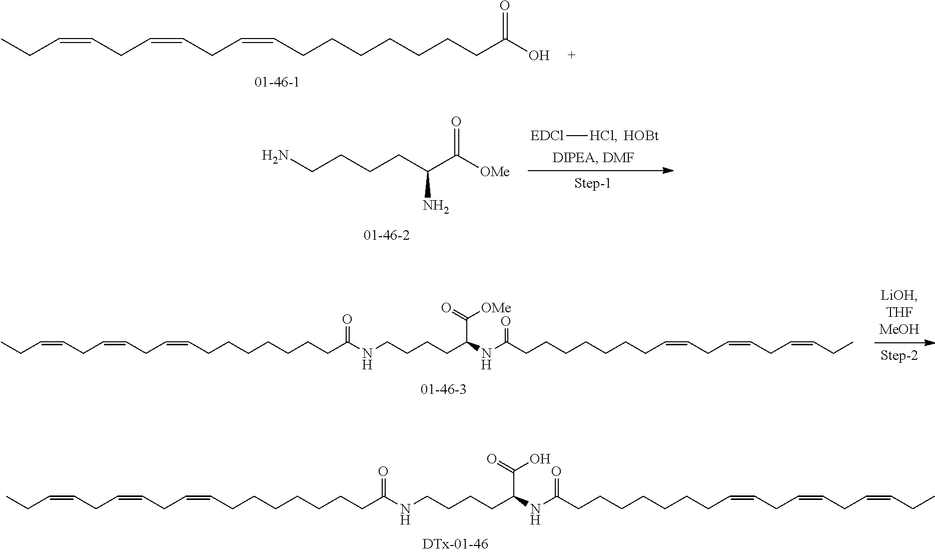

Step 1: To a stirred solution of compound 01-46-1 (2.00 g, 0.0071 mol) in DMF (20 mL) was added DIPEA (2.6 mL, 0.0143 mol), compound 01-46-2 (0.67 g, 0.0029 mol) followed by EDCl (1.20 g, 0.0063 mol), HOBt (0.085 g, 0.0063 mol) at RT. The resulting mixture was stirred at RT for 16 h. The reaction was monitored by LCMS. The reaction mixture was quenched with ice water and extracted with DCM. The combined organic extract was washed with water, brine, dried over Na₂SO₄, evaporated to give crude product which was further purified by column chromatography using 3% MeOH in DCM as eluent to get the product 01-46-3 as an off white solid. (1.8 g, 78%)

Step 2: To a stirred solution of compound 01-46-3 (2.4 g, 0.0035 mol) in MeOH, THF (75 mL; 1:1) and H₂O (2.5 mL) was added LiOH·H₂O (0.0288 g, 0.0070 mol) and the reaction mixture was stirred at RT for 16 h. The reaction mixture was monitored by LCMS, the reaction mixture was concentrated under vacuum to give crude which was neutralized with 1.5 N HCl. Precipitated solid was extracted with DCM. The combined organic extract was washed with water, brine, dried over Na₂SO₄, evaporated to give crude product which was further purified by column chromatography using 3% MeOH in DCM as eluent to get the product DTx-01-46 as pale yellow viscous liquid. (1.5 g, 64%)

### Analytics of DTx-01-46

¹H-NMR- (400 MHz, DMSO-d6): δ 0.91 (t, J=7.6 Hz, 6H), 1.24-1.68 (m, 31H), 2.01-2.10 (m, 10H), 2.78 (t, J=6.0 Hz, 4H), 2.88-2.99 (m, 3H), 5.27-5.36 (m, 1H), 5.29-5.36 (m, 12H), 7.71 (t, J=5.2 Hz, 1H), 7.96 (d, J=8.0 Hz, 1H). LCMS: 668.6 (M+1).

### Synthesis of DTx-08-01

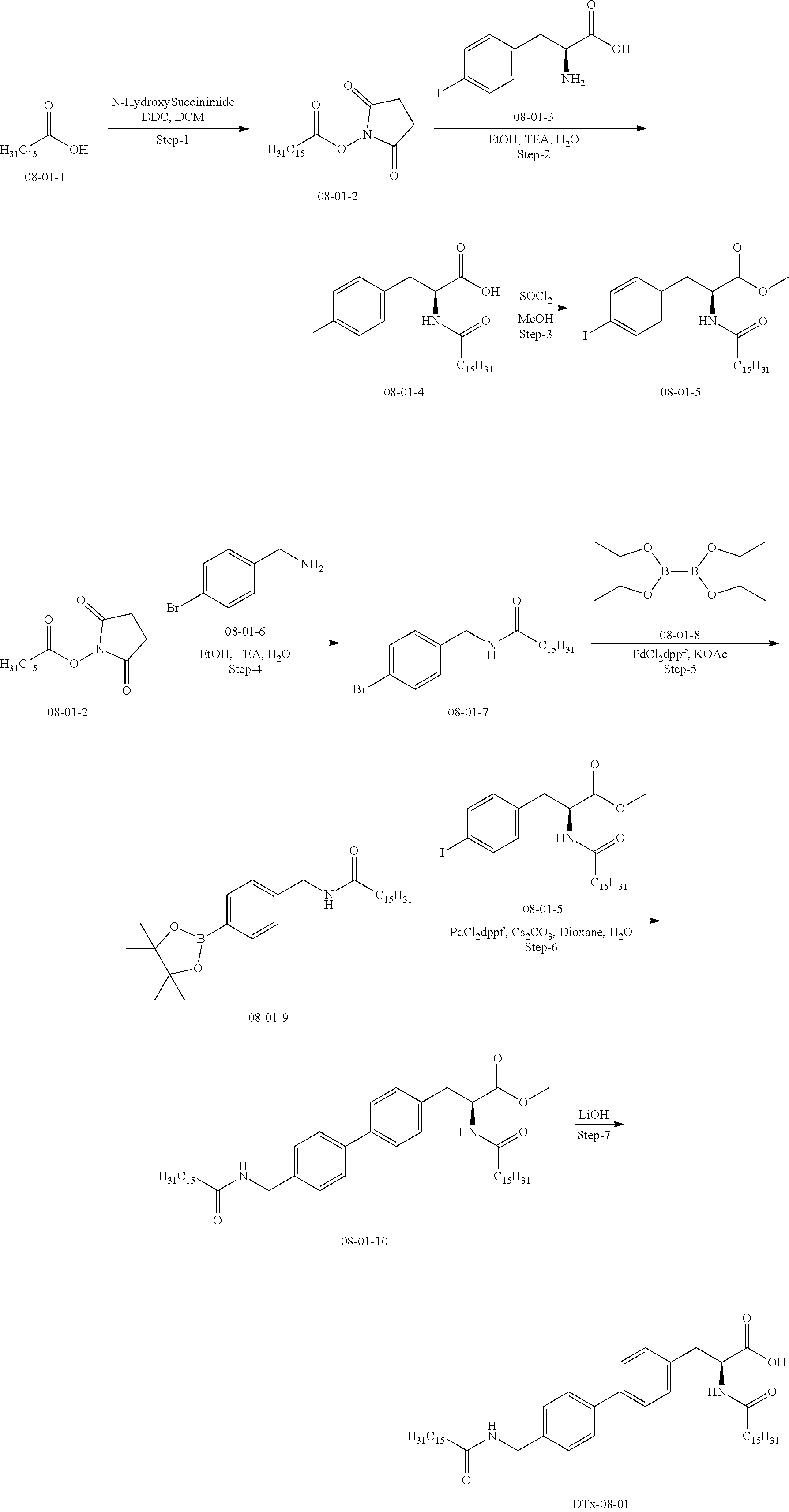

Step 1: To a stirred solution of compound 08-01-1 (10 g, 0.0389 mol) in DCM (200 mL) was added DMAP (0.47 g, 0.0038 mol), DCC (8.04 g, 0.0389 mol) followed by N-hydroxysuccinimide (4.48 g, 0.0389 mol) at RT. The resulting mixture was stirred at RT for 16 h. The reaction was monitored by LCMS. The reaction mixture was filtered through sintered funnel, the filtrate was evaporated to give crude product 08-01-02 as an off white solid which was directly proceeded for next step (10 g, 72%).

Step 2: To a stirred solution of compound 08-01-2 (10 g, 0.0283 mol) in 65% aq. ethanol (100 mL) was added Et₃N (11.8 mL, 0.0849 mol), compound 08-01-3 (10.6 g, 0.0368 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-4 as an off white solid. (11 g, 73%)

Step 3: To a stirred solution of compound 08-01-4 (11 g, 0.0207 mol) in methanol (110 mL) was added thionyl chloride (44 mL) slowly at RT. The resulting mixture was stirred at RT for 16 h. The reaction was monitored by LCMS. The reaction mixture was concentrated under reduced pressure to get crude product which was triturated with diethyl ether to get pure compound of 08-01-5 as an off white solid (9 g, 80%).

Step 4: To a stirred solution of compound 08-01-2 (5 g, 0.0141 mol) in 65% aq. ethanol (50 mL) was added Et₃N (6 mL, 0.0424 mol), compound 08-01-6 (3.3 g, 0.0184 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 08-01-7 as an off white solid. (5.1 g, 85%)

Step 5: To a stirred solution of compound 08-01-7 (5 g, 0.0117 mol) in dioxane (100 mL) was added 08-01-8 ((4,4,4′,4′,5,5,5′,5′-octamethyl-2,2′-bi(1,3,2-dioxaborolane) (4.4 g, 0.0176 mol)) and AcOK (3.4 g, 0.0353 mol). After degassing with nitrogen, Pd(dppf)Cl₂ (0.48 g, 0.0005 mol) was added to the reaction mixture. The resulting mixture was stirred at 90° C. for 12 h. The reaction mixture was monitored by LCMS, the reaction mixture was filtered through celite bed and concentrated under vacuum to give crude product which was further purified by column chromatography using 3% MeOH in DCM as eluent to get the product 01-08-9 as brown solid. (4.8 g, 86%)

Step 6: To a stirred solution of compound 01-08-5 (4.5 g, 0.0082 mol) in dioxane (90 mL) and water (9 mL) was added compound 01-08-9 (4.68 g, 0.0099 mol) and Cs₂CO₃ (8.1 g, 0.0248 mol). After degassing with nitrogen, Pd(dppf)Cl₂ (0.67 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 01-08-10 as brown solid. (1 g, 14.2%)

Step 7: To a stirred solution of compound 01-08-10 (1 g, 0.0013 mol) in MeOH, THF (6.5 mL; 13 mL) and H₂O (6.5 mL) was added LiOH·H₂O (0.16 g, 0.0039 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-08-01 as off white solid (0.5 g, 51%).

### Analytics of DTx-08-01

¹H-NMR- (400 MHz, TFA-d1): δ 0.78-0.79 (m, 6H), 1.08-1.49 (m, 48H), 1.49-1.50 (m, 2H), 1.72-1.83 (m, 2H), 2.69-2.71 (m, 2H), 5.77-2.82 (m, 2H), 3.41 (d, J=14.8 Hz, 1H), 3.53 (d, J=14.4 Hz, 1H), 4.66 (s, 2H), 5.16-5.18 (m, 1H), 7.23 (d, J=8.0 Hz, 2H), 7.33 (d, J=8.0 Hz, 2H), 7.58 (t, J=2.4 Hz, 4H). LCMS: 748.6 (M+1).

### Synthesis of DTx-09-01

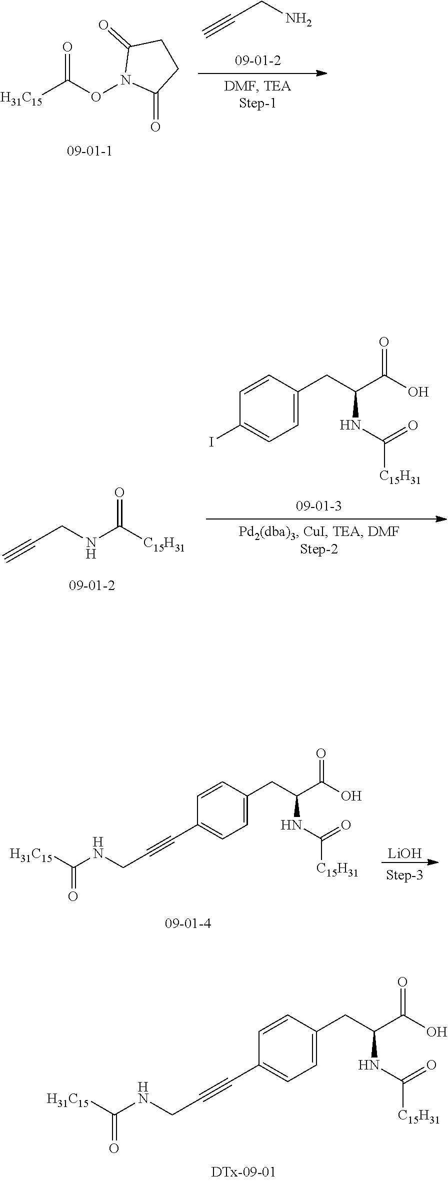

Step 1: To a stirred solution of compound 09-01-1 (10 g, 0.0283 mol) in DMF (100 mL) was added Et₃N (11.7 mL, 0.0849 mol), compound 09-01-2 (2.02 g, 0.0368 mol) slowly at RT. The resulting mixture was stirred at 50° C. for 16 h. The reaction was monitored by LCMS. The reaction mixture was neutralized with 1.5 N HCl, precipitated solid was filtered, washed with water and dried to get the product 09-01-3 as an off white solid. (4.5 g, 55%)

Step 2: To a stirred solution of compound 09-01-4 (5 g, 0.092 mol) in DMF (50 mL) was added compound 09-01-3 (3.5 g, 0.0119 mol), TEA (15 mL) and CuI (0.20 g, 0.0011 mol). After degassing with nitrogen, Pd₂(dba)₃ (0.67 g, 0.0007 mol) was added to the reaction mixture. The resulting mixture was stirred at 50° C. for 3 h. The reaction mixture was monitored by LCMS, the reaction mixture was filtered through celite bed and concentrated under vacuum to give crude product which was further purified by column chromatography using 25% EtOAc in Hexane as eluent to get the product 09-01-5 as off white solid. (1 g, 15.6%)

Step 3: To a stirred solution of compound 09-01-5 (1 g, 0.0014 mol) in MeOH, THF (6.5 mL; 13 mL) and H₂O (6.5 mL) was added LiOH·H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3% MeOH in DCM as eluent to get the product DTx-09-01 as off pale brown solid (0.5 g, 51%).

### Analytics of DTx-09-01

¹H-NMR- (400 MHz, TFA-d1): δ 0.89-0.92 (m, 6H), 1.20-1.40 (m, 49H), 1.67-1.70 (m, 2H), 1.82-1.86 (m, 2H), 2.71-2.75 (m, 2H), 5.91-2.95 (m, 2H), 3.47 (d, J=14.8 Hz, 1H), 3.61 (d, J=14.8 Hz, 1H), 4.52 (s, 2H), 7.25 (d, J=8.0 Hz, 2H), 7.50 (d, J=8.0 Hz, 2H). LCMS: 696.5 (M+1).

### Synthesis of DTx-10-01

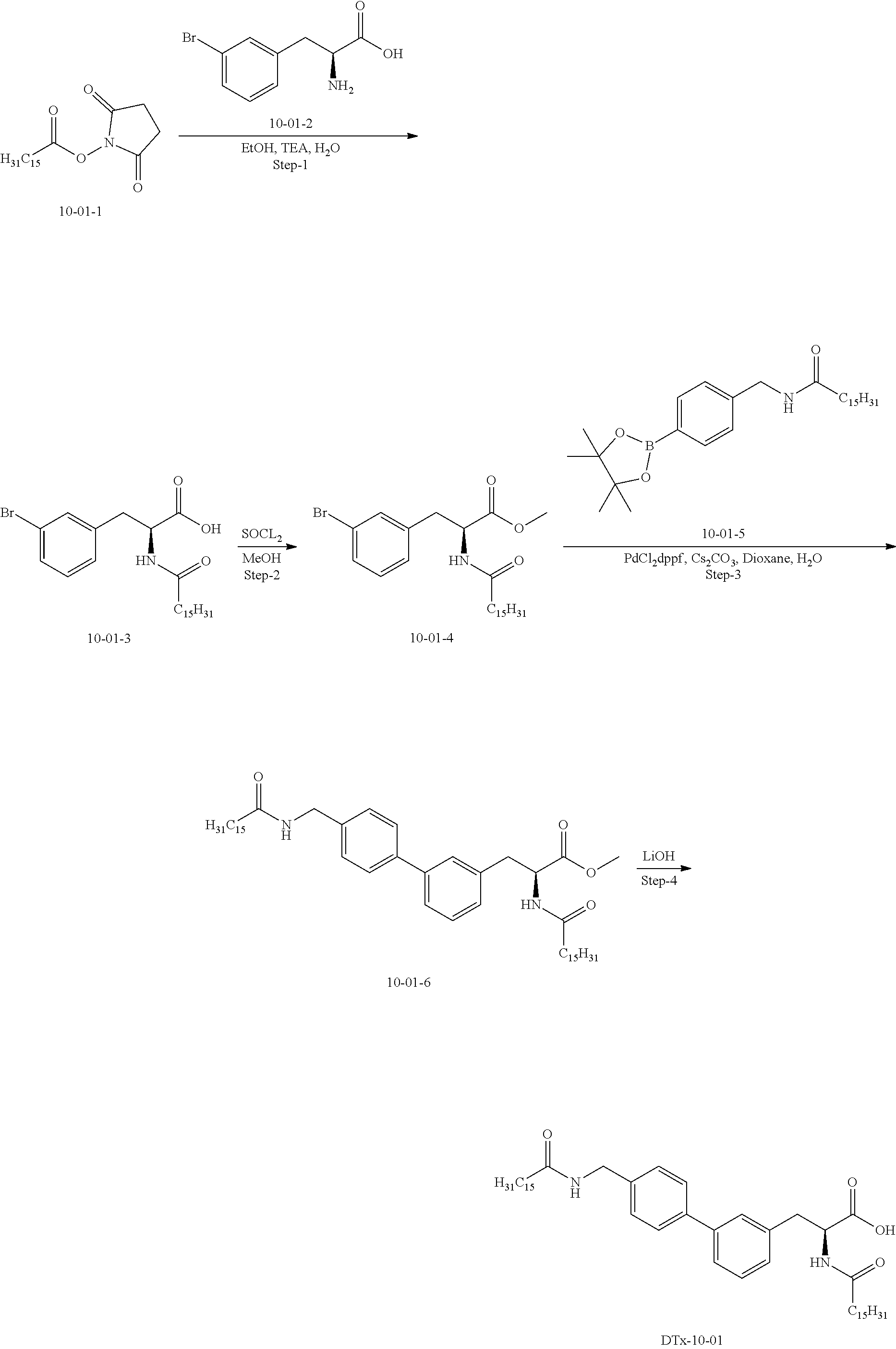

Step 1: To a stirred solution of compound 10-01-1 (5 g, 0.0141 mol) in 65% aq. ethanol (50 mL) was added Et₃N (10 mL, 0.0707 mol), compound 10-01-2 (3.45 g, 0.0141 mol) slowly at RT. The resulting mixture was stirred at 75° C. for 16 h. The reaction was monitored by LCMS. The reaction mixture was neutralized with 1.5 N HCl, precipitated solid was filtered, washed with water and dried to get the product 10-01-3 as an off white solid. (5.5 g, 80.6%)

Step 2: To a stirred solution of compound 10-01-3 (5.5 g, 0.0113 mol) in methanol (550 mL) was added thionyl chloride (22 mL) slowly at RT. The resulting mixture was stirred at RT for 16 h. The reaction was monitored by LCMS. The reaction mixture was concentrated under reduced pressure to get crude product which was triturated with diethyl ether to get pure compound of 10-01-4 as an off white solid (4.3 g, 76%).

Step 3: To a stirred solution of compound 10-01-4 (4.3 g, 0.0086 mol) in dioxane (90 mL) and water (9 mL) was added compound 10-01-5 (4.5 g, 0.00952 mol) and Cs₂CO₃ (8.4.6 g, 0.0259 mol). After degassing with nitrogen, Pd(dppf)Cl₂ (0.7 g, 0.0008 mol) was added to the reaction mixture. The resulting mixture was stirred at 90° C. for 3 h. The reaction mixture was monitored by LCMS, the reaction mixture was filtered through celite bed and concentrated under vacuum to give crude product which was further purified by column chromatography using 3% MeOH in DCM as eluent to get the product 10-01-6 as brown solid. (1.1 g, 16.68%)

Step 4: To a stirred solution of compound 10-01-6 (1.1 g, 0.0014 mol) in MeOH, THF (6.5 mL; 13 mL) and H₂O (6.5 mL) was added LiOH·H₂O (0.18 g, 0.0042 mol) and the reaction mixture was stirred at 50° C. for 3 h. The reaction mixture was monitored by LCMS, the reaction mixture was concentrated under vacuum. The resultant product was neutralized with 1.5 N HCl, the solid which was precipitated was filtered, washed with water and dried under vacuum to get the crude product. The crude product was triturated with MeOH to obtained pure DTx-10-01 as off white solid (0.7 g, 64%).

### Analytics of DTx-10-01

¹H-NMR- (400 MHz, TFA-d1): δ 0.78-0.80 (m, 6H), 1.13-1.45 (m, 50H), 1.73-1.75 (m, 2H), 2.39-2.43 (m, 1H), 2.70-2.74 (m, 2H), 3.14-3.20 (m, 1H), 3.46-3.51 (m, 2H), 4.68 (s, 2H), 5.17-5.20 (m, 1H), 7.17 (d, J=7.2 Hz, 1H), 7.33-7.43 (m, 4H), 7.50 (d, J=7.6 Hz, 1H), 7.57-7.58 (m, 2H). LCMS: 748.5 (M+1)

### Synthesis of DTx-11-01

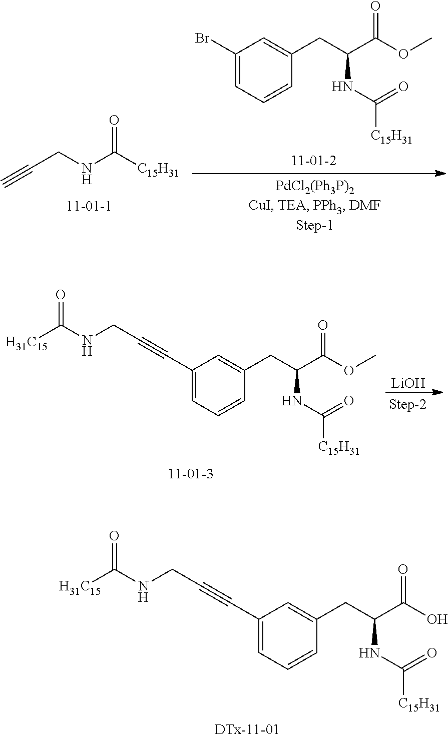

Step 1: To a stirred solution of compound 11-01-1 (2.68 g, 0.0091 mol) in DMF (35 mL) in a sealed tube was added compound 11-01-2 (3.5 g, 0.0070 mol), TEA (18 mL), PPh₃ (0.18 g, 0.0007 mol) and CuI (0.16 g, 0.0008 mol). After degassing with nitrogen, PdCl₂(Ph₃P)₂ (0.39 g, 0.0005 mol) was added to the reaction mixture. The resulting mixture was stirred at 110° C. for 3 h. The reaction mixture was monitored by LCMS, the reaction mixture was filtered through celite bed and concentrated under vacuum to give crude product which was further purified by column chromatography using 25% EtOAc in Hexane as eluent to get the product 11-01-3 as off white solid. (1 g, 20%)

Step 2: To a stirred solution of compound 11-01-3 (1 g, 0.0014 mol) in MeOH, THF (6.5 mL; 13 mL) and H₂O (6.5 mL) was added LiOH·H₂O (0.17 g, 0.0042 mol) and the reaction mixture was stirred at 50° C. for 2 h. The reaction mixture was monitored by LCMS, the reaction mixture was concentrated under vacuum to give crude which was neutralized with 1.5 N HCl, precipitated solid was filtered, washed with water and dried under vacuum to get the crude product. The crude product was further purified by column chromatography using 3% MeOH in DCM as eluent to get the product DTx-11-01 as off pale brown solid (0.7 g, 71%).

### Analytics of DTx-11-01

¹H-NMR- (400 MHz, TFA-d1): δ 0.87-0.90 (m, 6H), 1.31-1.47 (m, 48H), 1.65-1.68 (m, 2H), 1.81-1.85 (m, 2H), 2.71-2.74 (m, 2H), 2.89-2.95 (m, 2H), 3.42 (d, J=14.8 Hz, 1H), 3.57 (d, J=14.8 Hz, 1H), 4.50 (s, 2H), 5.20-5.24 (m, 1H), 7.25 (d, J=7.6 Hz, 1H), 7.34 (s, 1H), 7.39 (t, J=8.0 Hz, 1H), 7.47 (d, J=7.6 Hz, 1H). LCMS: 696.5 (M+1).

### Synthesis of DTx-04-01

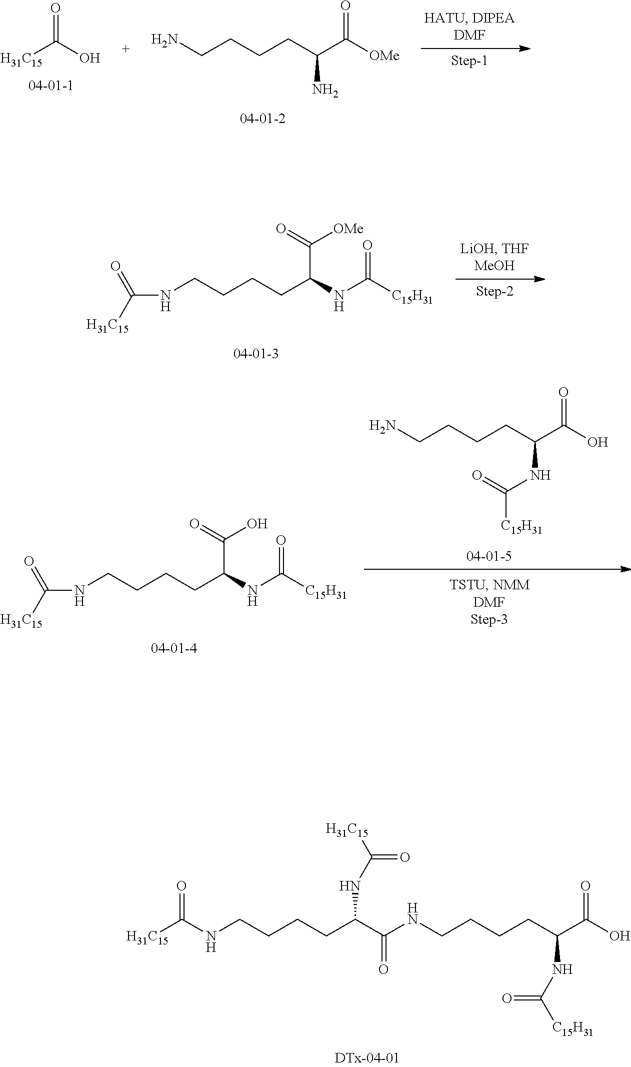

Step 1: To a stirred solution of compound 04-01-2 (5 g, 0.021 mol) in DMF (100 mL) was added DIPEA (19.7 mL, 0.107 mol), compound 04-01-1 (13.73 g, 0.053 mol) HATU (12.23 g, 0.032 mol) slowly at RT. The resulting mixture was stirred at RT for 16 h. The reaction was monitored by LCMS. The reaction mixture was quenched with ice cold water and filtered the solid, dried the solid under the vacuum to get the product 04-01-3 as off white solid (9.1 g, 67%).

Step 2: To a stirred solution of compound 04-01-3 (5 g, 0.0078 mol) in MeOH, THF (100 mL; 1:1) and H₂O (5 mL) was added LiOH·H₂O (0.660 g, 0.0157 mol) and the reaction mixture was stirred at RT for 16 h. The reaction mixture was monitored by LCMS, the reaction mixture was concentrated under vacuum to give crude which was neutralized with 1.5 N HCl, the solid which was precipitated was filtered, washed with water and dried under vacuum to get the product 04-01-4 as off white solid (3.9 g, 80%).

Step 3: To a stirred solution of compound 04-01-4 (3.0 g, 0.0048 mol) in DMF (60 mL) was added NMM (15 mL), followed by TSTU (2.18 g, 0.0096 mol) at RT. The resulting mixture was stirred at RT for 2 h. Compound 5 (3.69 g, 0.0096 mol) was added to the reaction mixture at 0° C. and then stirred at RT for 16 h. The reaction mixture was neutralized with 1.5 N HCl, precipitated solid was filtered, washed with water and dried. The crude product was triturated with MeOH to get the product DTx-04-01 as an off white solid. (2.8 g, 58%).

### Analytics of DTx-04-01

¹H-NMR- (400 MHz, TFA-d): δ 1.09-1.13 (m, 9H), 1.57-2.16 (m, 84H), 2.38-2.44 (m, 3H), 2.77-2.94 (m, 4H), 3.18-3.31 (m, 5H), 3.69-3.81 (m, 5H), 4.87-4.92 (m, 1H). LCMS: 990.8 (M+1).

### Synthesis of DTx-05-01

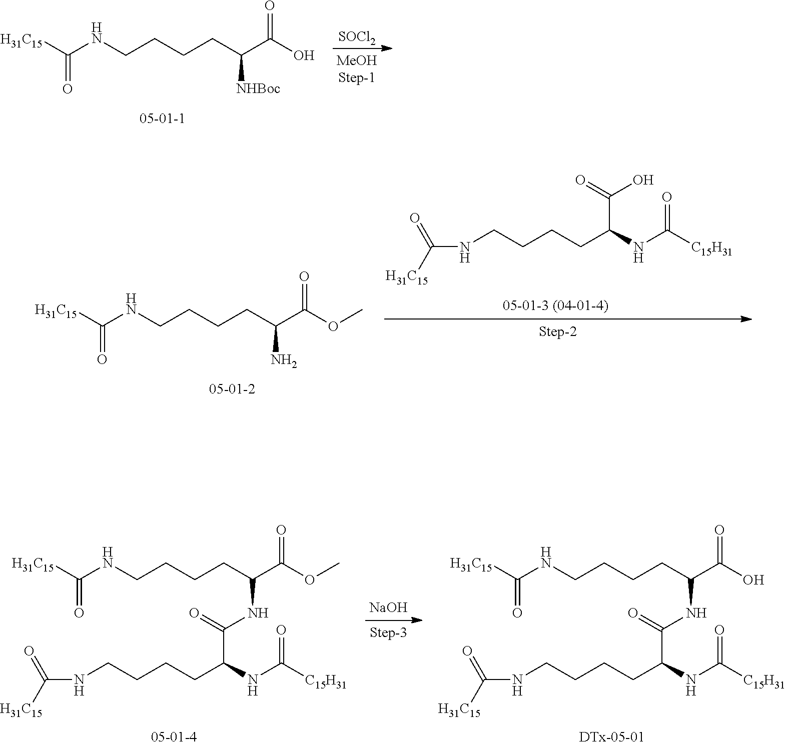

Step 1: To a stirred solution of compound 05-01-1 (5 g, 0.0103 mol) in methanol (50 mL) was added thionyl chloride (3.8 mL, 0.0516 mol) slowly at 0° C. The resulting mixture was stirred at RT for 16 h. The resulting mixture was evaporated and triturated with diethyl ether to give compound 05-01-2 as an off white solid which was directly proceeded for next step (3.5 g, 85%).

Step 2: To a stirred solution of compound 05-01-2 (2.89 g, 0.0067 mol) in DMF (35 mL) was added DIPEA (1.55 mL, 0.0084 mol), compound 05-01-3 (3.5 g, 0.0056 mol) and HBTU (2.12 g, 0.0056 mol) slowly at 0° C. The resulting mixture was stirred at 50° C. for 16 h. The reaction was monitored by LCMS. The reaction mixture was neutralized with 1.5 N HCl, precipitated solid was filtered, washed with water and dried to give compound 05-01-4 as pale brown solid. (3.2 g, 69%).

Step 3: To a stirred solution of compound 05-01-4 (3.2 g, 0.0031 mol) in MeOH, THF (60 mL; 1:1) and H₂O (3 mL) was added NaOH (0.25 g, 0.0062 mol) and the reaction mixture was stirred at 50° C. for 16 h. The reaction mixture was monitored by LCMS, the reaction mixture was concentrated and neutralized with 1.5 N HCl. The precipitated solid was filtered, washed with water and dried. The crude product was triturated with MeOH to give DTx-05-01 as pale brown solid. (2.3 g, 73%).

### Analytics of DTx-05-01

¹H-NMR- (400 MHz, TFA-d): δ 0.87-0.89 (m, 9H), 1.60-1.80 (m, 76H), 1.94-2.14 (m, 15H), 2.55-2.59 (m, 2H), 2.70-2.75 (m, 4H), 3.59-3.60 (m, 4H), 4.73-4.76 (m, 1H). LCMS: 990.8 (M+1).

### Synthesis of DTx-01-50 & DTx-01-52

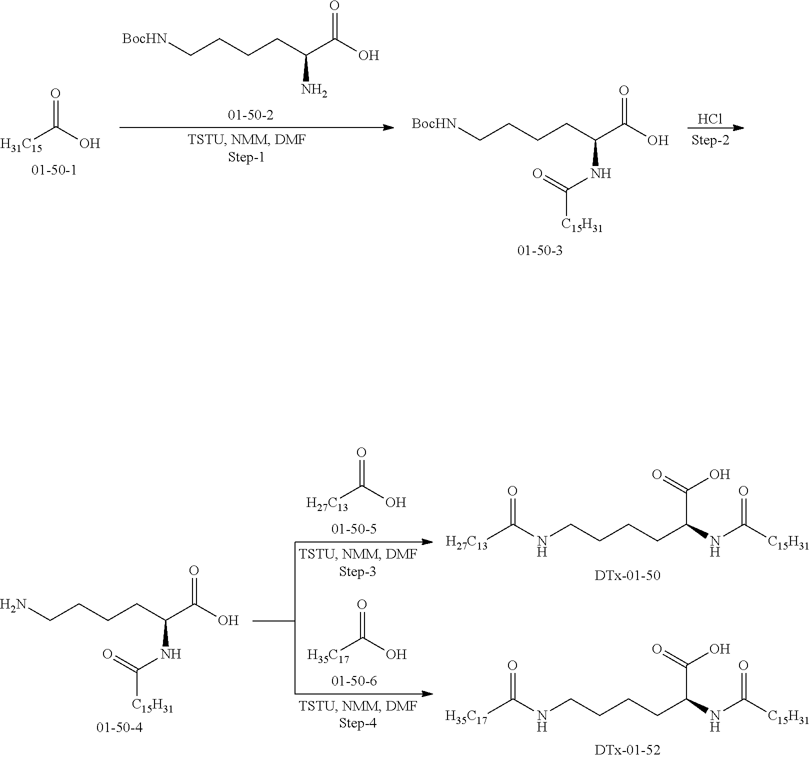

Step 1: To a stirred solution of 01-50-1 (5.0 g, 0.019 mol) in DMF (50 mL) was added NMM (25 mL), followed by TSTU (6.46 g, 0.021 mol) at RT. The resulting mixture was stirred at RT for 2 h. 01-50-2 (7.2 g, 0.029 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-50-3 as brown solid. (9.1 g, 96%).

Step 2: To a stirred solution of compound 01-50-3 (9.1 g, 0.018 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-50-4 as an off white solid (6.5 g, 82%).

Step 3: To a stirred solution of compound 01-50-5 (1.5 g, 0.0065 mol) in DMF (45 mL) was added NMM (23 mL), followed by TSTU (2.17 g, 0.0072 mol) at RT. The resulting mixture was stirred at RT for 2 h. 01-50-4 (3.32 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-50 as pale brown solid. (2.1 g, 53%). LCMS: 595.5 (M+1). ¹H-NMR- (400 MHz, TFA-d): δ 0.93-0.95 (m, 6H), 1.38-1.65 (m, 44H), 1.65-1.69 (m, 2H), 1.84-2.06 (m, 7H), 2.20-2.24 (m, 1H), 2.67 (t, J=7.6 Hz, 2H), 2.82 (t, J=7.9 Hz, 2H), 3.68 (t, J=6.8 Hz, 2H), 4.93 (t, J=8.0 Hz, 1H).

Step 4: To a stirred solution of compound 6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 4 (2.66 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-52 as pale brown solid. (2.2 g, 64%). LCMS: 652.5 (M+1).

¹H-NMR- (400 MHz, TFA-d): δ 0.93-0.94 (m, 6H), 1.37-1.59 (m, 52H), 1.66-1.68 (m, 2H), 1.84-2.05 (m, 7H), 2.20-2.23 (m, 1H), 2.67 (t, J=7.3 Hz, 2H), 2.81 (t, J=7.5 Hz, 2H), 3.69 (t, J=6.2 Hz, 2H), 4.92 (t, J=4.9 Hz, 1H).

### Synthesis of DTx-01-51 & DTx-01-54

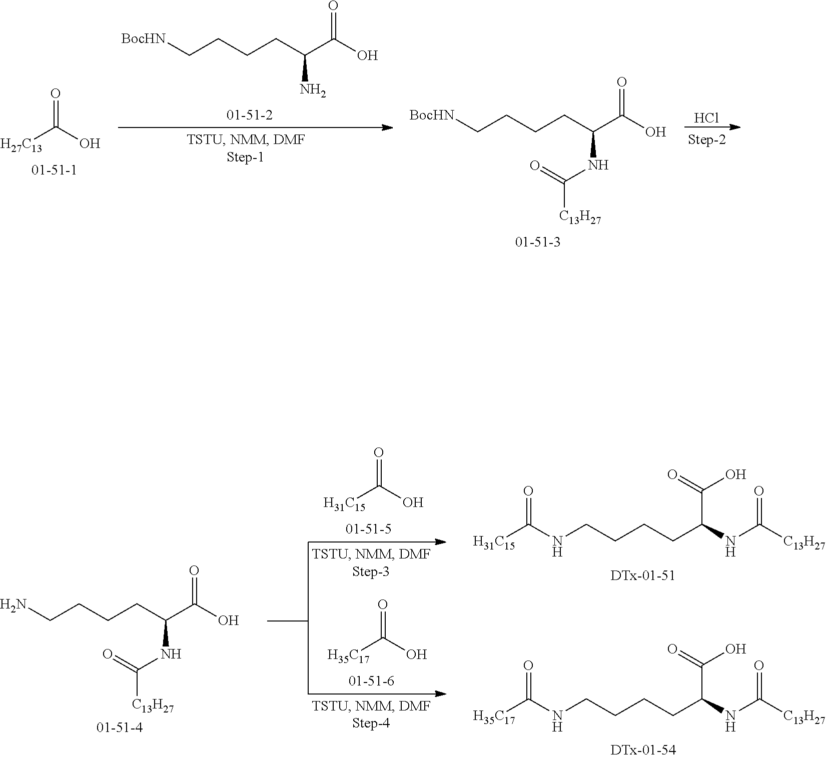

Step 1: To a stirred solution of 01-51-1 (5.0 g, 0.021 mol) in DMF (50 mL) was added NMM (25 mL), followed by TSTU (7.25 g, 0.024 mol) at RT. The resulting mixture was stirred at RT for 2 h. Compound 01-51-2 (8.09 g, 0.032 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product 01-51-3 as brown solid. (9 g, 90%).

Step 2: To a stirred solution of compound 01-51-3 (9 g, 0.014 mol) in 1,4 dioxane (45 mL) was added 4 M HCl in dioxane (45 mL) slowly at RT. The resulting mixture was stirred at RT for 16 h. The reaction mixture was concentrated under reduced pressure to get crude product which was triturated with diethyl ether to get pure compound of 01-51-4 as an off white solid (6.6 g, 810%).

Step 3: To a stirred solution of compound 01-51-5 (1.5 g, 0.0058 mol) in DMF (45 mL) was added NMM (23 mL), followed by TSTU (1.93 g, 0.0064 mol) at RT. The resulting mixture was stirred at RT for 2 h. Compound 01-51-4 (2.76 g, 0.0070 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-51 as pale brown solid. (2.4 g, 68%). LCMS: 595.5 (M+1). ¹H-NMR- (400 MHz, TFA-d): δ 0.89-0.92 (m, 6H), 1.34-1.50 (m, 44H), 1.63-1.65 (m, 2H), 1.81-2.08 (m, 7H), 2.20-2.21 (m, 1H), 2.63 (t, J=7.3 Hz, 2H), 2.78 (t, J=7.4 Hz, 2H), 3.65 (t, J=6.4 Hz, 2H), 4.89 (t, J=7.1 Hz, 1H).

Step 4: To a stirred solution of compound 01-51-6 (1.5 g, 0.0052 mol) in DMF (45 mL) was added NMM (23 mL), followed by TSTU (1.74 g, 0.0058 mol) at RT. The resulting mixture was stirred at RT for 2 h. Compound 01-51-4 (2.49 g, 0.0063 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-54 as pale brown solid. (2.2 g, 66%). LCMS: 624.6 (M+1).

¹H-NMR- (400 MHz, TFA-d): δ 0.89-0.90 (m, 6H), 1.32-1.57 (m, 49H), 1.62-1.64 (m, 2H), 1.74-1.99 (m, 6H), 2.14-2.18 (m, 1H), 2.61 (t, J=7.6 Hz, 2H), 2.76 (t, J=7.6 Hz, 2H), 3.62 (t, J=7.0 Hz, 2H), 4.85-4.88 (m, 1H).

### Synthesis of DTx-01-53 & DTx-01-55

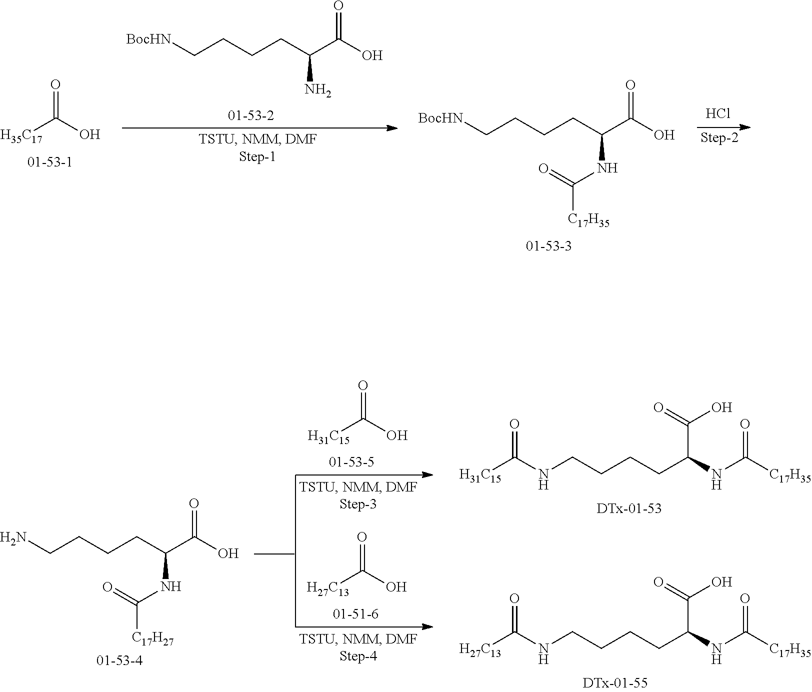

Step 1: To a stirred solution of compound 1 (5.0 g, 0.017 mol) in DMF (50 mL) was added NMM (25 mL), followed by TSTU (5.82 g, 0.019 mol) at RT. The resulting mixture was stirred at RT for 2 h. Compound 2 (5.18 g, 0.021 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product 3 as brown solid. (8.6 g, 95%).

Step 2: To a stirred solution of compound 3 (8.6 g, 0.016 mol) in 1,4 dioxane (43 mL) was added 4 M HCl in dioxane (43 mL) slowly at RT. The resulting mixture was stirred at RT for 16 h. The reaction mixture was concentrated under reduced pressure to get crude product which was triturated with diethyl ether to get pure compound of 4 as an off white solid (7 g, 93%).

Step 3: To a stirred solution of compound 5 (1.5 g, 0.0058 mol) in DMF (45 mL) was added NMM (23 mL), followed by TSTU (1.94 g, 0.0064 mol) at RT. The resulting mixture was stirred at RT for 2 h. Compound 4 (3.15 g, 0.0070 mol) was added to the reaction mixture at 0° C. and then stirred at 70° C. for 5 h and then concentrated. The reaction mixture was neutralized with 1.5 N HCl, precipitated solid was filtered, washed with water and dried. The crude product was triturated with MeOH to get the product DTx-01-53 as pale brown solid. (2.2 g, 57%). LCMS: 652.6 (M+1). ¹H-NMR- (400 MHz, TFA-d): δ 0.82-0.85 (m, 6H), 1.27-1.50 (m, 52H), 1.54-1.58 (m, 2H), 1.73-1.94 (m, 7H), 2.07-2.14 (m, 1H), 2.56 (t, J=8.0 Hz, 2H), 2.71 (t, J=8.0 Hz, 2H), 3.58 (t, J=6.8 Hz, 2H), 4.81-4.84 (m, 1H).

Step 4: To a stirred solution of compound 6 (1.5 g, 0.0065 mol) in DMF (45 mL) was added NMM (23 mL), followed by TSTU (2.17 g, 0.0072 mol) at RT. The resulting mixture was stirred at RT for 2 h. Compound 4 (3.53 g, 0.0078 mol) was added to the reaction mixture at 0° C. and then stirred at 70° C. for 5 h and then concentrated. The residue was neutralized with 1.5 N HCl, precipitated solid was filtered, washed with water and dried. The crude product was triturated with MeOH to get the product DTx-01-55 as pale brown solid. (2.3 g, 56%). LCMS: 624.6 (M+1).

¹H-NMR- (400 MHz, TFA-d): δ 0.90-0.93 (m, 6H), 1.35-1.49 (m, 48H), 1.60-1.63 (m, 2H), 1.77-2.02 (m, 7H), 2.17-2.21 (m, 1H), 2.64 (t, J=7.6 Hz, 2H), 2.78 (t, J=7.7 Hz, 2H), 3.65 (t, J=7.0 Hz, 2H), 4.88-4.91 (m, 1H).

The motifs presented in the above synthesis schemes, as well as additional motifs, are listed in Table 1.

The synthesis of certain motifs produces a motif comprising a methyl ester protecting group. For example, synthesis of the motif DTx-01-12 produces DTx-01-12-OMe, the methyl ester of DTx-01-12. Following conjugation to a nucleic acid compound, the methyl ester protecting group is removed and no longer present in the lipid motif. Thus, as illustrated in Table 1, FIGS. 1 through 12, and FIGS. 80 through 83, these certain motifs are shown without a methyl ester protecting group.

TABLE 1
DTx Motifs

| Motif Name | Structure |
|---|---|
| DTx-01-01 | 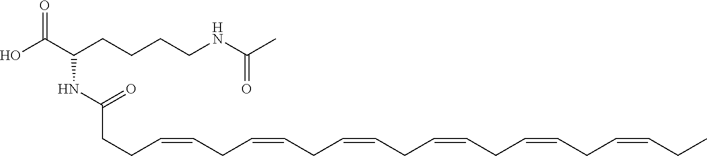 |
| DTx-01-03 | 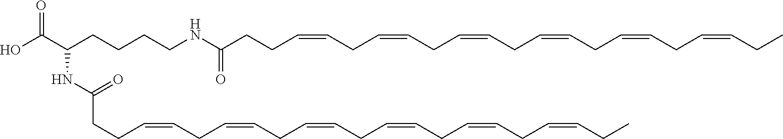 |
| DTx-01-06 | 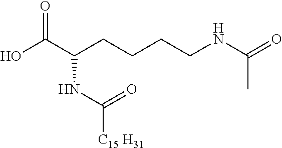 |
| DTx-01-07 | 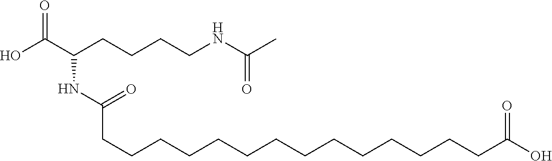 |
| DTx-01-08 | 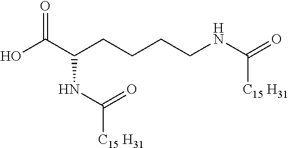 |
| DTx-01-09 | 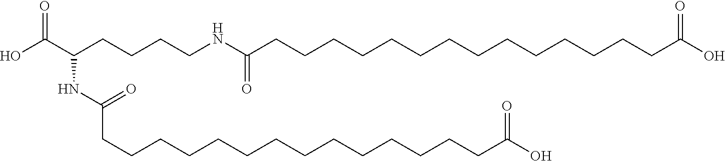 |
| DTx-01-11 | 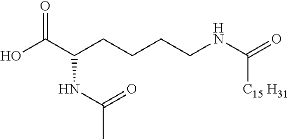 |
| DTx-01-12 | 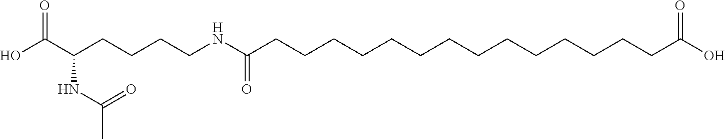 [SMILES: CC(=O)N[C@@H](CCCCNC(=O)CCCCCCCCCCCCCCC(=O)O)C(=O)O] |
| DTx-01-13 | 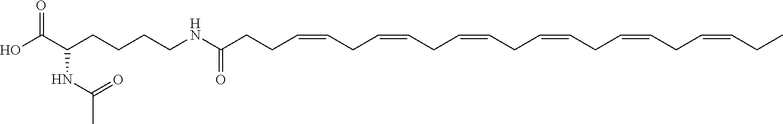 |
| DTx-01-30 | 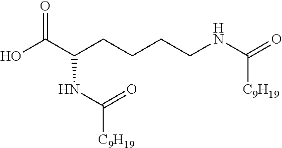 |
| DTx-01-31 | 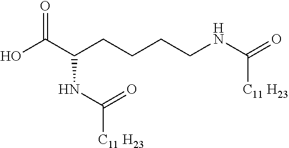 |
| DTx-01-32 | 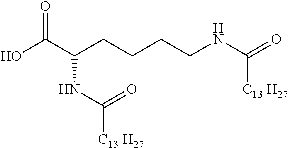 |
| DTx-01-33 | 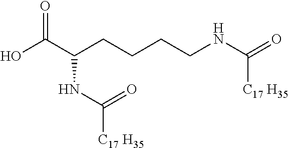 |
| DTx-01-34 | 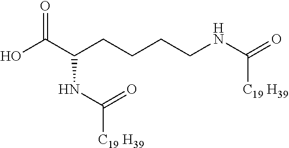 |
| DTx-01-35 | 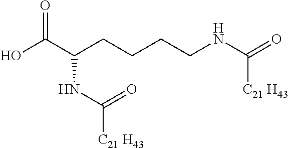 |
| DTx-01-36 | 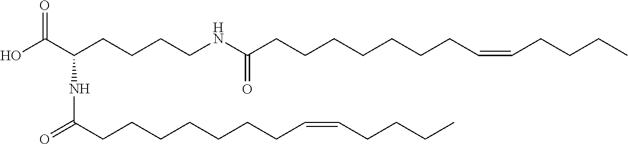 [SMILES: CCCC/C=C\CCCCCCCC(=O)NCCCC[C@H](NC(=O)CCCCCCC/C=C\CCCC)C(=O)O] |
| DTx-01-39 | 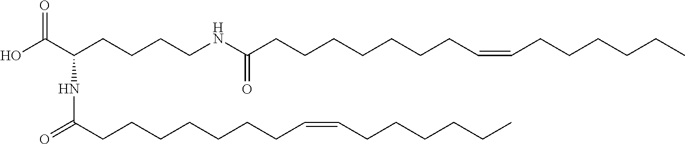 [SMILES: CCCCCC/C=C\CCCCCCCC(=O)NCCCC[C@H](NC(=O)CCCCCCC/C=C\CCCCCC)C(=O)O] |
| DTx-01-43 | 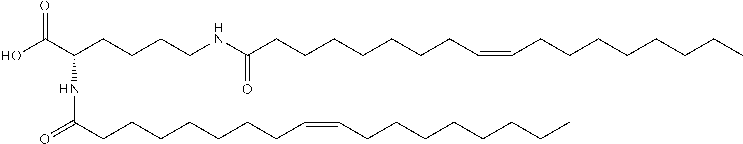 [SMILES: CCCCCCCC/C=C\CCCCCCCC(=O)NCCCC[C@H](NC(=O)CCCCCCC/C=C\CCCCCCCC)C(=O)O] |
| DTx-01-44 | 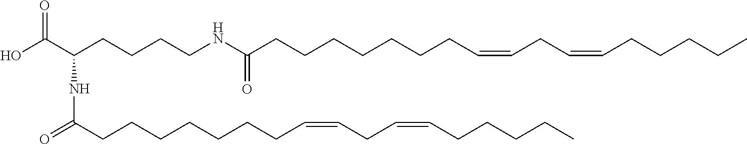 [SMILES: CCCCC/C=C\C/C=C\CCCCCCCC(=O)NCCCC[C@H](NC(=O)CCCCCCC/C=C\C/C=C\CCCCC)C(=O)O] |
| DTx-01-45 | 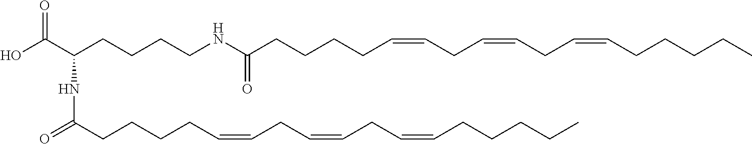 [SMILES: CCCCC/C=C\C/C=C\C/C=C\CCCCC(=O)NCCCC[C@H](NC(=O)CCCC/C=C\C/C=C\C/C=C\CCCCC)C(=O)O] |
| DTx-01-46 | 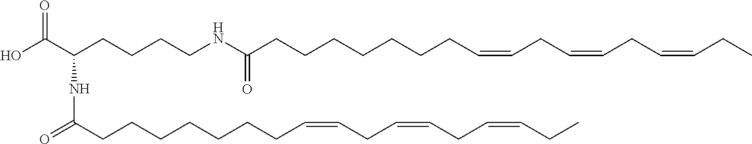 [SMILES: CC/C=C\C/C=C\C/C=C\CCCCCCCC(=O)NCCCC[C@H](NC(=O)CCCCCCC/C=C\C/C=C\C/C=C\CC)C(=O)O] |
| DTx-01-50 | 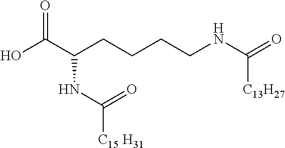 |
| DTx-01-51 | 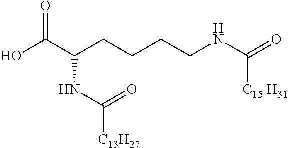 |
| DTx-01-52 | 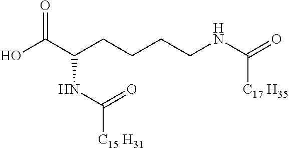 |
| DTx-01-53 | 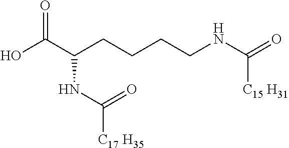 |
| DTx-01-54 | 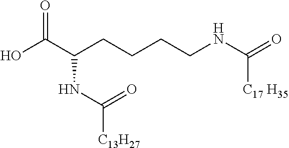 |
| DTx-01-55 | 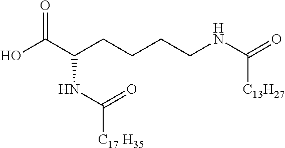 |
| DTx-03-06 | 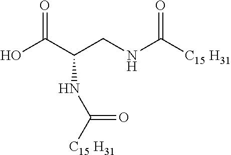 |
| DTx-03-50 | 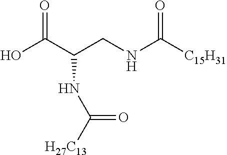 |
| DTx-03-51 | 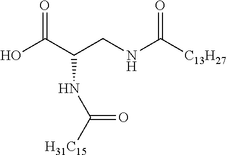 |
| DTx-03-52 | 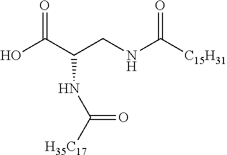 |
| DTx-03-53 | 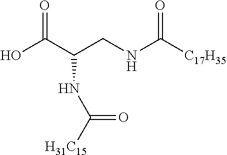 |
| DTx-03-54 | 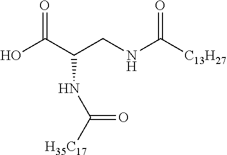 |
| DTx-03-55 | 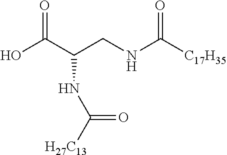 |
| DTx-04-01 | 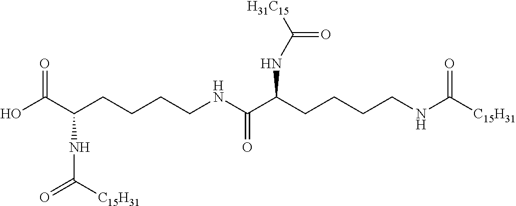 |
| DTx-05-01 | 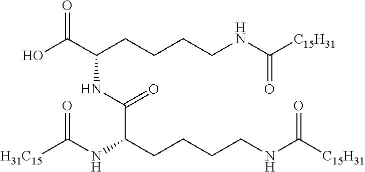 |
| DTx-06-06 | 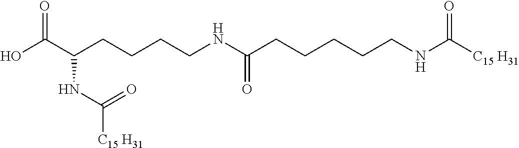 |
| DTx-06-50 | 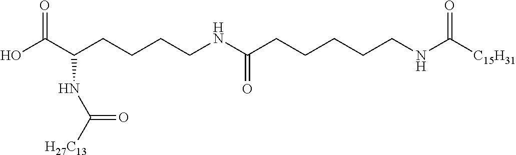 |
| DTx-06-51 | 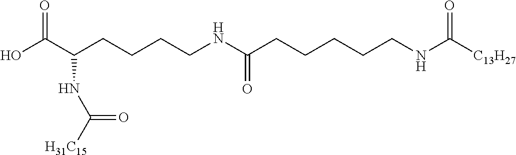 |
| DTx-06-52 | 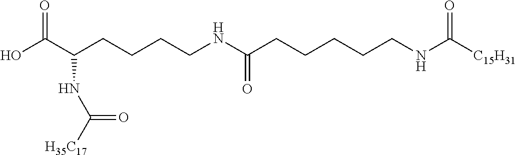 |
| DTx-06-53 | 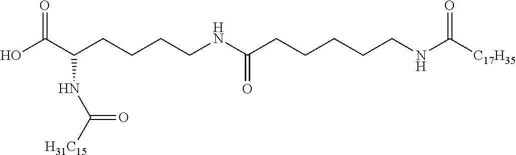 |
| DTx-06-54 | 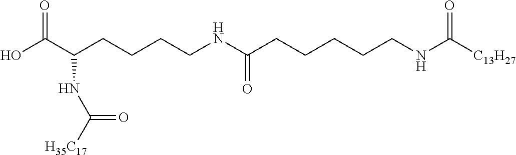 |
| DTx-06-55 | 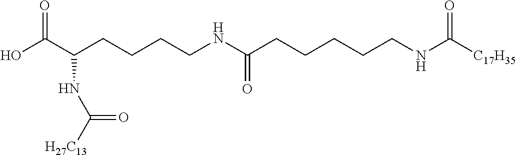 |
| DTx-08-01 | 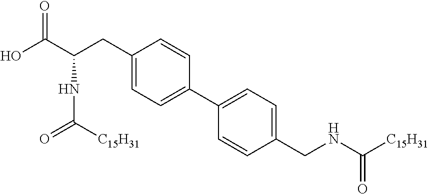 |
| DTx-09-01 | 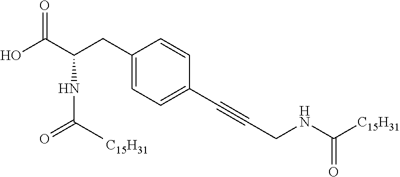 |
| DTx-10-01 | 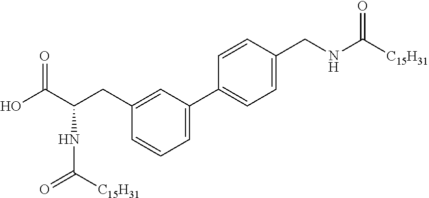 |
| DTx-11-01 | 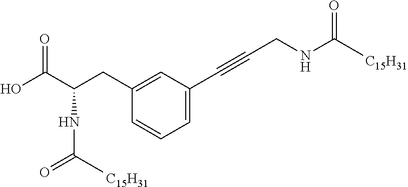 |
| DTx-01-60 | 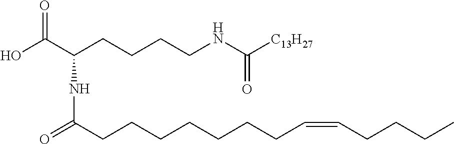 |
| DTx-01-61 | 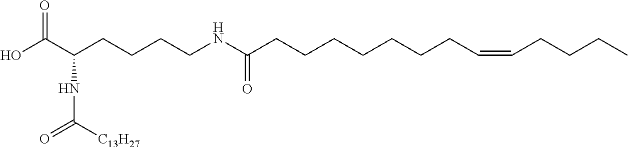 |
| DTx-01-62 | 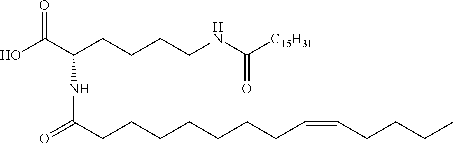 |
| DTx-01-63 | 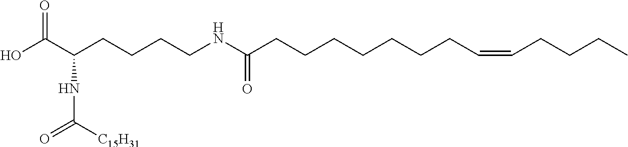 |
| DTx-01-64 | 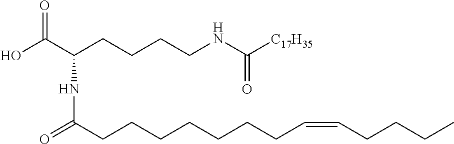 |
| DTx-01-65 | 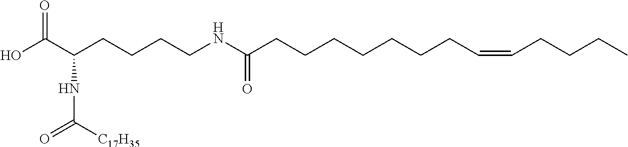 |
| DTx-01-66 | 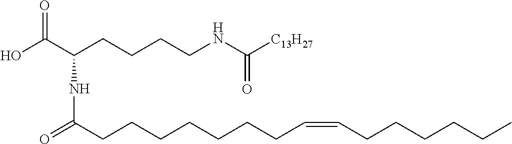 |
| DTx-01-67 | 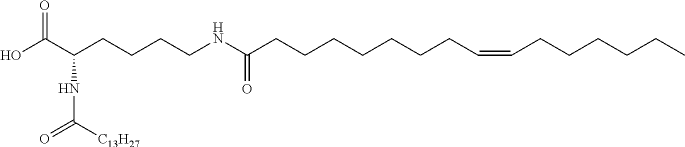 |
| DTx-01-68 | 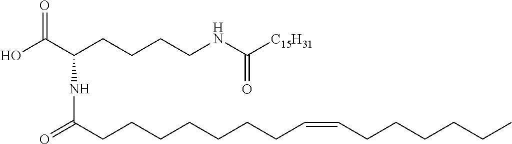 |
| DTx-01-69 | 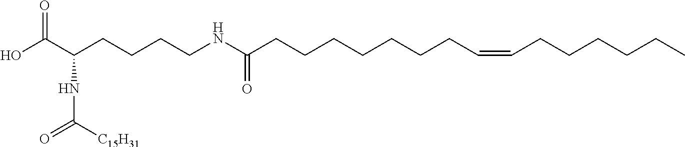 |
| DTx-01-70 | 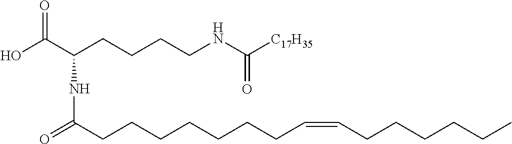 |
| DTx-01-71 | 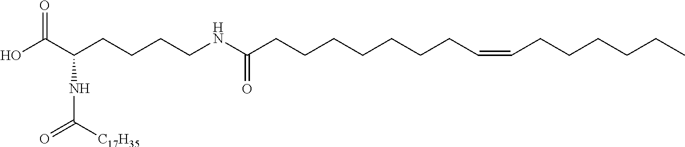 |
| DTx-01-72 | 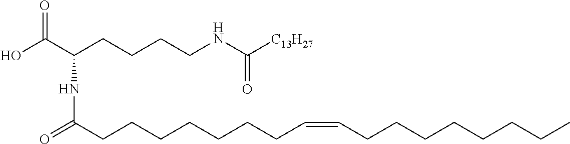 |
| DTx-01-73 | 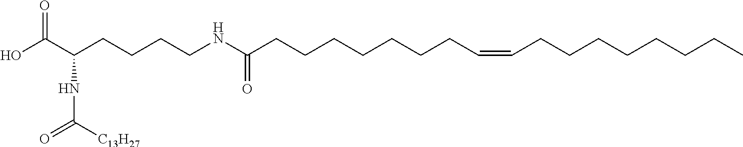 |
| DTx-01-74 | 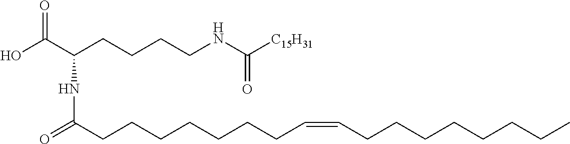 |
| DTx-01-75 | 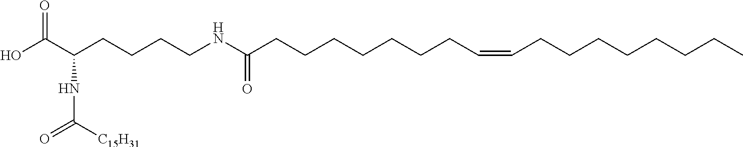 |
| DTx-01-76 | 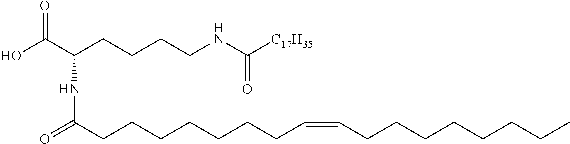 |
| DTx-01-77 | 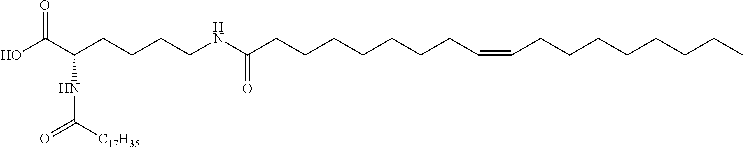 |
| DTx-01-78 | 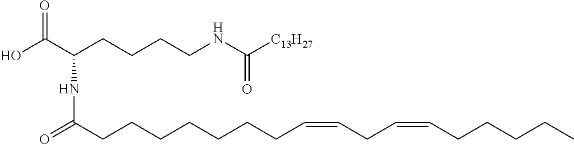 |
| DTx-01-79 | 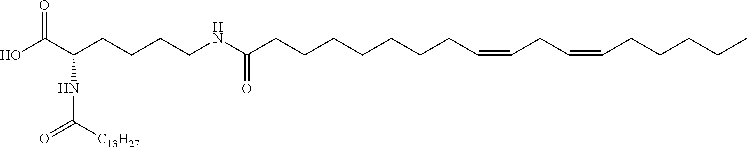 |
| DTx-01-80 | 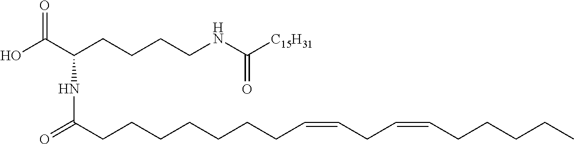 |
| DTx-01-81 | 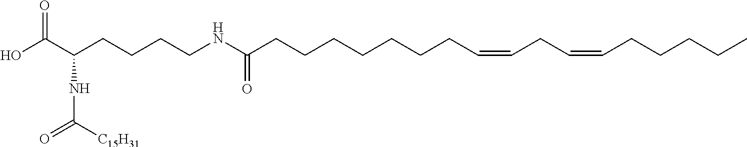 |
| DTx-01-82 | 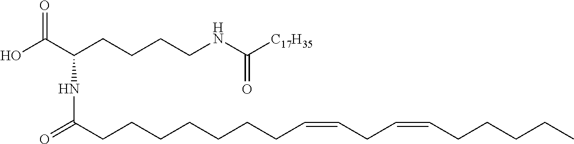 |
| DTx-01-83 | 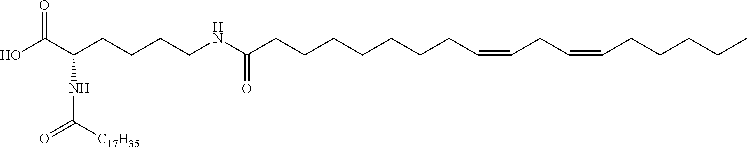 |
| DTx-01-84 | 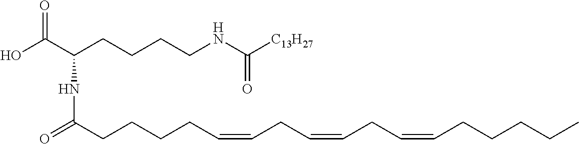 |
| DTx-01-85 | 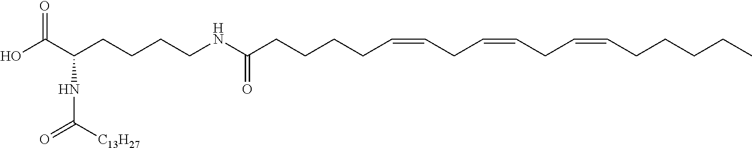 |
| DTx-01-86 | 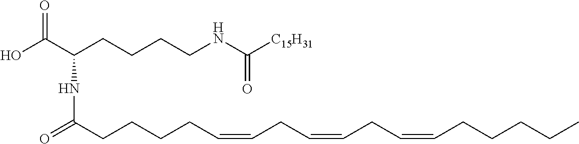 |
| DTx-01-87 | 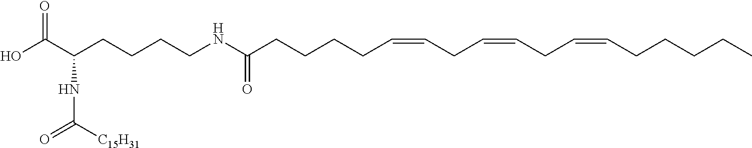 |
| DTx-01-88 | 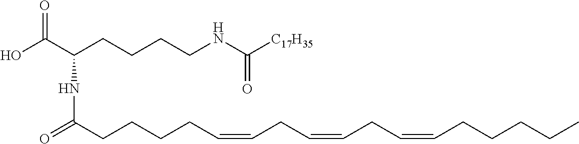 |
| DTx-01-89 | 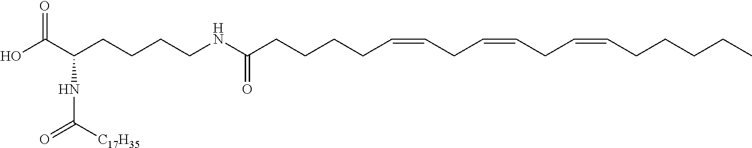 |
| DTx-01-90 | 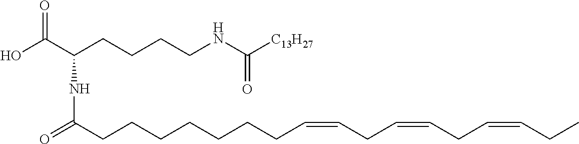 |
| DTx-01-91 | 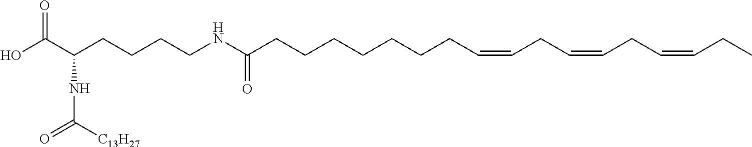 |
| DTx-01-92 | 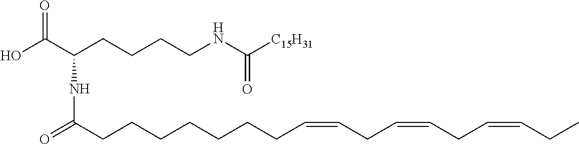 |
| DTx-01-93 | 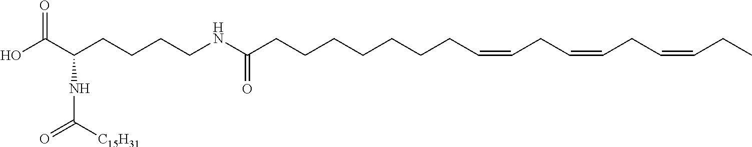 |
| DTx-01-94 | 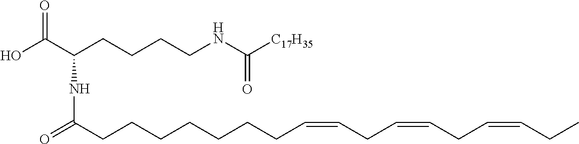 |
| DTx-01-95 | 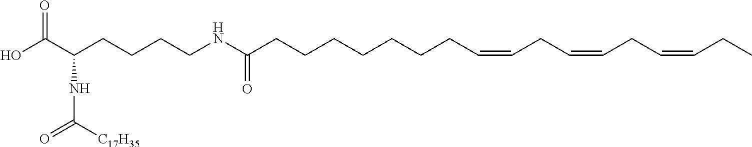 |
| DTx-01-96 | 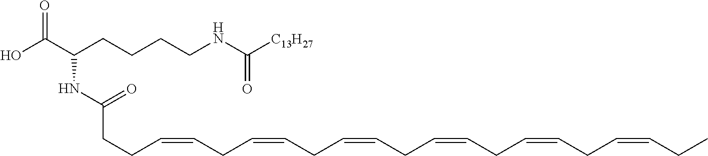 |
| DTx-01-97 | 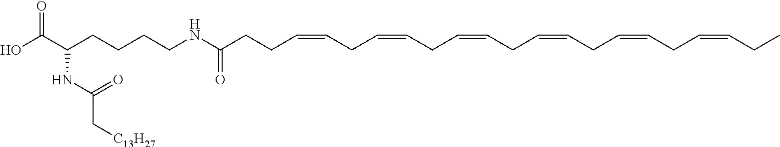 |
| DTx-01-98 | 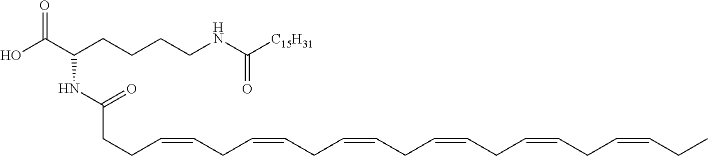 |
| DTx-01-99 | 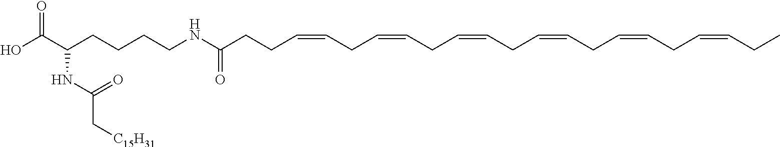 |
| DTx-01-100 | 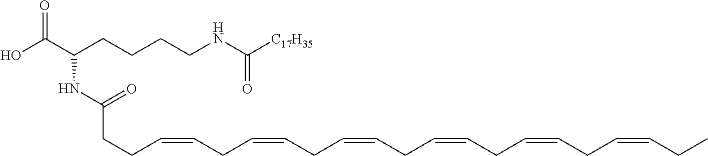 |
| DTx-01-101 | 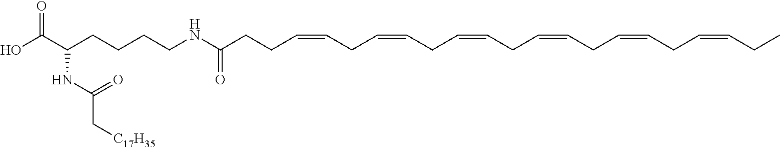 |

### Conjugating the Lipid Motifs to Modified Double-Stranded Oligonucleotides

As described in Schemes I, II, and III below, various lipid motifs were conjugated to siRNA using low-molecular-weight linkers. Table 2 below provides the siRNAs chosen for experimentation. Within the sequences given, the designations “in”, “f,” and “*” denote 2′-O-methyl residues, 2′-deoxy-2′-fluoro residues, and phosphorothioate linkages, respectively.

TABLE 2
siRNA Molecules Used

| siRNA Name | siRNA Properties | |
|---|---|---|
| Compound 1 | Target | Passenger Sequence (5′ to 3′) |
| (DTxO-0003) | PTEN | fG mA fU mG fA mU fG mU fU fU fG mA |
| | | fA mA fC mU fA mU fU*T*T |
| | | (SEQ ID NO: 1) |
| | | Guide Sequence (5′ to 3′) |
| | | PO4-mA fA mU fA mG fU mU fU mC mA |
| | | mA fA mC fA mU fC mA fU mC*T*T |
| | | (SEQ ID NO: 2) |
| Compound 3 | Target | Passenger Sequence (5′ to 3′) |
| (DTxO-0016) | VEGFR1 | fG mC fU mA fC mU fC mG fU fU fA mA |
| | | fU mU fA mU fC mA fA*T*T |
| | | (SEQ ID NO: 3) |
| | | Guide Sequence (5′ to 3′) |
| | | PO4-mU fU mG fA mU fA mA fU mU mA |
| | | mA fC mG fA mG fU mA fG mC*T*T |
| | | (SEQ ID NO: 4) |
| Compound 5 | Target | Passenger Sequence (5′ to 3′) |
| (DTxO-0021) | VEGFR2 | fC mC fA mA fA mU fU mC fC fA fU mU |
| | | fA mU fG mA fC mA fA*T*T |
| | | (SEQ ID NO: 5) |
| | | Guide Sequence (5′ to 3′) |
| | | PO4-mU fU mG fU mC fA mU fA mA mU |
| | | mG fG mA fA mU fU mU fG mG*T*T |
| | | (SEQ ID NO: 6) |
| Compound 27 | Target | Passenger Sequence (5′ to 3′) |
| (DTxO-0037) | HTT | fC*mA*fG mU fA mA fA mG fA mG fA |
| | | mU fU*mA*fA (SEQ ID NO: 7) |
| | | Guide Sequence (5′ to 3′) |
| | | PO4-mU*fU*mA fA mU fC mU fC mU fU |
| | | mU fA mC*fU*mG*fA*mU*fA*mU*fA |
| | | (SEQ ID NO: 8) |
| Compound 30 | Target | Passenger Sequence (5′ to 3′) |
| (DTxO-0038) | PTEN | fA*mC*fC mU fG mA fU mC fA mU fU |
| | | mA fU mA fG mA fU*mA*fA |
| | | (SEQ ID NO: 9) |
| | | Guide Sequence (5′ to 3′) |
| | | PO4- eT*fU*mA fU mC fU mA fU mA fA |
| | | mU fG mA fU mC fA mG fG mU *T *T |
| | | (SEQ ID NO: 10) |
| Compound 31 | Target | Passenger Sequence (5′ to 3′) |
| (DTxO-0033) | VEGFR2 | fC*mC*fA mA fA mU fU mC fC fA fU |
| | | mU fA mU fG mA fC mA fA*T*T |
| | | (SEQ ID NO: 5) |
| | | Guide Sequence (5′ to 3′) |
| | | PO4- mU*fU*mG fU mC fA mU fA mA |
| | | mU mG fG mA fA mU fU mU fG mG*T*T |
| | | (SEQ ID NO: 6) |
| Compound 32 | Target | Passenger Sequence (5′ to 3′) |
| (DTxO-0034) | VEGFR2 | fG*mG*fU mU fG mU fA mG fG fA fU |
| | | mA fU mA fG mG fA mU fU*T*T |
| | | (SEQ ID NO: 10) |
| | | Guide Sequence (5′ to 3′) |
| | | PO4- mA*fA*mU fC mC fU mA fU mA |
| | | mU mC fC mU fA mC fA mA fC mC*T*T |
| | | (SEQ ID NO: 11) |

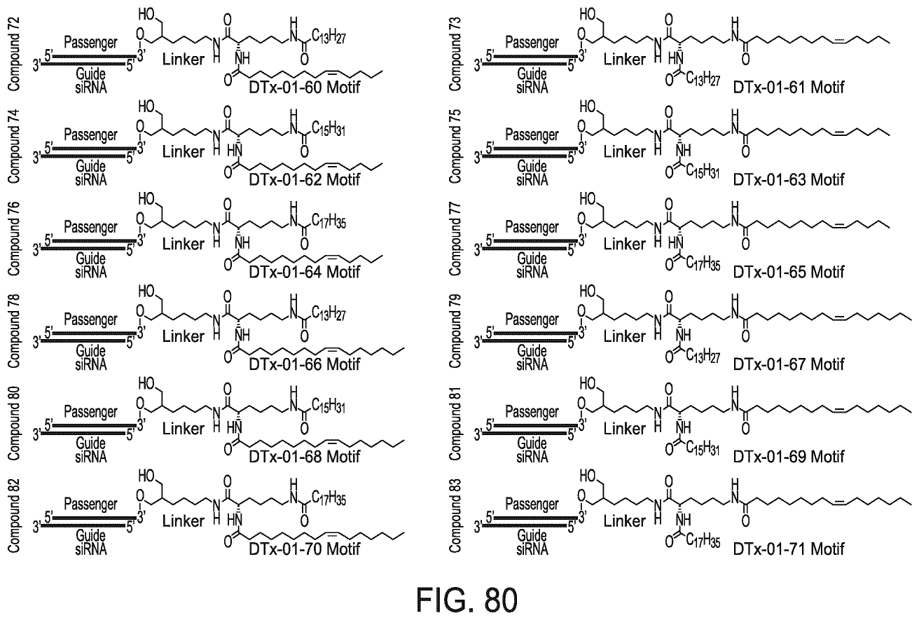

Table 3 lists lipid-modified nucleic acid compounds. The synthesis Scheme I, II, or III is indicated as appropriate for each compound. Certain compounds were prepared, as indicated by the presence of data in the “LCMS m/z (M+H)+” column in Table 3. Compounds in addition to those prepared are shown in Table 3. The structures of lipid-modified nucleic acid compounds are also illustrated in FIGS. 1 through 12 and in FIGS. 80 through 83.

TABLE 3
Lipid-Modified Nucleic Acid Compounds

| Lipid- | | | | | LCMS | |
|---|---|---|---|---|---|---|
| Modified | | 5′ | 3′ | | m/z | |
| Nucleic Acid | Strand | Modification | Modification | SIRNA | (M + H)⁺ | Synthesis |
| Compound 2 | Passenger | — | DTx-01-08 | Compound | 7636.1 | Scheme I |
| | Guide | — | — | 1 | 6864.1 | |
| Compound 4 | Passenger | — | DTx-01-08 | Compound | 7557.8 | Scheme I |
| | Guide | — | — | 3 | 6962.6 | |
| Compound 6 | Passenger | — | DTx-01-08 | Compound | 7563.3 | Scheme I |
| | Guide | — | — | 5 | 6956.2 | |
| Compound 7 | Passenger | — | DTx-01-06 | Compound | 7441.8 | Scheme I |
| | Guide | — | — | 1 | 6866.1 | |
| Compound 8 | Passenger | — | DTx-01-11 | Compound | 7441.2 | Scheme I |
| | Guide | — | — | 1 | 6866.1 | |
| Compound 9 | Passenger | DTx-01-06 | DTx-01-06 | Compound | 8029.5 | Scheme II |
| | Guide | — | — | 1 | 6864.3 | |
| Compound 10 | Passenger | — | DTx-01-30 | Compound | 7468.5 | Scheme I |
| | Guide | — | — | 1 | 6866.2 | |
| Compound 11 | Passenger | — | DTx-01-31 | Compound | 7525.7 | Scheme I |
| | Guide | — | — | 1 | 6866.2 | |
| Compound 12 | Passenger | — | DTx-01-32 | Compound | 7581.4 | Scheme I |
| | Guide | — | — | 1 | 6866.2 | |
| Compound 13 | Passenger | — | DTx-01-33 | Compound | 7694.6 | Scheme I |
| | Guide | — | — | 1 | 6864.0 | |
| Compound 14 | Passenger | — | DTx-01-34 | Compound | 7749.8 | Scheme I |
| | Guide | — | — | 1 | 6864.0 | |
| Compound 15 | Passenger | — | DTx-01-35 | Compound | 7806.5 | Scheme I |
| | Guide | — | — | 1 | 6864.0 | |
| Compound 16 | Passenger | — | DTx-01-01 | Compound | 7514.3 | Scheme I |
| | Guide | — | — | 1 | 6866.2 | |
| Compound 17 | Passenger | — | DTx-01-03 | Compound | 7780.8 | Scheme I |
| | Guide | — | — | 1 | 6866.2 | |
| Compound 18 | Passenger | — | DTx-01-13 | Compound | 7513.2 | Scheme I |
| | Guide | — | — | 1 | 6866.2 | |
| Compound 20 | Passenger | — | DTx-03-06 | Compound | 7595.0 | Scheme I |
| | Guide | — | — | 1 | 6866.2 | |
| Compound 21 | Passenger | — | DTx-06-06 | Compound | 7751.1 | Scheme I |
| | Guide | — | — | 1 | 6866.2 | |
| Compound 22 | Passenger | DTx-01-11 | DTx-01-11 | Compound | 8029.5 | Scheme II |
| | Guide | | | 1 | 6864.3 | |
| Compound 23 | Passenger | — | DTx-01-07 | Compound | 7471.5 | Scheme I |
| | Guide | — | — | 1 | 6864.1 | |
| Compound 24 | Passenger | DTx-01-09 | — | Compound | 7667.4 | Scheme |
| | Guide | — | — | 1 | 6866.8 | III |
| Compound 25 | Passenger | — | DTx-01-09 | Compound | 7697.3 | Scheme I |
| | Guide | — | — | 1 | 6866.8 | |
| Compound 26 | Passenger | — | DTx-01-12 | Compound | 7471.9 | Scheme I |
| | Guide | — | — | 1 | 6866.1 | |
| Compound 28 | Passenger | — | DTx-01-13 | Compound | 5699.3 | Scheme I |
| | Guide | — | — | 27 | 6621.5 | |
| Compound 29 | Passenger | — | DTx-01-08 | Compound | 5824.0 | Scheme I |
| | Guide | — | — | 27 | 6621.5 | |
| Compound 33 | Passenger | — | DTx-01-08 | Compound | 7040.7 | Scheme I |
| | Guide | — | — | 30 | 6977.5 | |
| Compound 34 | Passenger | — | DTx-01-08 | Compound | 7595.0 | Scheme I |
| | Guide | — | — | 31 | 6986.1 | |
| Compound 35 | Passenger | — | DTx-01-08 | Compound | 7765.6 | Scheme I |
| | Guide | — | — | 32 | 6833.1 | |
| Compound 38 | Passenger | — | DTx-01-36 | Compound | 7578.1 | Scheme I |
| | Guide | — | — | 1 | 6866.0 | |
| Compound 39 | Passenger | — | DTx-01-39 | Compound | 7633.4 | Scheme I |
| | Guide | — | — | 1 | 6866.0 | |
| Compound 40 | Passenger | — | DTx-01-43 | Compound | 7690.7 | Scheme I |
| | Guide | — | — | 1 | 6866.0 | |
| Compound 41 | Passenger | — | DTx-01-44 | Compound | | Scheme I |
| | Guide | — | — | 1 | | |
| Compound 42 | Passenger | — | DTx-01-45 | Compound | 7682.0 | Scheme I |
| | Guide | — | — | 1 | 6866.0 | |
| Compound 43 | Passenger | — | DTx-01-46 | Compound | | Scheme I |
| | Guide | — | — | 1 | | |
| Compound 44 | Passenger | — | DTx-08-01 | Compound | 7762.2 | Scheme I |
| | Guide | — | — | 1 | 6866.0 | |
| Compound 45 | Passenger | — | DTx-09-01 | Compound | | Scheme I |
| | Guide | — | — | 1 | | |
| Compound 46 | Passenger | — | DTx-10-01 | Compound | | Scheme I |
| | Guide | — | — | 1 | | |
| Compound 47 | Passenger | — | DTx-11-01 | Compound | | Scheme I |
| | Guide | — | — | 1 | | |
| Compound 48 | Passenger | — | DTx-04-01 | Compound | 8005.0 | Scheme I |
| | Guide | — | — | 1 | 6866.0 | |
| Compound 49 | Passenger | — | DTx-05-01 | Compound | | Scheme I |
| | Guide | — | — | 1 | | |
| Compound 50 | Passenger | DTx-01-08 | — | Compound | 7607.8 | Scheme |
| | Guide | — | — | 1 | 6866.0 | III |
| Compound 51 | Passenger | DTx-01-08 | — | Compound | 7009.7 | Scheme |
| | Guide | — | — | 30 | 6976.5 | III |
| Compound 52 | Passenger | — | — | Compound | | Scheme I |
| | Guide | — | DTx-01-08 | 1 | | |
| Compound 53 | Passenger | — | — | Compound | | Scheme I |
| | Guide | — | DTx-01-08 | 30 | | |
| Compound 54 | Passenger | — | DTx-01-50 | Compound | 7608.8 | Scheme I |
| | Guide | — | — | 1 | 6864.7 | |
| Compound 55 | Passenger | — | DTx-01-51 | Compound | 7610.2 | Scheme I |
| | Guide | — | — | 1 | 6864.7 | |
| Compound 56 | Passenger | — | DTx-01-52 | Compound | 7666.3 | Scheme I |
| | Guide | — | — | 1 | 6864.7 | |
| Compound 57 | Passenger | — | DTx-01-53 | Compound | 7665.1 | Scheme I |
| | Guide | — | — | 1 | 6864.7 | |
| Compound 58 | Passenger | — | DTx-01-54 | Compound | 7636.3 | Scheme I |
| | Guide | — | — | 1 | 6864.7 | |
| Compound 59 | Passenger | — | DTx-01-55 | Compound | 7636.5 | Scheme I |
| | Guide | — | — | 1 | 6864.7 | |
| Compound 60 | Passenger | — | DTx-03-50 | Compound | | Scheme I |
| | Guide | — | — | 1 | | |
| Compound 61 | Passenger | — | DTx-03-51 | Compound | | Scheme I |
| | Guide | — | — | 1 | | |
| Compound 62 | Passenger | — | DTx-03-52 | Compound | | Scheme I |
| | Guide | — | — | 1 | | |
| Compound 63 | Passenger | — | DTx-03-53 | Compound | | Scheme I |
| | Guide | — | — | 1 | | |
| Compound 64 | Passenger | — | DTx-03-54 | Compound | | Scheme I |
| | Guide | — | — | 1 | | |
| Compound 65 | Passenger | — | DTx-03-55 | Compound | | Scheme I |
| | Guide | — | — | 1 | | |
| Compound 66 | Passenger | — | DTx-06-50 | Compound | | Scheme I |
| | Guide | — | — | 1 | | |
| Compound 67 | Passenger | — | DTx-06-51 | Compound | | Scheme I |
| | Guide | — | — | 1 | | |
| Compound 68 | Passenger | — | DTx-06-52 | Compound | | Scheme I |
| | Guide | — | — | 1 | | |
| Compound 69 | Passenger | — | DTx-06-53 | Compound | | Scheme I |
| | Guide | — | — | 1 | | |
| Compound 70 | Passenger | — | DTx-06-54 | Compound | | Scheme I |
| | Guide | — | — | 1 | | |
| Compound 71 | Passenger | — | DTx-06-55 | Compound | | Scheme I |
| | Guide | — | — | 1 | | |
| Compound 72 | Passenger | — | DTx-01-60 | Compound | | Scheme I |
| | Guide | — | — | 1 | | |
| Compound 73 | Passenger | — | DTx-01-61 | Compound | | Scheme I |
| | Guide | — | — | 1 | | |
| Compound 74 | Passenger | — | DTx-01-62 | Compound | | Scheme I |
| | Guide | — | — | 1 | | |
| Compound 75 | Passenger | — | DTx-01-63 | Compound | | Scheme I |
| | Guide | — | — | 1 | | |
| Compound 76 | Passenger | — | DTx-01-64 | Compound | | Scheme I |
| | Guide | — | — | 1 | | |
| Compound 77 | Passenger | — | DTx-01-65 | Compound | | Scheme I |
| | Guide | — | — | 1 | | |
| Compound 78 | Passenger | — | DTx-01-66 | Compound | | Scheme I |
| | Guide | — | — | 1 | | |
| Compound 79 | Passenger | — | DTx-01-67 | Compound | | Scheme I |
| | Guide | — | — | 1 | | |
| Compound 80 | Passenger | — | DTx-01-68 | Compound | | Scheme I |
| | Guide | — | — | 1 | | |
| Compound 81 | Passenger | — | DTx-01-69 | Compound | | Scheme I |
| | Guide | — | — | 1 | | |
| Compound 82 | Passenger | — | DTx-01-70 | Compound | | Scheme I |
| | Guide | — | — | 1 | | |
| Compound 83 | Passenger | — | DTx-01-71 | Compound | | Scheme I |
| | Guide | — | — | 1 | | |
| Compound 84 | Passenger | — | DTx-01-72 | Compound | | Scheme I |
| | Guide | — | — | 1 | | |
| Compound 85 | Passenger | — | DTx-01-73 | Compound | | Scheme I |
| | Guide | — | — | 1 | | |
| Compound 86 | Passenger | — | DTx-01-74 | Compound | | Scheme I |
| | Guide | — | — | 1 | | |
| Compound 87 | Passenger | — | DTx-01-75 | Compound | | Scheme I |
| | Guide | — | — | 1 | | |
| Compound 88 | Passenger | — | DTx-01-76 | Compound | | Scheme I |
| | Guide | — | — | 1 | | |
| Compound 89 | Passenger | — | DTx-01-77 | Compound | | Scheme I |
| | Guide | — | — | 1 | | |
| Compound 90 | Passenger | — | DTx-01-78 | Compound | | Scheme I |
| | Guide | — | — | 1 | | |
| Compound 91 | Passenger | — | DTx-01-79 | Compound | | Scheme I |
| | Guide | — | — | 1 | | |
| Compound 92 | Passenger | — | DTx-01-80 | Compound | | Scheme I |
| | Guide | — | — | 1 | | |
| Compound 93 | Passenger | — | DTx-01-81 | Compound | | Scheme I |
| | Guide | — | — | 1 | | |
| Compound 94 | Passenger | — | DTx-01-82 | Compound | | Scheme I |
| | Guide | — | — | 1 | | |
| Compound 95 | Passenger | — | DTx-01-83 | Compound | | Scheme I |
| | Guide | — | — | 1 | | |
| Compound 96 | Passenger | — | DTx-01-84 | Compound | | Scheme I |
| | Guide | — | — | 1 | | |
| Compound 97 | Passenger | — | DTx-01-85 | Compound | | Scheme I |
| | Guide | — | — | 1 | | |
| Compound 98 | Passenger | — | DTx-01-86 | Compound | | Scheme I |
| | Guide | — | — | 1 | | |
| Compound 99 | Passenger | — | DTx-01-87 | Compound | | Scheme I |
| | Guide | — | — | 1 | | |
| Compound | Passenger | — | DTx-01-88 | Compound | | Scheme I |
| 100 | Guide | — | — | 1 | | |
| Compound | Passenger | — | DTx-01-89 | Compound | | Scheme I |
| 101 | Guide | — | — | 1 | | |
| Compound | Passenger | — | DTx-01-90 | Compound | | Scheme I |
| 102 | Guide | — | — | 1 | | |
| Compound | Passenger | — | DTx-01-91 | Compound | | Scheme I |
| 103 | Guide | — | — | 1 | | |
| Compound | Passenger | — | DTx-01-92 | Compound | | Scheme I |
| 104 | Guide | — | — | 1 | | |
| Compound | Passenger | — | DTx-01-93 | Compound | | Scheme I |
| 105 | Guide | — | — | 1 | | |
| Compound | Passenger | — | DTx-01-94 | Compound | | Scheme I |
| 106 | Guide | — | — | 1 | | |
| Compound | Passenger | — | DTx-01-95 | Compound | | Scheme I |
| 107 | Guide | — | — | 1 | | |
| Compound | Passenger | — | DTx-01-96 | Compound | | Scheme I |
| 108 | Guide | — | — | 1 | | |
| Compound | Passenger | — | DTx-01-97 | Compound | | Scheme I |
| 109 | Guide | — | — | 1 | | |
| Compound | Passenger | — | DTx-01-98 | Compound | | Scheme I |
| 110 | Guide | — | — | 1 | | |
| Compound | Passenger | — | DTx-01-99 | Compound | | Scheme I |
| 111 | Guide | — | — | 1 | | |
| Compound | Passenger | — | DTx-01-100 | Compound | | Scheme I |
| 112 | Guide | — | — | 1 | | |
| Compound | Passenger | — | DTx-01-101 | Compound | | Scheme I |
| 113 | Guide | — | — | 1 | | |

### Scheme I: Conjugation of Lipid Moieties to the 3′ End of a Passenger Strand of a Modified Double-Stranded Oligonucleotide

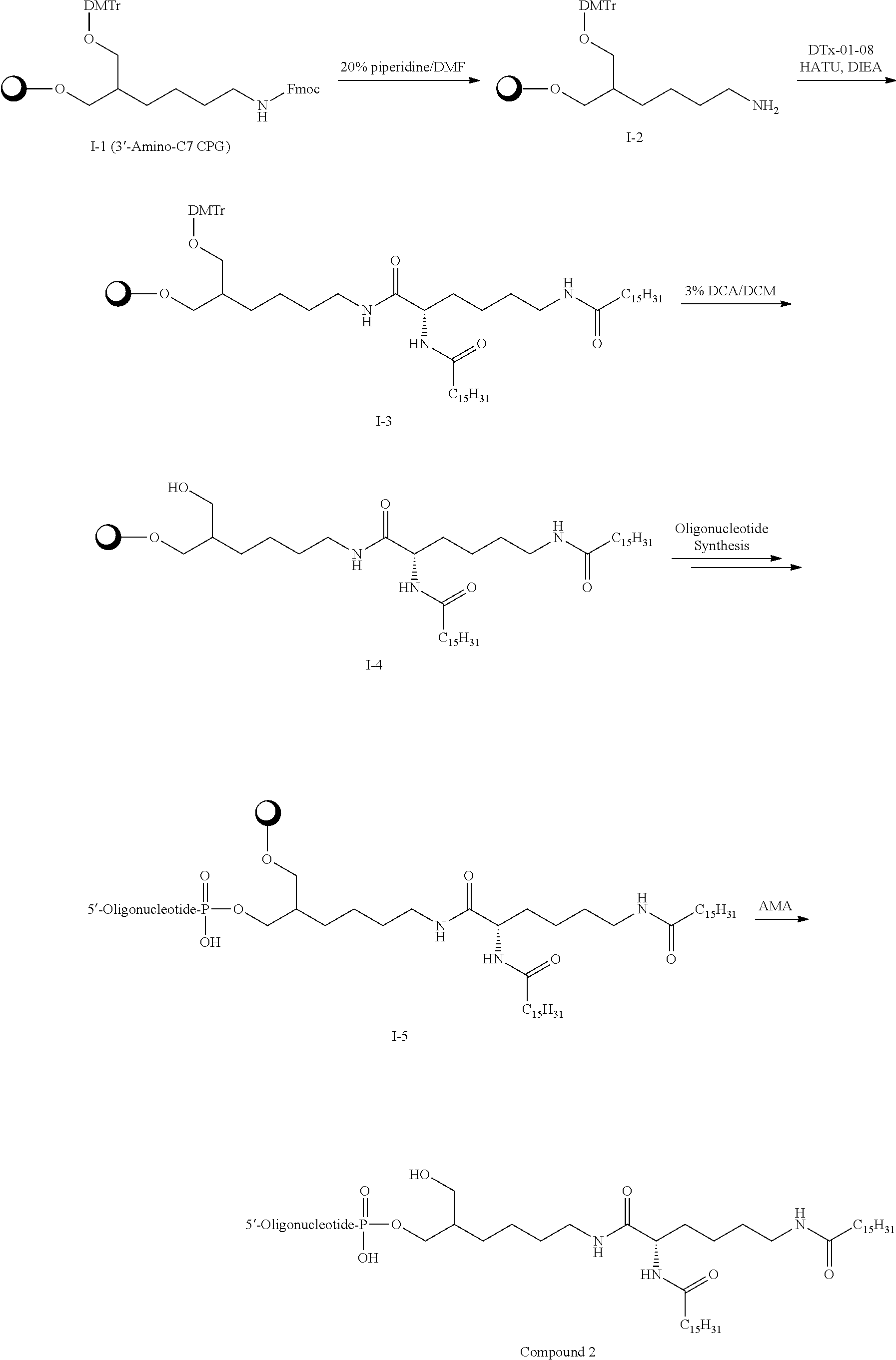

Scheme I above illustrates the preparation of a passenger strand of a modified double-stranded oligonucleotide conjugated with a lipid moiety at the 3′ end of the passenger strand, using the passenger strand of Compound 2 as an example. In summary, 3′-amino CPGbeads I-1 (Glen Research, Catalog No. 20-2958) modified with the DMT and Fmoc-protected C7 linker illustrated above were treated with 20% piperidine/DMF to afford Fmoc-deprotected amino C7 CPG beads I-2. Lipid motif DTx-01-08 was then coupled to I-2 using HATU and DIEA in DMF to produce lipid-loaded CPG beads I-3, which were treated by 3% dichloroacetic acid (DCA) in DCM to remove the DMT protecting group and afford I-4. Oligonucleotide synthesis of the passenger strand of DTxO-0003 si-RNA on I-4 was accomplished via standard phosphoramidite chemistry and yielded modified oligonucleotide-bounded CPG beads I-5. At this point, if applicable, beads I-5 containing methyl ester-protected lipid motifs (e.g., DTx-01-07-OMe, DTx-01-09-OMe, and DTx-01-12-OMe) were saponified to their respective carboxylic acid using 0.5 M LiOH in 3:1 v/v methanol/water. Subsequent treatment of I-5 with AMA [ammonium hydroxide (28%)/methylamine (40%) (1:1, v/v)] cleaved the DTx-01-08-conjugated modified oligonucleotide from the beads. The passenger strand of Compound 2 was then purified by RP-HPLC and characterized by MALDI-TOF MS using the [M+H] peak.

Scheme II: Conjugation of Lipid Moieties to both the 3′ and the 5′ Ends of a Passenger Strand of a Modified Double-Stranded Oligonucleotide

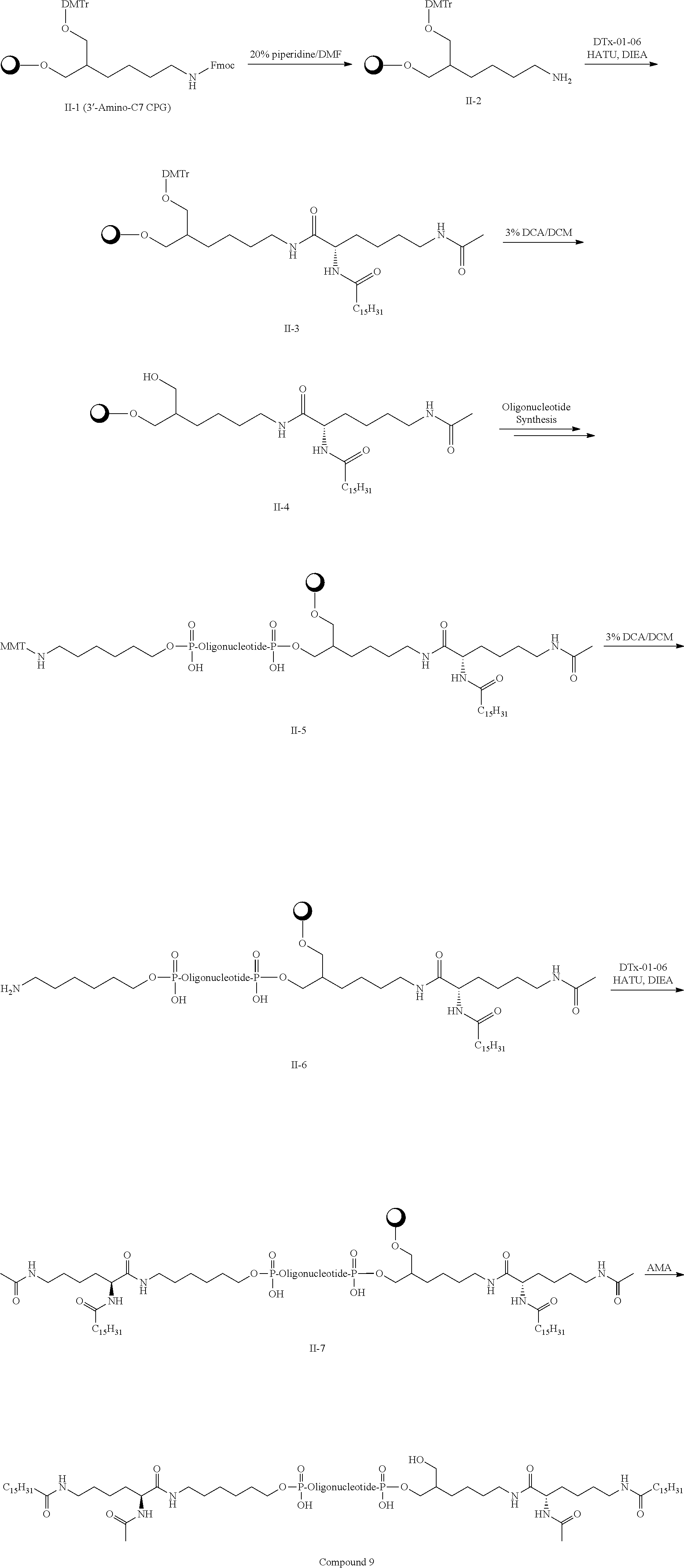

Scheme II above illustrates the preparation of a passenger strand of a modified double-stranded oligonucleotide conjugated with lipid moieties at both the 3′ and 5′ ends of the passenger strand, using the passenger strand of Compound 9 as an example. In summary, 3′-amino CPG beads II-1 (Glen Research, Catalog No. 20-2958) modified with the DMT and Fmoc-protected C7 linker illustrated above were treated with 20% piperidine/DMF to afford Fmoc-deprotected amino C7 CPG beads II-2. Lipid motif DTx-01-06 was then coupled to II-2 using HATU and DIEA in DMF to produce lipid-loaded CPG beads II-3, which were treated by 3% dichloroacetic acid (DCA) in DCM to remove the DMT protecting group and afford II-4. Oligonucleotide synthesis of the passenger strand of DTxO-0003 si-RNA was performed on II-4 via standard phosphoramidite chemistry. In the last nucleotide coupling of the automated sequence, a nucleotide modified with the MMT-protected C6 linker illustrated above (Glen Research, Catalog No. 10-1906) was used, yielding modified oligonucleotide-bounded CPG beads II-5. After removal of MMT with 3% dichloroacetic acid (DCA) in DCM, II-6 was coupled to DTx-01-16 using HATU and DIEA in DMF to yield II-6. Subsequent treatment of II-6 with AMA [ammonium hydroxide (28%)/methylamine (40%) (1:1, v/v)] cleaved the DTx-01-06-conjugated modified oligonucleotide from the beads. The passenger strand of Compound 9 was then purified by RP-HPLC and characterized by MALDI-TOF MS using the [M+H] peak.

### Scheme III: Conjugation of Lipid Moieties to the 5′ End of a Passenger Strand of a Modified Double-Stranded Oligonucleotide

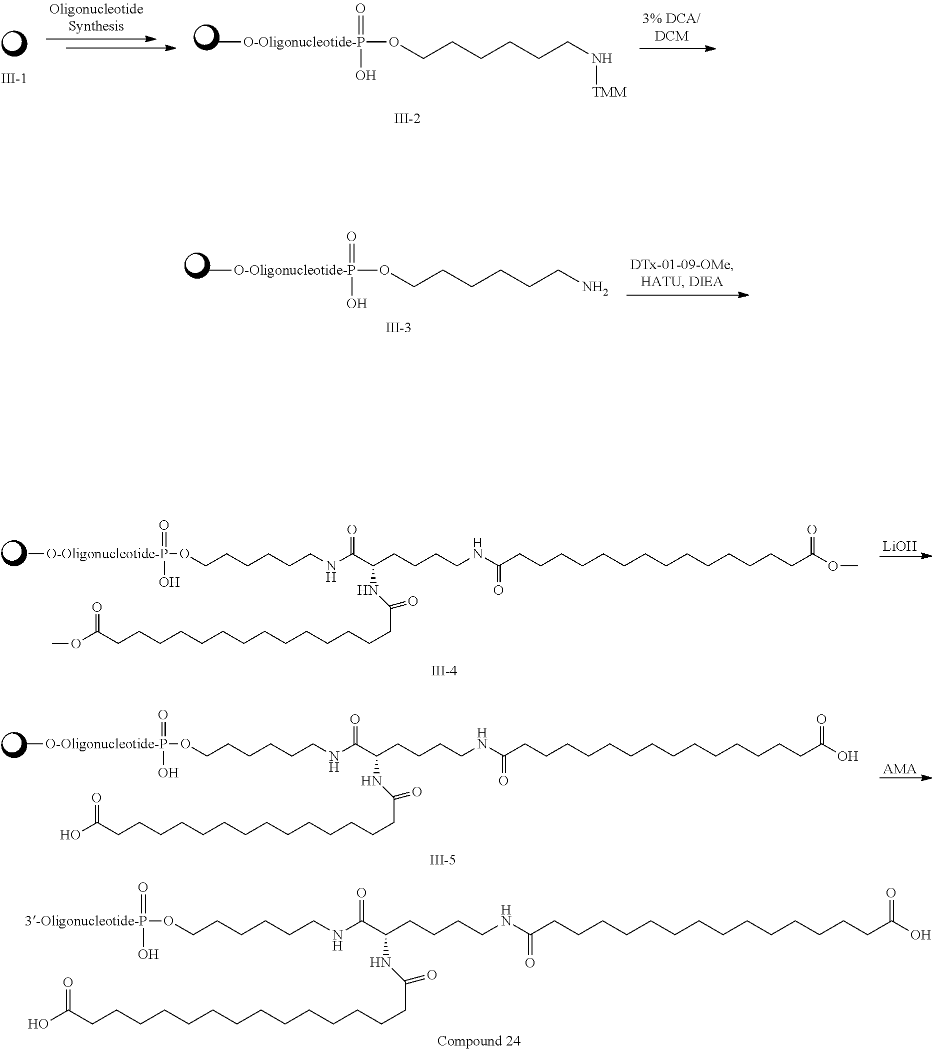

Scheme III above illustrates the preparation of a passenger strand of a modified double-stranded oligonucleotide conjugated with a lipid moiety at the 5′ end of the passenger strand, using the passenger strand of Compound 24 as an example. In summary, oligonucleotide synthesis of the passenger strand of DTxO-0003 siRNA was performed on CPG beads III-1 (Glen Research, Catalog No. 20-5041-xx) via standard phosphoramidite chemistry. In the last nucleotide coupling of the automated sequence, a nucleotide modified with the MMT-protected C6 linker illustrated above (Glen Research, Catalog No. 10-1906) was used, yielding modified oligonucleotide-bounded CPG beads III-2. After removal of MMT with 3% dichloroacetic acid (DCA) in DCM, III-2 was coupled to DTx-01-09-OMe using HATU and DIEA in DMF to yield III-4. III-4 was saponified with 0.5 M LiOH in 3:1 v/v methanol/water, affording III-5. Subsequent treatment of III-5 with AMA [ammonium hydroxide (28%)/methylamine (40%) (1:1, v/v)] cleaved the DTx-01-09-conjugated modified oligonucleotide from the beads. The passenger strand of Compound 24 was then purified by RP-HPLC and characterized by MALDI-TOF MS using the [M+H] peak.

### Duplex Formation

For each of the passenger strands synthesized by Schemes I, II, or III and listed above, the complementary guide strand was prepared via standard phosphoramidite chemistry, purified by IE-HPLC, and characterized by MALDI-TOF MS using the [M+H] peak. The duplex was formed by mixing equal molar equivalents of the passenger strand and guide strand, heating to 90° C. for 5 minutes, and then slowly cooling to room temperature. Duplex formation was confirmed by non-denature PAGE.

### Conjugating the Lipid Motifs to Modified Single-Stranded Oligonucleotides

Table 4 below provides a modified antisense oligonucleotide chosen for experimentation. Within the sequence given, the designation “e” denotes 2′-O-methoxyethyl residues and the remaining residues are 2′-deoxy residues, and the designation “*” denotes phosphorothioate linkages.

TABLE 4
Antisense molecules

| Antisense | | |
|---|---|---|
| Name | Antisense Properties | |
| Compound 37 | Target | Antisense Sequence (5′ to 3′) |
| | PTEN | eC*eT*eG*eC*eT*A*G*C*C*T*C*T* |
| | | G*G*A*eT*eT*eT*eG*eA |
| | | (SEQ ID NO: 13) |

### Biological Data

### General Procedures and Methods

In embodiments, provided herein are methods of contacting a cell with a compound or composition comprising a compound as described herein. In embodiments, provided herein are methods of evaluating mRNA expression relative to a PBS control in a cell after exposing said cell to a compound or compositions comprising a compound as described herein. In embodiments, the cell is a primary cell from an animal, e.g., a mammal, or a human. In embodiments, the cell from a human.

In embodiments, provided herein is a method of co-administering a compound and/or composition described herein, with an additional compound and/or composition to a cell. By “co-administration,” it is meant that the two or more agents may be found in the cell at the same time, regardless of when or how they are actually administered. In one embodiment, the agents are administered simultaneously. In one such embodiment, administration in combination is accomplished by combining the agents in a single form. In another embodiment, the agents are administered sequentially. In one embodiment the agents are administered through the same route, such as under free uptake conditions or transfection. In another embodiment, the agents are administered through different routes, such as one being administered by transfection and another being administered under free uptake conditions.

The following examples should not, of course, be construed as specifically limiting. Variations of these examples within the scope of the claims are within the purview of one skilled in the art and are considered to fall within the scope of the embodiments as described and claimed herein. The reader will recognize that the skilled artisan, armed with the present disclosure, and skill in the art is able to prepare and use the invention without exhaustive examples.

### Cell Culture

HEK293, NIH3T3, and Bend.3 cells were purchased from ATCC and, RAW264.7 cells and SH-SY5Y cells from Sigma-Aldrich. HEK293, NIH3T3 and RAW264.7 cells were cultured in DMEM containing 10% Fetal Bovine Serum (FBS), 2 mM L-glutamine, 1× non-essential amino acids, 100 U/mL penicillin and 100 mg/mL streptomycin in a humidified 37 C incubator with 5% CO₂. Undifferentiated SH-SY5Y cells were cultured in DMEM/F12 (1:1) medium containing 10% FBS 2 mM L-glutamine, 1× non-essential amino acids, 100 U/mL penicillin and 100 mg/mL streptomycin in a humidified 37 C incubator with 5% CO₂ (“maintenance media”).

SH-SY5Y cells were differentiated by plating 5000 cells/well in maintenance media in a 96 well plate. 24-48 hours following plating, the medium was replaced with differentiation medium consisting of Neurobasal medium supplemented with 2 mM L-glutamine, B27 supplement and 10 uM all-trans-retionic acid (ATRA). Cells were differentiated for 4 days prior to initiation of free uptake experiments.

3T3L1 cells were purchased from Sigma-Aldrich and maintained in 10% Fetal Calf Serum (FCS). For differentiation, confluent 3T3L1 cells plated on 96 well collagen coated plates were cultured for 5 days in differentiation medium (DMEM/F12 containing 10% FBS, 2 mM L-glutamine, 100 U/mL penicillin, 100 mg/mL streptomycin 1.5 ug/mL insulin, 1 uM dexamethasone, 500 uM IBMX and 1 uM rosiglitazone). The differentiation media was then replaced with maintenance media (DMEM/F12 medium containing 10% FBS, 2 mM L-glutamine, 100 U/mL penicillin, 100 mg/mL streptomycin and 1.5 ug/mL insulin). The maintenance media was replaced every 2 days thereafter. Free uptake experiments were initiated 10 days post differentiation.

HUVEC cells were purchased from Cell Applications (San Diego, CA) and cultured in their proprietary HUVEC cell media containing 2% serum, 100 U/mL penicillin and 100 mg/mL streptomycin.

Primary rat cortical neurons, human trabecular meshwork cells and primary human skeletal muscle cells were obtained from Cell Applications (San Diego, CA). They were cultured and/or differentiated in proprietary media, and according to the instructions, supplied by the vendor. In some cases, the proprietary media was obtained without FBS. FBS was added typically to a concentration of 2%.

Primary human adipocytes from lean donors were obtained from ZenBio in 96 well plates. They were cultured in ZenBio proprietary media containing 2% FBS.

Primary human hepatocytes were obtained from Thermo Fisher, thawed and plated at 10,000 cells per well in Thermo Fisher proprietary plating media. Six hours following plating, the plating medium was removed and replaced with Thermo Fisher proprietary maintenance medium.

Primary human stellate cells were purchased from ZenBIO and cultured in ZenBio proprietary human stellate growth medium (Catalog #HSGM-500).

Primary human T cells were purchased from Cell Applications in 96-well plates at a density of 20,000 cells/well and cultured in Cell Applications proprietary T cell expansion medium with reduced Cellastim (0.25 g/mL).

Primary human skeletal muscle cells were purchased from Cell Applications and cultured in Cell Applications proprietary Skeletal Muscle Cell Growth Medium. For differentiation, 10,000 cells were plated in each well of a 96-well plate in Skeletal Muscle Cell Growth Medium. After reaching confluency, skeletal differentiation medium was added to drive differentiation to myotubes.

### Transfection Experiments

24 hours prior to transfection, HEK293 cells, NIH3T3 cells and SH-SY5Y cells were plated into 96 well plates at 10,000 cells/well, 20,000 cells/well and 10,000 cells/well, respectively, in 90 μL of antibiotic free media. The oligonucleotide or oligonucleotide conjugates were diluted in PBS to 100× of the desired final concentration. Separately, Lipofectamine RNAiMax (Life Technologies) was diluted 1:66.7 in media lacking supplements (e.g. FBS, antibiotic etc.). The 100× oligonucleotide in PBS was then complexed with RNAiMAX by adding 1 part oligonucleotide in PBS to 9 parts lipofectamine/media. Following incubation for 20 minutes, 10 μL of the oligonucleotide:RNAiMAX complexes were added to the cells plated 24 hours prior containing 90 μL of antibiotic free media. The complexes were removed 24 hours following and replaced with media containing antibiotics. RNA was isolated 48 hours following transfection.

HUVEC cells were transfected utilizing lipofectamine RNAiMAX via reverse transfection. The oligonucleotide or oligonucleotide conjugates were diluted in PBS to 100× of the desired final concentration. Separately, lipofectamine RNAiMax was diluted 1:66.7 in media lacking supplements (e.g. FBS, antibiotic etc.). The 100× oligonucleotide in PBS was then complexed with RNAiMAX by adding 1 part oligonucleotide in PBS to 9 parts lipofectamine/media. The oligonucleotide and RNAiMAX were incubated for 20 minutes. In the interim, HUVEC cells were plated into 96 well plates at 10,000 cells per well in 90 μL of antibiotic free media and 10 μL of the oligonucleotide:RNAiMAX complexes were immediately added to the media. The complexes were removed 24 hours following plating and replaced with media containing antibiotics. RNA was isolated 48 hours following transfection.

24 hours prior to transfection, BEND.3 cells were plated into 96 well plates at 10,000 cells/well in 90 μL of antibiotic free media. The cells were transfected utilizing Cytofect (Cell Applications) according to manufacturer's instructions. As above, the complexes were removed 24 hours following and replaced with media containing antibiotics. RNA was isolated 48 hours following transfection.

### Free Uptake Experiments

HEK293 cells were plated at 20,000 cells/well, HUVEC cells at 10,000 cells/well, primary human trabecular meshwork cells at 10,000 cells/well and primary human skeletal muscle cells at 10,000 cells/well on 96 well collagen-coated plates. Primary human skeletal muscles were differentiated for 3 days in proprietary differentiation media supplied by Cell Applications. Primary neurons and adipocytes were supplied by the vendor, Cell Applications or ZenBio, as differentiated cells in 96 well plates. NIH3T3 cells were plated at 15,000 cells/well on tissue-culture treated 96 well plates. T cells were supplied by the vendor in 96 well plates containing 20,000 cells/well.

The day after plating for HEK293, HUVEC, trabecular meshwork, NIH3T3 cells and hepatocytes, the media was removed and the cells were washed twice with PBS containing calcium and magnesium. For skeletal muscle cells, differentiated SH-SY5Y cells and 3T3L1 adipocytes, media removal and PBS washing were performed 4, 4 and 11 days, respectively, following the initiation of differentiation. For adipocytes and primary neurons, media removal and PBS washing were performed 1 day following receipt from Cell Applications or ZenBio. Following the last wash, all of the cell types were incubated with compounds at various concentrations in their preferred medium containing 2% serum for 48 hours unless otherwise noted. In some cases, the serum concentration of proprietary formulations was not disclosed by the vendor. For experiments in HEK293, NIH3T3 and HUVEC cells with 96-hour time points, the compound-containing media was removed at 48 hours and replaced with complete media lacking compounds. For primary cells other than HUVEC, compounds were included when the media was replaced. RNA was isolated 48, 96 hours or 7 days following treatment. For adipocytes, primary neurons and T cells, media removal and PBS washing were performed 1 day following treatment.

### RNA Isolation, Reverse Transcription and Quantitative PCR

RNA was isolated utilizing the RNeasy 96 kit (Qiagen) according to the manufacturer's protocol. It was reverse transcribed to cDNA utilizing random primers and the high-capacity cDNA reverse transcription kit (ThermoFisher Scientific) in a SimpliAmp thermal cycler (ThermoFisher Scientific) according to manufacturer's instructions. Quantitative PCR was performed utilizing gene-specific primers (Thermofisher Scientific; IDTDNA), TaqMan probes (Thermofisher Scientific; IDTDNA) and TaqMan fast universal PCR master mix (Thermofisher scientific) on a StepOnePlus real-time PCR system (Thermofisher scientific) according to manufacturer's instructions. For analysis of quantitative PCR, PTEN or FLT1 mRNA expression was normalized to the expression of either 18s rRNA or HPRT1 mRNA (housekeeping genes) utilizing the relative CT method according to the best practices proposed in Nature Protocols (Schmittgen, T.D. & Livak, K.J. Analyzing real-time PCR data by the comparative C(T) method. Nat Protoc 3, 1101-1108 (2008)).

### Intravitreal Injection

Following acclimatization for 7 days, the mice or rats were weighed the night before the study and sorted into groups based on body weight. The day of study initiation, the mice were anesthetized with injectable anesthesia, 100 mg/kg ketamine and 5 mg/kg xylazine via intraperitoneal injection. Deep anesthesia is confirmed via toe pinch. One or both eyes was injected intravitreally under a dissecting scope with up to 1 μL (in mice) or up to 5 μL (in rats) of the compound of interest using a Hamilton syringe. Following injection, antibiotic (e.g. terramycin) was placed on eye. The animal was then allowed to recover from anesthesia in the home cage on a water-recirculating heating pad. The righting reflex was confirmed prior to removing the heat pad and before returning the animal to the holding room. Seven days following injection, the mice or rats were euthanized by CO₂ asphyxiation followed by secondary confirmation of euthanasia via cervical dislocation. The eyes were then removed and the regions of interest dissected and prepared for RNA isolation. The regions of interest were placed in RNALater immediately following dissection. 24 hours later, the tissue in RNALater was flash frozen and stored at −80 degrees Celsius until RNA isolation. Prior to RNA isolation and following thawing, the RNALater was removed and the tissue washed 2× in PBS. Trizol was then added and RNA isolated using the RNeasy 96 kit via manufacturer's instructions.

### RNAscope (Quantitative In Situ Hybridization)

As above, mice were injected intravitreally. Seven days following injection, the mice were euthanized and their eyes removed. The eyes were then formalin fixed, embedded in paraffin and sectioned at 5 μm thickness. RNA in situ hybridization for Mus musculus PTEN mRNA was performed manually using the RNAscope®2.5 HD Red Reagent Kit (Advanced Cell Diagnostics, Inc., Newark, CA) according to the manufacturer's instructions. Briefly, 5 μm formalin fixed, paraffin embedded (FFPE) tissue sections were pretreated with heat for 15 minutes at 100 degrees Celsius and protease plus for 15 minutes at 40 degrees Celsius prior to hybridization with the target oligo probes. Preamplifier, amplifier and AP-labeled oligos were then hybridized sequentially, followed by chromogenic precipitate development. Each sample was quality controlled for RNA integrity with an RNAscope® probe specific to PPIB RNA and for background with a probe specific to bacterial dapB RNA. Specific RNA staining signal was identified as red, punctate dots. Samples were counterstained with Gill's Hematoxylin. Brightfield images were acquired using an AperioAT2 digital slide scanner equipped with a 40× objective.

### Systemic Delivery Studies

Following acclimatization for 7 days, the mice were weighed the night before the study and sorted into groups based on body weight. The day of study initiation, the mice were injected with PBS or the compound of interest via intravenous or subcutaneous injection. The mice were euthanized by CO₂ asphyxiation followed by secondary confirmation of euthanasia via cervical dislocation seven days following either a single injection or seven days following the last dose when repeated injections were utilized. The tissues of interest were then removed and 30-300 mg placed in RNALater immediately following dissection. 24 hours later, the tissue was removed from the RNALater, blotted dry and placed into trizol in tubes containing lysing matrix D beads from MPBiomedical. The tissue was homogenized using the MPBio FastPrep-24 system. Chloroform extraction was then performed by adding 0.2 mL per 1 mL of Trizol. Samples were mixed thoroughly, spun at max speed in a microcentrifuge at 4 degrees Celsius for 15 minutes and the aqueous layer. The RNA was then precipitated by adding 1.5 volumes of absolute ethanol to the aqueous phase. The precipitated RNA was then purified utilizing the RNeasy 96 kit from Qiagen according to the manufacturer's instructions, substituting RLT buffer for RW1 buffer.

### Results

Selection of PTEN as siRNA Target for Proof of Concept Confirmation that LCFA Conjugation Does Not Interfere with siRNA Activity

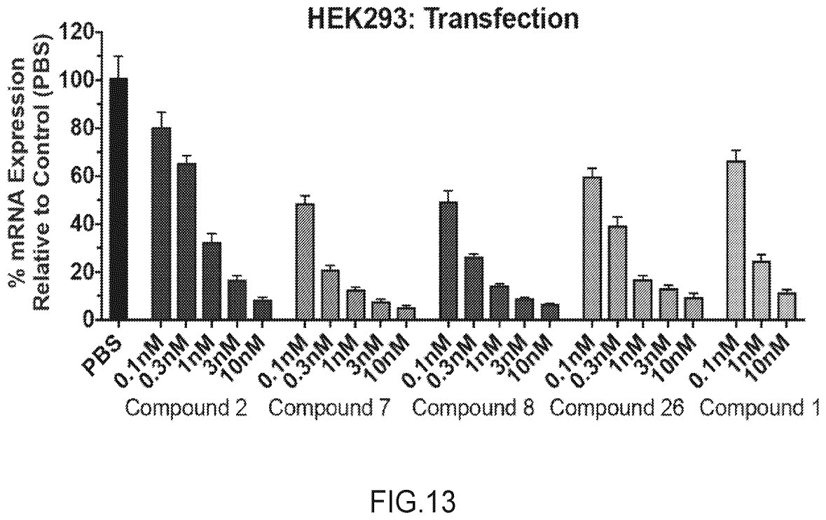

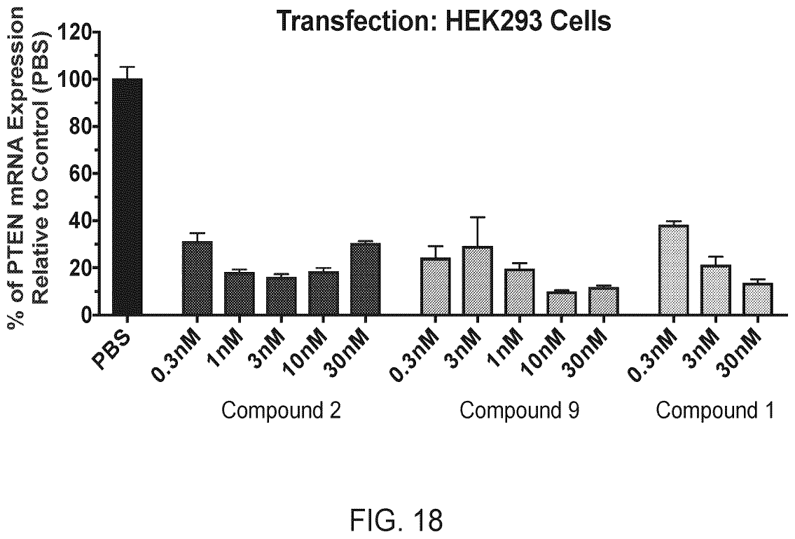

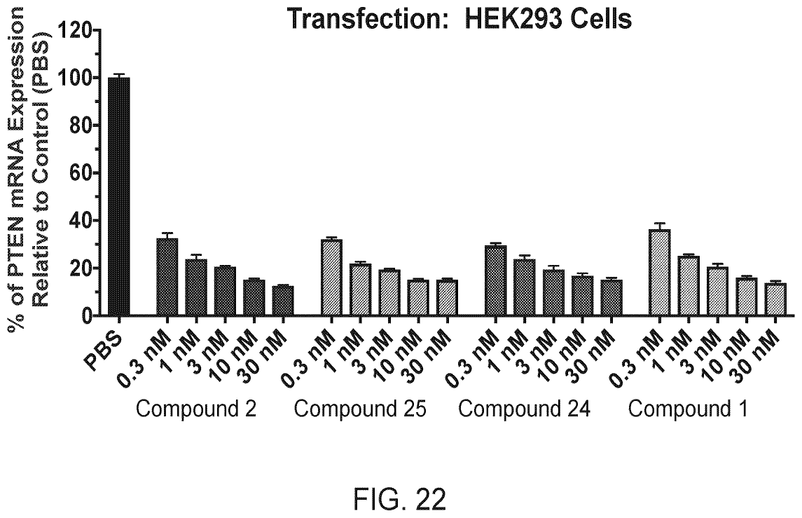

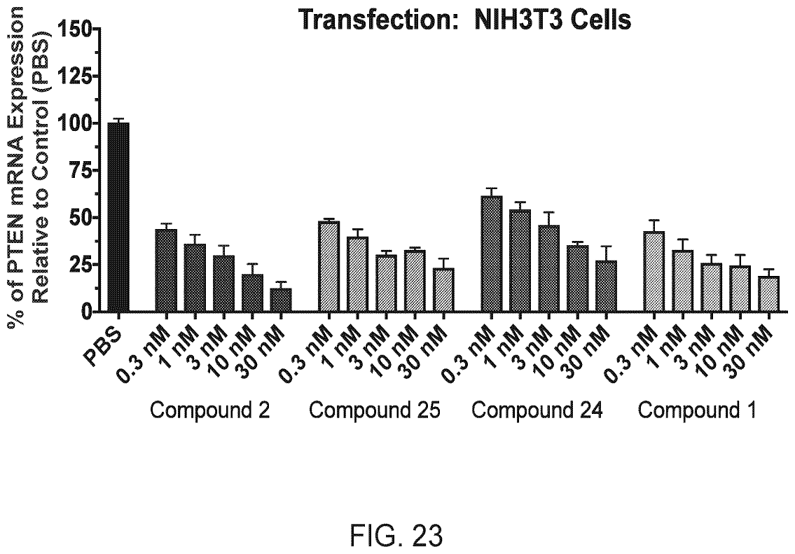

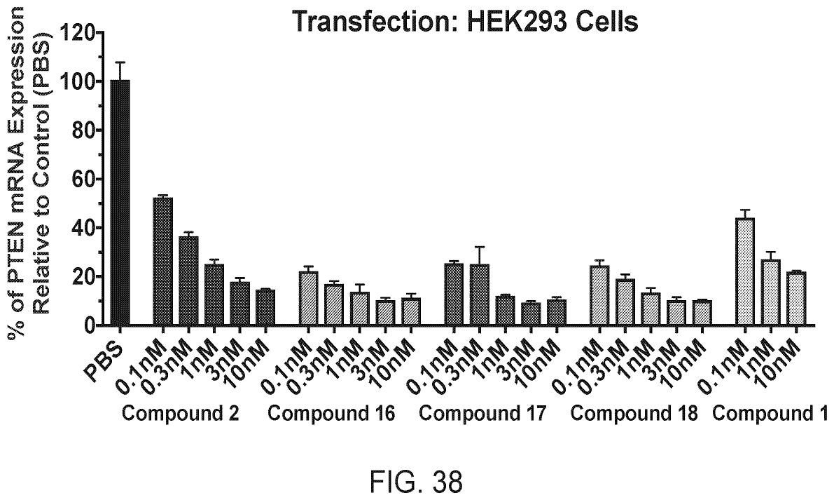

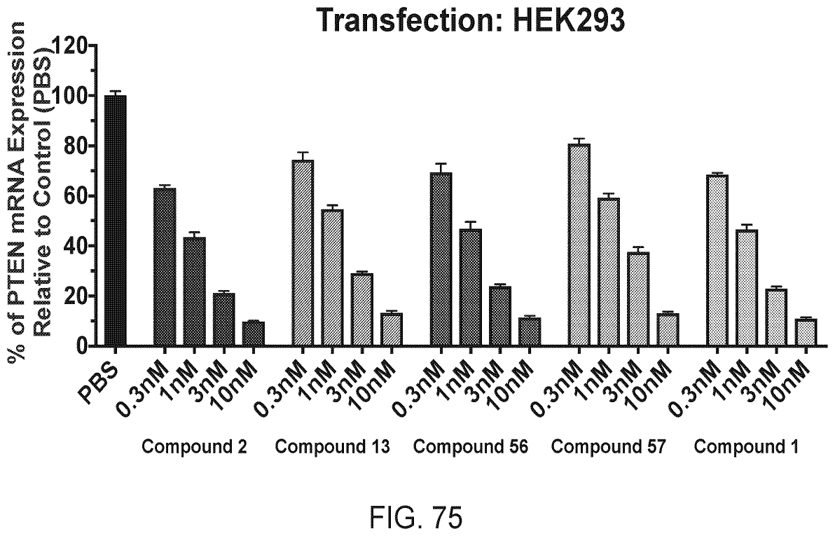

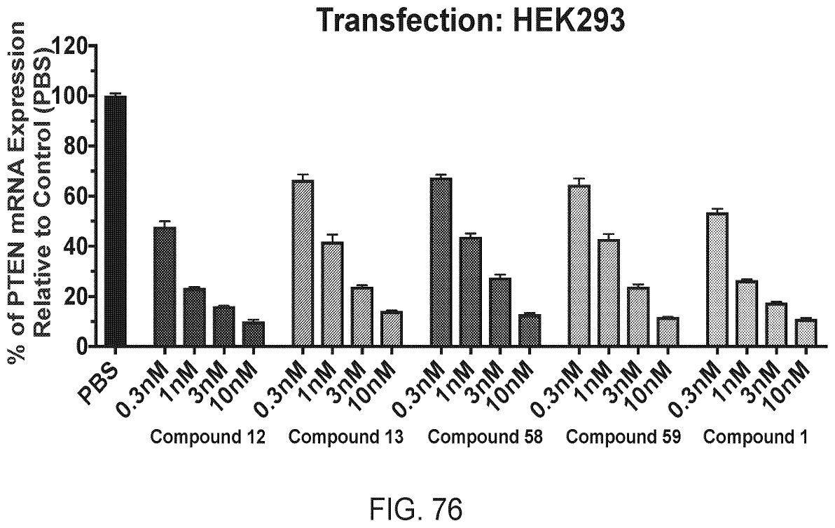

PTEN was chosen as the siRNA target because it is ubiquitously expressed across all cells and tissues and is a target that is commonly used to characterize new delivery technologies for siRNA and antisense molecules. To confirm that the conjugation of long-chain fatty acids (LCFA) does not interfere with the ability of a PTEN siRNA to incorporate into the RISC complex, each of the LCFA-conjugated PTEN siRNAs, i.e., Compounds 2, 7-18, 20, 21, 23-26, 33, 38, 39, 40, 42, 44, 48, 50, 51, 54, 55, 56, 57, 58, and 59 (See FIGS. 1-12A), and unconjugated PTEN siRNA (Compound 1) were transfected into HEK293 and/or NIH3T3 cells. Each of the RNA was isolated 24-48 hours later, and PTEN mRNA was quantified by QT-PCR. Irrespective of the LCFA motif, the conjugation site on the siRNA (i.e., 5′ or 3′) or the number of sites conjugated on the siRNA, all of the compounds retained their ability to inhibit PTEN mRNA expression following transfection (See FIGS. 13, 16, 18, 20, 22, 23, 30, 32, 34, 35, 38, 74, 75, and 76). While each compound demonstrated some inhibitory effect when introduced into cells with a transfection reagent, there were differences in activity observed across compounds tested that were related to, for example, the nature of the LCFA conjugate, or the absence of transfection reagent. Certain of these effects are presented in more detail in the following examples.

### Impact of LCFA Number and Positioning

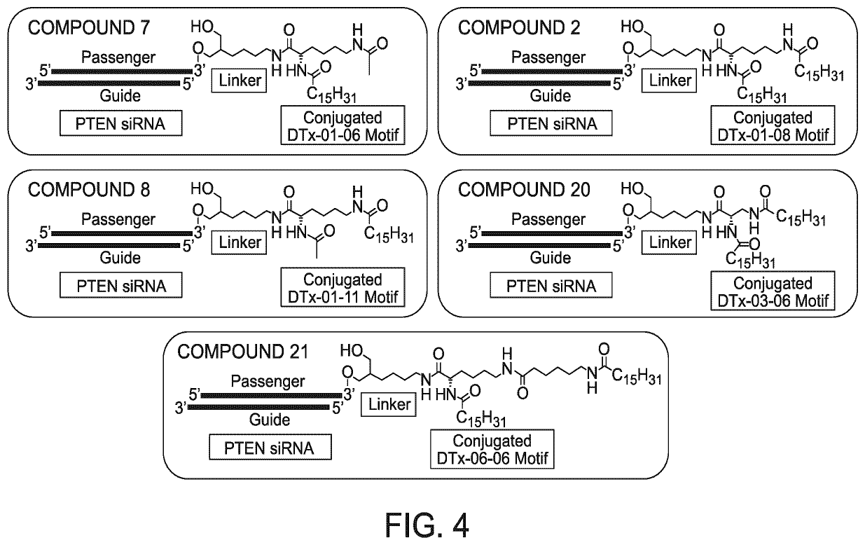

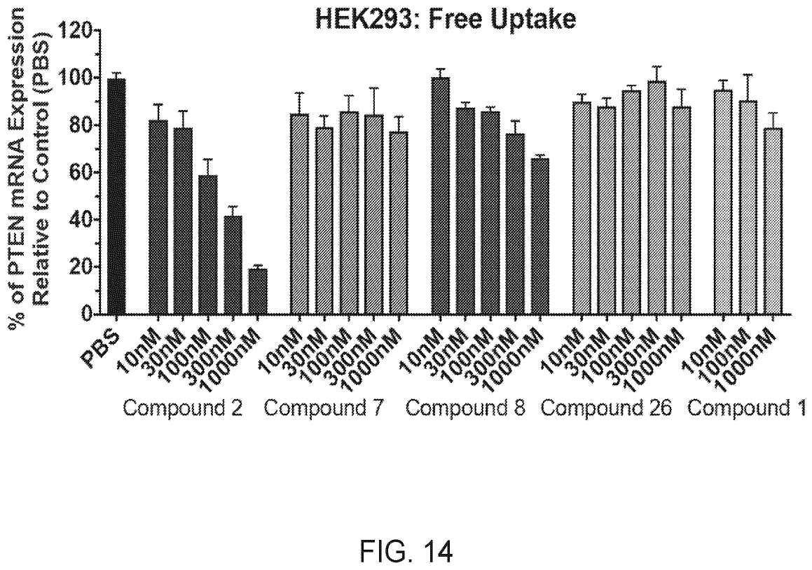

Compounds 2, 7, and 8 provide insight on the conjugation of two C16 LCFA to a single siRNA conjugation site allowing evaluation of the uptake and activity of siRNA relative to the conjugation of one C16 LCFA (See FIG. 4). A lysine scaffold was used to conjugate one or two LCFAs in a single lipid motif, and a C7 linker was used to attach the lipid motif to PTEN siRNA. Compound 2 contains a C16 LCFA attached at each of the α and ξ amino groups of the lysine. Compound 7 contains a C16 LCFA attached to the α amino group of the lysine and an acetyl group attached to the ξ amino group of the lysine. Compound 8 contains a C16 LCFA attached to the ξ amino group of the lysine and an acetyl group attached to the α amino group of the lysine. These compounds were incubated, along with unconjugated PTEN siRNA (Compound 1), on HEK293 or HUVEC cells for 48 hours in media containing 2% serum. RNA was isolated 48 hours later, and PTEN mRNA quantified by QT-PCR. In both cell types, Compound 2 inhibited PTEN mRNA expression more potently and efficaciously than Compound 7, Compound 8, or Compound 1 (See FIGS. 14&15). These data demonstrate that the conjugation of two C16 LCFAs to a single siRNA conjugation site enables siRNA uptake and activity more effectively than conjugation of one C16 LCFA.

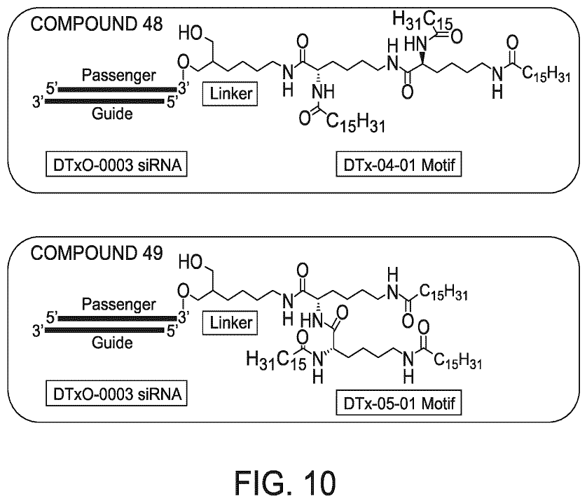

To evaluate the effects of the presence of three LCFAs, conjugate motifs comprising three fatty acid chains were designed (FIG. 10). Compound 48 was selected for in vitro testing under both transfection and free uptake conditions.

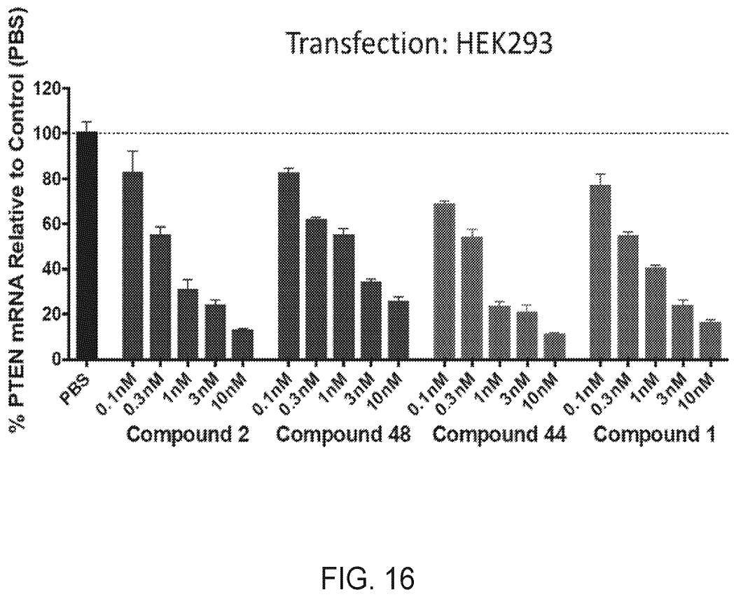

Compounds 2 and 48 were transfected into HEK293 cells. Compound 1, the unconjugated PTEN siRNA, was also transfected into HEK293 cells. PBS-treated cells served as a control. RNA was isolated from the cells 48 hours later, and PTEN mRNA was quantified by QT-PCR and normalized to a housekeeping gene. The potency of Compound 48 was relatively similar, and perhaps slightly less than, that of Compounds 1 and 2 (FIG. 16).

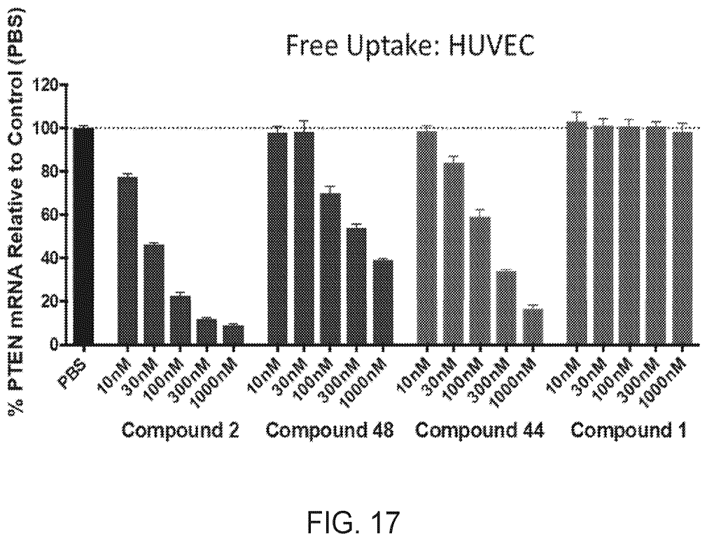

To evaluate the activity of the same compounds under free uptake conditions, the same compounds were incubated with HUVEC cells in media containing 2% serum. RNA was isolated from the cells 48 hours later, and PTEN mRNA was quantified by QT-PCR and normalized to a housekeeping gene. Following free uptake, Compound 48 was markedly less potent relative to Compound 2. Compound 1 had no effect on PTEN mRNA expression under these free uptake conditions (FIG. 17).

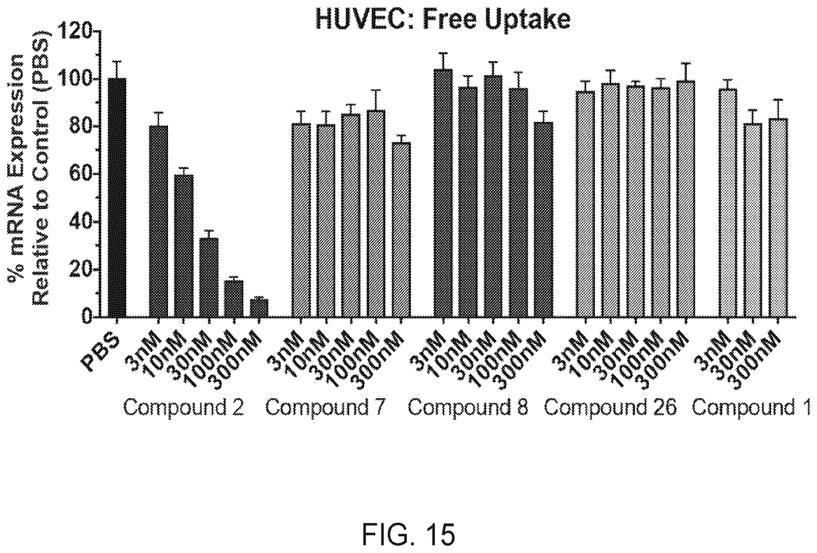

These data demonstrate that in the present context, while a conjugate moiety with three C16 LCFAs is more effective than the conjugation of a single C16 LCFA (compare to Compounds 7 and 8 in FIGS. 14 and 15) it is markedly less effective than a conjugate moiety with two C16 LCFAs for enabling siRNA uptake and activity.

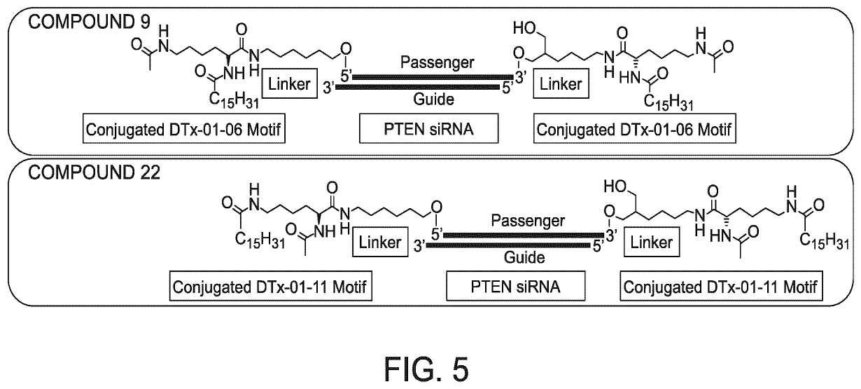

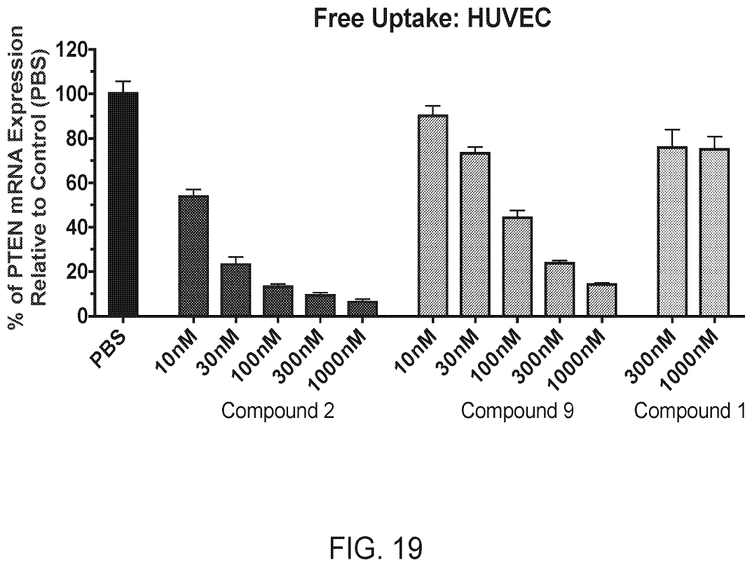

Compound 9 provides insight on the relative positioning of each of two conjugated C16 LCFA on siRNA allowing determination of uptake and activity (See FIG. 5). As detailed above, Compound 2 contains a C16 LCFA attached at each of the α and ξ amino groups of the lysine (See FIG. 4). Compound 9, at the 3′ position of the PTEN RNA, contains a C7 linker covalently bonded to a lysine scaffold with a C16 LCFA attached to the α amino group of the lysine and an acetyl group attached to the ξ amino group of the lysine. At the 5′ position, Compound 9 contains a C6 linker covalently bonded to a lysine scaffold with a C16 LCFA attached to the α amino group of the lysine and an acetyl group attached to the ξ amino group of the lysine. Compound 2, Compound 9, and unconjugated PTEN siRNA (Compound 1) were incubated on HUVEC cells for 48 hours in media containing 2% serum. RNA was isolated and PTEN mRNA quantified by QT-PCR. Compound 2 was about 10-fold more potent at inhibiting PTEN mRNA expression relative to Compound 9 (See FIG. 19). These data demonstrate that the context in which two C16 LCFA are conjugated to the same siRNA affects siRNA uptake and activity.

### Conjugation to 3′ or 5′ End of Passenger Strand

In compounds described herein, for example Compound 2, the conjugate moiety was attached to the 3′ end of the DTxO-0003 PTEN siRNA passenger strand. To understand if the site of conjugation of the DTx-01-08 moiety on the siRNA passenger strand impacted activity, the site of conjugation was varied. In Compounds 50 and 51, the DTx-01-08 was conjugated to the 5′ end of the passenger strand two different PTEN siRNAs, DTxO-0003 and DTxO-0038, respectively. These compounds were tested under both transfection and free uptake conditions.

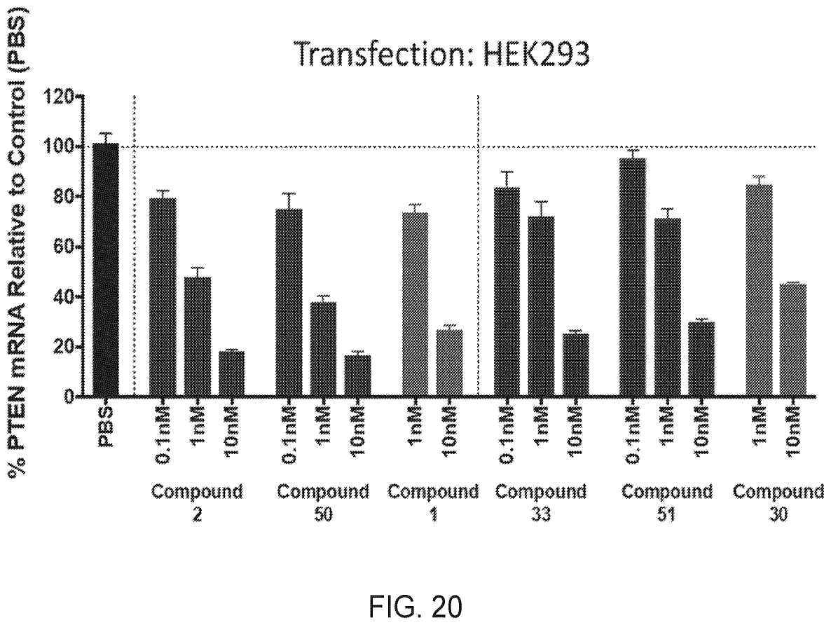

The DTxO-0003-related Compounds 1 (unconjugated DTxO-0003 siRNA), Compound 2 (DTxO-0003 with conjugate at 3′ end of passenger strand) and Compound 50 (DTxO-0003 with conjugate at 5′ end of passenger strand) were transfected into HEK293 cells. The DTxO-0038-related Compound 30 (unconjugated DTxO-0038), Compound 33 (DTxO-0038 with conjugate at 3′ end of passenger strand) and Compound 51 (DTxO-0038 with conjugate at the 5′ end of the passenger strand) were also transfected into HEK293 cells. PBS-treated cells served as a control. RNA was isolated from the cells 48 hours later, and PTEN mRNA was quantified by QT-PCR and normalized to a housekeeping gene. Compound 50 was as active as Compounds 1 (unconjugated DTxO-0003) and 2 (DTxO-0003 with conjugate at 3′ end of passenger strand), and Compound 51 was as active as Compounds 30 (unconjugated DTxO-0038) and 33 (DTxO-0038 with conjugate at 3′ end of passenger strand) (See FIG. 20).

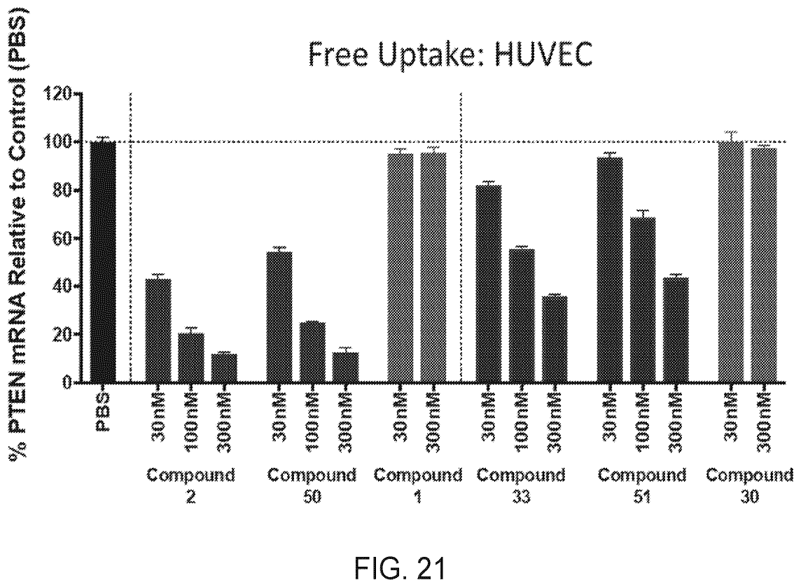

The same compounds were tested in a free uptake experiment in HUVEC cells. PBS-treated cells served as a control. RNA was isolated from the cells 48 hours later, and PTEN mRNA was quantified by QT-PCR and normalized to a housekeeping gene. In this experiment in HUVEC cells, both Compound 2 and Compound 50 inhibited PTEN mRNA expression whereas Compound 1 had no effect (See FIG. 21). Similarly, both Compound 33 and Compound 51 inhibited PTEN mRNA expression, whereas Compound 30 did not.

These data indicate that conjugation at the 5′ or 3′ terminus of the passenger strand similarly enables siRNA uptake and activity.

### The Effect of Exposed COOH Moieties

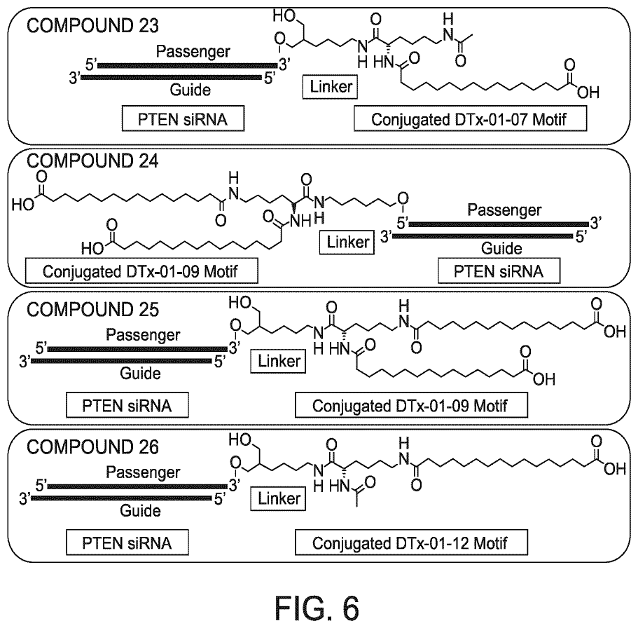

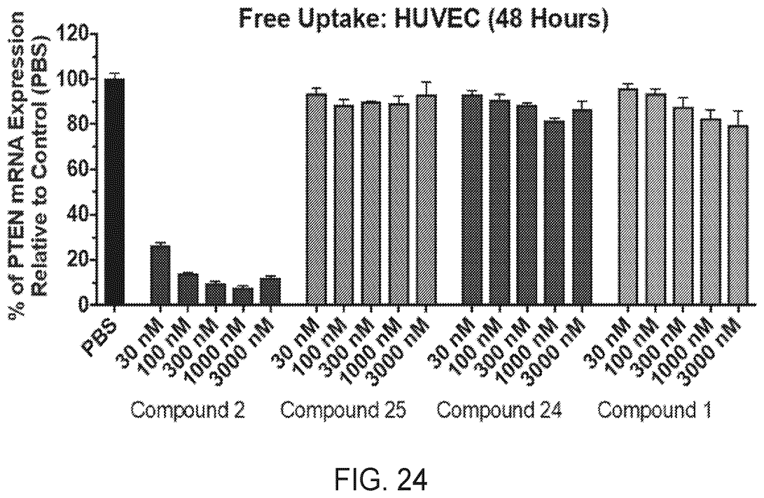

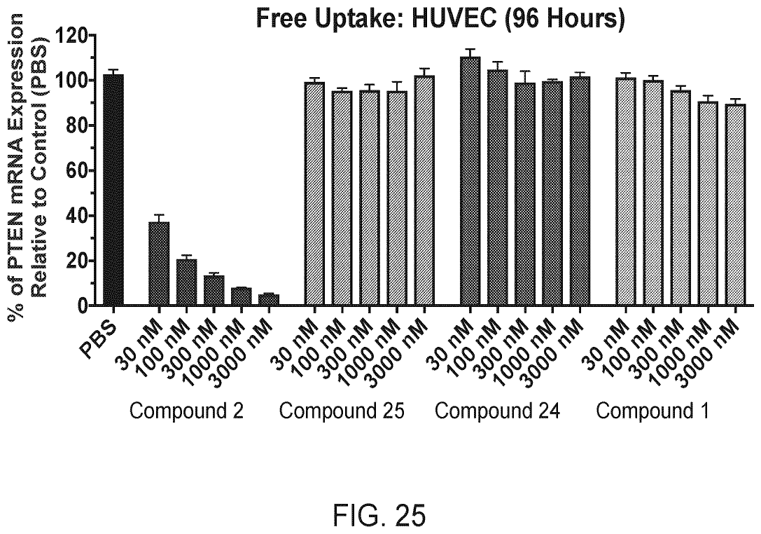

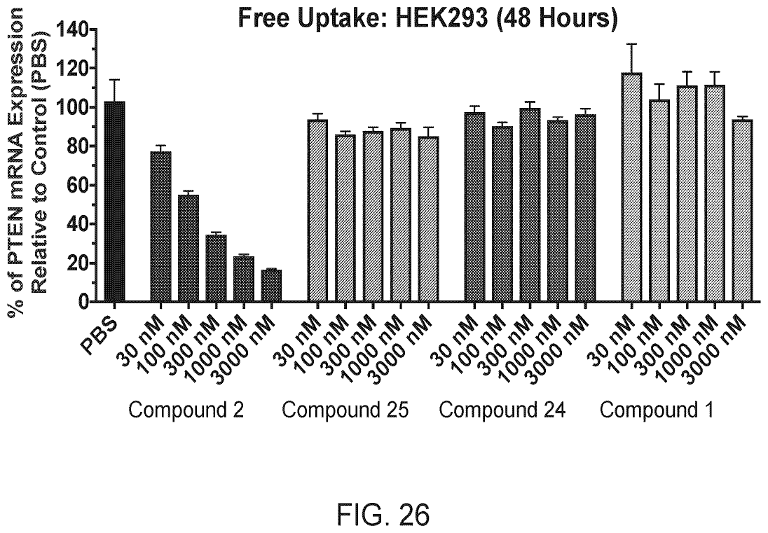

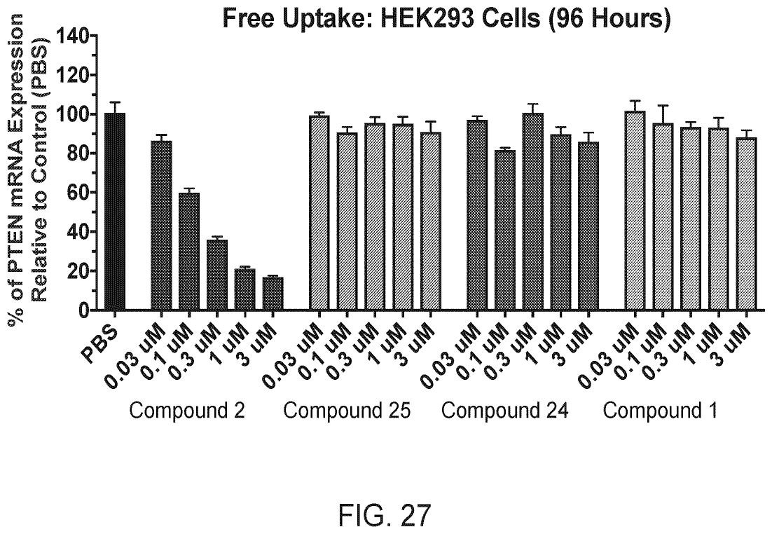

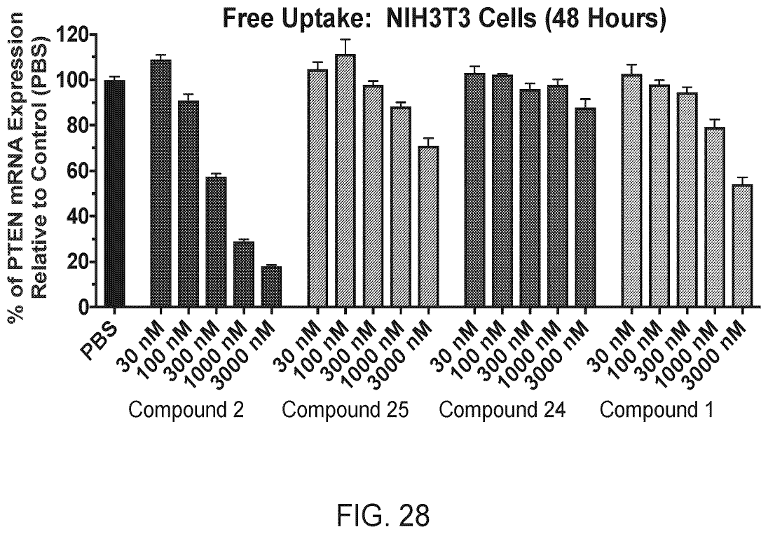

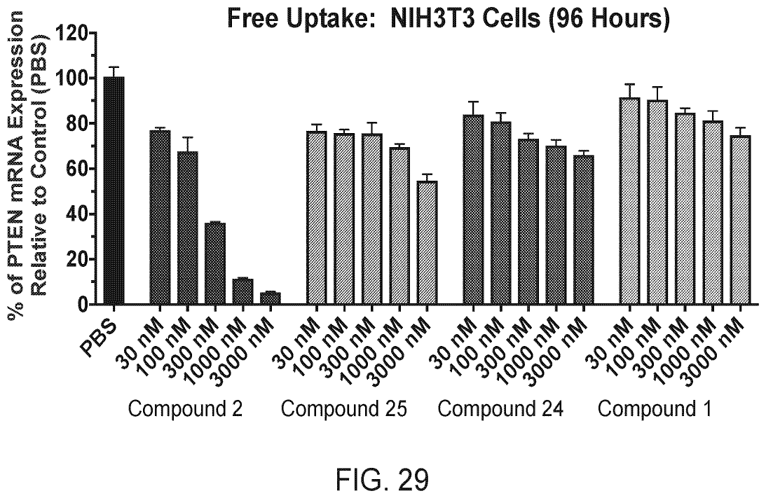

To investigate LCFA-siRNA conjugates with exposed COOH groups that might be available for receptor/transporter interaction, Compounds 24-26 were synthesized (See FIG. 6). Compound 24 and Compound 25 each contain two C16 LCFA terminating in an exposed COOH, one attached at each of the α and ξ amino groups of a lysine scaffold. The fatty acid motif of Compound 24 is conjugated to the 5′ end of PTEN siRNA via a C6 linker. The fatty acid motif of Compound 25 is conjugated to the 3′ end of PTEN siRNA via a C7 linker. Like Compound 25, Compound 26 is conjugated to the 3′ end of PTEN siRNA via a C7 linker and contains a lysine scaffold; however, Compound 26 contains a C16 LCFA with an exposed COOH attached to the ξ amino group of the lysine and an acetyl group attached to the α amino group of the lysine. These compounds, Compound 2, and unconjugated PTEN siRNA (Compound 1) were incubated on HEK293, NIH3T3 or HUVEC cells for 48 or 96 hours in media containing 2% serum in free uptake assays. RNA was isolated, and PTEN mRNA quantified by QT-PCR. In all 3 cell types, Compound 2 inhibited PTEN mRNA expression much more potently and efficaciously than any of Compounds 24-26 (See FIGS. 14, 15, 24-29). Compounds 24-26 exerted little or no effect in inhibiting PTEN mRNA expression. At least in the cell lines and conditions evaluated in these in vitro experiments, these LCFA-conjugated siRNAs with exposed COOH group(s) did not promote siRNA uptake and activity.

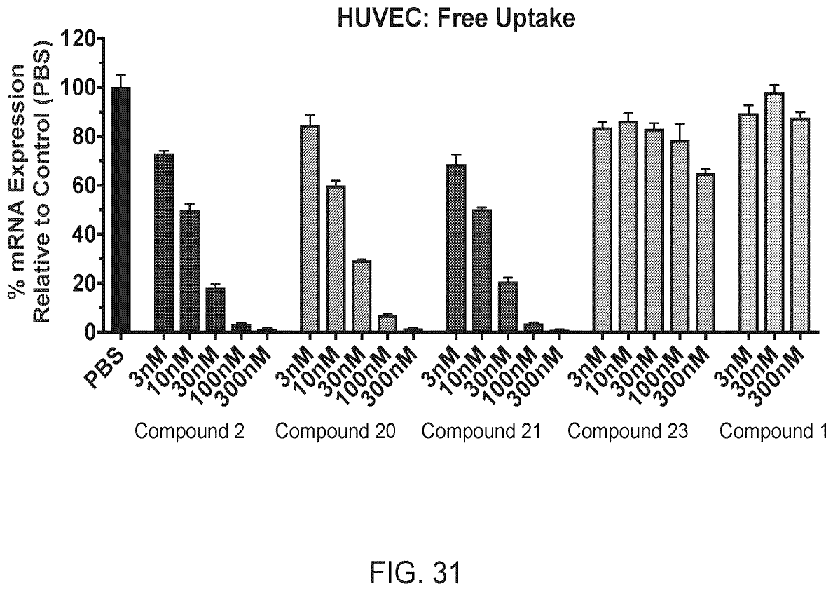

Like Compound 26, the fatty acid motif of Compound 23 is conjugated to the 3′ end of the PTEN siRNA via a C7 linker and contains a lysine scaffold; however, Compound 23 contains a C16 LCFA with an exposed COOH group attached to the α amino group of the lysine and an acetyl group attached to the ξ amino group of the lysine. The activity of this compound was evaluated in a separate free uptake experiment in HUVEC cells. Compound 23, along with Compound 2 and Compound 1, were incubated on HUVEC cells for 48 hours. RNA was then isolated and PTEN mRNA quantified by QT-PCR. Compound 23 and Compound 1 had little or no effect to inhibit PTEN mRNA expression whereas Compound 2 dose-dependently inhibited PTEN mRNA expression (FIG. 31).

### The Effect of LCFA Length

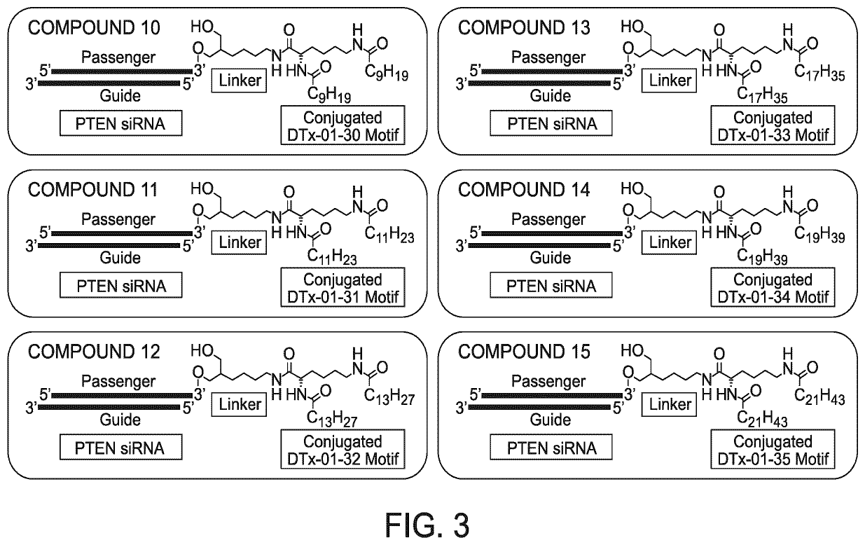

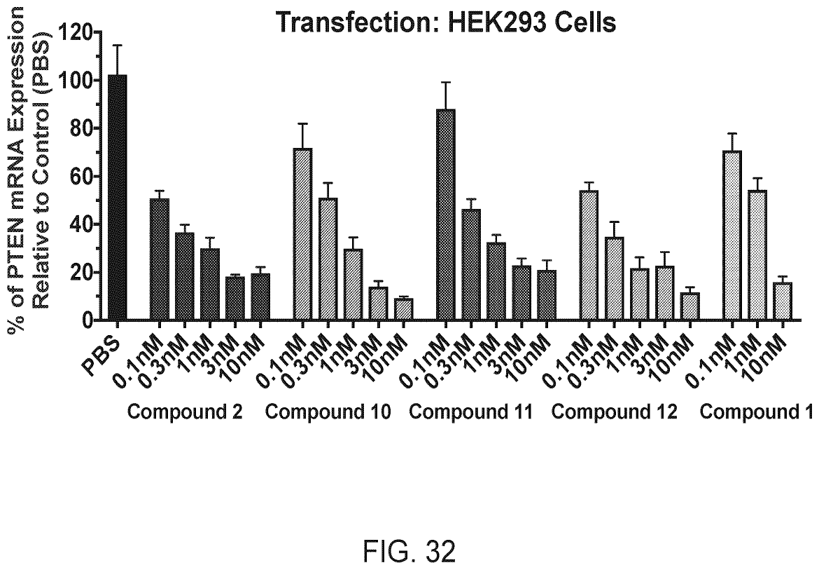

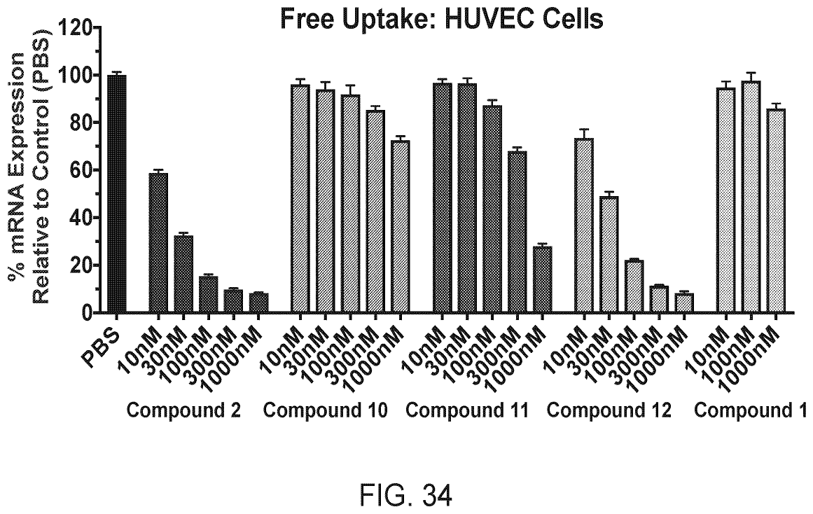

To understand the effect of fatty acid chain length on siRNA uptake and activity, Compounds 10-15 were synthesized (See FIG. 3). Each of Compounds 10-15 contained a lysine scaffold to conjugate two LCFAs in a single lipid motif, and a C7 linker to attach the lipid motif to the PTEN siRNA. Compounds 10-15 contain C10, C12, C14, C18, C20 or C22 LCFAs, respectively, attached to the amino groups on the lysine. Transfection experiments confirmed that Compounds 10-15 inhibited PTEN mRNA expression in HEK293 cells (See FIGS. 32 & 33). To determine their activity under free uptake conditions, Compounds 10-15, Compound 2, and unconjugated PTEN siRNA (Compound 1) were incubated on HUVEC cells for 48 hours in media containing 2% serum. RNA was isolated, and PTEN mRNA quantified by QT-PCR. Compounds 2 and Compound 12 inhibited PTEN mRNA expression more potently than Compound 10, Compound 11, Compound 13, and Compound 14 (See FIGS. 34 & 35). At least in HUVEC cells, Compound 2 was modestly more potent than Compound 12. These data demonstrate that fatty acid length affects siRNA uptake and activity, with a decrease in activity for saturated fatty acids shorter than 12 carbons and longer than 18, when such fatty acids are conjugated to siRNA via the disclosed C7 linker and lysine.

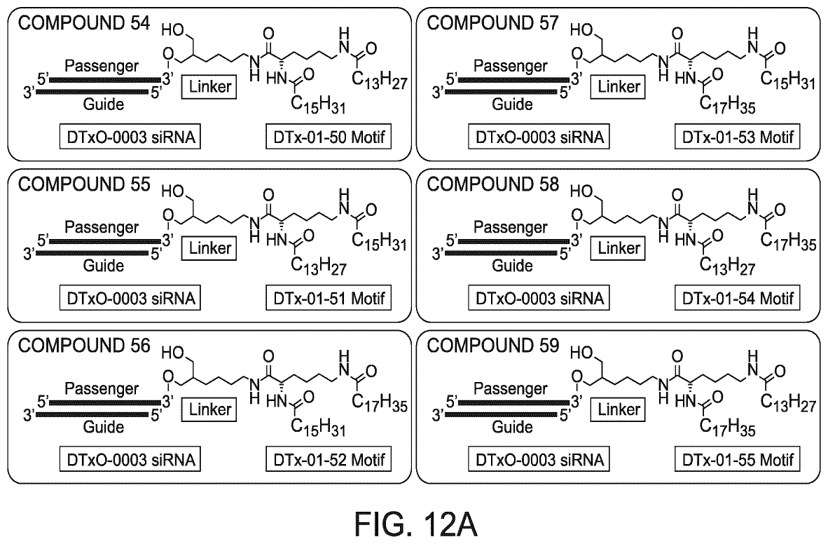

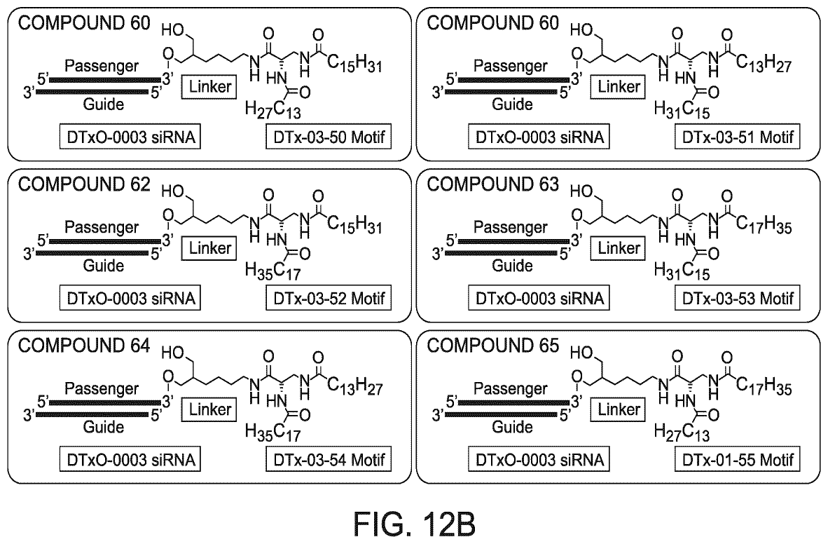

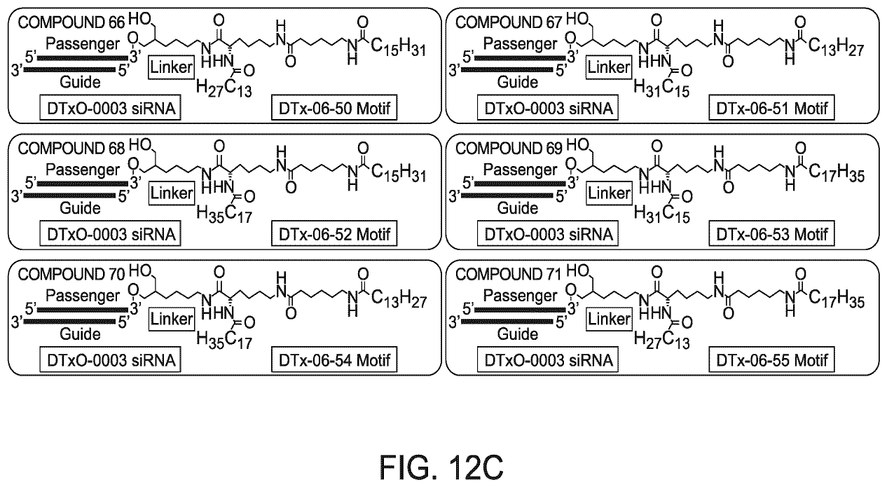

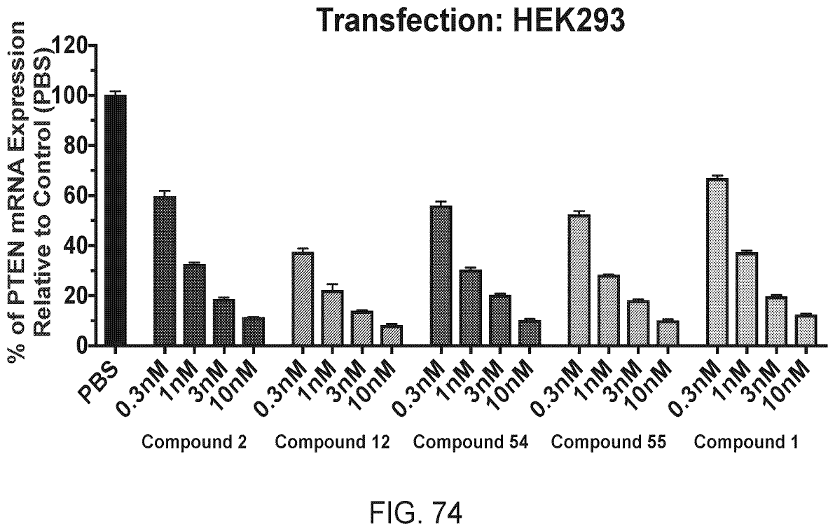

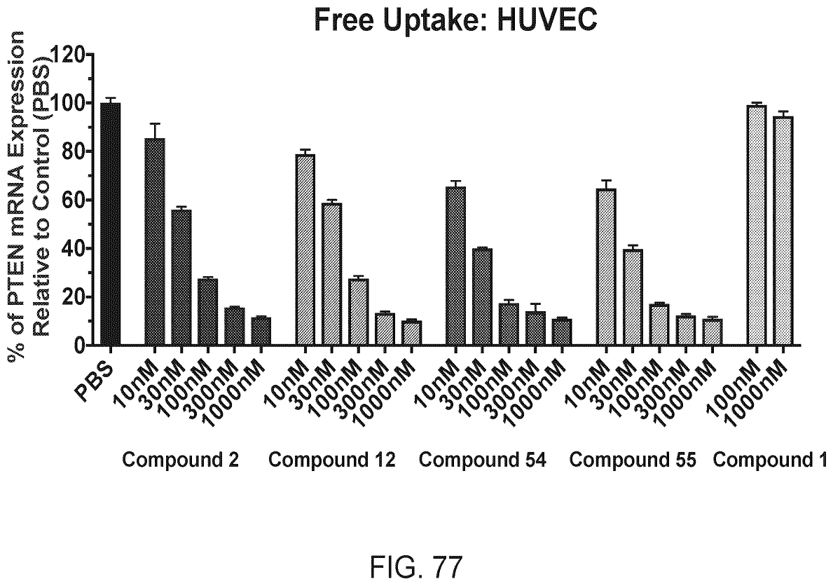

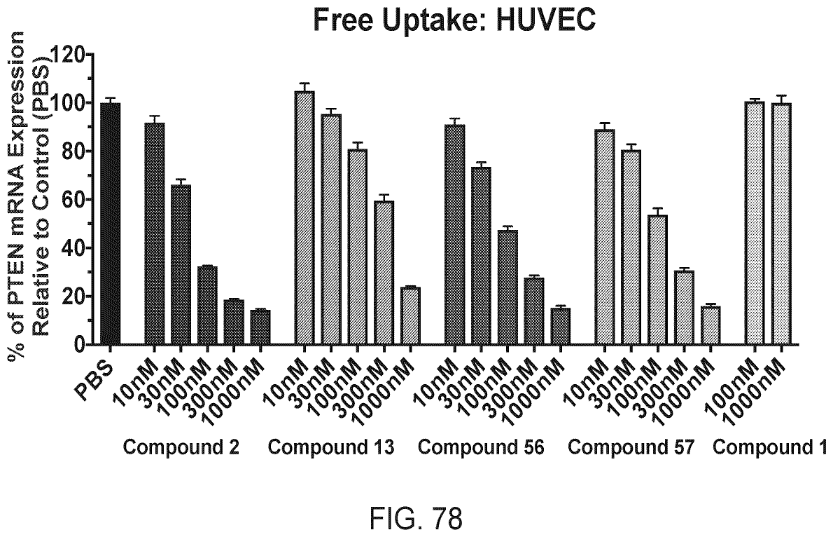

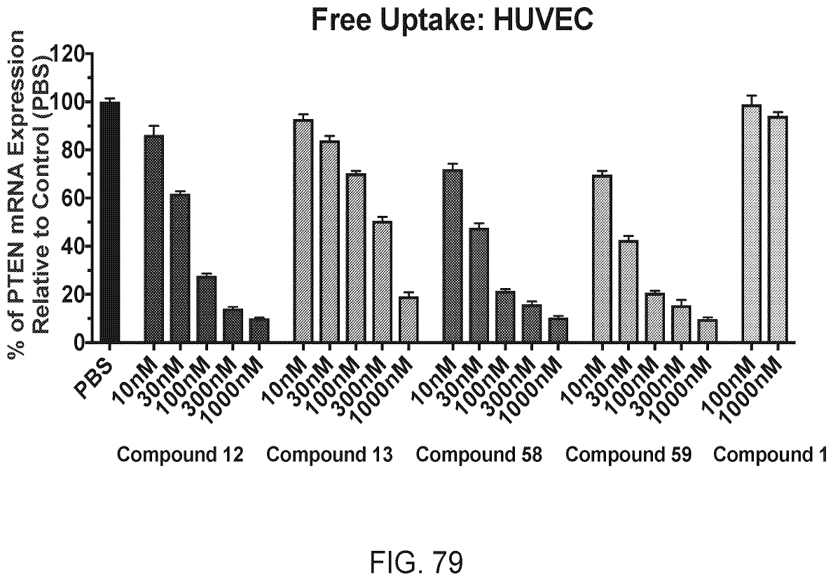

As described herein, compounds containing two C14 saturated LCFAs, two C16 saturated LCFAs, or two C18 LCFAs are active in free uptake experiments. To understand whether compounds containing certain combinations of C14, C16 and C18 saturated LCFAs enable cellular uptake and activity, compounds 54-59 were designed (See FIG. 12A). Transfection experiments confirmed that Compounds 54-59 inhibited PTEN mRNA expression in HEK293 cells (See FIGS. 74, 75, and 76). To determine their activity under free uptake conditions, Compounds 54-59, Compound 2, Compound 12-13 and unconjugated PTEN siRNA (Compound 1) were incubated on HUVEC cells for 48 hours in media containing 2% serum. RNA was isolated, and PTEN mRNA quantified by QT-PCR. Compound 54 and Compound 55 inhibited PTEN mRNA expression as, or slightly more, effectively than Compound 2 and Compound 12 (FIG. 77). Compound 56 and Compound 57 inhibited PTEN mRNA expression to a greater extent than Compound 13 but, was slightly less effective to inhibit PTEN mRNA expression than Compound 2 (FIG. 78). The activity of both Compound 58 and 59 appeared to be as, or slightly more, effective to inhibit PTEN mRNA expression as Compound 12 but, less effective to inhibit PTEN mRNA expression relative to Compound 13 (FIG. 79). Compound 1 had little or no effect to inhibit PTEN mRNA expression (FIGS. 77, 78 and 79). These data demonstrate that the conjugation of certain combinations of saturated fatty acids can be utilized to promote siRNA uptake and activity. Strikingly, compounds containing either a C14 or a C16 LCFA with a C18 LCFA are more potent and efficacious than compounds containing two identical C18 LCFAs.

### Effects of Conjugation of Motifs Comprising Unsaturated Fatty Acids

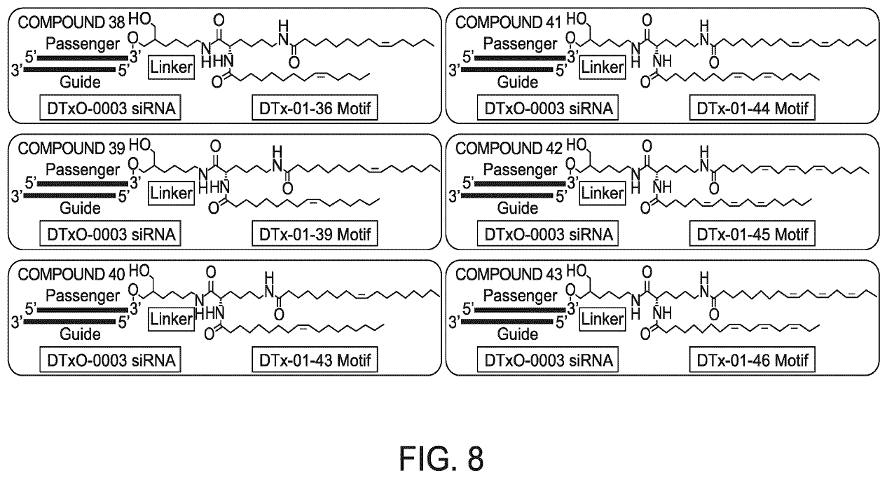

As described herein, compounds containing two C14 saturated LCFAs, two C16 saturated LCFAs or two C18 LCFAs are active in free uptake experiments. To understand whether the degree of saturation effects siRNA uptake and activity, compounds comprising a PTEN siRNA linked to a conjugate moiety containing an unsaturated LCFA were designed (FIG. 8). Compound 38 contains two C14 unsaturated LCFAs, Compound 39 contains two C16 unsaturated LCFAs and Compounds 40 and 42 each contain two C18 unsaturated LCFAs. The LCFAs of Compound 40 each have one unsaturated carbon-carbon bond, and the LCFAs of Compound 42 each have three unsaturated carbon-carbon bonds.

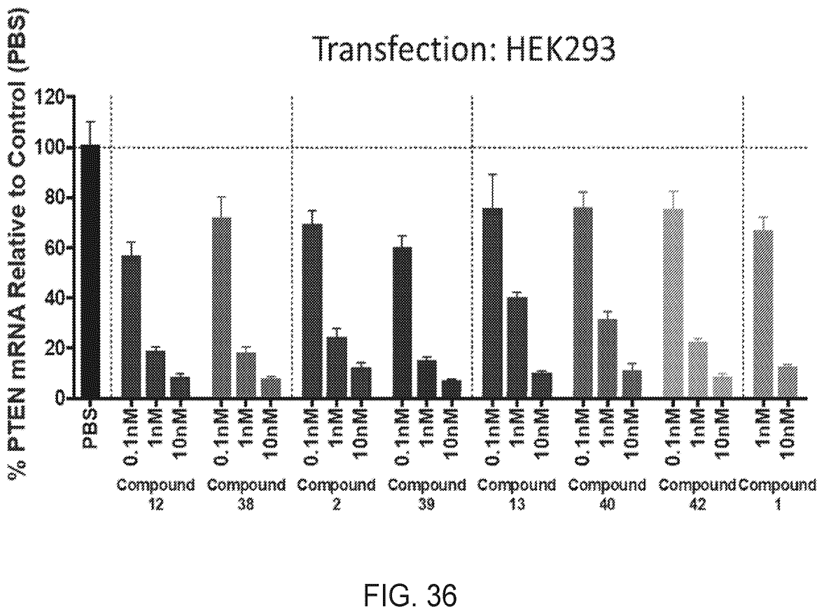

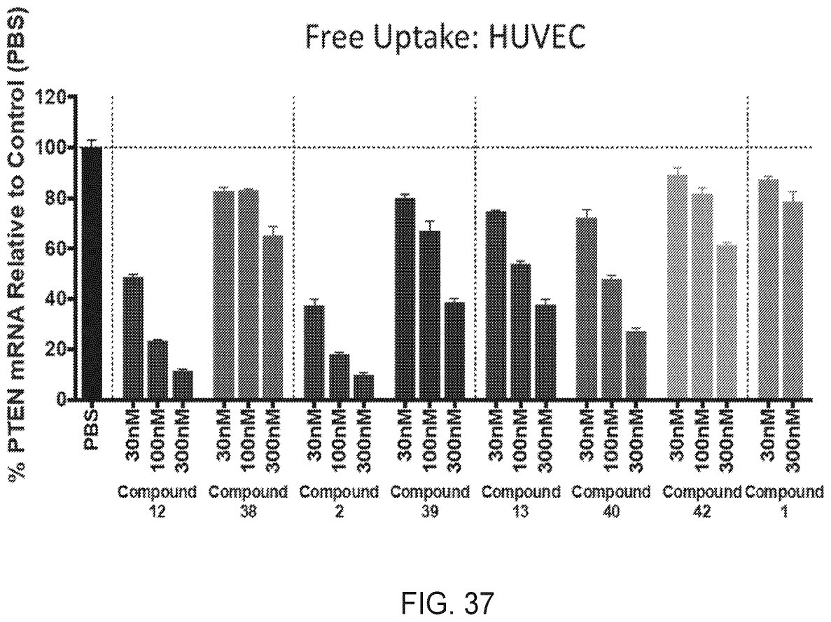

Compounds 38, 39, 40, and 42 were evaluated under transfection conditions in HEK293 cells. Compounds 1, 2, 12, and 13 were included for comparison of activity. PBS-treated cells served as a control. RNA was isolated from the cells 48 hours later, and PTEN mRNA was quantified by QT-PCR and normalized to a housekeeping gene. Following transfection, each unsaturated LCFA conjugate was similarly potent in repressing PTEN mRNA expression relative to the equivalent length saturated LCFA conjugate (compare Compound 12 to 38; 2 to 39; and 13 to 40 and 42 in FIG. 36).

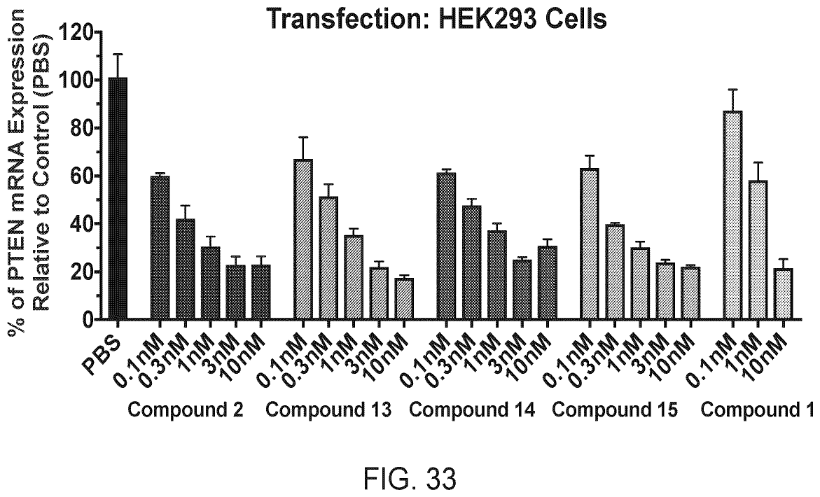

To evaluate the activity of the same compounds under free uptake conditions, the compounds were incubated with HUVEC cells in media containing 2% serum. RNA was isolated from the cells 48 hours later, and PTEN mRNA was quantified by QT-PCR and normalized to a housekeeping gene. Following free uptake, differences were observed in the activity of the various compounds (FIG. 33). As illustrated by differences in reducing PTEN mRNA expression, the C14 unsaturated LCFA conjugate Compound 38, the C16 unsaturated LCFA conjugate Compound 39, and the C18 unsaturated LCFA conjugate Compound 42 were less potent relative to their respective saturated LCFAs of the same length. (Compare Compound 12 to 38; 2 to 39; and 13 to 42). The exception to this trend is Compound 40, a C18 unsaturated LCFA conjugate that is similarly active as the C18 saturated LCFA conjugate Compound 13.

These data demonstrate that the degree of saturation and the length of the LCFA impact siRNA uptake and activity.

Compound 2's siRNA Uptake and Activity Relative to DHA

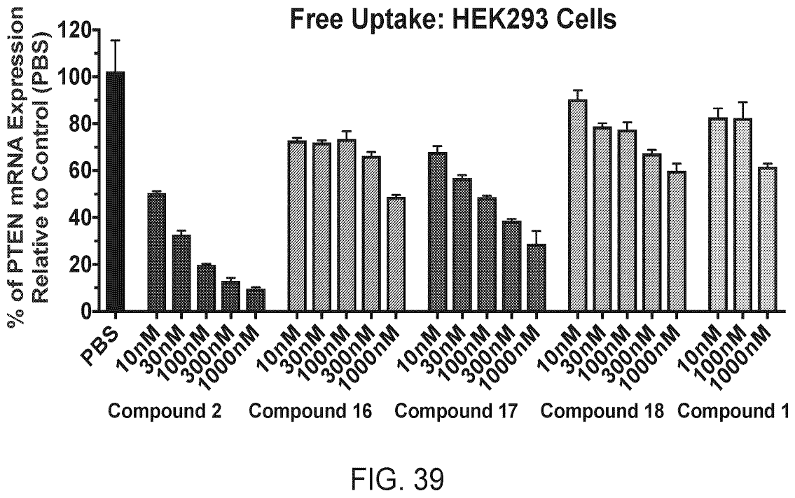

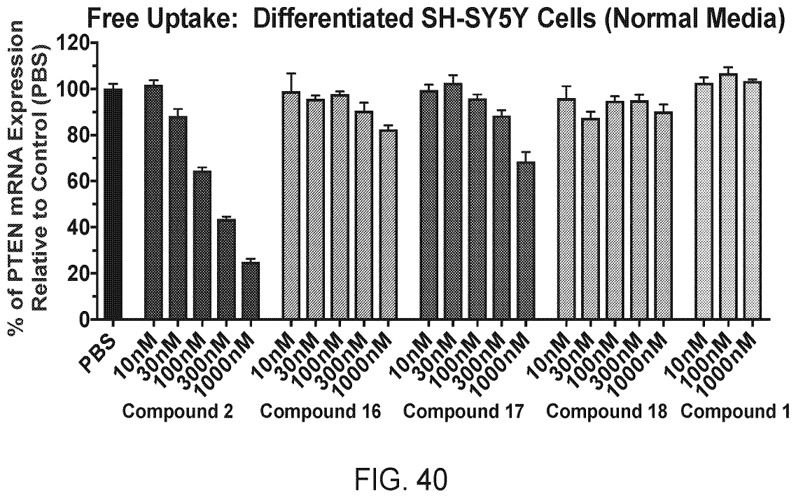

Highlighting the potential of DHA to specifically target neurons, research has demonstrated that high doses of DHA-conjugated siRNA enabled knockdown of huntingtin mRNA in the brain. PTEN siRNA conjugated to either one or two DHA were synthesized (See Compounds 16-18 in FIG. 1). As above, a C7 linker and a lysine scaffold were utilized to attach the fatty acids covalently to the siRNA. Compound 17 contains DHA attached to each of the amino groups on the lysine. Compound 16 contains DHA attached to the α amino group of the lysine and an acetyl group attached to the ξ amino group of the lysine, whereas Compound 18 contains DHA attached to the ξ amino group of the lysine and an acetyl group attached to the α amino group of the lysine. These compounds, Compound 2, and unconjugated PTEN siRNA (Compound 1) were incubated on HEK293 and differentiated SH-SY5Y cells for 48 hours in media containing 2% serum. RNA was isolated, and PTEN mRNA quantified by QT-PCR. In both cell types, Compound 2 was more potent and effective at inhibiting PTEN mRNA expression than any of DHA-conjugated Compounds 16-18 (See FIGS. 39&40). In HEK293 cells, Compound 17, containing 2 DHAs, was more potent and efficacious than the compounds containing a single DHA. In SH-SY5Y cells, Compound 17 at the highest dose exhibited more activity than the compounds containing a single DHA, but the effect was small.

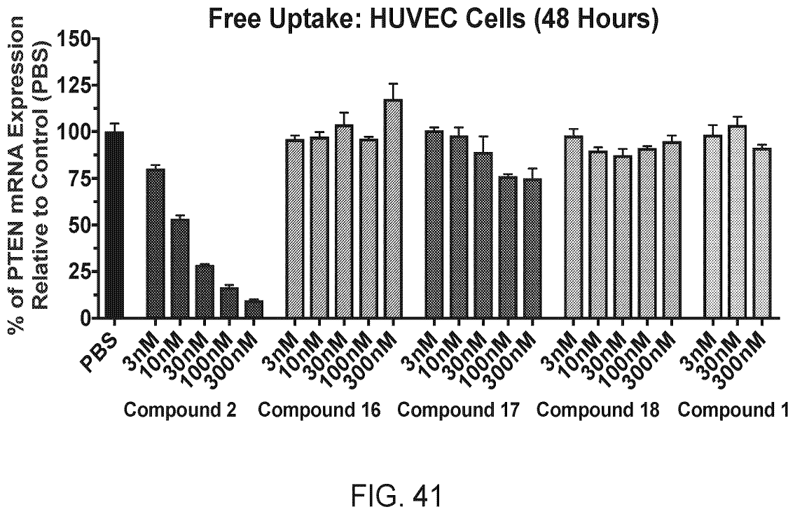

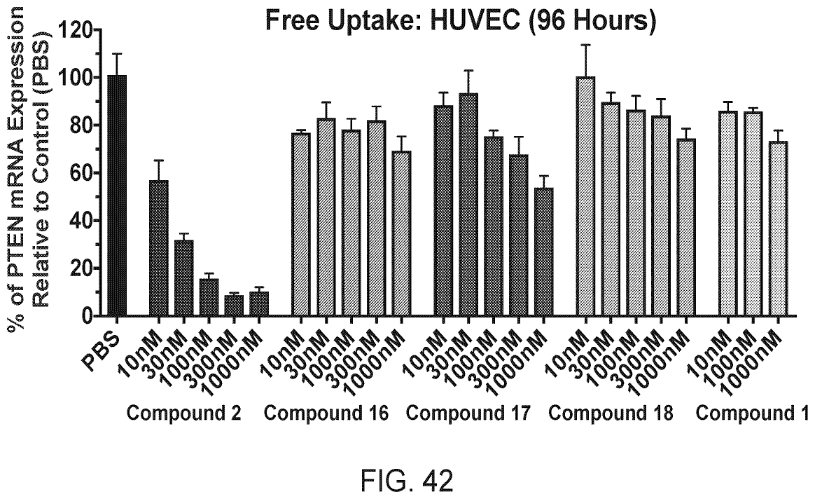

A similar experiment was performed in HUVEC cells with the exception that the compounds were incubated for both 48 and 96 hours in 2% serum. Compound 2 was more potent and effective at inhibiting PTEN mRNA expression in HUVEC cells than any of DHA-conjugated Compounds 16-18 at both 48 and 96 hours (See FIGS. 41&42). Compound 17 exhibited some inhibition of PTEN mRNA expression at the highest dose with a 96-hour treatment.

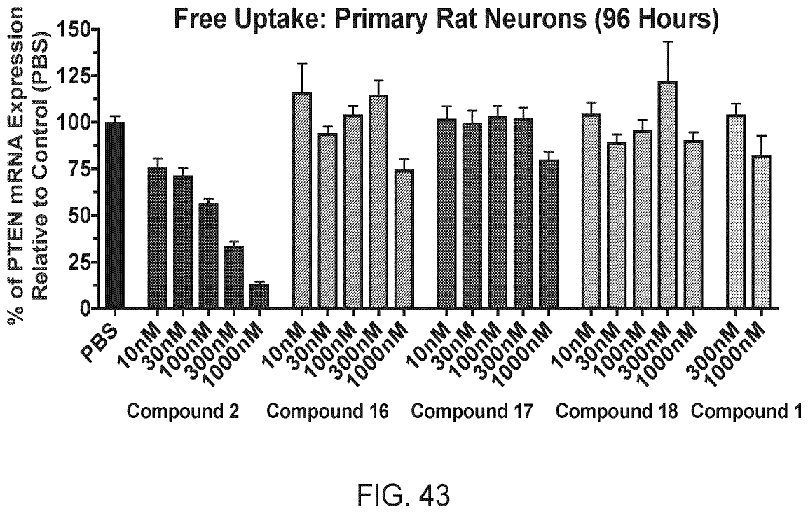

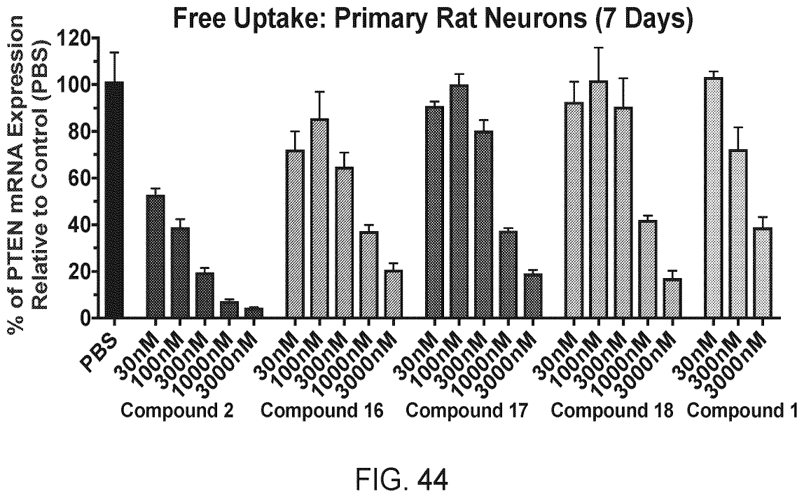

Compounds 16-18, Compound 2, and unconjugated PTEN siRNA (Compound 1) also were incubated on primary rat cortical neurons for 96 hours and 7 days (See FIGS. 43&44). At 96 hours, Compound 2 was more potent and effective at inhibiting PTEN mRNA expression than any of DHA-conjugated Compounds 16-18. In fact, Compounds 16-18, as well as control Compound 1, exhibited little if any inhibitory activity. After 7 days of incubation, all compounds dose-dependently inhibited PTEN mRNA expression; however, Compound 2 was approximately an order of magnitude more potent than Compounds 16-18 or control Compound 1. These data demonstrate that the conjugation of two C16 LCFAs to siRNA promotes siRNA uptake and activity more effectively than conjugation of either one or two DHA across HEK293 cells, HUVEC cells, SH-SY5Y cells, and primary rat cortical neurons.

Conjugation of the DTx-01-08 Motif Enables the Uptake and Activity of other siRNAs

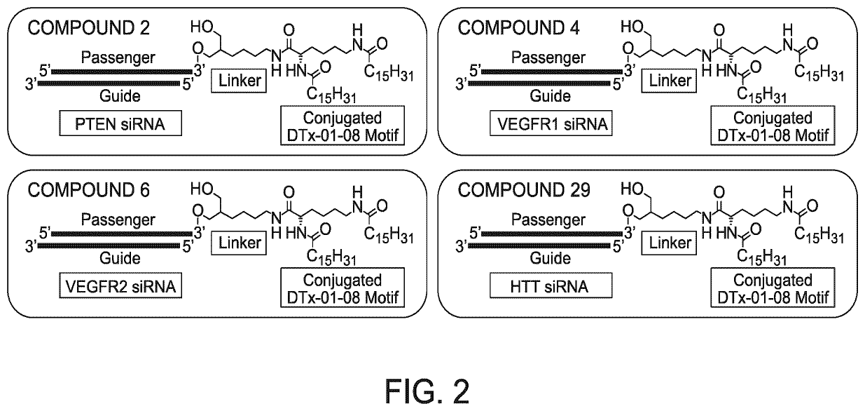

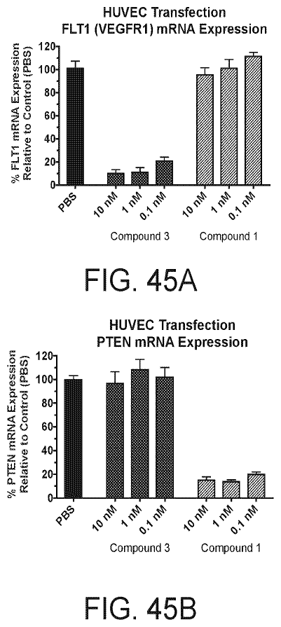

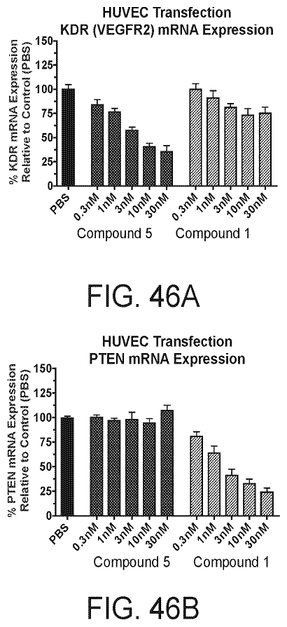

Unconjugated siRNAs targeting FLT1 (VEGFR1) and KDR (VEGFR2) mRNAs, Compounds 3 and 5 respectively, were identified and their inhibitory activity confirmed 48 hours following transfection into HUVEC cells (See FIGS. 45&46). As with Compound 2, a lysine scaffold was used to conjugate two C16 LCFAs in a single fatty acid motif, and a C7 linker was used to attach the fatty acid motif to the siRNA of interest, affording VEGFR1-siRNA Compound 4 and VEGFR2 siRNA Compound 6 (See FIG. 2).

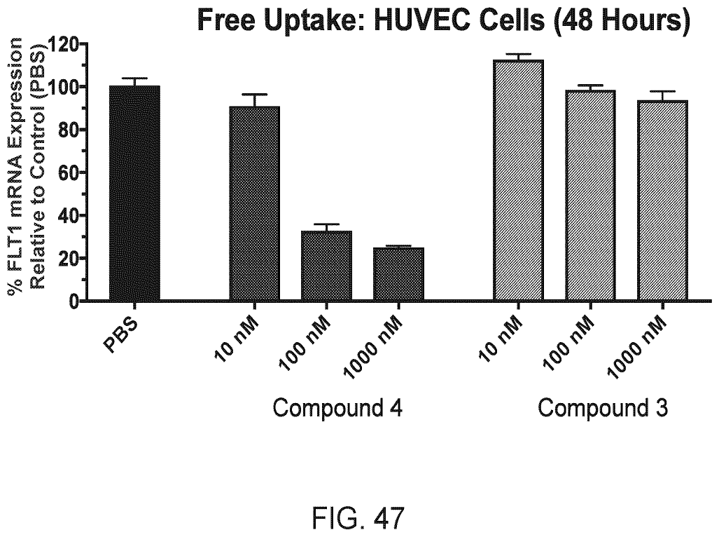

To confirm that the DTx-01-08 motif enabled VEGFR1 siRNA uptake into cells, Compound 4 and unconjugated VEGFR1 siRNA (Compound 3) were incubated on HUVEC cells for 48 hours in media containing 2% serum. RNA was isolated, and VEGFR1 mRNA expression quantified by QT-PCR. Compound 4 inhibited VEGFR1 expression, whereas Compound 3 had little or no effect (See FIG. 47).

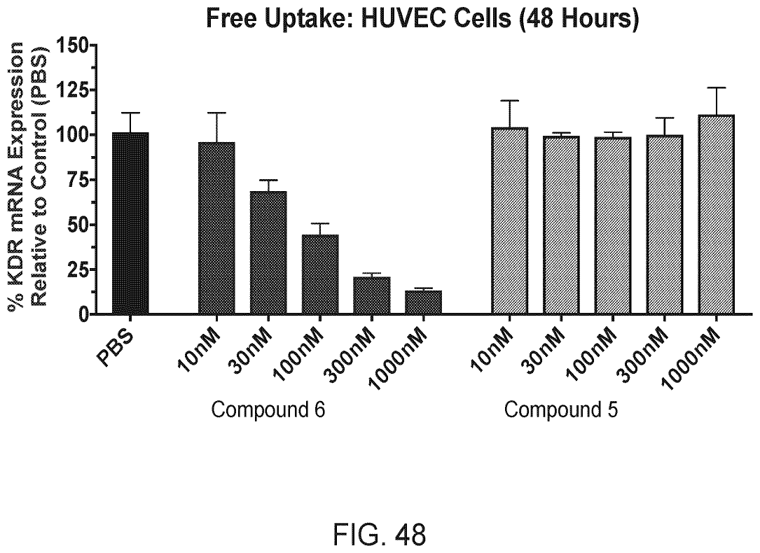

Similarly, to confirm that DTx-01-08 motif enabled VEGFR2 siRNA uptake into cells, Compound 6 and unconjugated VEGFR2 siRNA (Compound 5) were incubated on HUVEC cells for 48 hours in media without serum. RNA was then isolated, and VEGFR2 mRNA expression quantified by QT-PCR. Compound 6 inhibited VEGFR2 expression, whereas Compound 5 had little or no effect (See FIG. 48).

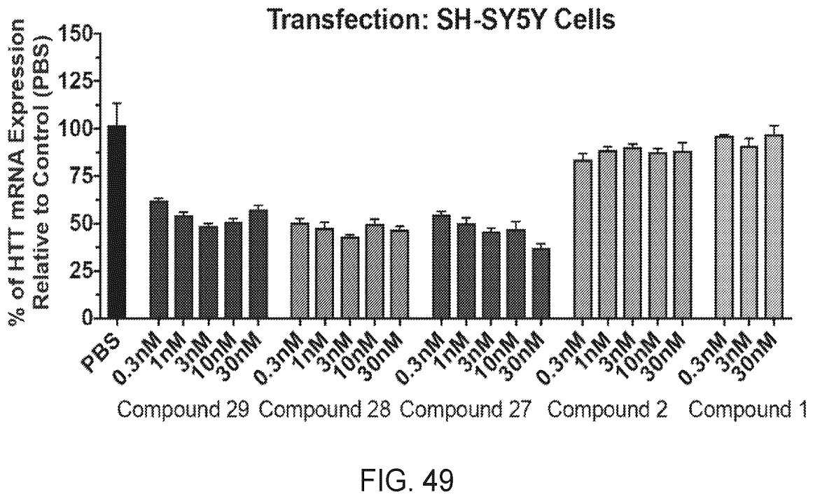

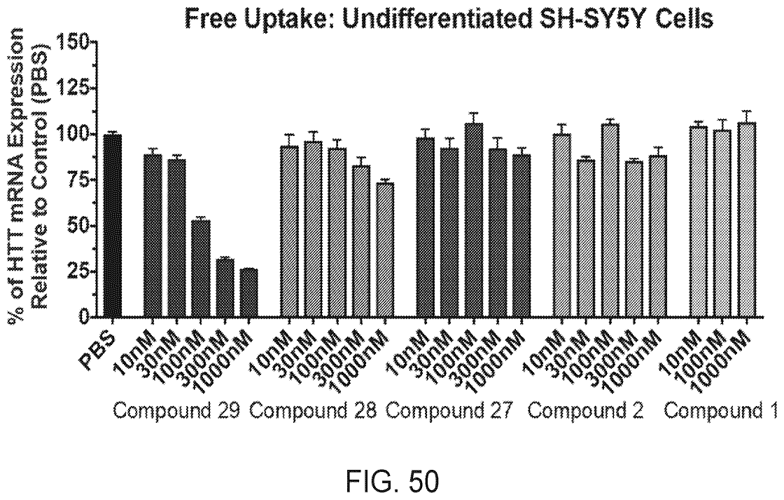

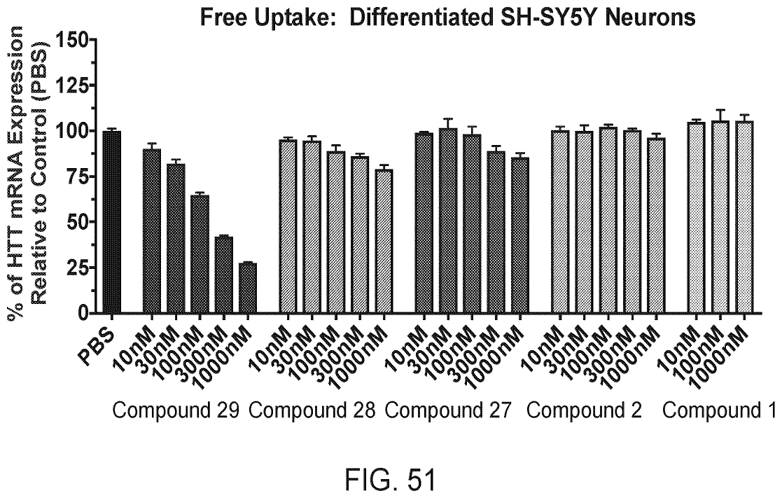

In another example, a known siRNA targeting HTT mRNA was obtained, herein referred to as Compound 27, and its inhibitory activity confirmed 48 hours following transfection into SH-SY5Y cells (See FIG. 49). As with Compound 2, a lysine scaffold was used to conjugate two C16 LCFAs in a single fatty acid motif, and a C7 linker was used to attach the fatty acid motif to the HTT siRNA, affording Compound 29 (See FIG. 2). Compound 28 was synthesized using the same HTT siRNA, C7 linker, and lysine scaffold, but with DHA attached to the ξ amino group of the lysine and an acetyl group attached to the α amino group of the lysine (See FIG. 1). Both compounds, Compound 29 and Compound 28, inhibited HTT mRNA expression as effectively as unconjugated siRNA Compound 27 following transfection into SH-SY5Y cells (FIG. 49). Compounds 29, Compound 28, Compound 27, Compound 2 and Compound 1 were incubated on both undifferentiated and differentiated SH-SY5Y cells for 48 hours in media containing 2% serum. RNA was isolated, and HTT mRNA expression quantified via QT-PCR. Under both conditions, Compound 29 dose-dependently inhibited HTT mRNA expression (See FIGS. 50&51). By contrast, Compound 28, Compound 27, Compound 2, and Compound 1 exerted little or no inhibition of HTT mRNA expression. These data demonstrate that the DTx-01-08 motif will likely enable the uptake and activity of any siRNA conjugated at the 3′ position with it. These data also provide further evidence that the DTx-01-08 motif is superior to DHA.

### Activity of Compound 2 in Other Cell Types

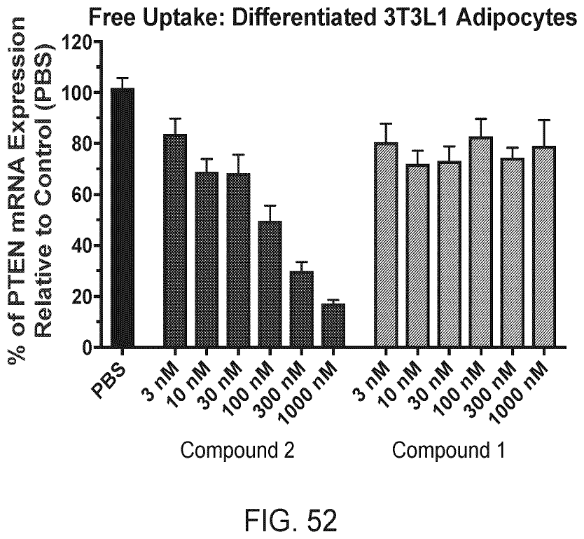

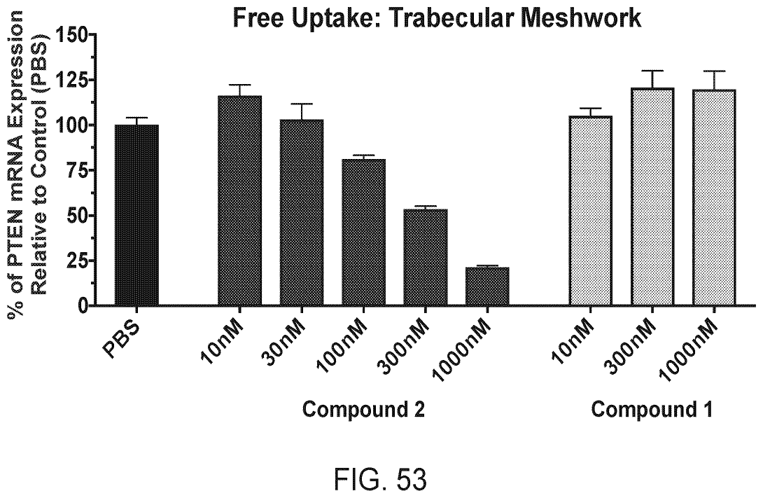

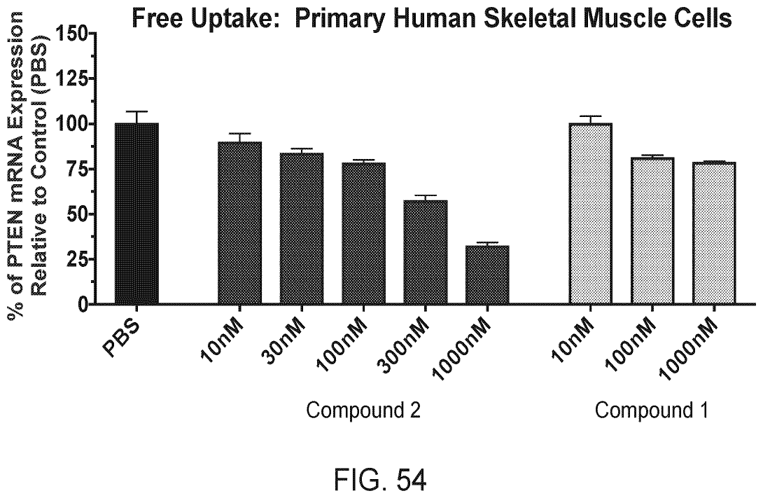

The ability of Compound 2 to inhibit PTEN mRNA expression following incubation on either differentiated 3T3L1 adipocytes, differentiated primary human skeletal muscle cells, and primary human trabecular meshwork cells was evaluated. Both Compound 2 and unconjugated PTEN siRNA (Compound 1) were incubated on differentiated 3T3L1 adipocytes for 48 hours and on primary human trabecular meshwork cells and differentiated primary human skeletal muscle cells for 96 hours. RNA was isolated, and PTEN mRNA quantified by QT-PCR. Compound 2 inhibited PTEN mRNA expression in all 3 cell types whereas Compound 1, the unconjugated PTEN siRNA, had little or no effect (See FIGS. 52-54).

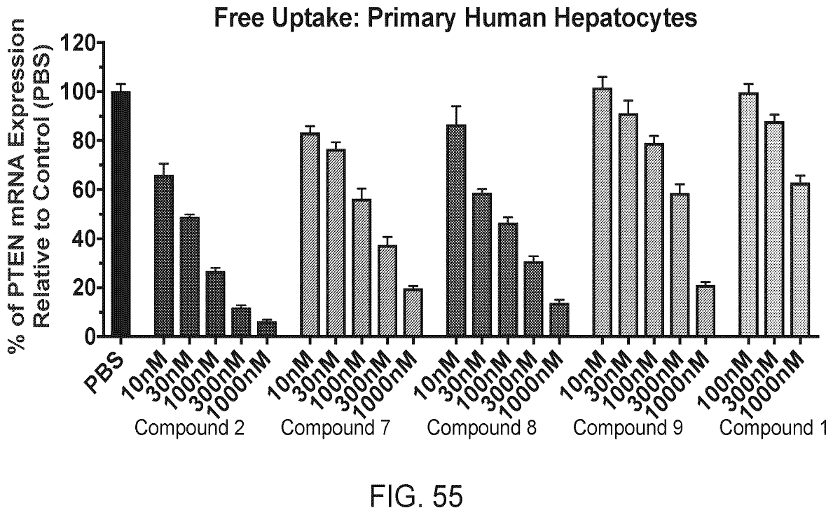

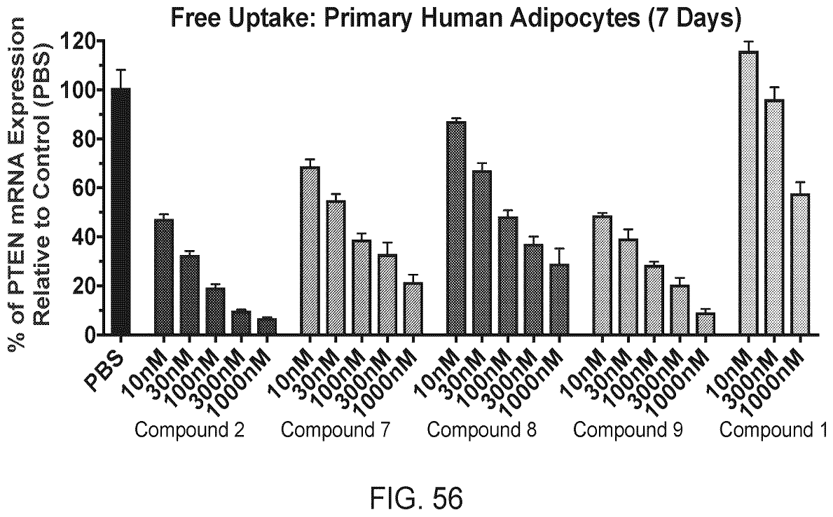

The effect of the number of C16 LCFAs in a conjugate moiety was evaluated. The ability of Compound 2 (two C16 LCFAs), as well as Compound 7 (one C16 LCFA; DTx-01-06 motif), Compound 8 (one C16 LCFA; DTx-01-11 motif), Compound 9 (two C16 LCFA, one at the 5′ terminus of the passenger strand and one at the 3′ terminus of the passenger strand) and Compound 1 (unconjugated), to inhibit PTEN mRNA expression following incubation on primary human hepatocytes and primary human adipocytes were evaluated. All compounds were incubated on hepatocytes for 48 hours and adipocytes for 7 days. RNA was then isolated and PTEN mRNA quantified by QT-PCR. In hepatocytes, all compounds dose dependently inhibited the expression of PTEN mRNA. Compound 2 was significantly more potent than either unconjugated Compound 1 or Compound 7, Compound 8 and Compound 9 (FIG. 55). In adipocytes, again, all compounds dose dependently inhibited the expression of PTEN mRNA. Compound 2 and Compound 9 were more potent and efficacious than Compound 7, Compound 8 or Compound 1 at inhibiting PTEN mRNA expression. Compound 2 appeared to be slightly more potent than Compound 9 at inhibiting PTEN mRNA expression following incubation on adipocytes (FIG. 56).

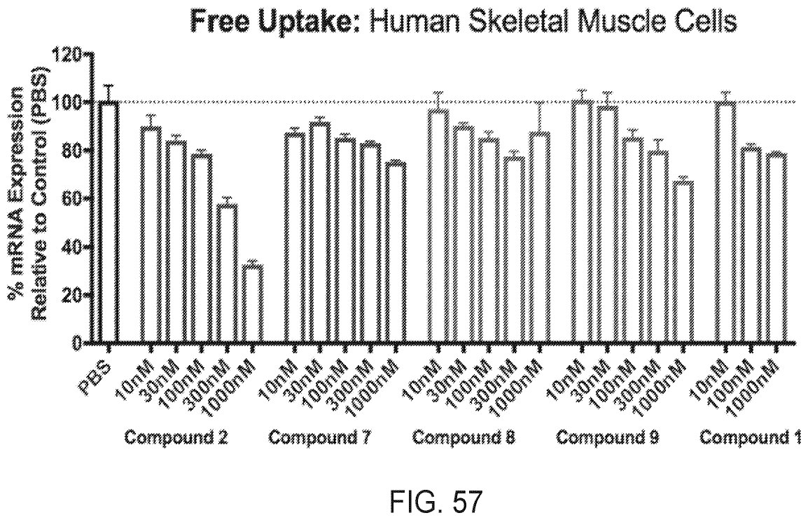

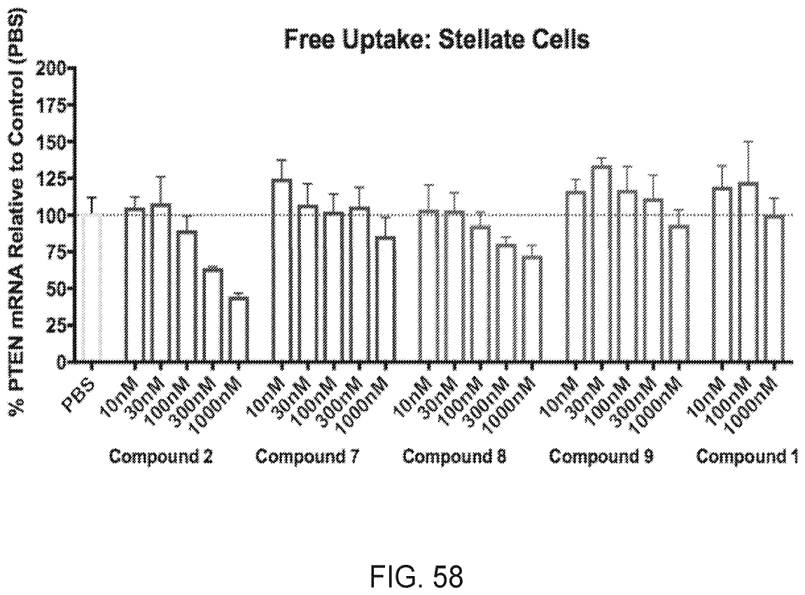

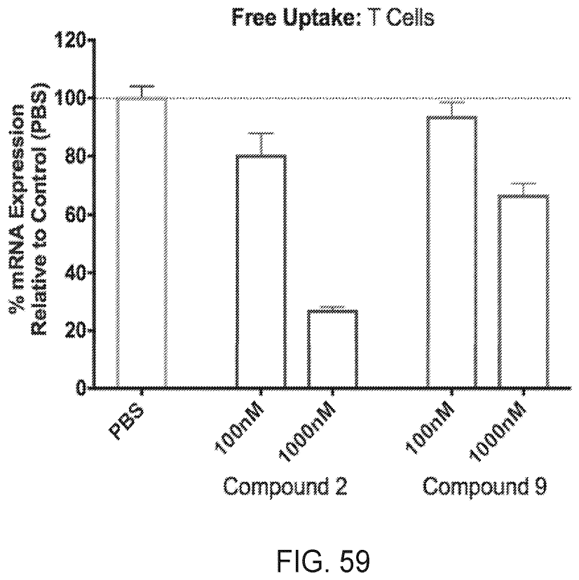

The ability of Compound 2, as well as Compound 7, Compound 8, Compound 9 and Compound 1, to inhibit PTEN mRNA expression following incubation on differentiated primary human skeletal muscle cells and primary human stellate cells was evaluated. All compounds were incubated on differentiated muscle cells for 96 hours and stellate cells for 48 hours. RNA was then isolated and PTEN mRNA quantified by QT-PCR and normalized to a housekeeping gene. In both cell types, Compound 2 was significantly more potent in repressing PTEN mRNA expression than either unconjugated Compound 1 or conjugated compounds Compound 7, Compound 8 and Compound 9 (See FIGS. 57 and 58). Compound 2 and Compound 9 were also incubated on human T cells for 96 hours. Compound 2 was significantly more potent in repressing PTEN mRNA expression than Compound 9 (See FIG. 59).

### Additional Dual-C16 Examples

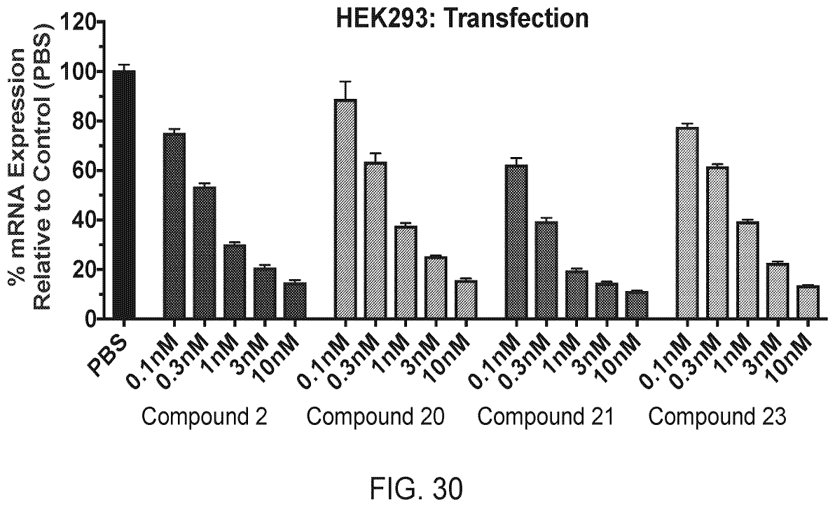

To explore the effect of the relative positioning of two C16 LCFAs in a conjugate moiety, additional molecules, Compounds 20 and 21, were synthesized with a single motif containing two C16 LCFAs conjugated to the 3′ end of the oligonucleotide. In the case of Compound 20, the C16 LCFAs were designed to be closer together than as presented in Compound 2 and in the case of Compound 21, further apart than Compound 2. Transfection of Compound 20, Compound 21 and Compound 2 into HEK293 cells demonstrated that all 3 compounds were active at repressing PTEN mRNA expression (FIG. 30). Free uptake experiments in HUVEC cells, where Compound 2, Compound 20, Compound 21 and Compound 1 (unconjugated PTEN siRNA) were incubated in the media for 48 hours, revealed that Compound 20 and Compound 21 were similarly potent and efficacious at inhibiting PTEN mRNA expression as Compound 2. Compound 1 had little or no effect to inhibit PTEN mRNA expression in HUVEC cells (FIG. 31).

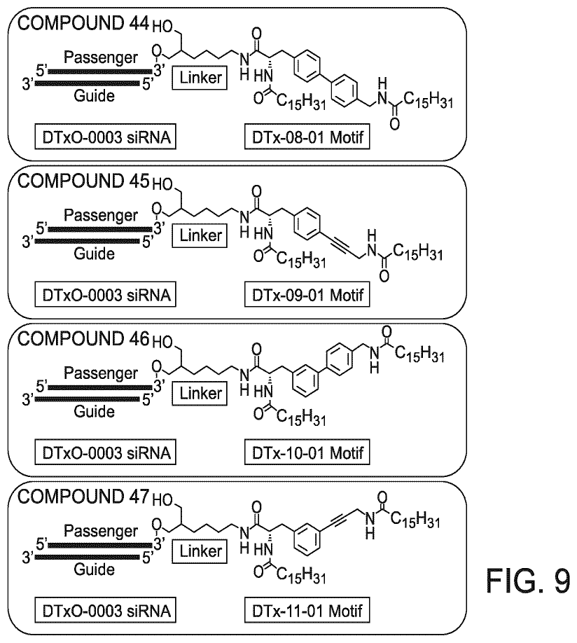

As distance between the attachment sites of two C16 LCFAs in the context of structurally flexible linkers did not seem to markedly affect activity of conjugate moieties, compounds with structurally rigid linkers were synthesized (FIG. 9). Compound 44 was selected for in vitro testing under both transfection and free uptake conditions.

Compounds 2 and 44 were transfected into HEK293 cells. Compound 1, the unconjugated PTEN siRNA, was also transfected into HEK293 cells. PBS-treated cells served as a control. RNA was isolated from the cells 48 hours later, and PTEN mRNA was quantified by QT-PCR and normalized to a housekeeping gene. Following transfection, the PTEN siRNA conjugates, Compounds 1, 2, and 44 were similarly effective in repressing PTEN mRNA expression (FIG. 16).

To evaluate the activity of the same compounds under free uptake conditions, the same compounds were incubated with HUVEC cells in media containing 2% serum. RNA was isolated from the cells 48 hours later, and PTEN mRNA was quantified by QT-PCR and normalized to a housekeeping gene. Following free uptake, Compound 2 exhibited the greatest potency as measured by reduction in PTEN mRNA expression, relative to the rigid lipid containing Compound 44, and the unconjugated Compound 1 (FIG. 17).

These data illustrate that the structural context in which the two C16 LCFAs are presented to cells significantly effects siRNA uptake and activity.

### Conjugation of the DTx-01-08 Motif Enables Activity and Uptake in the Retina

To evaluate the activity and uptake in the retina, Compound 2 was administered to mice or rats via intravitreal injection.

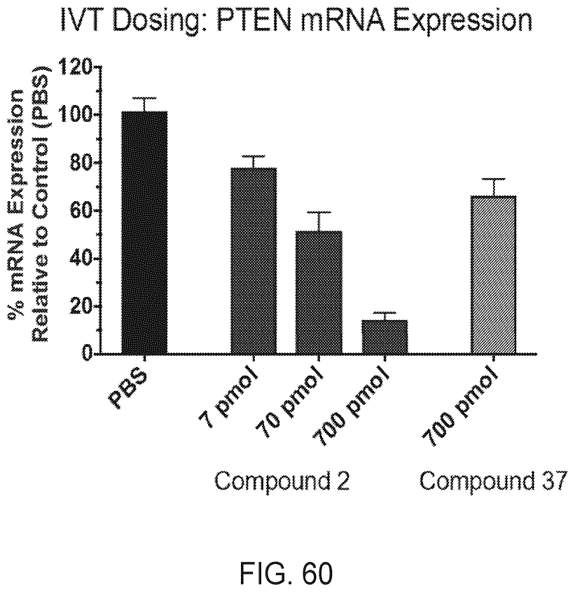

C57BL/6 mice were injected via intravitreal injection with either PBS or 7 μmol, 70 pmol or 700 pmol of Compound 2 (DTx-01-08-conjugated siRNA targeted to PTEN). As a control, a previously published unconjugated, modified single-stranded oligonucleotide targeted to PTEN, Compound 37, was dosed at 700 pmol (Butler et al., Diabetes, 2002, 51(4): 1028-1034). Seven days following injection, the mice were euthanized and the retina isolated. RNA was isolated from the retina and PTEN mRNA expression quantified relative to a housekeeping gene by QT-PCR. Relative to PBS, Compound 2 dose-dependently inhibited PTEN mRNA expression in the retina and was more effective than the unconjugated, modified single-stranded Compound 37 (See FIG. 60).

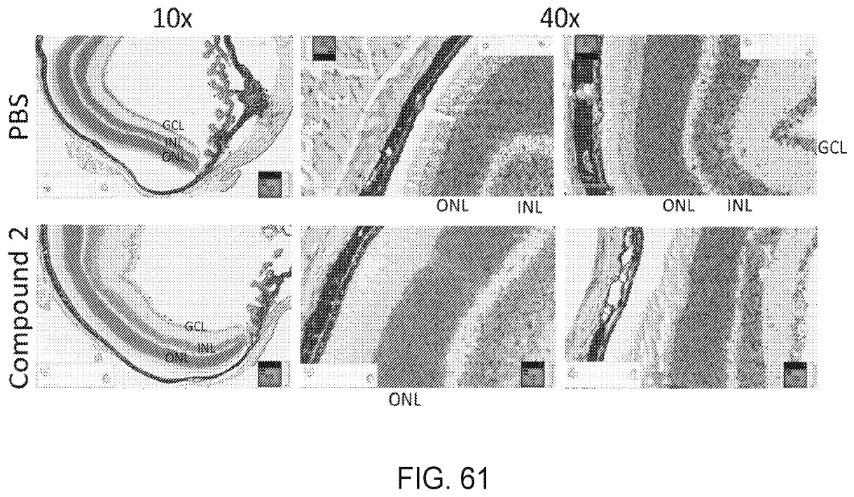

To understand the cell types within the retina in which PTEN expression is inhibited following exposure to Compound 2, Brown Norway rats were injected via intravitreal injection with either PBS or 700 pmol of Compound 2. Seven days post-dose, eyes were collected and quantitative in situ hybridization was performed via RNAscope to understand the cell types in the retina where Compound 2 inhibited PTEN mRNA expression (See FIG. 61). Relative to PBS, Compound 2 inhibited PTEN expression, as evidenced by a substantial reduction in pink dots (PTEN mRNA transcripts), across all of the cell types within the retina including the outer nuclear layer, the inner nuclear layer and the ganglion cell layer (See FIG. 61).

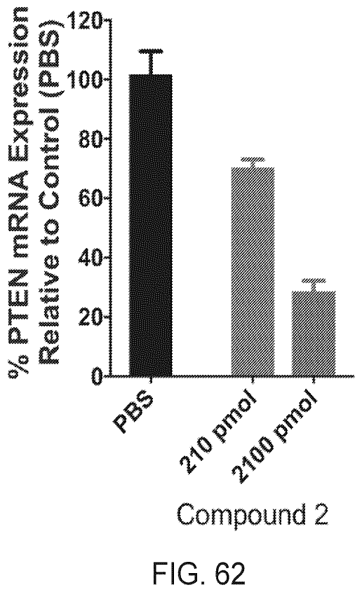

The activity of Compound 2 was also evaluated in rats. Brown Norway rats were injected via intravitreal injection with either PBS or 210 pmol or 2100 pmol of Compound 2. Seven days following injection, the rats were euthanized and the retina isolated. RNA was isolated from the retina and PTEN mRNA expression quantified relative to a housekeeping gene by QT-PCR. Relative to PBS, Compound 2 dose-dependently inhibited PTEN mRNA expression in the retina (See FIG. 62).

Conjugation of the DTx-01-08 Motif Enables the Activity of siRNAs to Distinct Targets Following Intravitreal Injection

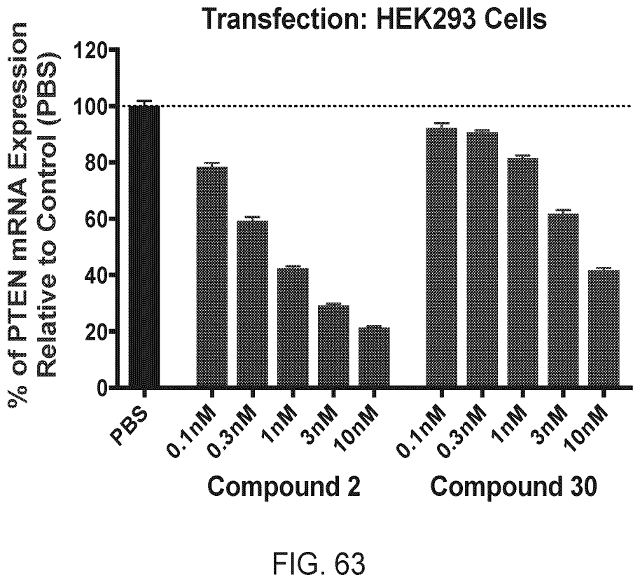

To test the effects of conjugation of the DTx-01-08 motif in the context of different siRNAs, additional siRNAs sequences were synthesized and conjugated to the DTx-01-08 motif. The compounds were Compound 30, a previously published siRNA to PTEN, distinct from the siRNA of Compound 2 (Prakash et al., Bioorganic & Medicinal Chemistry Letters, 2016, 26(9):2194-2197) and Compound 27 (Nikan et al., Molecular Therapy—Nucleic Acids, 2016, 5, e344). To confirm the activity of Compound 30, HEK293 cells were transfected with Compound 2 and Compound 30. Both Compound 2 and Compound 30 inhibited PTEN mRNA expression, with Compound 2 demonstrating greater activity (FIG. 63). Compound 27 inhibited HTT mRNA expression in SH-SY5Y cells (FIG. 49).

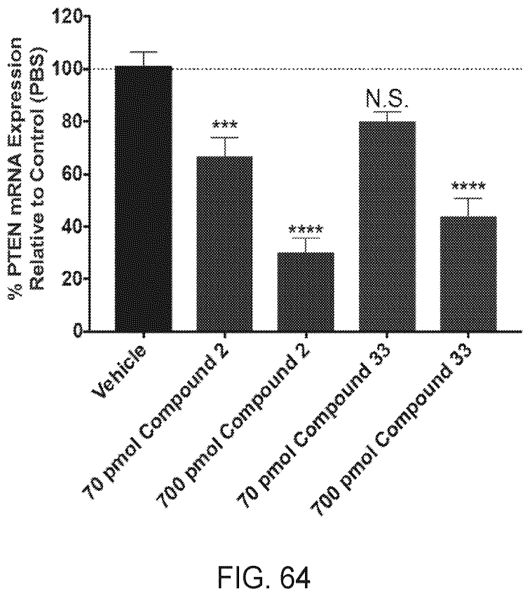

Compound 30 (PTEN) and Compound 27 (HTT) were conjugated to DTx-01-08 to generate Compound 33 (PTEN) and Compound 29 (HTT). C57BL/6 mice were injected via intravitreal injection with either PBS, 70 pmol or 700 pmol of Compound 2, and 70 pmol or 700 pmol of Compound 33. Seven days following injection, the mice were euthanized and the retina isolated. RNA was isolated from the retina, QT-PCR was performed and PTEN mRNA expression quantified relative to a housekeeping gene by QT-PCR. Both compounds dose-dependently inhibited PTEN mRNA expression relative to PBS (FIG. 64).

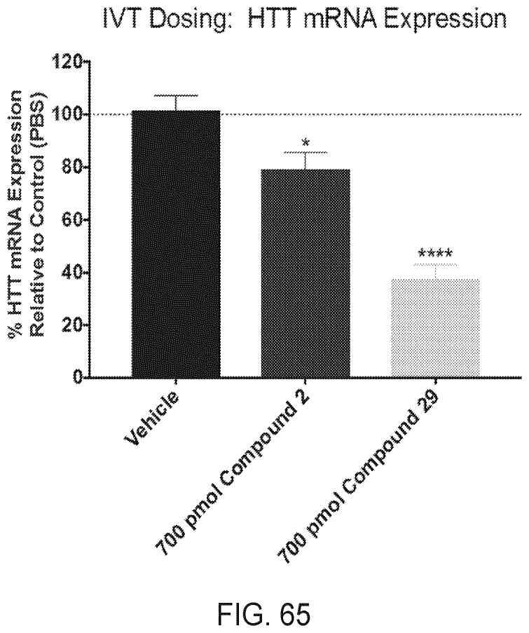

In a similarly designed experiment, C57BL/6 mice were injected via intravitreal injection with either PBS, 700 pmol of Compound 29 or 700 pmol of Compound 2. RNA was isolated from the retina, QT-PCR was performed and HTT mRNA expression quantified relative to a housekeeping gene. Relative to PBS or the PTEN-targeting siRNA conjugate Compound 2, the HTT-targeting siRNA conjugate, Compound 29, significantly inhibited HTT mRNA expression in the retina 7 days following intravitreal injection (FIG. 65).

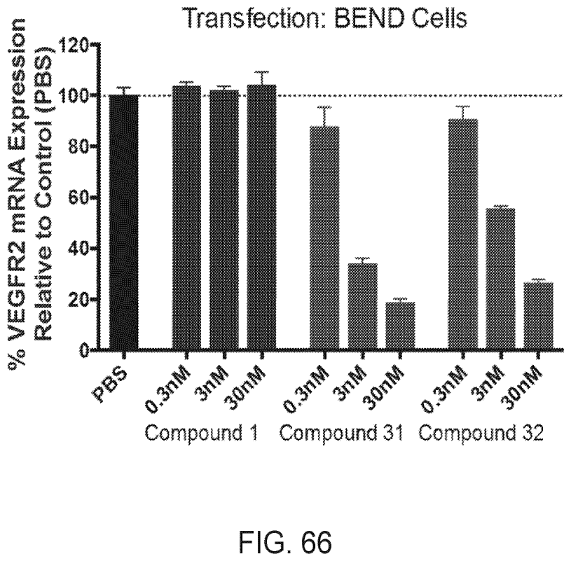

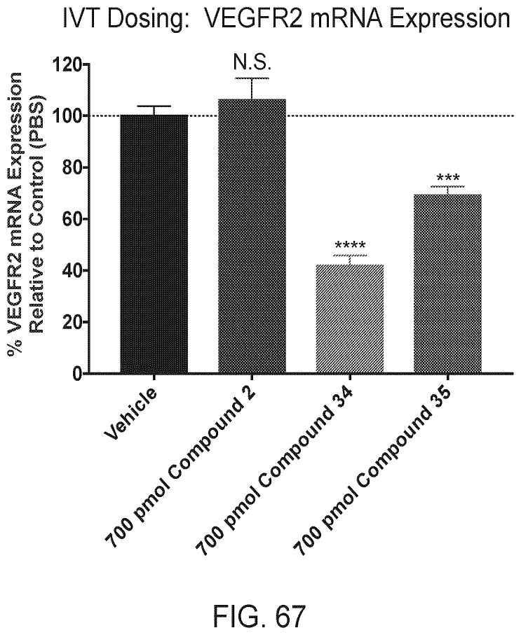

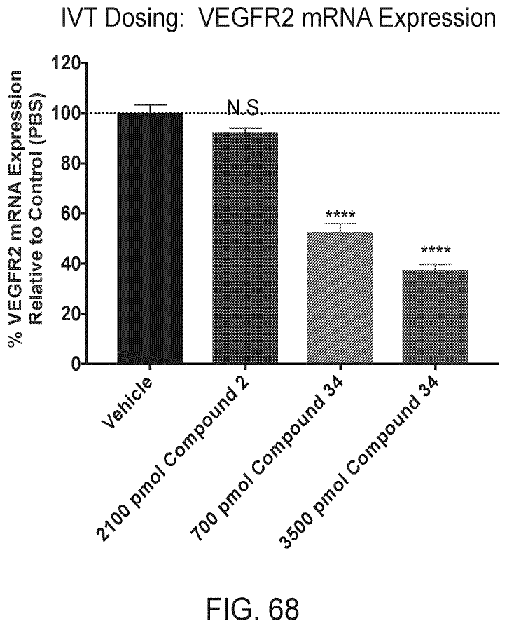

Two different siRNAs targeting the VEGFR2 mRNA were also tested. Unconjugated versions of the siRNAs, Compound 31 and Compound 32, were transfected along with PTEN siRNA Compound 1 into BEND cells. RNA was isolated 48 hours following and VEGFR2 expression evaluated by QT-PCR. Compound 31 and Compound 32 dose-dependently inhibited VEGFR2 expression relative to PBS. As expected, the PTEN-targeting siRNA Compound 1 did not affect VEGFR2 mRNA expression. (FIG. 66). Each of Compounds 31 and 32 were then conjugated to DTx-01-08 to generate Compound 34 and Compound 35, respectively. C57BL/6 mice were then injected via intravitreal injection with either PBS, 700 pmol of Compound 34, 700 pmol of Compound 35, or 700 pmol of Compound 2 (also a conjugated siRNA targeting PTEN). Seven days following injection, the mice were euthanized and the retina isolated. RNA was isolated from the retina and VEGFR2 mRNA expression quantified relative to a housekeeping gene. Relative to PBS and the PTEN-targeting siRNA conjugate Compound 2, Compound 34 and Compound 35 significantly inhibited VEGFR2 mRNA expression (FIG. 67). Compound 34 was also evaluated in rats. PBS, 700 or 3500 pmol of Compound 34 and 2100 pmol of Compound 2 were intravitreally injected into rat eyes. Seven days following injection, the rats were euthanized and the retina isolated. RNA was isolated from the retina and VEGFR2 mRNA expression quantified relative to a housekeeping gene by QT-PCR. Relative to PBS and the PTEN-targeting siRNA conjugate Compound 2, Compound 34 significantly inhibited VEGFR2 mRNA expression (FIG. 68).

### Dual-C16 Motifs Are Active In Vivo

Also tested were compounds designed with a single motif containing two C16 LCFAs conjugated to the 3′ end of the passenger strand of an siRNA targeting PTEN. In the case of Compound 20, the C16s were designed to be closer together than in Compound 2 and in the case of Compound 21, further apart than Compound 2 (FIG. 4).

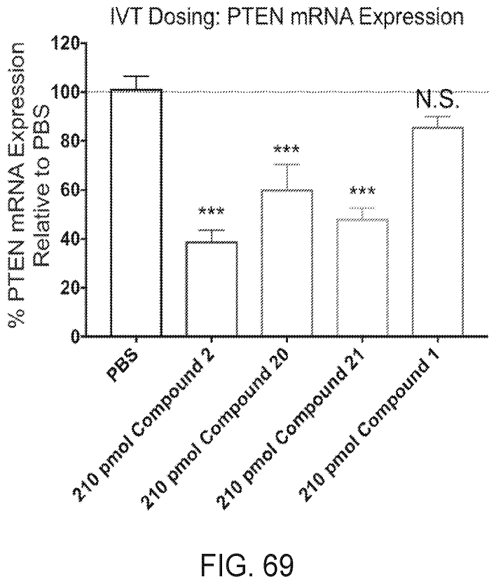

Compound 20, Compound 21, Compound 2 and Compound 1 were each injected into C57BL/6 mice eyes at a dose of 210 pmol via intravitreal injection. PBS was injected as a control. Seven days following injection, the mice were euthanized and the retina isolated. RNA was isolated from the retina and PTEN mRNA expression quantified relative to a housekeeping gene by QT-PCR. Relative to PBS and Compound 1 (unconjugated PTEN siRNA) which did not significantly inhibit the expression of PTEN mRNA in this experiment, each of the PTEN siRNA conjugates, Compound 20, Compound 21, and Compound 2 significantly inhibited PTEN mRNA expression (FIG. 69).

### The Effect of LCFA Lengths In Vivo

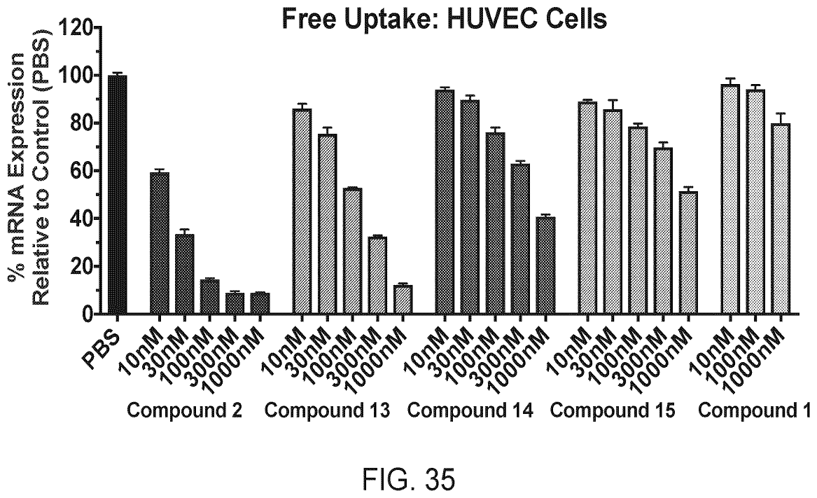

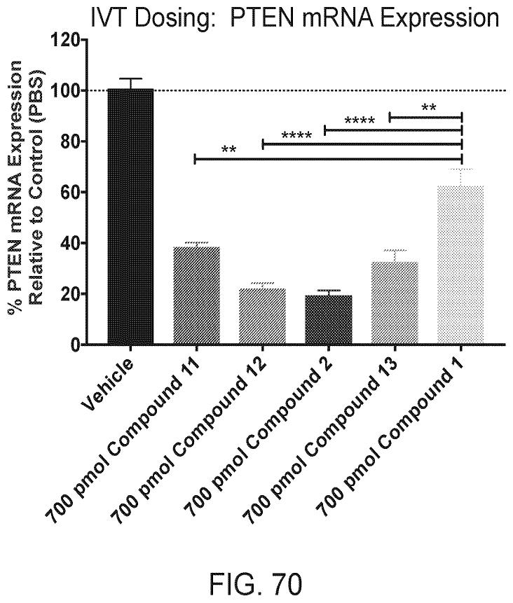

A series of compounds was designed to evaluate whether the conjugation of multiple saturated LCFAs of distinct lengths might promote uptake and activity more potently than the two saturated C16 LCFAs conjugated to the PTEN siRNA in Compound 2. A non-cleavable C7/lysine linker was utilized to covalently link saturated LCFAs ranging in length from 12 carbons to 18 carbons to the PTEN siRNA. Two each of C12, C14 and C18 saturated LCFA were attached to the amino groups on the lysine to generate Compound 11, Compound 12 and Compound 13, respectively (See FIG. 3). As demonstrated herein, transfection experiments confirmed that Compounds 11-13 inhibited PTEN mRNA expression to similar extents in HEK293 cells (FIG. 34 and FIG. 35). C57Bl/6 mice were injected via intravitreal injection with either water or 700 pmol of Compound 2, Compound 11, Compound 12, Compound 13 or Compound 1. Compound 13 was not soluble in PBS and was thus solubilized in water. In order to compare the data for each compound, in this experiment, each compound was solubilized in water. Seven days following injection, the mice were euthanized and the retina isolated. RNA was isolated from the retina, QT-PCR was performed and PTEN mRNA expression quantified relative to a housekeeping gene. Compound 2, Compound 11, Compound 12, and Compound 13 all inhibited PTEN mRNA expression more effectively than PBS or Compound 1 (unconjugated PTEN siRNA) (FIG. 70). As in free uptake experiments in vitro and ex vivo (FIGS. 36&37), Compound 2 and Compound 12 appeared to be modestly more effective at repressing PTEN mRNA expression than Compound 11 and Compound 13 (FIG. 70).

It was observed that in this experiment, Compound 1 was slightly more active than in other experiments (see, for example, FIG. 69). While solubilization in water may enhance uptake and/or have adverse effects in vivo, in this experiment the relative levels of PTEN mRNA expression across compounds are consistent with previous experiments and thus the fact that the compounds were solubilized in water is not believed to have had a significant effect on the relative results. Importantly, the correlation between in vitro and in vivo activity was observed.

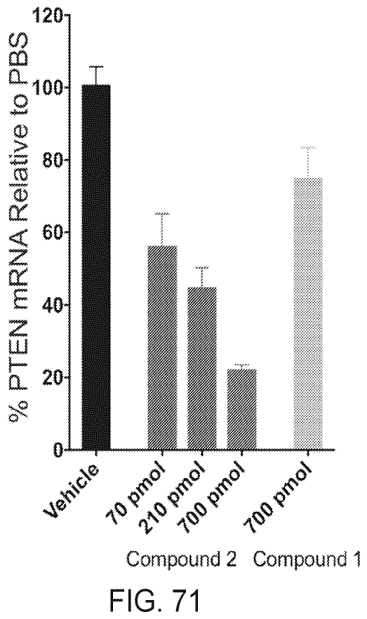

To confirm the advantage of conjugated siRNA over unconjugated siRNA and that the observed inhibition of PTEN mRNA expression was not related to solubilization of compounds in water, an additional intravitreal injection experiment was performed in mice. C57Bl/6 mice were injected via intravitreal injection with either PBS, Compound 1 dissolved in PBS or Compound 2 dissolved in PBS. Compound 1 was tested at a dose of 700 μmol, and Compound 2 was tested at doses of 70 μmol, 210 μmol, and 700 pmol. Seven days following injection, the mice were euthanized and the retina isolated. RNA was isolated from the retina, QT-PCR was performed and PTEN mRNA expression quantified relative to a housekeeping gene. Compound 2 inhibited PTEN mRNA expression in a dose-dependent manner, and more effectively than PBS or Compound 1 (FIG. 71).

Conjugation of the DTx-01-08 Motif Enables the Activity of siRNAs to Distinct Targets Following Systemic Administration

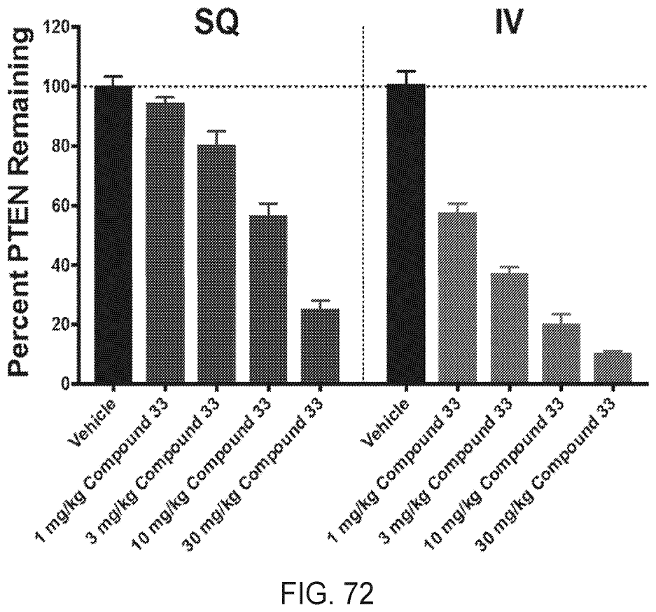

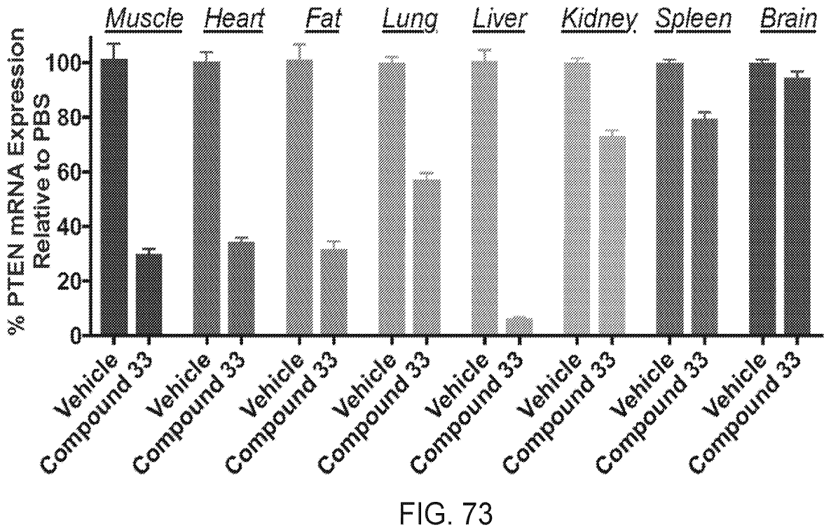

Mice were subcutaneously or intravenously injected with a single dose of either PBS or 1, 3, 10 or 30 mg/kg of the PTEN-targeting siRNA conjugated to the DTx-01-08 motif, Compound 33. Liver was collected 7 days following injection, RNA isolated and reverse transcribed. QT-PCR was then performed to quantify PTEN mRNA expression relative to a housekeeping gene. Compound 33 dose-dependently repressed PTEN mRNA expression in the liver relative to PBS following both subcutaneous and intravenous administration (FIG. 72). A follow up study was performed to understand whether Compound 33 was able to repress PTEN mRNA expression in tissues outside of the liver. C57Bl/6 mice were injected intravenously every other day for 3 doses with either PBS or 30 mg/kg of Compound 33. Seven days following the last dose, tissues were collected, RNA isolated and reverse transcribed. QT-PCR was then performed to evaluate PTEN mRNA expression relative to a housekeeping gene. Compound 33 inhibited PTEN mRNA expression in muscle, heart, fat, lung, liver, kidney and spleen (FIG. 73).

In summary, the results of the transfection and free uptake experiments demonstrate that conjugating siRNA at the 3′ position with two LCFAs between 12 and 18 carbons in length significantly promotes siRNA uptake and activity. The experiments show that this increased ability to enter a cell does not dependent on either cell type or the specific siRNA. Surprisingly, when incubated on neuronal cells, siRNA conjugated with the C16 DTx-01-08 motif enabled significantly greater uptake and activity than siRNA conjugated with one or more DHA, a reported experimental approach for targeting neurons of the CNS.

The increased siRNA uptake and activity was observed for siRNAs targeted to different mRNAs, siRNAs having different nucleoside sugar modification motifs, demonstrating that the improved uptake and activity are independent of the nucleotide sequence and chemical modifications of the siRNA to which the lipid moiety is conjugated. Importantly, the DTx-01-08 motif and other lipid motifs improved siRNA uptake in vivo following either local or systemic administration.

Although the disclosure has been described with reference to embodiments and examples, it should be understood that numerous and various modifications can be made without departing from the spirit of the present disclosure.

What is claimed:

1. A compound, or pharmaceutically acceptable salt thereof, having the structure: 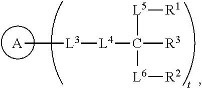
wherein
A is a double-stranded oligonucleotide, or single-stranded oligonucleotide;
L³ is independently a bond, —NH—, bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁴ is independently -L⁷-NH—C(O)— or -L⁷-C(O)—NH—, wherein L⁷ is independently substituted or unsubstituted alkylene;
L⁵ is -L^{5A}-L^{5B}-L^{5C}-L^{5D}-L^{5E}- wherein:
L^{5A} is independently a bond or unsubstituted alkylene;
L^{5B} is independently a bond, —NHC(O)—, —NH—, —O—, —S—, —C(O)—, —NHC(O)NH—, —C(O)O—, —OC(O)—, —C(O)NH—, or unsubstituted phenylene;
L^{5C} is independently a bond, unsubstituted alkynylene, or unsubstituted phenylene;
L^{5D} is independently a bond or unsubstituted alkylene;
L^{5E} is independently a bond, —NHC(O)—, —NH—, —O—, —S—, —C(O)—, —NHC(O)NH—, —C(O)O—, —OC(O)—, or —C(O)NH—;
L⁶ is -L^{6A}-L^{6B}-L^{6C}-L^{6D}-L^{6E}- wherein:
L^{6A} is independently a bond or unsubstituted alkylene;
L^{6B} is independently a bond, —NHC(O)—, —NH—, —O—, —S—, —C(O)—, —NHC(O)NH—, —C(O)O—, —OC(O)—, —C(O)NH—, or unsubstituted phenylene;
L^{6C} is independently a bond, unsubstituted alkynylene, or unsubstituted phenylene;
L^{6D} is independently a bond or unsubstituted alkylene; and
L^{6E} is independently a bond, —NHC(O)—, —NH—, —O—, —S—, —C(O)—, —NHC(O)NH—, —C(O)O—, —OC(O)—, or —C(O)NH—;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
and
t is an integer from 1 to 5.

2. The compound of claim 1, or pharmaceutically acceptable salt thereof, wherein t is 1, 2 or 3.

3. The compound of claim 1, or pharmaceutically acceptable salt thereof, wherein the double-stranded oligonucleotide, or single-stranded oligonucleotide is an siRNA, a microRNA mimic, a stem-loop structure, a single-stranded siRNA, an RNaseH oligonucleotide, an anti-microRNA oligonucleotide, a steric blocking oligonucleotide, a CRISPR guide RNA, or an aptamer.

4. The compound of claim 1, or pharmaceutically acceptable salt thereof, wherein the double-stranded oligonucleotide, or single-stranded oligonucleotide of A comprises a locked nucleic acid (LNA) residue, bicyclic nucleic acid (BNA) residue, constrained ethyl (cEt) residue, unlocked nucleic acid (UNA) residue, phosphorodiamidate morpholino oligomer (PMO) monomer, peptide nucleic acid (PNA) monomer, 2′-O-methyl (2′-OMe) residue, 2′-O-methyoxyethyl residue, 2′-deoxy-2′-fluoro residue, 2′-O-methoxy ethyl/phosphorothioate residue, phosphoramidate, phosphorodiamidate, phosphorothioate, phosphorodithioate, phosphonocarboxylic acid, phosphonocarboxylate, phosphonoacetic acid, phosphonoformic acid, methyl phosphonate, boron phosphonate, or O-methylphosphoroamidite.

5. The compound of claim 1, or a pharmaceutically acceptable salt thereof, wherein A is a double-stranded oligonucleotide or single-stranded oligonucleotide, and
i. one L³ is attached to a 3′ carbon of the double-stranded oligonucleotide or single-stranded oligonucleotide;
ii. one L³ is attached to a 5′ carbon of the double-stranded oligonucleotide or single-stranded oligonucleotide;
iii. L³ is attached to a 2′ carbon of a nucleotide of the double-stranded oligonucleotide or the single-stranded oligonucleotide;
and/or
iv. one L³ is attached to a nucleobase of the double-stranded oligonucleotide or single-stranded oligonucleotide.

6. The compound of claim 1, or pharmaceutically acceptable salt thereof, wherein the double-stranded oligonucleotide, or single-stranded oligonucleotide of A comprises a 5′-(E)-vinylphosphonate group at a terminus.

7. The compound of claim 1, or a pharmaceutically acceptable salt thereof, wherein A is a single-stranded oligonucleotide comprising a 5′-(E)-vinylphosphonate group at the 5′ end of the oligonucleotide.

8. The compound of claim 1, or a pharmaceutically acceptable salt thereof, wherein A is a double-stranded oligonucleotide comprising the 5′-(E)-vinylphosphonate group at the 5′ end of the guide strand.

9. The compound of claim 4, or a pharmaceutically acceptable salt thereof, wherein the double-stranded oligonucleotide is an siRNA comprising a 5′-(E)-vinylphosphonate group at the 5′ end of the guide strand.

10. The compound of claim 8, or a pharmaceutically acceptable salt thereof, wherein one L₃ is attached to
i. a 3′ carbon of the guide strand
ii. a 3′ carbon of the passenger strand
or
iii. a 5′ carbon of the passenger strand.

11. The compound of claim 1, or pharmaceutically acceptable salt thereof, wherein L³ is independently —OPO₂—O—.

12. The compound of claim 1, or pharmaceutically acceptable salt thereof, wherein L³ is independently —O—.

13. The compound of claim 1, or pharmaceutically acceptable salt thereof, wherein L⁴ is independently -L⁷-NH—C(O)—, wherein L⁷ is independently hydroxymethyl-substituted C₁-C₁₂ alkylene or unsubstituted C₁-C₁₂ alkylene.

14. The compound of claim 5, or pharmaceutically acceptable salt thereof, wherein L⁴ is independently

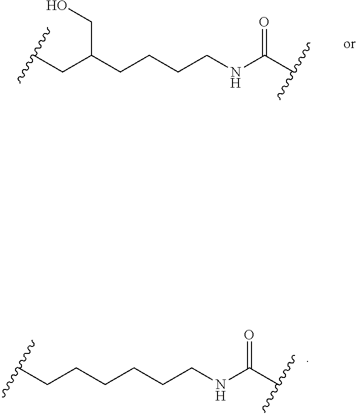 [SMILES: *CC(CO)CCCCNC(*)=O ; *CCCCCCNC(*)=O]

15. The compound of claim 1, or pharmaceutically acceptable salt thereof, wherein -L³-L⁴- is independently —O-L⁷-NH—C(O)— or —O-L⁷-C(O)—NH—, wherein L⁷ is independently substituted or unsubstituted alkylene.

16. The compound of claim 1, or pharmaceutically acceptable salt thereof, wherein -L³-L⁴- is independently —O-L⁷-NH—C(O)—, wherein L⁷ is independently hydroxymethyl-substituted C₅-C₈ alkylene or unsubstituted C₅-C₈ alkylene.

17. The compound of claim 8, or pharmaceutically acceptable salt thereof, wherein -L³-L⁴- is

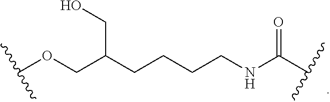 [SMILES: *OCC(CO)CCCCNC(*)=O]

18. The compound of claim 1, or pharmaceutically acceptable salt thereof, wherein -L³-L⁴- is independently —OPO₂—O-L⁷-NH—C(O)— or —OPO₂—O-L⁷-C(O)—NH—, wherein L⁷ is independently substituted or unsubstituted alkylene.

19. The compound of claim 1, or pharmaceutically acceptable salt thereof, wherein -L³-L⁴- is independently —OPO₂—O-L⁷-NH—C(O)—, wherein L⁷ is independently hydroxymethyl-substituted C₅-C₈ alkylene or unsubstituted C₅-C₈ alkylene.

20. The compound of claim 11, or pharmaceutically acceptable salt thereof, wherein -L³-L⁴- is

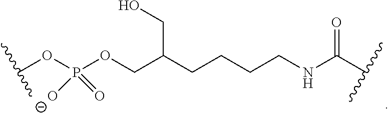 [SMILES: *OP(=O)([O-])OCC(CO)CCCCNC(*)=O]

21. The compound of claim 12, or pharmaceutically acceptable salt thereof, wherein an -L₃-L⁴- is independently 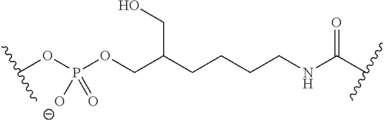 [SMILES: *OP(=O)([O-])OCC(CO)CCCCNC(*)=O]
and L³ is attached to a 3′ carbon of the double-stranded oligonucleotide or single-stranded oligonucleotide.

22. The compound of claim 12, or pharmaceutically acceptable salt thereof, wherein an -L³-L⁴- is independently, 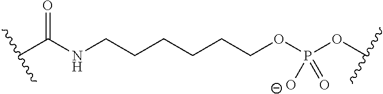 [SMILES: *OP(=O)([O-])OCCCCCCNC(*)=O]
and L³ is attached to a 5′ carbon of the double-stranded oligonucleotide or single-stranded oligonucleotide.

23. The compound of claim 1, or pharmaceutically acceptable salt thereof, wherein R³ is independently hydrogen.

24. The compound of claim 1, or pharmaceutically acceptable salt thereof, wherein L⁶ is -L^{6A}-L^{6B}-L^{6C}-L^{6D}-L^{6E}- and wherein L^{6A} is a bond, L^{6B} is a bond, L^{6C} is a bond, L^{6D} is a bond, and L^{6E} is a bond is —NHC(O)—.

25. The compound of claim 1, or pharmaceutically acceptable salt thereof, wherein L⁶ is independently a bond,

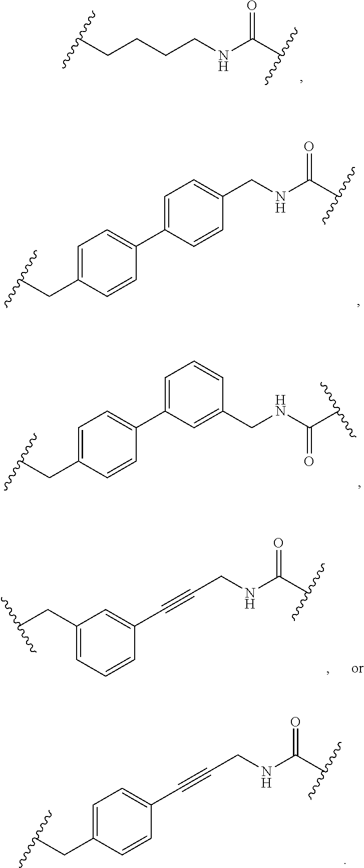 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

26. The compound of claim 1, or a pharmaceutically acceptable salt thereof, wherein L⁵ is -L^{5A}-L^{5B}-L^{5C}-L^{5D}-L^{5E}- and wherein L^{5A} is a bond, L^{5B} is a bond, L^{5C} is a bond, L^{5D} is a bond, and L^{5E} is a bond is —NHC(O)—.

27. The compound of claim 1, or a pharmaceutically acceptable salt thereof, wherein L⁵ is independently a bond,

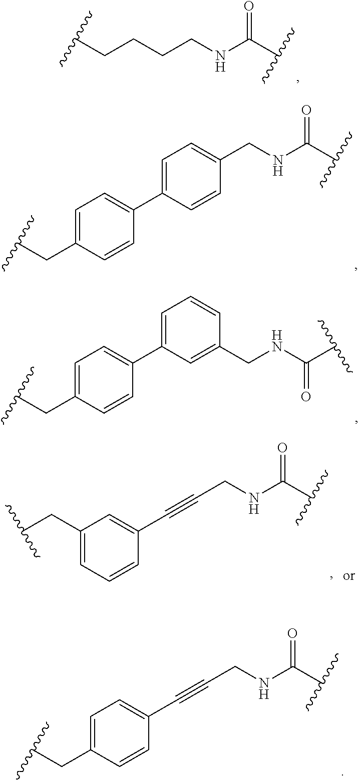 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

28. The compound of claim 1, or a pharmaceutically acceptable salt thereof, wherein R¹ is unsubstituted C₁-C₁₇ alkyl.

29. The compound of claim 1, or a pharmaceutically acceptable salt thereof, wherein R¹ is unsubstituted C₁₁-C₁₇ alkyl.

30. The compound of claim 1, or a pharmaceutically acceptable salt thereof, wherein R¹ is unsubstituted C₁₅ alkyl.

31. The compound of claim 1, or a pharmaceutically acceptable salt thereof, wherein R² is unsubstituted C₁-C₁₇ alkyl.

32. The compound of claim 1, or a pharmaceutically acceptable salt thereof, wherein R² is unsubstituted C₁₁-C₁₇ alkyl.

33. The compound of claim 1, or a pharmaceutically acceptable salt thereof, wherein R² is unsubstituted C₁₅ alkyl.

34. The compound of claim 1, or a pharmaceutically acceptable salt thereof, wherein the compound is a lipid-conjugated compound having the structure of Formula I: 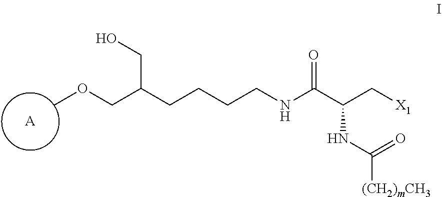
or a pharmaceutically acceptable salt thereof, wherein:
A is a modified double-stranded oligonucleotide or modified single-stranded oligonucleotide, wherein the modified double-stranded oligonucleotide or modified single-stranded oligonucleotide is conjugated to a lipid-containing moiety at the 3′ end of one strand of the modified double-stranded oligonucleotide or the 3′ end of the modified single-stranded nucleic acid;
X₁ is 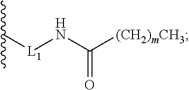
L₁ is —(CH₂)ₙ—, —(CH₂)ₙL₂(CH₂)ₙ—, or a bond;
L₂ is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur),
and wherein each m is independently an integer from 10 to 18 and wherein each n is independently an integer from 1 to 6.

35. The compound of claim 34, or a pharmaceutically acceptable salt thereof, wherein each m is independently an integer from 12 to 16; and wherein each n is independently an integer from 1 to 6.

36. The compound of claim 34, or a pharmaceutically acceptable salt thereof, wherein each m is 14, L₁ is —(CH₂)ₙ—, and n is 3.

37. The compound of claim 34, or a pharmaceutically acceptable salt thereof, wherein the modified double-stranded oligonucleotide or modified single-stranded oligonucleotide contains
i. at least one phosphorothioate linkage
ii. at least one 2′-O-methyl residue
or
iii. at least one 2′-deoxy-2′-fluoro residue.

38. A method of introducing an oligonucleotide into a cell in vitro, comprising contacting the cell with the compound of claim 1, or a pharmaceutically acceptable salt thereof, under free uptake conditions.

39. The method of claim 38, wherein the method is ex vivo and the cell is a primary cell or an immortalized cell.

40. The method of claim 39, wherein the cell is an adipocyte cell, a hepatocyte cell, a fibroblast cell, an endothelial cell, a kidney cell, a human umbilical vein endothelial cell (HUVEC), an adipose cell, a macrophage cell, a neuronal cell, a rat neuron, a muscle cell, or a differentiated primary human skeletal muscle cell.

41. The method of claim 38, wherein the cell is a NIH3T3 cell, a differentiated 3T3L1 cell, a RAW264.7 cell, or a SH-SY5Y cell.

42. A method of treating a subject in need thereof, the method comprising administering to the subject a compound of claim 1, or a pharmaceutically acceptable salt thereof, wherein the subject has a disease or disorder of the eye, liver, kidney, heart, adipose tissue, lung, muscle or spleen.

43. A method of introducing an oligonucleotide into a cell within a subject, the method comprising administering to the subject a compound of claim 1, or a pharmaceutically acceptable salt thereof.

44. A pharmaceutical composition comprising a pharmaceutically acceptable excipient and a compound of claim 1, or a pharmaceutically acceptable salt thereof.
